(19) United States
(12) Patent Application Publication
(10) Pub. No.: US 2024/0382677 A1
(43) Pub. Date: Nov. 21, 2024

(54) AUTO-INJECTOR AND RELATED METHODS OF USE

(71) Applicant: Regeneron Pharmaceuticals, Inc., Tarrytown, NY (US)

(72) Inventors: Bryan C. GRYGUS, Clifton Park, NY (US); Trevor Langley, Rensselaer, NY (US); Daniel Halbig, Ballston Lake, NY (US); Ross Kenyon, Saratoga Springs, NY (US); Alfred Marino, Housatonic, MA (US); Wayne Phillips, Hudson, WI (US); Jeff Moore, Woodbury, MN (US); Sten Nygaard, Struer (DK); Jan Norup, Struer (DK); David Farrage, Hudson, WI (US); Jeremy Odegard, Hudson, WI (US); Jeremy McNamara, Albany, NY (US)

(73) Assignee: Regeneron Pharmaceuticals, Inc., Tarrytown, NY (US)

(21) Appl. No.: 18/666,371

(22) Filed: May 16, 2024

Related U.S. Application Data
(60) Provisional application No. 63/502,883, filed on May 17, 2023.

(51) Int. Cl. A61M 5/172; A61M 5/20; A61M 5/32

(52) U.S. Cl. CPC A61M 5/172; A61M 5/20; A61M 5/3243; A61M 2205/3303; A61M 2205/3553

(57) ABSTRACT

An injection device may include a housing, a container disposed within the housing, the container enclosing a fluid and having a first end and a second end, a conduit movable relative to the container, wherein the conduit is not in fluid communication with the fluid enclosed by the container while in a first position, and is in fluid communication with the fluid enclosed by the container and configured to deliver the fluid from the container to a patient while in a second position, and a lock that is removable from the housing, the lock having a first portion and a second portion.

### CROSS-REFERENCE TO RELATED APPLICATION(S)

This application claims priority to U.S. Provisional Patent Application No. 63/502,883, filed on May 17, 2023, which is hereby incorporated by reference in its entirety.

### SEQUENCE LISTING

This application contains a sequence listing, submitted electronically in XML format under the filename “Sequence Listing.xml”, which is incorporated by reference herein in its entirety. The XML copy of the sequence listing was created on Jun. 11, 2024, and is 448,000 bytes in size.

### TECHNICAL FIELD

This disclosure is directed to an auto-injector and related methods of use.

### INTRODUCTION

In various available auto-injectors, upon activation by a user, a needle is deployed, and fluid is delivered from the needle into the user. After completion of fluid delivery, the needle may be retracted for user comfort, needle safety, and positive perception of the product. However, many auto-injectors may be inadvertently triggered when dropped or vibrated. Additionally, many auto-injectors may lack suitable control logic for stopping an injection when appropriate.

### SUMMARY OF THE DISCLOSURE

In one aspect, the present disclosure is directed to an injection device. The injection device may include: a housing; a container disposed within the housing, the container enclosing a fluid and having a first end and a second end; a conduit movable relative to the container, wherein the conduit is not in fluid communication with the fluid enclosed by the container while in a first position, and is in fluid communication with the fluid enclosed by the container and configured to deliver the fluid from the container to a patient while in a second position; and a lock that is removable from the housing, the lock having a first portion and a second portion. In a first configuration where the lock is coupled to the housing, the first portion of the lock may be disposed exterior of the housing and the second portion of the lock may be disposed within the housing between the container and the conduit; in the first configuration, the conduit may be prevented from moving into fluid communication with the fluid enclosed by the container by the second portion of the lock; and in a second configuration where the lock is removed from the injection device, the conduit may be able to move into fluid communication with the fluid enclosed by the container.

In another aspect, the injection device may include: a housing; a plunger coupled to the housing and movable relative to the housing; one or more electronics components used during an injection performed by the injection device, the one or more electronics components being formed within an electrical circuit. In a first configuration, a first portion of the plunger may be disposed within the housing, the electrical circuit may be open, and the one or more electronics components may be in a low-power sleep mode; in a second configuration, the plunger may move outward relative to the housing, and the first portion of the plunger may extend exterior of the housing; and in the second configuration, the electrical circuit may be closed, and the one or more electronics components may be transitioned from the low-power sleep mode, to an active mode.

In another aspect, the injection device may include: a housing, wherein the housing includes a curved bottom surface that is concave when viewed from a point external to the housing that is closer to the bottom surface of the housing than a top surface of the housing; a circuit board positioned adjacent to the bottom surface of the housing, wherein the circuit board includes a skin sensor configured to sense a presence of skin in contact with the bottom surface of the housing; and a controller coupled to the circuit board, wherein the controller is configured to initiate an injection by the injection device only after the skin sensor senses the presence of skin in contact with the bottom surface of the housing.

In another aspect, the present disclosure is directed to a method of manufacturing an injection device. The method may include: depositing a first material onto a mold, the first material having a first opacity; depositing a second material around the mold and the first material, the second material having a second opacity that is higher than the first opacity; and positioning a container enclosing a medicament within the injection device and adjacent to a first portion of the injection device formed by the first material.

In another aspect, the injection device may include: a container disposed within the housing, the container having a first end and a second end; a piston configured to move from the first end of the container toward the second end of the container to dispense a medicament from the container; a drive member configured to drive the piston through the container; an emitter configured to emit a beam of light toward the container; a detector positioned on an opposing side of the container from the emitter, wherein the detector is configured to receive the beam of light emitted from the emitter; and a controller coupled to the drive member, the emitter, and the detector. The controller may be configured to: receive a first signal from the detector while the emitter is off, the first signal corresponding to an ambient level of light surrounding the injection device; receive a second signal from the detector while the emitter is on; calculate a difference between light values represented by the first signal and the second signal; and cease operation of the drive member when the difference is less than a threshold value.

In another aspect, the injection device may include: a container disposed within the housing, the container having a first end and a second end; a piston configured to move from the first end of the container toward the second end of the container to dispense a medicament from the container; a drive member configured to drive the piston through the container; an emitter configured to emit a beam of light toward the container; a detector positioned on an opposing side of the container from the emitter, wherein the detector is configured to receive the beam of light emitted from the emitter; and a controller coupled to the drive member, the emitter, and the detector. The controller may be configured to: initiate the drive member and the emitter; receive a first signal from the detector while the emitter is on, the first signal being representative of an amount of light received by the detector; allow for continued operation of the drive member for a first period of time immediately after initiation of the drive member; and cease operation of the drive member upon determining (1) that the amount of light received by the detector is less than a first threshold light value and (2) before the amount of light received by the detector subsequently rises to or above the first threshold light value, that a current of the drive member is greater than a first threshold current value.

In another aspect, the injection device may include: a container disposed within the housing, the having a first end and a second end; a piston configured to move from the first end of the container toward the second end of the container to dispense a medicament from the container; a drive member configured to drive the piston through the container; and a controller coupled to the drive member. The controller may be configured to: maintain a speed of the drive member until a current of the drive member exceeds a first threshold; and after the current of the drive member exceeds the first threshold, reduce a voltage of the drive member to maintain the current of the drive member below a second threshold that is greater than or equal to the first threshold.

### BRIEF DESCRIPTION OF THE FIGURES

The accompanying drawings, which are incorporated in and constitute a part of this specification, illustrate various examples and together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials and/or elements in different figures are labeled similarly. It is understood that various combinations of the structures, components, and/or elements, other than those specifically shown, are contemplated and are within the scope of the present disclosure.

Moreover, there are many embodiments described and illustrated herein. The present disclosure is neither limited to any single aspect nor embodiment thereof, nor to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIG. 1 is a perspective view of an auto-injector, according to an example of the disclosure.

FIG. 1A is a perspective view of a portion of a housing of an auto-injector according to the disclosure.

FIG. 1B is a perspective view of a portion of a housing of an auto-injector according to the disclosure.

FIG. 2 is a bottom view of an auto-injector according to the disclosure.

FIG. 3 is a side view of an auto-injector, showing an activating switch extending away from a tissue-facing surface, according to the disclosure.

FIG. 3A is a cross-sectional view of an auto-injector, showing an activating switch extending away from a tissue-facing surface, according to the disclosure.

FIG. 3B is a cross-sectional view of an auto-injector, showing an activating switch in a partially depressed position, according to the disclosure.

FIG. 3C is a cross-sectional view of an auto-injector, showing an activating switch in a fully depressed position, according to the disclosure.

FIG. 4 is an exploded view of an auto-injector, according to the disclosure.

FIG. 4A is a schematic illustration of a control system of an auto-injector according to the disclosure.

FIG. 4B is an exploded view of an auto-injector according to the disclosure.

FIG. 4C is a perspective view of a portion of a housing and an electronics board, according to an aspect of the disclosure.

FIG. 5 is an exploded view of a needle mechanism according to the disclosure.

FIG. 5A is a perspective view of a fluid conduit according to the disclosure.

FIG. 5B is a cross-sectional view of a needle of a fluid conduit according to the disclosure.

FIG. 6 is a perspective view of the needle mechanism of FIG. 5 in a first position according to the disclosure.

FIGS. 7-11 are side views of the needle mechanism of FIG. 5.

FIG. 12 is a side cross-sectional view of a portion of an auto-injector according to the disclosure.

FIG. 13 is a side cross-sectional view of a piercing mechanism according to the disclosure.

FIG. 13B is a side cross-sectional view of an auto-injector according to the disclosure.

FIG. 14 is a side cross-sectional view of a piercing mechanism according to the disclosure.

FIG. 14A is a side view of an outer screw of a screw assembly according to the disclosure.

FIG. 14B is bottom perspective view of an outer screw of a screw assembly according to the disclosure.

FIG. 14C is a top perspective view of an outer screw of a screw assembly according to the disclosure.

FIG. 14D is a cross-sectional view along line A-A of the outer screw of a screw assembly shown in FIG. 14A according to the disclosure.

FIG. 15 is a side view of a needle insert switch according to the disclosure.

FIG. 16A is a perspective view of a lock for an auto-injector according to an aspect of the disclosure.

FIG. 16B is a bottom view of an auto-injector and a lock according to the disclosure.

FIG. 16C is a cross-sectional view of an auto-injector and a lock according to the disclosure.

FIG. 16D is a cross-sectional view of an auto-injector and a lock according to the disclosure.

FIG. 16E is a perspective view of a lock for an auto-injector according to an aspect of the disclosure.

FIG. 16F is a side view of a lock for an auto-injector according to an aspect of the disclosure.

FIG. 17 is a bottom view of an electronics board for an auto-injector according to the disclosure.

FIG. 17A is a perspective view of an electronics board for an auto-injector according to the disclosure.

FIGS. 18-20 depict flowcharts of exemplary methods according to the disclosure.

FIGS. 20A and 20B depict graphs relating to electric controls for an auto-injector according to the disclosure.

FIGS. 21-23 depict flowcharts of exemplary methods according to the disclosure.

FIG. 24 is a schematic of the luciferase-based PD-1 bioassay described in Example 8 herein. Panel A: Inactive Jurkat cells; Panel B: Jurkat cells are activated by T-cell receptor (TCR) clustering through the CD3×CD20 bispecific antibody; Panel C: PD-1 activation attenuates response in activated Jurkat cells; Panel D: Blocking PD-1 rescues the response in activated Jurkat cells.

FIG. 25 illustrates tumor growth and survival results for mice implanted with Colon-26 tumor cells at Day 0 and treated with the indicated combinations of molecules by injection at Days 3, 6, 10, 13 and 19 (“early-treatment tumor model”). The graph depicts tumor volume (in mm3) for the different experimental groups at various time points after implantation. Upward arrows along the X-axis indicate the timing of treatment injections. “mIgG2a” is IgG2 isotype control; “Fc” is human Fc control; “VEGF Trap” is aflibercept; “anti-PD-1” is anti-mouse PD-1 clone RPMI-14; “anti-PD-L1” is an anti-PD-L1 monoclonal antibody as described elsewhere herein.

FIG. 26 illustrates tumor growth and survival results for mice implanted with Colon-26 tumor cells at Day 0 and treated with the indicated combinations of molecules by injection at Days 3, 6, 10, 13 and 19 (“early-treatment tumor model”). The graph shows the tumor volume (in mm3) of individual mice in each experimental group at Day 28 after implantation. “mIgG2a” is IgG2 isotype control; “Fc” is human Fc control; “VEGF Trap” is aflibercept; “anti-PD-1” is anti-mouse PD-1 clone RPMI-14; “anti-PD-L1” is an anti-PD-L1 monoclonal antibody as described elsewhere herein.

FIG. 27 is a table showing the effect of pH on the stability of 150 mg/ml mAb1 incubated at 45° C. for 28 days. aSE-UPLC and CEX-UPLC ‘Starting Material’ results are the average values of the starting material for all formulations.

FIGS. 28A, 28B, and 28C show storage stability of three formulations F1, F2 and F3, wherein F1 comprises 210 mg/ml mAb1, 10 mM histidine and 3% proline, at pH 6.0; F2 comprises 210 mg/mL mAb1, 10 mM histidine and 3% sucrose, at pH 6.0; and F3 comprises 210 mg/ml mAb1, 10 mM histidine and 5% sucrose, at pH 6.0. Storage stability is measured by % high molecular weight species (HMW) generated upon storage at −80° C. (FIG. 28A), −30° C. (FIG. 28B) and −20° C. (FIG. 28C) for up to 9 months and analyzed by size-exclusion chromatography (SEC).

FIGS. 29A, 29B, and 29C show storage stability of three formulations F1, F2 and F3, wherein F1 comprises 150 mg/ml mAb1, 10 mM histidine, 9% sucrose and 0.2% polysorbate 80 (PS80), at pH 6.0; F2 comprises 175 mg/mL mAb1, 10 mM histidine, 3% proline and 0.2% PS80, at pH 6.0; and F3 comprises 175 mg/ml mAb1, 10 mM histidine, 5% sucrose, 1.5% proline and 0.2% PS80, at pH 6.0. Storage stability is measured by % high molecular weight species (HMW) generated upon storage at −80° C. (FIG. 29A), −30° C. (FIG. 29B) and −20° C. (FIG. 29C) for up to 6 months and analyzed by size-exclusion chromatography (SEC).

FIG. 30 is a table showing viscosity of 150 mg/mL mAb1 with the addition of excipients and viscosity modifiers.

FIG. 31 is a table that shows effect of viscosity modifiers on the stability of 175 mg/mL mAb1 incubated at 45° C. for 14 days. apH, SE-UPLC and CEX-UPLC ‘Starting Material’ results are the average values of the starting material for all formulations.

Again, there are many embodiments described and illustrated herein. The present disclosure is neither limited to any single aspect nor embodiment thereof, nor to any combinations and/or permutations of such aspects and/or embodiments. Each of the aspects of the present disclosure, and/or embodiments thereof, may be employed alone or in combination with one or more of the other aspects of the present disclosure and/or embodiments thereof. For the sake of brevity, many of those combinations and permutations are not discussed separately herein.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the cross-sectional views are not drawn to scale and should not be viewed as representing proportional relationships between different components. The cross-sectional views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. In the discussion that follows, relative terms such as “about,” “substantially,” “approximately,” etc. are used to indicate a possible variation of +10% in a stated numeric value.

As described above, existing auto-injectors may be inadvertently triggered when dropped or vibrated. Additionally, existing auto-injectors may lack suitable control logic for stopping an injection when appropriate. These shortcomings may cause premature deployment of drugs, increase complexity of self-administration of drugs, introduce user errors, and cause user discomfort. Accordingly, the present disclosure is directed to various embodiments of an injection device (e.g., auto-injector) for self-administration of drugs, or other therapeutic agents, by a user. Specifically, according to certain embodiments, a likelihood of inadvertent triggering of the auto-injector may be reduced and the auto-injector may further incorporate control logic which improves operation of the auto-injector and user experience.

Additional details of auto-injectors in accordance with the present disclosure can be found in PCT/US2018/031077 to Arnott, et al., filed on Nov. 8, 2018, and published as WO 2018/204779 A1, and in U.S. application Ser. No. 18/055,895 to Grygus, filed on Nov. 16, 2022, the entireties of which are incorporated by reference herein. Additional details of vial piercing systems in accordance with the present disclosure can be found in U.S. Pat. No. 10,182,969, filed on Mar. 10, 2016, the entirety of which is incorporated by reference herein.

### Overall System

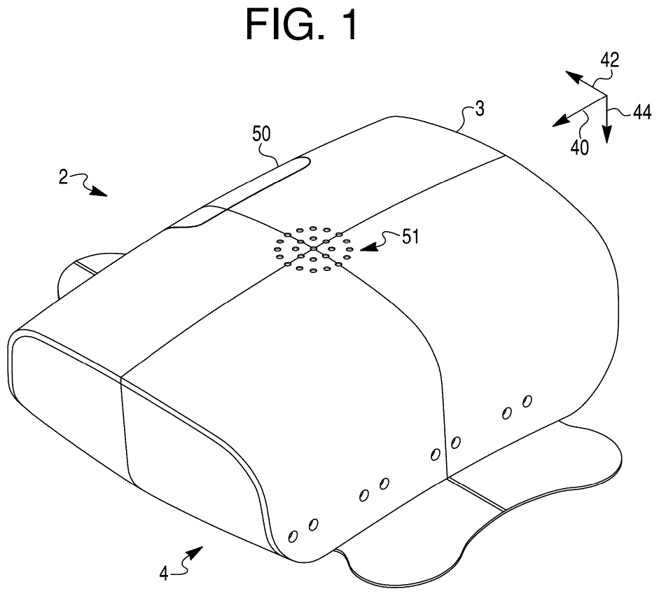

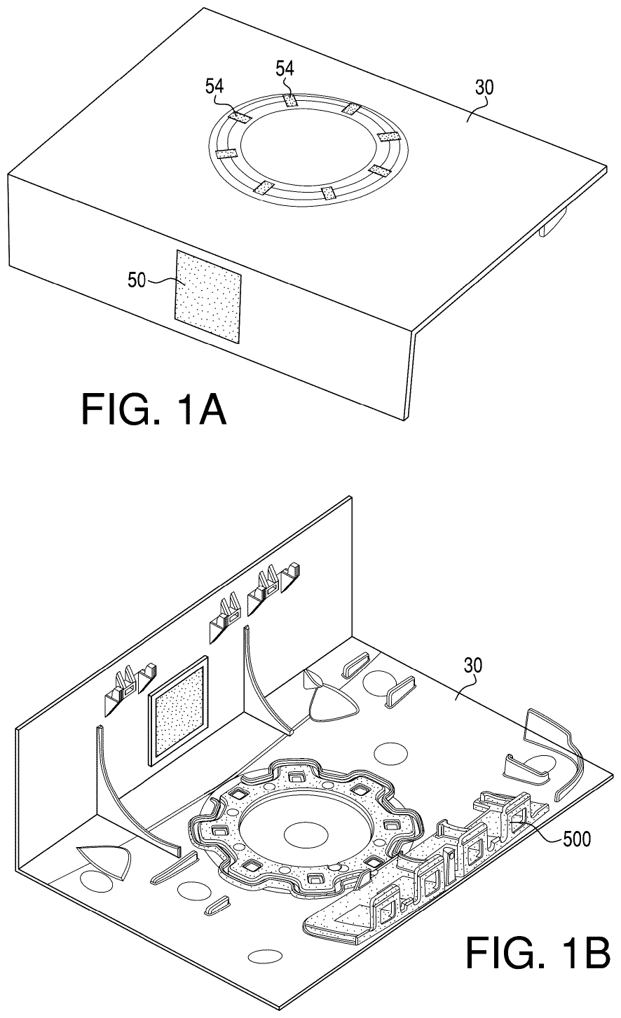

An example of such an auto-injector 2 is shown in FIGS. 1, 2, and 3. As shown in FIG. 1, auto-injector 2 may include a housing 3 having a tissue-engaging (e.g., bottom) surface 4 through which a needle may be deployed and retracted. As shown in FIGS. 1 and 1A, housing 3 may include a transparent window 50. Transparent window 50 may enable a viewer to visualize one or more displays or to visualize an interior of auto-injector 2 and components therein, such as a primary container and/or a drug product stored in the primary container.

In some embodiments, and as shown in FIG. 1, auto-injector 2 may include a plurality of openings 51 configured to facilitate the travel of sound generated within housing 3 (by, e.g., a speaker). Auto-injector 2 may have any suitable dimensions to enable portability and self-attachment by a user. In one example, auto-injector 2 may have a length of about 2.98 inches, a width of about 2.07 inches, and a height of about 1.07 inches. However, other suitable values also may be utilized, including, e.g., a length from about 0.5 inches to about 5.0 inches, a width of about 0.5 inches to about 3.0 inches, and a height from 0.5 inches to about 2.0 inches.

Auto-injector 2 may be oriented about a longitudinal axis 40 (e.g., an X axis), a lateral axis 42 (e.g., a Y axis) that is substantially perpendicular to longitudinal axis 40, and a vertical axis 44 (e.g., a Z axis) that is substantially perpendicular to both longitudinal axis 40 and lateral axis 42.

As shown in FIG. 1, an adhesive patch 12 may be coupled to tissue-engaging surface 4 to help secure auto-injector 2 to a user's body (e.g., skin). Adhesive patch 12 may be formed from fabric or any other suitable material, and may include an adhesive. The adhesive may be an aqueous or solvent-based adhesive, or may be a hot melt adhesive, for example. Suitable adhesives also include acrylic based, dextrin based, and urethane based adhesives as well as natural and synthetic elastomers. In some examples, the adhesive provided on patch 12 may be activated upon contact with a user's skin. In yet another example, patch 12 may include a non-woven polyester substrate and an acrylic or silicone adhesive. Patch 12 may be joined to housing 3 by, e.g., a double-sided adhesive, or by other mechanisms like ultrasonic welding. Patch 12 may have a length dimension greater than a width of auto-injector 2.

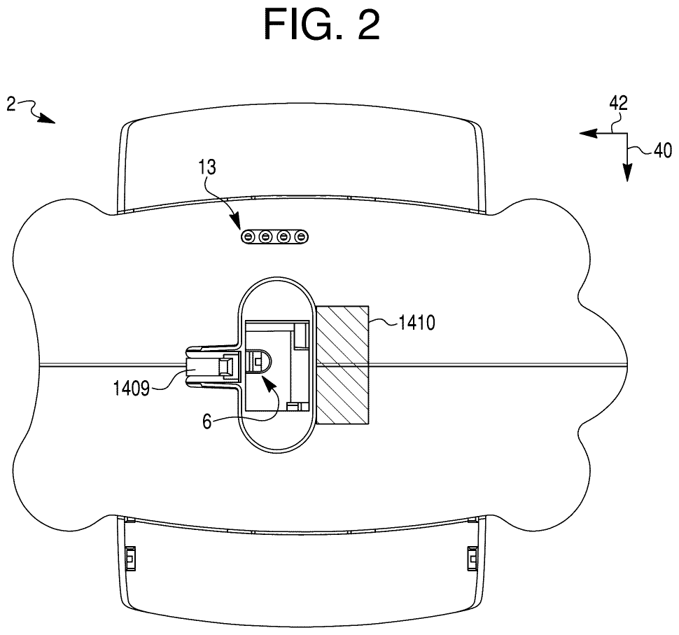

As shown in FIG. 2, auto-injector 2 may include an opening 6, through which the needle may be deployed and retracted. An activating switch 1409 may be disposed on tissue-engaging surface 4, and may be configured to activate auto-injector 2, or otherwise place auto-injector 2 in a “ready” mode. A touch sensor 1410 also may be disposed on tissue-engaging surface 4, and may be configured to help a controller of auto-injector 2 determine whether auto-injector 2 is disposed on the skin of a user (indicating that the auto-injector should fire or otherwise deploy a needle), or whether activating switch 1409 was improperly triggered (indicating that operation of auto-injector 2 should be stopped). A connecting port 13 also may be disposed on tissue-engaging surface 4 to facilitate programming of auto-injector 2.

Auto-injector 2 may be configured to operate in three or more operation phases including, e.g., an injection sequence activation phase, an injection phase, and a retraction phase, each of which will be described in further detail herein. The injection sequence activation phase, injection phase, and retraction phase may collectively be referred to herein as an “injection sequence.”

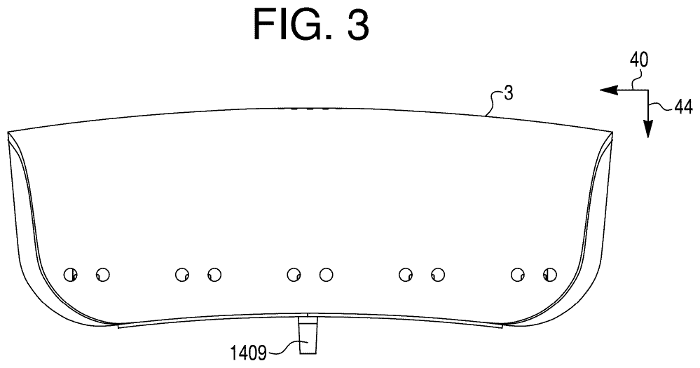

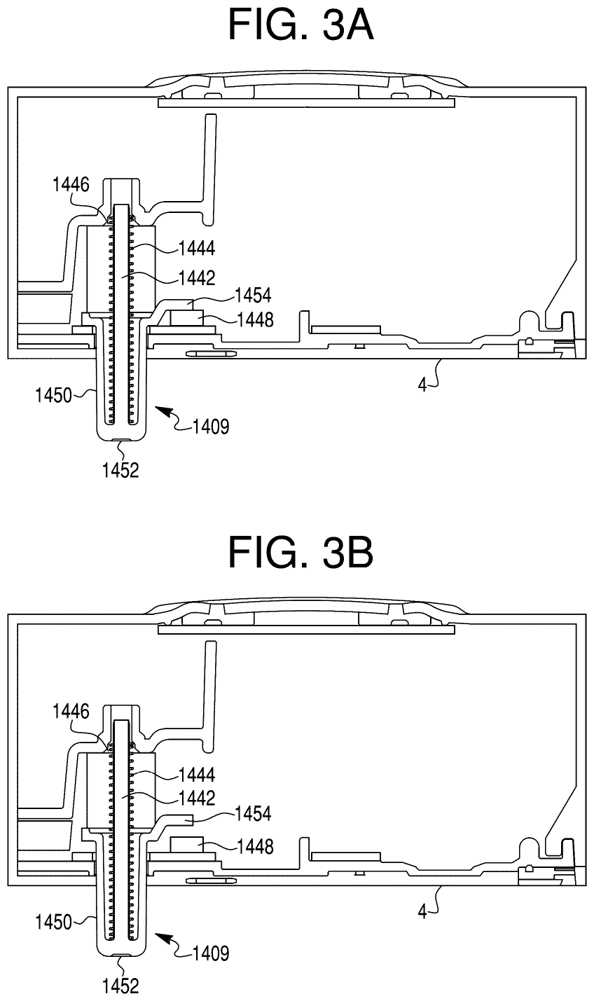

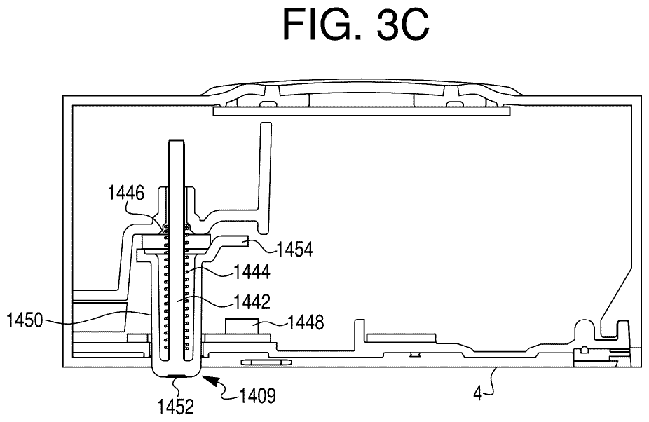

Referring to FIGS. 3A, 3B, and 3C which show cross-sections of an auto-injector 2, activating switch 1409 may be a mechanical plunger-type switch. For example, activating switch 1409 may include a plunger 1450 having a plunger contact surface 1452. Plunger contact surface 1452 may be generally circular in shape (or have another suitable shape) and may be large enough to be depressed comfortably by soft skin. In some embodiments, plunger contact surface 1452 may have a diameter or width ranging from about 2 mm to about 10 mm, a diameter ranging from about 4 mm to about 8 mm, or a diameter of about 6 mm. Activating switch 1409 may further include a shaft 1442, a biasing member 1444, a biasing collar 1446, and a plunger flange 1454. Biasing member 1444 may be a spring, for example, and may surround shaft 1442. Biasing member 1444 may be fixed, or otherwise prevented from moving, at one end by biasing collar 1446. Plunger flange 1454 may be configured to contact or otherwise depress a plunger switch 1448. For clarity, the term activating switch and/or reference to activating switch 1409, as used herein, should be understood to encompass any or all components of activating switch 1409, including shaft 1442, biasing member 1444, biasing collar 1446, plunger switch 1448, plunger 1450, plunger contact surface 1452, and plunger flange 1454.

In a free state, i.e., when plunger 1450 is not depressed, either by being pressed against the skin of a user or otherwise, plunger 1450 may extend outwardly from tissue-engaging surface 4 as shown in FIG. 3A. In the free state, plunger contact surface 1452 may be a distance from tissue-engaging surface 4 ranging from about 1 mm to about 16 mm, ranging from about 5 mm to about 12 mm, or a distance of about 8.5 mm. In the free state, biasing member 1444 may urge plunger 1450 to extend outwardly from tissue-engaging surface by pressing against biasing collar 1446. In the free state, plunger flange 1454 may be in contact with, or otherwise depress plunger switch 1448. When plunger flange 1454 is in contact with, or otherwise depresses plunger switch 1448, an electrical circuit associated with plunger switch 1448 may be complete, or closed.

When plunger 1450 is depressed either by being pressed against the skin of a user or otherwise, plunger 1450 may initially move to a partially depressed state, as shown in FIG. 3B. In the partially depressed state, biasing member 1444 may be compressed against biasing collar 1446. Plunger flange 1454 may further be out of contact with, or otherwise not depressing plunger switch 1448. When plunger flange 1454 is spaced apart from, not in contact with, or is otherwise not depressing plunger switch 1448, the electrical circuit associated with plunger switch 1448 may be broken, or open. By this configuration, the auto-injector 2 may be maintained in a reduced power state while plunger 1450 is depressed, such as when auto-injector 2 is in packaging.

As shown in FIG. 3B, plunger 1450 may not necessarily travel to the fully depressed state (shown in FIG. 3C) before plunger flange 1454 is out of contact with plunger switch 1448. As shown in FIG. 3C, on the other hand, in the fully depressed state, plunger 1450 may be depressed inwardly such that plunger contact surface 1452 is flush or nearly flush with tissue-engaging surface 4.

Plunger flange 1454 may be out of contact with plunger switch 1448, for example, after less than 5 mm of travel by plunger 1450, after less than 3 mm of travel by plunger 1450, after less than 1 mm of travel by plunger 1450, or after about. 0.75 mm of travel by plunger 1450—all when, for example, the maximum depression distance is 8.5 mm. In other words, plunger 1450 may transition from the free state, in which plunger flange 1454 is in contact with plunger switch 1448, to the partially depressed state, in which plunger flange 1454 is out of contact with plunger switch 1448, after moving only a portion of a maximum depression distance of plunger 1450 relative to housing 3 of auto-injector 2. For example, plunger 1450 may transition to the depressed state after moving only about 5%, about 10%, or about 20% of the maximum depression distance. Accordingly, the auto-injector 2 and plunger switch 1448 may be sufficiently responsive upon depressing plunger 1450 against a user's skin. For example, auto-injector 2 and plunger switch 1448 may be sufficiently responsive when pressed against skin of varying firmness or users having varying body fat content. While examples of travel distances for plunger 1450 are provided herein, it is to be understood that the present disclosure is not limited to any particular examples and any suitable travel distance may be used.

Biasing member 1444 may be sufficiently stiff such that in the free state, plunger flange 1454 stays in contact with or otherwise continuously depresses plunger switch 1448. Biasing member 1444 may also be of a stiffness such that plunger 1450 may be depressed comfortably when pressed against a user's skin. Biasing member 1444 may be biased to maintain plunger 1450 in the free state.

Though activating switch 1409 is shown in FIGS. 3A-3C as a mechanical plunger-type switch, it is to be understood that activating switch 1409 may be any other suitable type of switch, such as a rocker switch, throw switch, toggle switch, temperature switch, and the like. Additionally, while the electrical circuit associated with plunger switch 1448 is described herein as closed when plunger 1450 is in the free state and open when plunger 1450 is in the depressed state, it should be understood that an opposite configuration may be used. For example, the electrical circuit associated with plunger switch 1448 may be open when plunger 1450 is in the free state and closed when plunger 1450 is in the depressed state.

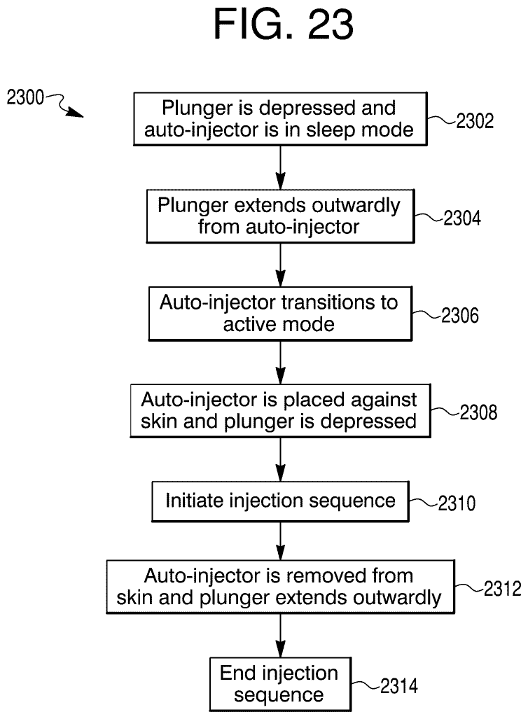

A method of controlling auto-injector 2 according to positions of activating switch 1409 will be described hereinafter in further detail with reference to FIG. 23.

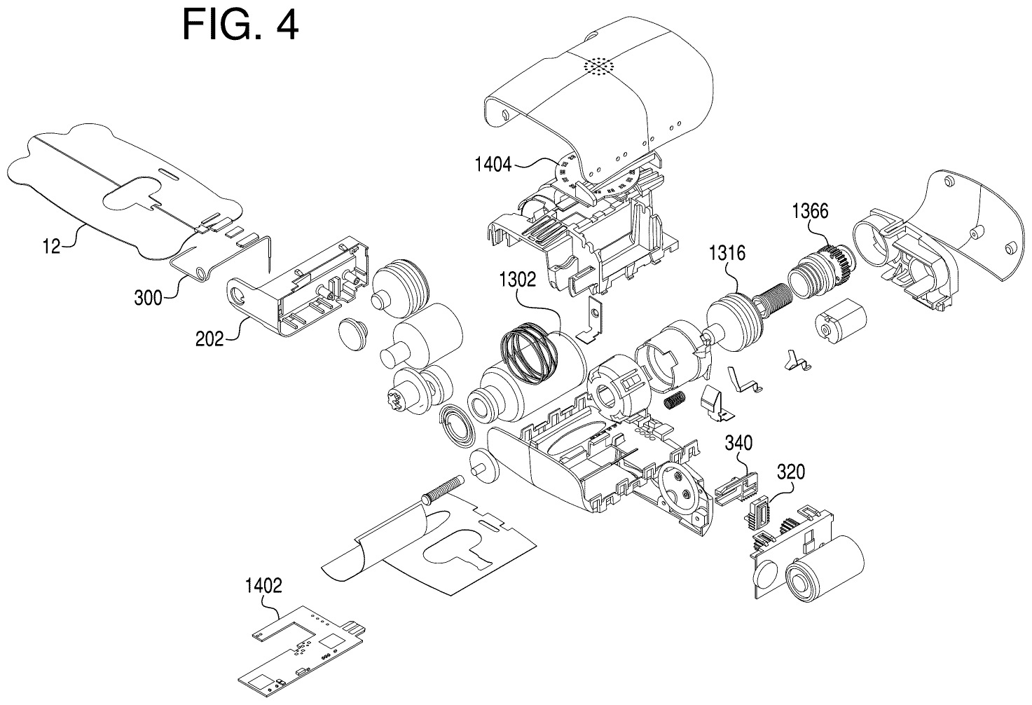

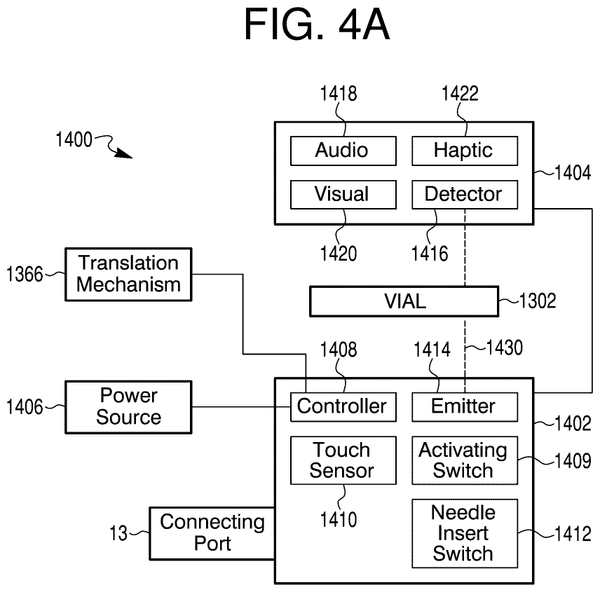

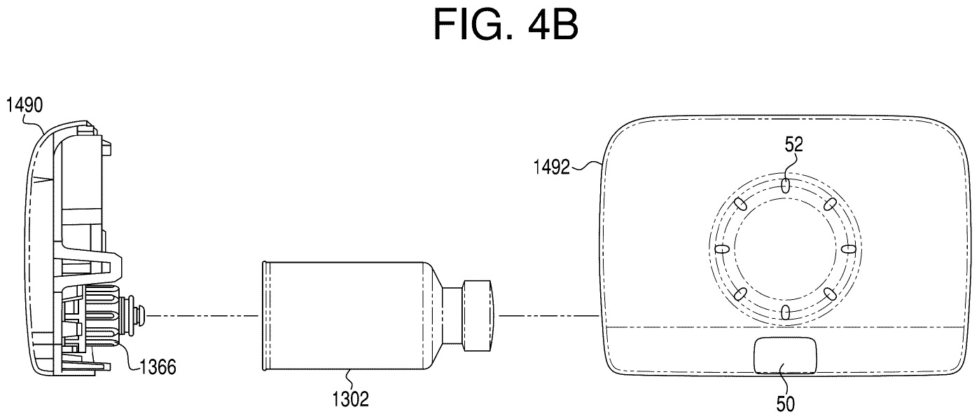

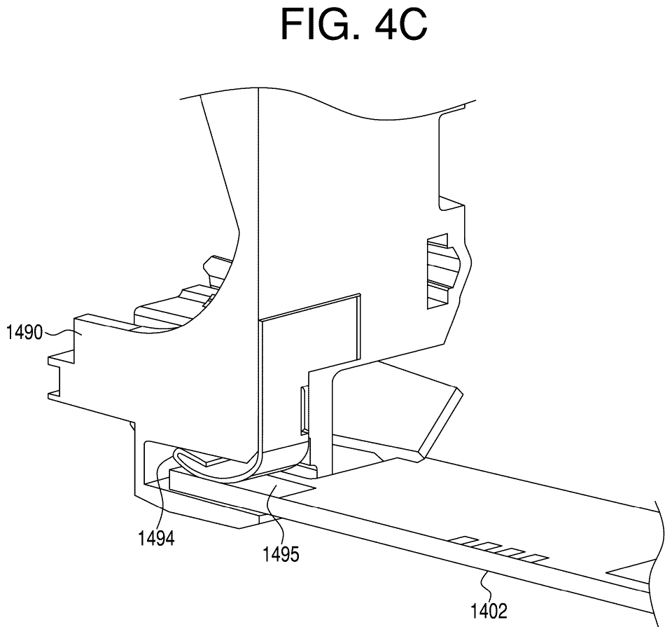

Further, as shown in FIG. 4B, in some embodiments, auto-injector 2 may include a plurality of LEDs 52. The LEDs 52 may be arranged in a ring-like formation, or any other suitable formation. As described in further detail hereinafter, light from the one or more LEDs 52 may be indicative of various operational states of the auto-injector 2.

### Auto-Injector Housing

Referring to FIGS. 1A and 1B, the housing 3 of auto-injector 2 may include an upper portion 30. The upper portion 30 may form a portion of the housing 3 opposite tissue-engaging surface 4. The upper portion 30 may include transparent window 50 through which a user may be able to see contents of the auto-injector 2, including a vial and/or a drug contained in the vial. The transparent window 50 may be positioned on a side of the upper portion 30 and may be formed such that it conforms to a rounded/curved contour of the upper portion 30, as shown in FIG. 1. The transparent window 50 may be generally rectangular in shape with rounded corners.

The upper portion 30 may also include a plurality of transparent windows 54. The transparent windows 54 may be formed on a top surface of upper portion 30 and may be arranged in any suitable configuration, such as a circular configuration, an oval configuration, a rectangular configuration, or a linear configuration, for example. Transparent windows 54 may be circumferentially spaced apart from one another, for example. The transparent windows 54 may allow light from one or more LEDs located within the housing 3 to be visible to a user. The light from the one or more LEDs may be indicative of various operational states of the auto-injector 2, as described herein.

The transparent window 50 and the transparent windows 54 may be integrally formed as part of the upper portion 30. As shown in FIG. 1B, the upper portion 30 may include a transparent portion 500 formed from a transparent material. The transparent portion 500 may be contiguous, such that the transparent window 50 and the transparent windows 54 are formed of one piece of transparent material. Moreover, the transparent portion 500 may be integrated into the upper portion 30 such that the upper portion 30, including the transparent window 50 and the transparent windows 54 is manufactured as a single part.

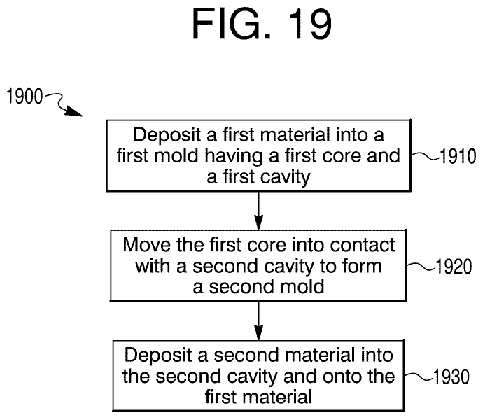

To form the upper portion 30 as a single part, the upper portion 30 may be manufactured using a double shot molding process, for example. FIG. 19 illustrates an exemplary method 1900 of molding the upper portion 30 using double shot molding or insert molding. At step 1910, a first material may be deposited into a first mold having a first core and a first mold cavity. The first material may have a low opacity and may be, for example, a transparent material for the transparent window 50 and the transparent windows 54. The first material may be, for example, transparent acrylic, clarified acrylonitrile butadiene styrene (ABS), polycarbonate, polyvinylchloride (PVC), or polyethylene terephthalate glycol (PETG). The first mold may be configured, for example, to form transparent portion 500.

At step 1920, the first core and the material in the first mold cavity, may be moved within a second mold cavity to form a second mold. When moved, the first core may retain the first material. The second mold may be configured, for example, to form the upper portion 30. At step 1930, a second material may be deposited into the second mold cavity in which the first material is contained. The second material may be deposited around the first material and first core in unoccupied space of the second mold cavity to form the upper portion 30. The second material may be a material with high opacity, such as white plastic. The second material may be, for example, ABS, polycarbonate, ABS-polycarbonate blend, PVC, or PETG.

Accordingly, the generally opaque upper portion 30, which includes the transparent window 50 and the transparent windows 54 may be formed from two different materials to form a single part. Forming the upper portion 30 as a single part may reduce an overall number of steps required to assemble auto-injector 2. For example, in some embodiments, no fastening or adhesive steps or materials are needed to join transparent and opaque portions of the housing. Avoiding unnecessary assembly steps may further improve the appearance of cosmetic surfaces of the auto-injector 2. Additionally, forming the upper portion 30 as a single part may improve the overall structural integrity of the auto-injector 2. Further, forming the upper portion 30 as a single part may reduce or eliminate sinks on cosmetic surfaces.

### Needle Mechanism

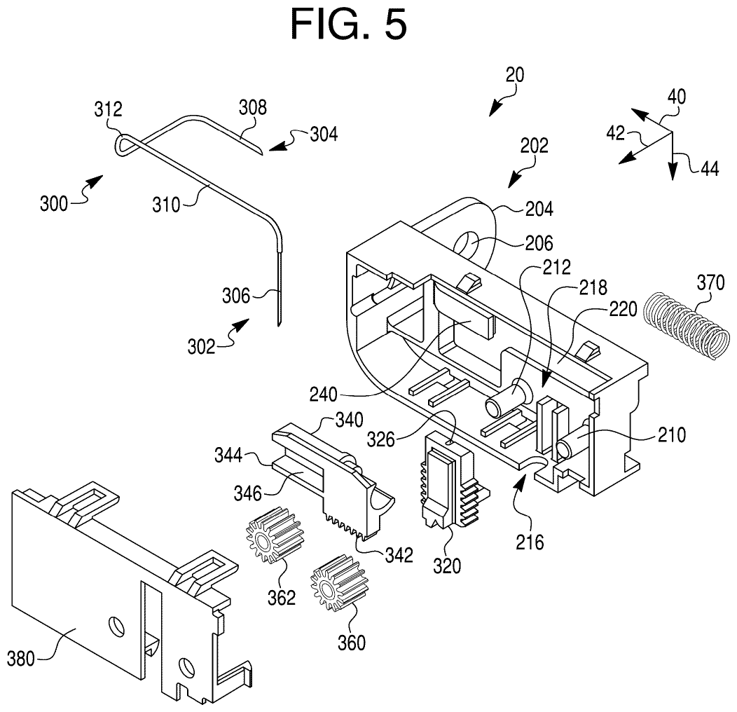

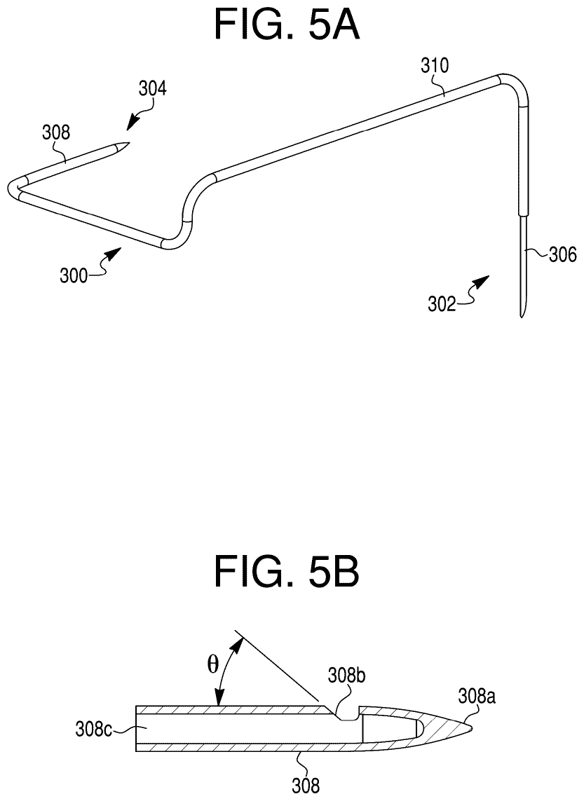

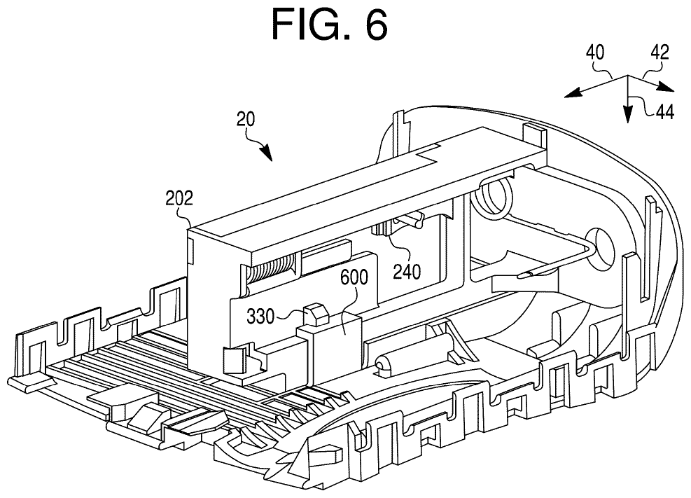

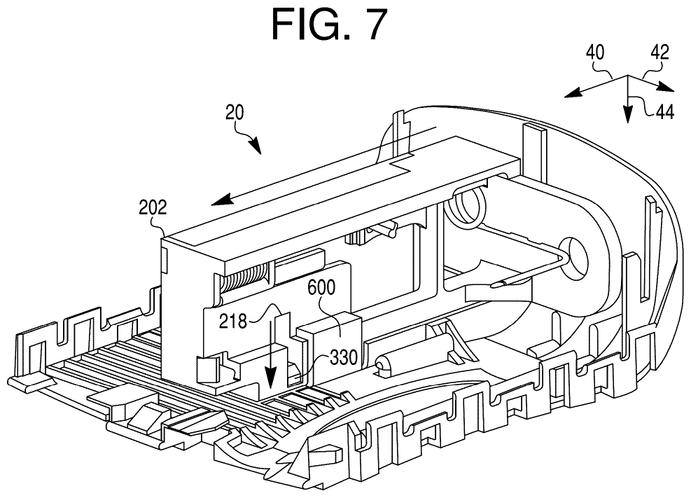

Referring to FIGS. 5-11, a needle mechanism 20 includes a carrier 202 that is movable (e.g., slidable) within housing 3 between a first position (FIG. 6) and a second position (FIG. 7). Needle mechanism 20 also may include a fluid conduit 300 that is mounted to carrier 202, and which may be deployed into a user, and retracted by a driver 320. A shuttle 340 (e.g., a shuttle actuator) may be configured to move driver 320 via a deployment gear 360, and a retraction gear 362. Shuttle 340 may be coupled to a resilient member (e.g., a spring 370). A cover 380 (FIG. 5) may be coupled to carrier 202 to enclose various components of needle mechanism 20.

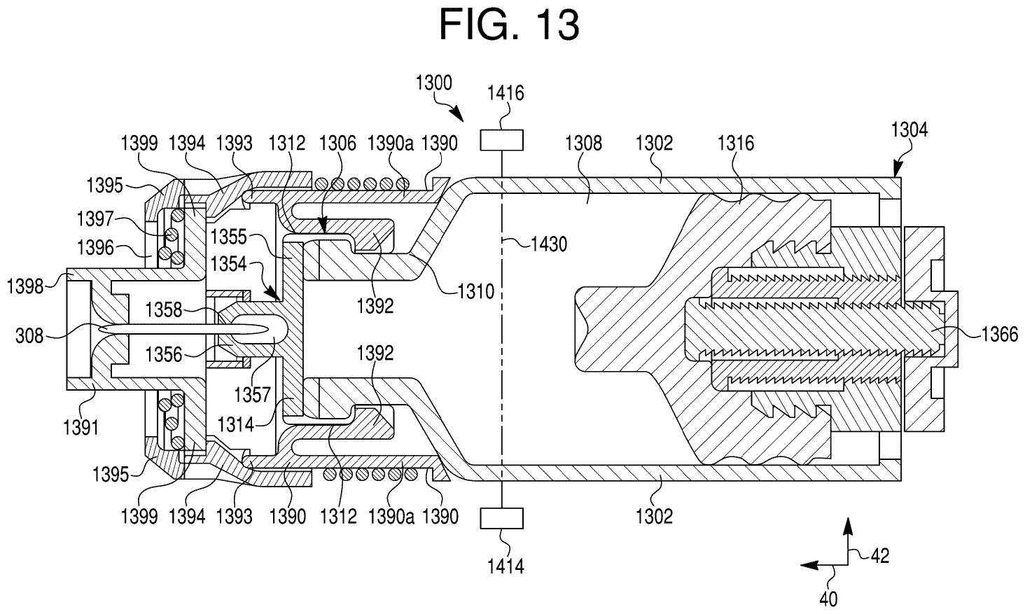

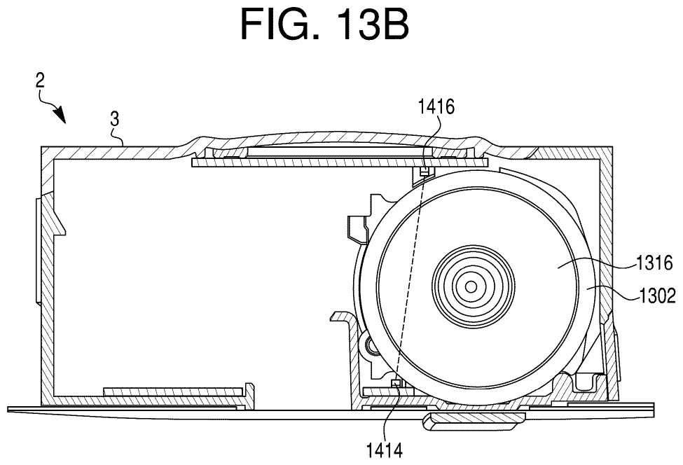

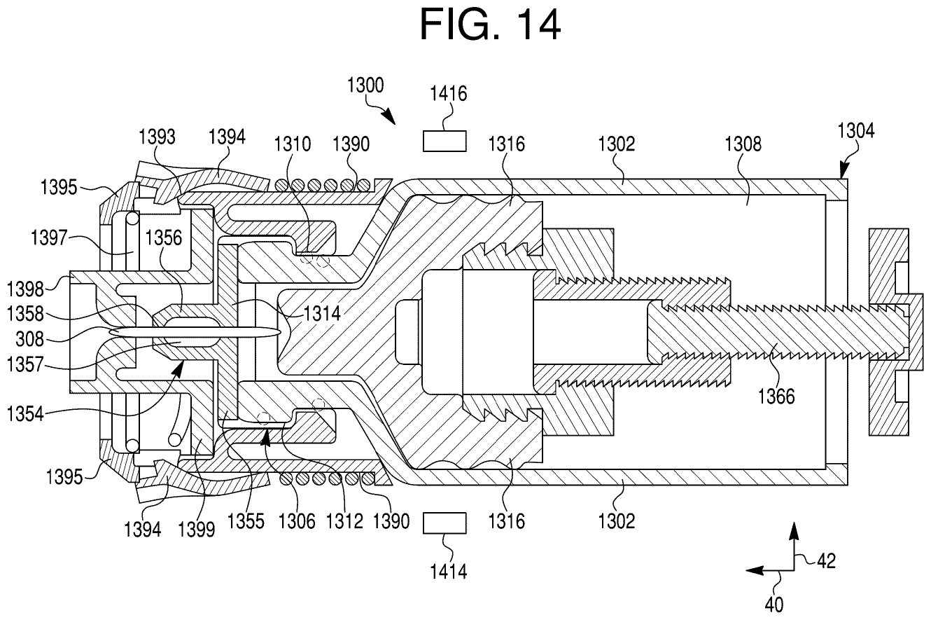

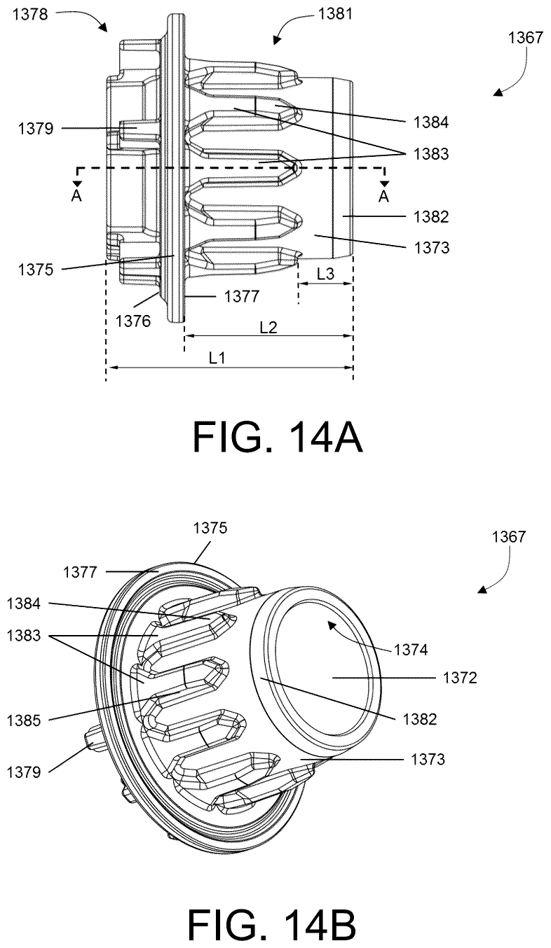

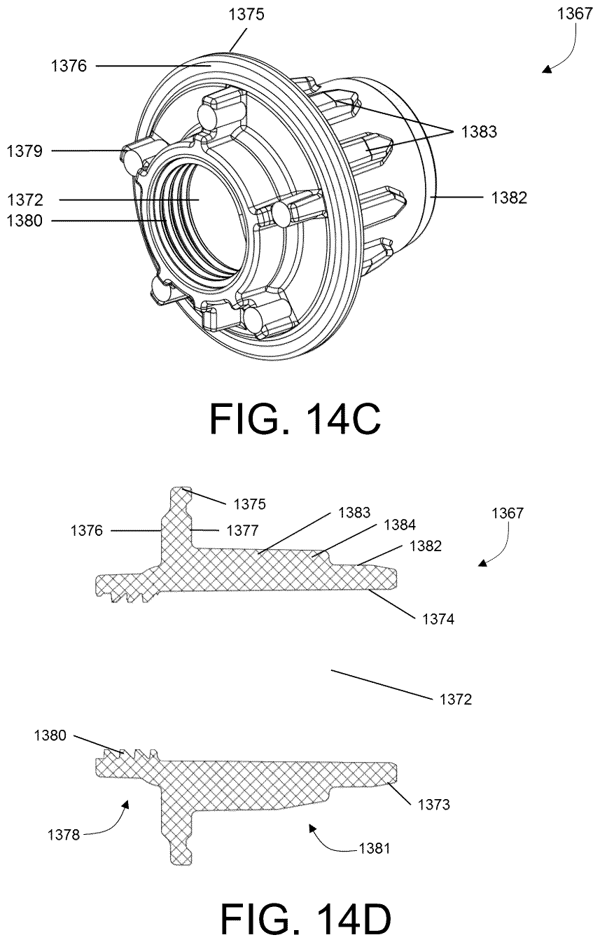

Referring to FIG. 5, fluid conduit 300 may extend from a first end 302 to a second end 304. As shown in further detail in FIG. 5A, first end 302 may include a needle 306 that is configured to be injected into a user. Needle 306 may include a sharp and/or beveled tip, and may extend generally along or parallel to axis 44. Second end 304 may include a needle 308 that is substantially similar to needle 306, but may be positioned within auto-injector 2 to penetrate a cartridge 1302 (shown in FIG. 13 and described in further detail below) to access drugs to be injected into the user. Fluid conduit 300 may include an intermediate section 310 including one portion extending along or parallel to axis 40, and a second portion extending along or parallel to axis 40. The first and second portions of intermediate section 310 may be joined in a serpentine section 312 that facilitates flexion of fluid conduit 300 and movement of needle 306 along axis 44 during deployment into the user, and during retraction out of the user. While a serpentine section 312 is shown, any other suitable shape, e.g., a coil, curved, or other shape that enables flexion of fluid conduit 300 is also contemplated. Serpentine section 312, or similar structure, may act as a cantilever when needle 306 is deployed and/or retracted. Serpentine section 312 also may bias fluid conduit 300 into the deployed configuration shown in FIG. 5. Once needle 308 penetrates and establishes fluid communication with cartridge 1302 (see, e.g., FIG. 14), drugs may travel from cartridge 1302, through needle 308, intermediate section 310, and needle 306 (pierced through the user's skin), and into the user. In some examples, fluid conduit 300 may include only metal or a metal alloy. In other examples, fluid conduit 300 may be any other suitable material, such as, e.g., polymers or the like. Needle 308 and intermediate portion 310 may define a 22 or 23 Gauge, thin-walled needle, while needle 306 may be a 27 Gauge, thin-walled needle. Other needle sizes ranging from, e.g., 6 Gauge to 34 Gauge, and other needle wall thicknesses, such as regular wall, extra-thin wall, and ultra-thin wall also may be utilized as appropriate. Fluid conduit 300 may reduce the amount of material that contacts the drugs, reduce joints and assembly steps, and require less sterilization than conventional devices.

As shown in FIG. 5B, needle 308 may be configured to include a needle tip 308a and a side port 308b. Side port 308b may be fluidly connected to fluid path 308c and allow fluid to enter fluid conduit 300 through a side of needle 308, as opposed to through the tip of needle 308. A rear wall of side port 308b may be inclined at an angle θ relative to a longitudinal axis of fluid path 308c. In some embodiments, the angle θ may be between about 20° and 60°, between about 30° and 50°, or about 40°. By configuring needle 308 in this way, needle 308 may be optimized for piercing the primary container of auto-injector 2, which may be a sealed cartridge or a vial. The relative positioning of needle tip 308a and side port 308b may allow piercing of the seal of the primary container without coring or otherwise cutting a portion of the seal with an opening to fluid path 308c. Thereby, entry of particles cored or cut from the seal into the fluid path 308c may be minimized or avoided.

Needle 306 may be configured substantially similarly to needle 308, as shown in FIG. 5B. Alternatively, in some embodiments, either or both of needles 306 and 308 may be a 3 bevel needle, a 5 bevel needle, or any other suitable type of needle. In some embodiments, one or both of needles 306 and 308 may be a pencil point needle having a round hole or any other suitably shaped hole.

Carrier 202 may be formed of plastic (e.g., injection-molded plastic), a metal, metal alloy, or the like, and may include a flange 204 with an opening 206, and posts 210 and 212. Carrier 202 also may include an opening 216 through which a needle or other fluid conduit may be deployed. Opening 216 may be a slot that is recessed from an end surface of carrier 202, or, in an alternative embodiment, an entirety of the perimeter of opening 216 may be defined by material of carrier 202. Carrier 202 also includes a driver path 218. Driver path 218 may be a slot in carrier 202 that extends along or parallel to axis 44. Driver path 218 may be configured to receive a protrusion of driver 320, such as, e.g., protrusion 330 discussed in further detail below. Carrier 202 also may include a shuttle path 220, along which shuttle 340 may move, as described in further detail below.

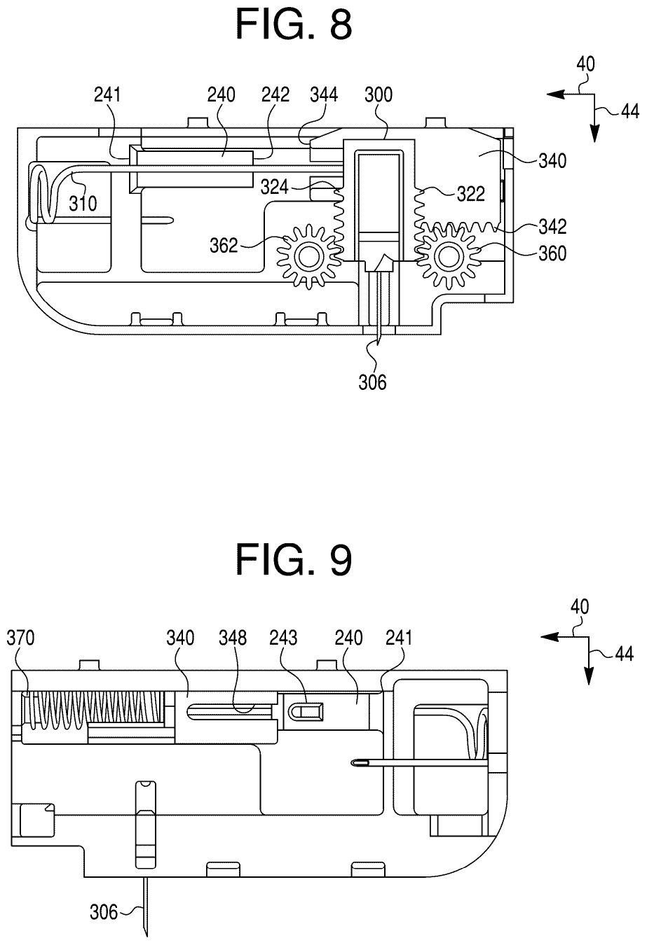

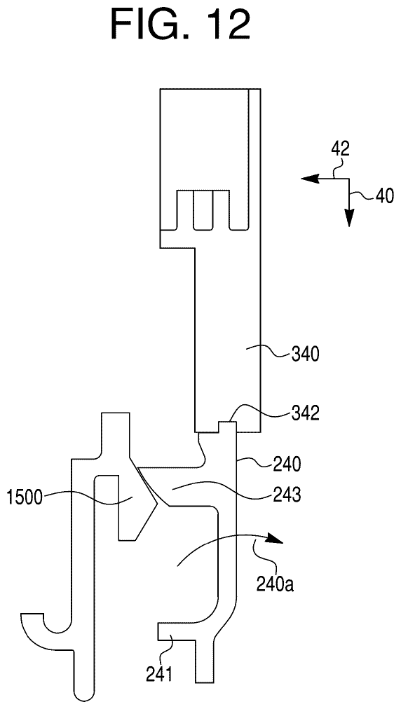

Carrier 202 also may include a stop 240 that is configured to engage shuttle 340. Stop 240 may be a cantilever having a fixed end 241 (FIG. 8) and a free end 242 (FIG. 8). Stop 240 may include an inclined ramp 243 (FIGS. 9 and 12) that, when engaged or pushed by a ramp 1500 (described with reference to FIG. 12), causes stop 240 to deflect about fixed end 241. In a first position, free end 242 may block or otherwise impede movement of shuttle 340, and in a second configuration, may permit movement of shuttle 340. The relationship between stop 240 and shuttle 340 will be discussed in further detail later in the application.

Driver 320 includes two racks 322 and 324 (shown in FIG. 8) parallel to one another and disposed on opposing sides of driver 320. Racks 322 and 324 may include teeth and may be configured to engage with and drive rotation of deployment gear 360 and retraction gear 362, respectively. Driver 320 may include a lumen 326 (or a track, recess, or other suitable structure) (FIG. 5) that is configured to receive needle 306 of fluid conduit 300. Driver 320 also may include protrusion 330 (FIGS. 6 and 7) that is configured to slide within driver path 218 of carrier 202. Protrusion 330 may include a hook-like configuration that can “catch” on impediment 600, as described in further detail below.

With continuing reference to FIG. 5, shuttle 340 may include a rack 342 configured to engage with gears 360 and 362. Shuttle 340 also may include an end surface 344, and a recess 346 that extends along a length of shuttle 340 in the same direction as rack 342. A slot 348 (FIG. 9) may extend along the length of recess 346. Slot 348 may extend through the middle of recess 346 and may extend along an entirety or substantial entirety of recess 346.

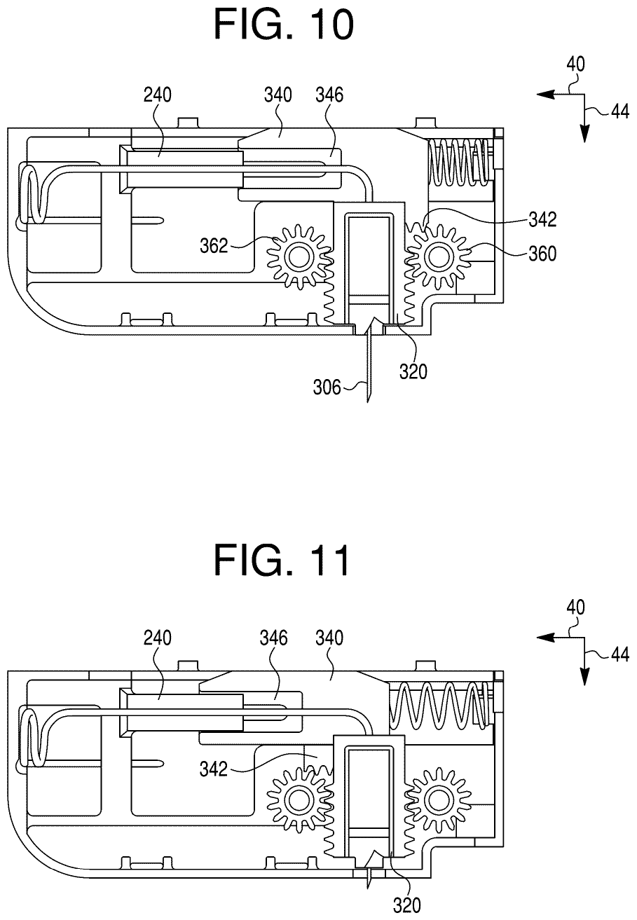

Shuttle 340 may move along track 220 from a first, starting position (FIG. 8), to a second, intermediate position (FIGS. 9 and 10), and from the second position to a third, final position (shown between the second and third configurations in FIG. 11). As shuttle 340 moves along track 220, rack 342 may first engage deployment gear 360, and then retraction gear 362. At certain times, rack 342 engages at most one of deployment gear 360 and retraction gear 362 at any given time. In some examples, such as when rack 342 is disposed longitudinally between deployment gear 360 and retraction gear 362, rack 342 is not engaged with either of deployment gear 360 and retraction gear 362. Shuttle 340 may be configured to move only along one axis (e.g., axis 40) and only in one direction along the one axis. The force required to move shuttle 340 along track 220 may be provided by expansion of spring 370. Spring 370 may be compressed from a resting state, and the expansion of spring 370 may move shuttle 340 along track 220 through the series of positions/configurations set forth above. At various positions of shuttle 340, different features of auto-injector 2 may directly or indirectly block movement of shuttle 340. Alternatively, it is contemplated that spring 370 may be biased to a compressed configuration. In this alternative embodiment, spring 370 may be expanded from a resting state, and compression of spring 370 may move shuttle 340 along track 220 through the series of positions/configurations set forth above.

The first position of shuttle 340, shown in FIG. 8, may correspond to an unused, undeployed, and/or new state of auto-injector 2. In this first position, driver 320 may be in an undeployed state. Shuttle 340 is maintained in the first position by the positioning of an impediment 600 in the path of driver 320 (FIG. 6). Impediment 600, which may be a shelf of housing 3, or another suitable blocking device, may prevent movement of driver 320 by engaging and/or retaining protrusion 330. Therefore, because driver 320, deployment gear 360, and rack 342 are coupled to one another, the blockage of driver 320 also prevents movement of shuttle 340. Shuttle 340 may move from the first position to the second position by moving impediment 600 relative to carrier 202 (or vice versa). In one example, carrier 202 is moved (e.g., to the left in FIG. 6) while impediment 600 remains stationary.

When the path of driver 320 is free from impediment 600 (FIG. 7), spring 370 may expand and move shuttle 340 along track 220. This linear movement of shuttle 340 may rotate deployment gear 360 counter-clockwise (or clockwise in other examples) via rack 342, and the rotation of deployment gear 360 may move driver 320 downward along axis 44, via rack 322 of driver 320. This downward movement of driver 320 may cause needle 306 to pierce through the skin of a user. In some examples, driver 320 may be configured to move, relative to carrier 202, along only axis 44.

Shuttle 340 may be moved by the expansion of spring 370 until its end surface 344 abuts free end 242 of stop 240 such that shuttle 340 is maintained in the second position shown in FIGS. 9 and 10. At this point, free end 242 may prevent further expansion of spring 370 and further movement of shuttle 340 along track 220. In this second position, fluid conduit 300 may be deployed within a user, and fluid from cartridge 1302 may be injected into the user via needle 306. Additionally, while shuttle 340 is in the second position, rack 342 may be engaged with deployment gear 360 to maintain needle 306 in the deployed configuration. Shuttle 340 may move from the second position to the third position by the flexion of stop 240 about its fixed end 241. Further details of this flexion are set forth below with respect to FIGS. 12-14. The flexion of stop 240 may allow spring 370 to continue expanding, urging shuttle 340 further along track 220. In some examples, stop 240 may be received by and/or within recess 346 of shuttle 340, and ramp 243 may slide within slot 348, as shuttle 340 moves from the second position to the third position.

The movement of shuttle 340 from the second position to the third position may correspond to the retraction of needle 306 from the user into housing 3. In particular, rack 342 may engage with and rotate retraction gear 362 in the same direction (e.g., counter-clockwise or clockwise) as deployment gear 360 was rotated. The rotation of retraction gear 362 may urge driver 320 back to a retracted position via rack 324. Shuttle 340 may reach the third position, where driver 320 is fully-retracted, when its end surface 344 engages a wall of carrier 202, when free end 242 of stop 240 reaches an end of recess 346, and/or when spring 370 reaches a resting state.

In some embodiments, once driver 320 moves from the deployed state back to the retracted state, it may be prevented from moving out of the retracted state. As a result, needle 306 will be prevented from re-deployment into the user. In this configuration, auto-injector 2 may be a single-use device (e.g., discarded after completing one injection). In other embodiments, auto-injector 2 may be reset and reused. Furthermore, deployment gear 360 and retraction gear 362 may be the only rotating gears disposed within auto-injector 2, in some examples.

### Piercing System and Sterile Connector

FIGS. 13 and 14 show features of a piercing system 1300 of auto-injector 2. Additional details of exemplary piercing systems can be found in U.S. Patent Application Publication No. 2016/0262984 A1 to Arnott et al., published on Sep. 15, 2016, the entirety of which is incorporated by reference herein. Piercing system 1300 includes a primary container, which may be a cartridge 1302 with a first end 1304 and a second end 1306. The primary container may alternatively be a chamber, syringe, vial, flexible sac, or any other suitable fluid containing structure.

Cartridge 1302 may include a cavity 1308 opened at first end 1304 and extending toward second end 1306. Second end 1306 may include a neck 1310 with a cap 1312 that engages neck 1310 to close second end 1306. A septum 1314 may be positioned between cartridge 1302 and cap 1312 to assist with closing second end 1306, and allow for needle 308 (e.g., a staked needle) to be inserted into cartridge 1302. Cavity 1308 may be closed at first end 1304 by a piston 1316.

Cartridge 1302 may have a 5 mL capacity in some examples, although any other suitable volume (e.g., from 1 mL to 50 mL, or from 2 mL to 10 mL, or from 3 mL to 6 mL, or from 2 mL to 5 mL, or from 10 mL to 20 mL, or from 10 mL to 30 mL, or another suitable range) also may be utilized depending on the drug to be delivered. In some examples, cartridge 1302 may have a capacity of 10 mL, 15 mL, 20 mL, 25 mL, 30 mL, 35 mL, 40 mL, 45 mL, or 50 mL In other examples, cartridge 1302 may have a capacity greater than or equal to 1 mL, or greater than or equal to 2 mL, or greater than or equal to 3 mL, or greater than or equal to 4 mL, or greater than or equal to 5 mL, or greater than or equal to 10 mL, or greater than or equal to 15 mL. Cartridge 1302 may contain and preserve a drug for injection into a user, and may help maintain sterility of the drug. Cartridge 1302 may have a 13 mm diameter neck, a 45 mm length, and an internal diameter of 19.05 mm. These values are merely exemplary, and other suitable dimensions may be utilized as appropriate. In some examples, cartridge 1302 may be formed using conventional materials, and may be shorter than existing devices, which can help auto-injector 2 remain cost-effective and small. Cartridge 1302 may be a shortened ISO 10 mL cartridge.

Septum 1314 may include an uncoated bromobutyl material, or another suitable material. Piston 1316 may include a fluoropolymer coated bromobutyl material, and also may include a conical nose 1316a to help reduce dead volume within cartridge 1302. Piston 1316 may include one or more rubber materials such as, e.g., halobutyls (e.g., bromobutyl, chlorobutyl, florobutyl) and/or nitriles, among other materials.

Piercing system 1300 also may include a top 1354 positioned at second end 1306. Top 1354 may include a base 1355 positioned over septum 1314 and the opening of cartridge 1302. Top 1354 may include a chamber 1356 extending from base 1355 in a direction away from piston 1316. Chamber 1356 defines a cavity 1357 and includes an opening 1358 in communication with cavity 1357. In some embodiments, top 1354 may be integrated with septum 1314 (e.g., integral or of one-piece construction). In alternative embodiments (not shown), top 1354 may be provided or initially assembled on fluid conduit 300 and not installed directly on/with cartridge 1302 and/or integrated with septum 1314.

A portion of fluid conduit 300, such as needle 308, a tube or the like, may extend through opening 1358 of chamber 1356 and into cavity 1357, but not through base 1355 in the pre-activated state. Opening 1358 may be pre-formed, or may be formed by the penetration of needle 308 through chamber 1356. Opening 1358 of chamber 1356 may form a sterile sliding seal about needle 308 such that pathogens or other contaminants are prevented from passing into cavity 1357. Needle 308 can move relative to top 1354 without disrupting the sterile seal therebetween. Cavity 1357 may be sterile or aseptic such that the inner surfaces of cavity 1357 and needle 308 are sterile. In another embodiment, cavity 1357 may be sterilized after needle 308 is inserted through opening 1358 and into cavity 1357. In alternative embodiments, rather than top 1354, a convoluted flexible (e.g., rubber) bellows or bladder member may form cavity 1357 and allow translation of cartridge 1302 relative to needle 308 (or vice versa). The flexible member also may seal or form cavity 1354 about needle 308 after sterilization.

Piston 1316 may be coupled to a translation mechanism 1366 that is configured to translate piston 1316 and cartridge 1302 in a direction toward second end 1306. The movement of piston 1316 toward second end 1306 causes piston 1316 to act against the contents within cartridge 1302 (e.g., drugs, medications), which ultimately transfers force against second end 1306 of cartridge 1302, causing cartridge 1302 to move along longitudinal axis 40. Translation mechanism 1366 may include a 12 mm motor with a five-stage gear reduction (360:1). Translation mechanism 1366 may have spring contacts that create an electrical connection with an associated printed circuit board (e.g., first electronic board 1402). The motor may be configured to generate a torque of about 136 mN*m at 36 rpm. These design parameters of the motor are merely exemplary, and any other suitable motor also may be utilized.

Translation mechanism 1366 may include a leadscrew mechanism coupled to piston 1316 that extends axially upon relative rotation about longitudinal axis 40. This telescoping leadscrew may have a 100 N output, a 20 mm stroke, and a 7°/45° buttress thread shape with a 0.75 mm pitch. The materials for the leadscrew mechanism may include acetal and polybutylene terephthalate. The leadscrew mechanism may extend within piston 1316 to reduce dead space behind piston 1316. While piston 1316 is shown in FIGS. 13 and 14 with longitudinally spaced threads, in some examples, such threads may not be present. In another exemplary embodiment (not shown), translation mechanism 1366 may include a manually engageable surface or member that is manually manipulated by a user to move piston 1316. For example, piercing system 1300 may include a cartridge or a plunger coupled to the back side of piston 1316. In another exemplary embodiment (not shown), translation mechanism 1366 may include a pneumatic or hydraulic drive member that is actuated or initiated by a user to move piston 1316. The drive member may be in the form of expanding bellows, an expanding bladder, an expanding diaphragm, or a sliding seal or piston, for example. The direct pneumatic or hydraulic pressure may provide the force required to move piston 1316.

Piercing system 1300 also includes a collar 1390 coupled or fixed to second end 1306. Collar 1390 may include a plurality of circumferentially spaced apart fingers 1392 that engage and surround neck 1310. Collar 1390 may be fixed, or otherwise coupled to second end 1306. Collar 1390 may include a wall 1390a that extends at least partially about neck 1310, the opening of second end 1306, cap 1312, septum 1314, and/or top 1354. Wall 1390a of collar 1390 may be positioned radially or laterally outward of neck 1310 and extend longitudinally past neck 1310, cap 1312, and septum 1314.

In the pre-activated state of piercing system 1300 shown in FIG. 13, an edge 1393 of collar 1390 may engage a corresponding radially or laterally inwardly extending cam, latch or actuation portion 1394 of a driver retainer member 1395. Retainer member 1395 may be slidable relative to collar 1390. Collar 1390 and retainer member 1395 may be configured such that in the pre-activated state or arrangement shown in FIG. 13, at least a portion of the cam or actuation portion 1394 of retainer member 1395 is positioned directly behind a retaining portion 1399 of a driver 1398 slidable within retainer member 1395. A wall 1391 of driver 1398 may extend into and through an end cap portion 1396 of retainer member 1395 and into an interior portion of retainer member 1395, and retaining portion 1399 of driver 1398 may extend radially outward from wall 1391. In some embodiments, wall 1391 of driver 1398 may be substantially cylindrical and retaining portion 1399 of driver 1398 may be a flange extending about an end of the wall 1391.

In the pre-activated state of piercing system 1300, an elastically deformed biasing or resilient member 1397 may be positioned between cap portion 1396 of retainer member 1395 and retaining portion 1399 of driver 1398. Biasing member 1397 may exert a force against driver 1398 in the pre-activated state of piercing system 1300 acting in the direction towards cartridge 1302. Biasing member 1397 may be any member effective in applying the force in the pre-activated state, and then releasing such force upon activation, as discussed below with reference to FIG. 14. In some embodiments, biasing member 1397 may be a conical or flat spring.

Needle 308 of fluid conduit 300 may be fixed or coupled to driver 1398 such that fluid conduit 300 moves with driver 1398. In the pre-activated state of piercing system 1300, needle 308 may be positioned within the sterile cavity 1357, but not through base 1355 of top 1354, septum 1314, and/or into cavity 1308 of cartridge 1302.

In some embodiments, in lieu of cavity 1357, needle 308 may be positioned within a plug when the piercing system 1300 is the pre-activated state. The plug may be a solid plug which is devoid of any holes, cavities, or openings, and which may be formed of a first rubber material. The first rubber material may be permeable to a sterilizing gas, such as, e.g., ethylene oxide or vaporized hydrogen peroxide. The first rubber material may include one or more of isoprene, ethylene propylene diene monomer (M-class) rubber (EPDM), and styrene-butadiene, among others. The permeability of the first rubber material to a sterilizing gas may allow needle 308, when disposed within the plug, to be sterilized before use. The plug may be molded about needle 308, so that needle 308 is impaled into the plug.

To move piercing system 1300 from the pre-activated state of FIG. 13, translation mechanism 1366 may be activated to move piston 1316 towards second end 1306 and translate cartridge 1302 along longitudinal axis 40 toward driver 1398. Because the needle 308 is not yet in fluid communication with cartridge 1302, activation of translation mechanism 1366 applies a pressure against the fluid contained in cartridge 1302, which is then applied to cartridge 1302 itself. This pressure also causes edge 1393 to push against and deflect actuation portion 1394 radially outward. Without actuation portion 1394 blocking its path, retaining portion 1399 and needle 308 are moved toward cartridge 1302 by the expansion of biasing member 1397. Driver 1398 may be coupled to flange 204 of carrier 202, and thus, this movement of driver 1398 toward cartridge 1302 also may move carrier 202 in the same direction. This movement corresponds to the movement of carrier 202 relative to housing 3 in FIGS. 6 and 7, which enables protrusion 330 to clear impediment 600 to inject needle 306.

The movement of needle 308 toward second end 1306 of cartridge 1302 also causes needle 308 to pierce through base 1355 of top 1354, septum 1314, and cavity 1308, into fluid communication with the contents of cartridge 1302. Once needle 308 is in fluid communication with cartridge 1302, further movement of piston 1316 toward second end 1306 urges fluid through needle 308 and a remainder of fluid conduit 300. In some embodiments, piercing system 1300 may be configured such that, after activation, no more of needle 308 than the portion that was already positioned within sterile cavity 1357 extends into cavity 1308. This may help prevent contamination of the contents of cartridge 1302 with non-sterile portions of needle 308.

Biasing member 1397 may be configured to expand such that fluid conduit 300 pierces top 1354 and/or septum 1314 at a high speed, such as at a speed of at least about 10 mm/sec, or at least about 40 mm/sec. The relatively quick piercing of top 1354 and/or septum 1314 via biasing member 1397 may help prevent leakage of the contents of cavity 1308 which may be under pressure via piston 1316.

After drugs have been delivered to the user via needle 306, needle 306 may be automatically withdrawn from the user. Referring to FIGS. 12-14, translation mechanism 1366 may be operated in a reverse mode such that the rotation of the lead screw is in an opposite direction compared to the insertion step. This counter-rotation may cause piston 316 to move back toward first end 1304, and also cause cartridge 1302 to move in an opposite direction along axis 40 (as compared to during fluid delivery and insertion of needle 306). The movement of cartridge 1302 in the opposing direction may cause ramp 1500 in FIG. 12 (which is attached to wall 1391) to push against ramp 243 of stop 240. This may cause stop 240 to deflect about its fixed end 241 in the direction of arrow 240a, and allow shuttle 340 to move from its second position to its third position to retract needle 306 as set forth above. In this way, withdrawal and insertion of the needle into a patient can both be accomplished with a single spring within the device.

It is further contemplated that fluid conduit 300 may be the only fluid conduit of auto-injector 2 configured to be in fluid communication with cartridge 1302. Thus, drugs from cartridge 1302 may be deployed only through fluid conduit 300 and into the user during normal operation of auto-injector 2. Additionally, needle 306 may be the only needle of auto-injector 2 configured to be deployed into a patient. In this way, a single piece of metal or plastic can be used to carry the fluid from cartridge 1302 to a patient.

FIGS. 14A-14D depict an exemplary configuration of an outer screw 1367 that may be incorporated in translation mechanism 1366 and that may interface with piston 1316. The outer screw 1367 may generally comprise an upper portion 1378, a lower portion 1381, and a flange portion 1375. The flange portion 1375 may have a first surface 1376 and a second surface 1377 and separates the upper and lower portions 1378, 1381 of the outer screw 1367. A conduit 1372 extends the length of the outer screw 1367 and generally has a uniform diameter.

The upper portion 1378 may generally comprise one or more projections 1379 that protrude from the first surface 1376 of the flange portion 1375. In some embodiments, the one or more projections 1379 may be formed or partially formed by ejector pins during a molding process and are generally configured to fit into corresponding features of an output gear of a motor assembly. It should be understood that there may be any number of the one or more projections 1379 and that the one or more projections 1379 may be of any suitable shape and/or size. In some embodiments, there are at least two projections, four projections, or five projections.

The lower portion 1381 may generally comprise a nose region 1382 and one or more protrusions 1383 that extend radially outward from the exterior surface 1373 of the conduit 1372 and longitudinally from the second surface 1377 of the flange portion 1375. Each of the one or more protrusions 1383 generally comprises a tip 1384 and a surrounding side edge 1385. The one or more protrusions 1383 are configured to interlock with corresponding features found on the interior of piston 1316. The side edge 1385 features a chamfered design and the tip is generally tapered to facilitate nesting of the outer screw 1367 into the piston 1316 during the assembly process. The nose region 1382 extends from the tips 1384 of the one or more protrusions 1383 to the end of the lower portion 1381. The nose region 1382 may include a chamfer toward the end of the lower portion 1381 to further facilitate nesting of the outer screw 1367 within piston 1316.

The configuration of the outer screw 1367, including dimensions and tolerances thereof may be selected to balance the ease of assembly, the strength and durability of the coupling mechanisms, and the degree of deformation of the piston 1316 when under external pressure. For example, nose region 1382 may be configured to facilitate insertion of outer screw 1367 into piston 1316. Moreover, tips 1384 may be shaped and sized so as to facilitate mating with features of piston 1316. For example, tips 1384 may generally be triangular in shape with smooth edges, which may facilitate advancing of the protrusions 1383 past internal features of piston 1316. By facilitating insertion of outer screw 1367 into piston 1316 during assembly, dislodging of piston 1316 from its position within cartridge 1302 may be inhibited and an integrity of the seal of cartridge 1302 may be maintained during assembly.

The length L1 of the outer screw 1367 may range from about 12 mm to about 16 mm, including all sub-ranges and values there-between. In some embodiments, the length L1 of the outer screw 1367 may range from about 12.5 mm to about 15.5 mm; from about 12.75 mm to about 15.25 mm; from about 13 mm to about 15 mm; or from about 14 mm to about 15 mm. In certain embodiments, the length L1 of the outer screw 1367 may be about 14.42 mm, about 14.48 mm, about 14.54 mm, about 14.60 mm, about 14.66 mm, about 14.72 mm, about 14.78 mm, about 14.84 m, about 14.90 mm; or about 14.96 mm.

The length L2 of the lower portion 1381 of the outer screw 1367 may range from about 8.0 mm to about 13.0 mm, including all sub-ranges and values there-between. In some embodiments, the length L2 of the lower portion 1381 of the outer screw 1367 may range from about 8.0 mm to about 12.0 mm; from about 8.0 mm to about 11 mm; from about 8.0 mm to about 10.5 mm; from about 9.0 mm to about 12.0 mm; from about 9.0 mm to about 11.0 mm, from about 9.0 mm to about 10.5 mm; from about 9.5 mm to about 10.5 mm; or from about 9.9 mm to about 10.2 mm. In certain embodiments, the length L2 of the lower portion 1381 of the outer screw 1367 may be about 9.62 mm, about 9.68 mm, about 9.74 mm, about 9.80 mm, about 9.86 mm, about 9.92 mm, about 9.98 mm, about 10.04 mm, about 10.10 mm, about 10.16 mm, about 10.22 mm, about 10.28 mm, about 10.34 mm, or about 10.40 mm.

The length L3 of the nose region 1382 of the lower portion 1381 of the outer screw 1367 may range from about 0.5 mm to about 7.0 mm, including all sub-ranges and values there-between. In some embodiments, the length L3 of the nose region 1382 of the lower portion 1381 of the outer screw 1367 may range from about 1.0 mm to about 4.5 mm; from about 1.5 mm to about 4.0 mm; from about 1.5 mm to about 3.5 mm; from about 2.0 mm to about 5.0 mm; from about 2.5 mm to about 5.0 mm; or from about 3.0 mm to about 5.0 mm. In certain embodiments, the length L3 of the nose region 1382 of the lower portion 1381 of the outer screw 1367 may be about 5.41 mm, about 5.47 mm, about 5.53 mm, about 5.59 mm, about 5.65 mm, about 5.71 mm, about 5.77 mm, about 5.83 mm, about 5.89 mm, about 5.95 mm, about 6.01 mm, about 6.07 mm, about 6.13 mm, about 6.19 mm, about 6.25 mm, about 6.31 mm, or about 6.37 mm. The tolerances of the lengths described herein may be about 0.2 mm or less, about 0.15 mm or less, about 0.12 mm or less, about 0.10 or less, about 0.08 mm or less, or about 0.06 mm or less.

The threaded region 1380 of conduit 1372 may generally be characterized by several diameter measurements. For example, the threaded region 1380 may be defined by a minor diameter measured between two opposing crests of the threading, a major diameter measured between two opposing roots of the threading, or a pitch diameter measured between the midpoints of the threading on either side of the threaded region 1380. In some embodiments, the threaded region 1380 of the conduit 1372 may have a minor diameter ranging from about 6.75 mm to about 7.60 mm, a major diameter ranging from about 7.75 mm to about 8.40 mm, and/or a pitch diameter ranging from about 7.00 mm to 8.15 mm, including all sub-ranges and values there-between.

The minor diameter may range from about 6.75 mm to about 7.50 mm; from about 6.75 mm to about 7.40 mm, from about 6.75 mm to about 7.30 mm; from about 7.00 mm to about 7.50 mm; from about 7.00 mm to about 7.40 mm; from about 7.00 mm to about 7.30 mm; from about 7.15 mm to about 7.50 mm; from about 7.15 mm to about 7.40 mm; or from about 7.15 mm to about 7.30 mm. The major diameter may range from about 7.75 mm to about 8.30 mm; from about 7.75 mm to about 8.20 mm, from about 7.75 mm to about 7.10 mm; from about 7.90 mm to about 8.30 mm; from about 7.90 mm to about 8.20 mm; from about 7.90 mm to about 8.10 mm; from about 8.05 mm to about 8.30 mm; from about 8.05 mm to about 8.25 mm; from about 8.05 mm to about 8.20 mm; from about 8.05 mm to about 8.15 mm; or from about 8.08 mm to about 8.15 mm. The pitch diameter may range from about 7.00 mm to about 8.10 mm; from about 7.25 mm to about 8.00 mm, from about 7.25 mm to about 7.90 mm; from about 7.25 mm to about 7.75 mm; from about 7.25 mm to about 7.60 mm; from about 7.45 mm to about 8.10 mm; from about 7.45 mm to about 8.00 mm; from about 7.45 mm to about 7.90 mm; from about 7.45 mm to about 7.75 mm; from about 7.45 mm to about 7.60 mm; or from about 7.50 mm to about 7.60 mm. In certain embodiments, the minor diameter may be about 6.93 mm, about 7.00 mm, about 7.07 mm, about 7.14 mm, about 7.21 mm, about 7.28 mm, about 7.35 mm, about 7.42 mm, or about 7.49 mm; the major diameter may be about 7.78 mm, about 7.86 mm, about 7.94 mm, about 8.02 mm, about 8.10 mm, about 8.18 mm, about 8.26 mm, or about 8.34 mm, and the pitch diameter may be about 7.20 mm, about 7.26 mm, about 7.32 mm, about 7.38 mm, about 7.44 mm, about 7.50 mm, about 7.56 mm, about 7.62 mm, about 7.68 mm, about 7.74 mm, about 7.80 mm, or about 7.86 mm.

### Locking Component (Drop Pin)

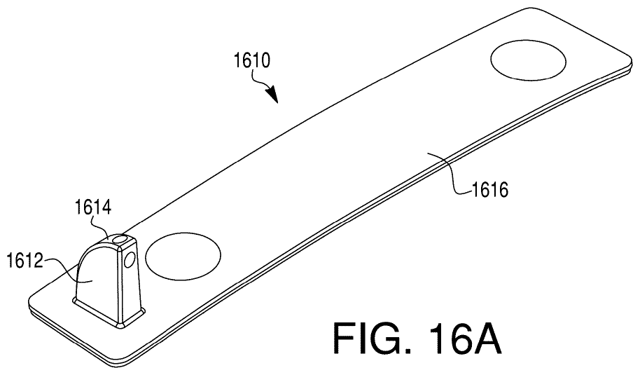

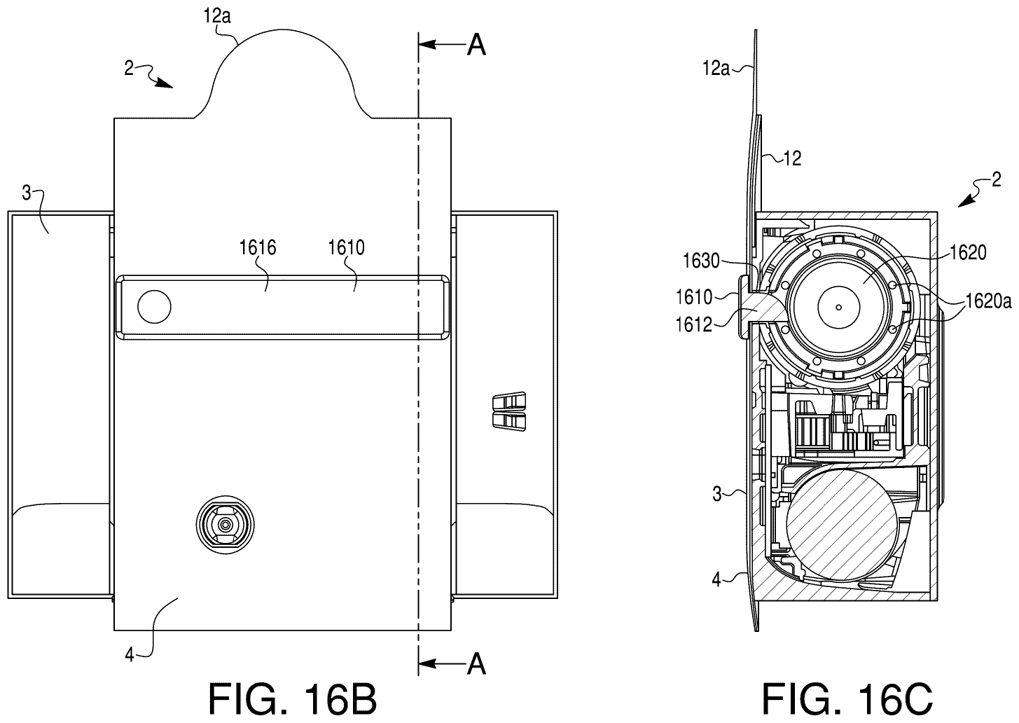

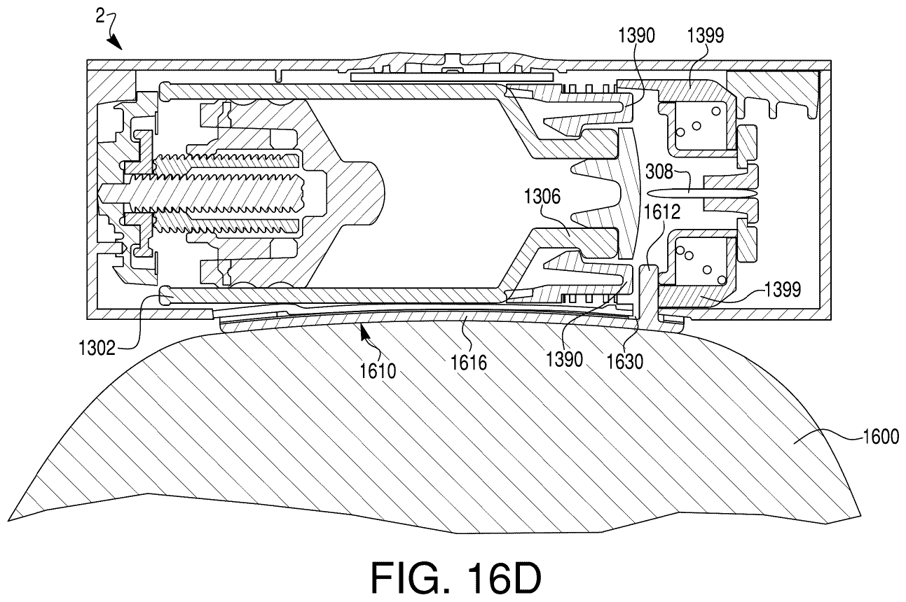

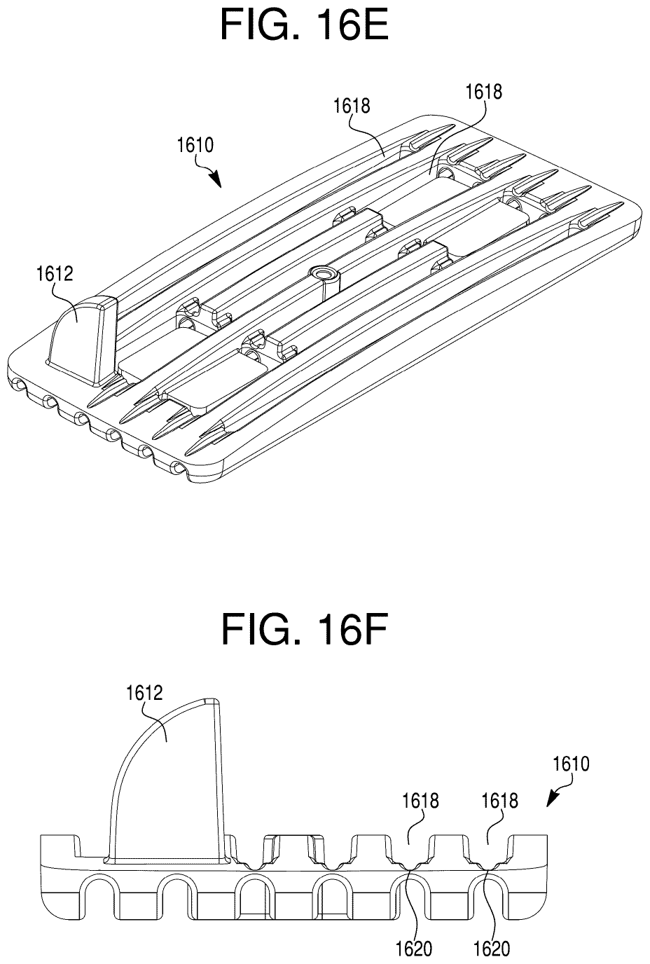

Referring to FIGS. 16A-16D, auto-injector 2 may include a locking component 1610. As shown in FIG. 16A, locking component 1610 may include a lock (e.g., a protrusion) 1612 having a curved surface 1614 and may further include a cover portion 1616. Cover portion 1616 may be shaped to conform to tissue-engaging surface 4 of auto-injector 2, as shown in FIG. 16D. Cover portion 1616 and tissue-engaging surface 4 may be concave to receive an anatomical portion 1600 of a user. Anatomical portion 1600 may be, for example, a thigh, hip, arm, posterior, or any other area of the body suitable for injection. Lock 1612 may be connected to cover portion 1616. In some embodiments, locking component 1610 may be formed as a single piece, such that lock 1612 and cover portion 1616 are integrally connected. Locking component 1610 may be formed from any suitable rigid or semi-rigid material. Locking component 1610 may, for example, be formed of acrylonitrile butadiene styrene (ABS) and may further have a frosted clear appearance indicating that locking component 1610 is disposable.

As shown in FIGS. 16B-16C, locking component 1610 may be disposed on or adjacent tissue-engaging surface 4 of the auto-injector 2 such that lock 1612 may extend into auto-injector 2. Locking component 1610 may further be disposed on or adjacent a liner 12a which may initially cover adhesive patch 12 prior to use of auto-injector 2. Locking component 1610 may be positioned relative to liner 12a such that upon removal of liner 12a from adhesive patch 12, locking component 1610 may also be removed from tissue-engaging surface 4. Lock 1612 may extend into auto-injector 2 via lock opening 1630 formed in tissue-engaging surface 4. When locking component 1610 is disposed on or adjacent tissue-engaging surface 4, cover portion 1616 may be attached to tissue-engaging surface 4 via an adhesive disposed between cover portion 1616 and tissue-engaging surface 4. Locking component 1610 may be disposed on or adjacent tissue-engaging surface 4 such that it is selectively removable by a user.

Referring to FIGS. 16C-D, when locking component 1610 is disposed on or adjacent tissue-engaging surface 4, lock 1612 may extend into auto-injector 2 such that it prevents movement of one or more internal mechanisms of auto-injector 2. For example, when locking component 1610 is disposed on or adjacent auto-injector 2, lock 1612 may extend into auto-injector 2 such that lock 1612 engages with one or more internal components of auto-injector 2, preventing those components from moving and/or being activated.

Referring to FIG. 16D, when locking component 1610 is disposed on or adjacent tissue-engaging surface 4, lock 1612 may extend into auto-injector 2 such that it is disposed within piercing system 1300. As described herein previously, collar 1390 may be coupled or fixed to second end 1306 of cartridge 1302. As also described herein previously, when piercing system 1300 is moved from the pre-activated state, cartridge 1302 and consequently collar 1390 may translate in a direction parallel to a longitudinal axis of cartridge 1302 toward retaining portion 1399. When lock 1612 extends into auto-injector 2 and is adjacent to collar 1390, lock 1612 may prevent cartridge 1302 from translating toward retaining portion 1399 or otherwise prevent cartridge 1302 and collar 1390 from applying a force against actuation portion 1394. Thus, even if the motor were somehow activated while lock 1612 is disposed in its locking position, fluid communication between needle 308 and cartridge 1302 could not be established and needle 306 could not be deployed outside of housing 3. Furthermore, when in the locking position, lock 1612 may prevent the movement of cartridge 1302 toward needle 308, thereby preventing deflection of actuation portion 1394 and consequently preventing retaining portion 1399 and needle 308 from moving toward cartridge 1302. In the event of auto-injector 2 being dropped or being subject to vibrations, lock 1612 may further prevent piercing system 1300 from being moved from the pre-activated state and consequently may prevent cartridge 1302 from being pierced by the needle 308.

When locking component 1610 is disposed on or adjacent tissue-engaging surface 4, locking component 1610 may additionally serve as a spacer between a user's skin and tissue-engaging surface 4. For example, locking component 1610 may have a thickness such that touch sensor 1410, described in greater detail hereinafter, is unable to detect the user's skin thereby avoiding inadvertent activation of auto-injector 2. Locking component 1610 may have a thickness, for example, from about 1 mm and about 5 mm, or about 3 mm.

Accordingly, locking component 1610 may act as an effective safety mechanism to prevent inadvertent activation of auto-injector 2. When locking component 1610 is disposed on or adjacent the tissue-engaging surface 4, lock 1612 may prevent various internal components of auto-injector 2 from moving. In the event auto-injector 2 is dropped on the floor prior to use, for example, locking component 1610 may prevent inadvertent piercing of cartridge 1302 and/or inadvertent initiation of an injection sequence. Locking component 1610 may also prevent such movement and/or inadvertent initiation of an injection sequence should auto-injector 2 be subjected to vibration during transport.

If a user wishes to use and/or is ready to use auto-injector 2, the user may separate locking component 1610 from tissue-engaging surface 4, thereby removing lock 1612 from lock opening 1630. The user may, for example, peel cover portion 1616 off of tissue-engaging surface 4. Alternatively, the user may peel liner 12a away from adhesive patch 12, thereby removing locking component 1610 from tissue-engaging surface 4. When separating locking component 1610 from tissue-engaging surface 4, curved surface 1614 may allow lock 1612 to rock within lock opening 1630, thereby allowing lock 1612 to be easily removed from lock opening 1630. With lock 1612 removed from lock opening 1630, auto-injector 2 may be in a state in which it is ready to be used such that, e.g., an injection sequence may be initiated.

FIGS. 16E and 16F depict locking component 1610 according to some embodiments. As shown in FIGS. 16E and 16F, locking component 1610 may have an increased width (relative to the depiction of locking component 1610 in FIGS. 16A-16C) to ensure locking component 1610 extends over touch sensor 1410 when locking component 1610 is disposed on auto-injector 2. Moreover, locking component 1610 may have a ribbed structure and may include air gaps or recesses 1618 and hinges 1620. Air gaps 1618 may inhibit conduction of a capacitive field between the user's skin and touch sensor 1410 when the auto-injector 2 is placed near the user with locking component 1610 in place. Hinges 1620 may allow locking component 1610 to flex when locking component 1610 is peeled from auto-injector 2. In some embodiments, a removable cover other than and/or separate from locking component 1610 may extend over touch sensor 1410 to inhibit inadvertent skin detection.

### Electronics

FIG. 4A shows a control system 1400 of auto-injector 2. Control system 1400 may include components positioned on a first electronics board 1402 and a second electronics board 1404, and also may include a power source 1406. First electronics board 1402 may include a controller 1408, an activating switch 1409, a touch sensor 1410, a needle insert switch 1412, and an emitter 1414. Second electronics board 1404 may include a detector 1416, an audio module 1418, a visual module 1420, and a haptic module 1422. Though FIG. 4A depicts audio module 1418, visual module 1420, and haptic module 1422 as included in second electronics board 1404, in some embodiments, one or more of the foregoing modules may be included on the first electronics board 1402. One or more of the components of first electronics board 1402 and second electronics board 1404 may be operatively coupled to controller 1408, and powered by power source 1406. Controller 1408 also may be operatively coupled to translation mechanism 1366, and may be configured to control operation of translation mechanism 1366 to initiate and control needle insertion and retraction as set forth above. Translation mechanism 1366 may be coupled to first electronics board 1402 via one or more spring contacts during a final assembly step where cartridge 1302 is inserted into housing 3. As described herein previously, translation mechanism 1366 may include a motor, gearing, and a leadscrew mechanism.

The majority of the assembly of auto-injector 2 may occur, e.g., on an assembly line at a manufacturing facility. Then, two device halves (or portions) may be shipped to a drug filling or final assembly facility. Indeed, the two separate portions 1490 and 1492 need not be the same size, as illustrated in FIG. 4B. Once a drug vial, e.g., cartridge 1302, is filled with a drug or other medicament, cartridge 1302 may be assembled with a remainder of auto-injector 2. For example, the two device halves (portions 1490 and 1492) may be assembled together with the filled drug cartridge 1302 therein. In one example, portion 1490 and translation mechanism 1366 may be snapped in place behind cartridge 1302. Portion 1490 may be part of housing 3 including a base or module configured to contain translation mechanism 1366 and its associated electronics. Portion 1492 may be a part of housing 3 containing substantially all of the other components described herein, including, e.g., the needle mechanism, sterile connector, and piercing mechanisms described herein. In this example, an electrical connection of the motor of translation mechanism 1366 must be made during the snapping of translation mechanism 1366 behind cartridge 1302 (i.e., during the assembly step where portions 1490 and 1492, and cartridge 1302 are combined to form a complete and functional auto-injector 2). To accommodate such an electrical connection, the drivetrain of translation mechanism 1366 may include one or more spring contacts 1494 (referring to FIG. 4C) that will contact pads 1495 (also referring to FIG. 4C) on the first electronics board 1402 upon assembly. Though not depicted in FIG. 4C, the drivetrain of translation mechanism 1366 may include additional spring contacts that may contact additional pads on the first electronic board 1402 upon assembly. Such additional spring contacts and additional pads may serve to connect additional components of the translation mechanism 1366, such as a tachometer, a motor encoder, or any other sensors or devices, to the first electronics board 1402. Thus, the connection of translation mechanism 1366 to first electronics board 1402 (including controller 1408) may be made without any loose wires or other similar structures.

Such an assembly process may be relatively simpler than simpler devices (e.g., auto-injectors) with relatively more complex final assembly processes. As a result, the contemplated assembly process described herein may lead to a reduction of labor costs.

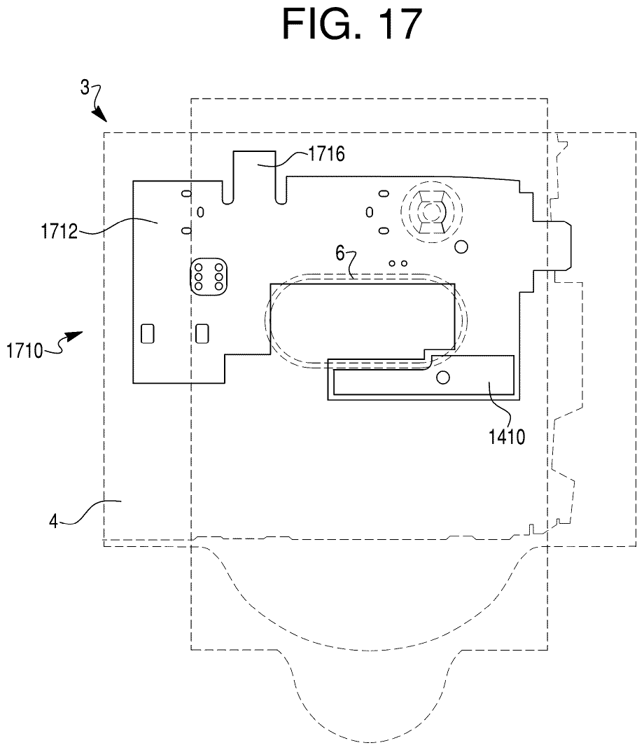

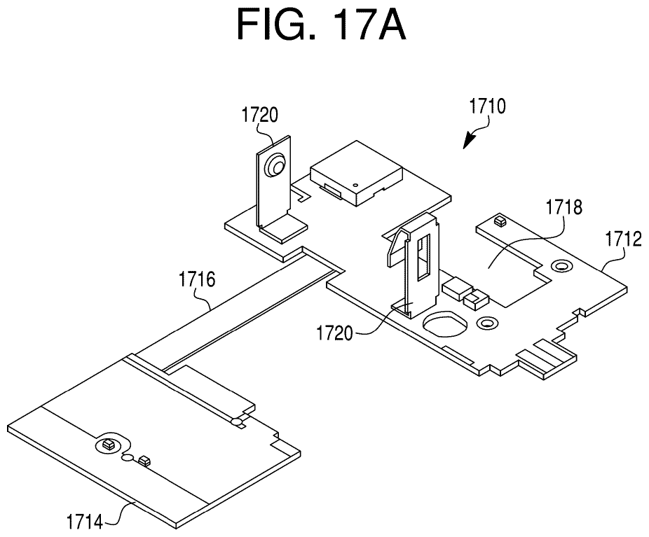

In some embodiments, auto-injector 2 may include a single (i.e., only or exactly one) electronics board 1710 as shown in FIGS. 17 and 17A, on which the components of control system 1400 described herein previously may be positioned. As shown in FIG. 17A, electronics board 1710 may include a first board segment 1712 and a second board segment 1714. The first board segment 1712 and the second board segment 1714 may be physically and electrically connected via a flexible segment 1716. Flexible segment 1716 may be, for example, a ribbon cable, a flexible conductive substrate, or the like. In some embodiments, flexible segment 1716 may be formed of fiberglass board that is machined thinly enough to flex, and is sometimes referred to as “semi-flex.” In some embodiments, flexible segment 1716 may be formed of a flexible polymer. The flexible polymer may be formed by a process sometimes referred to as “rigid-flex” in which a sandwich of a first portion fiberglass, flexible polymer, and second portion of fiberglass is first formed. The first and second portions of fiberglass may be subsequently removed to leave the thin flexible polymer portion.

Electronics board 1710 may include one or more brackets 1720 for mounting or otherwise securing electronics board 1710 to an interior of auto-injector 2. The first board segment 1712 may further include a cutout 1718. The cutout 1718 may be positioned such that first board segment 1712 may be positioned to allow the needle to pass through cutout 1718 when deployed.

In some embodiments, first board segment 1712 may correspond to first electronics board 1402 and second board segment 1714 may similarly correspond to second electronics board 1404, each as described herein previously. By connecting first board segment 1712 and second board segment 1714 via flexible segment 1716, first board segment 1712 may be positioned adjacent to tissue-engaging surface 4 of the auto-injector 2 whereas second board segment 1714 may be positioned on an opposite side of auto-injector 2 toward upper portion 30 of housing 3. Accordingly, the single electronics board 1710 may be utilized to both connect components located toward tissue-engaging surface 4 and connect components located toward upper portion 30. Such a configuration may allow for ease of assembly of the auto-injector 2 by obviating a need for complex wiring or soldering.

As shown in FIG. 17, electronics board 1710 may be positioned within housing 3. First board segment 1712 may be positioned adjacent to tissue-engaging surface 4 whereas second board segment 1714 may be positioned on an opposite side of auto-injector 2 (e.g. behind first board segment 1712 in FIG. 17). Flexible segment 1716 may be flexed or folded to maintain a connection between first board segment 1712 and second board segment 1714 in such positions.

Touch sensor 1410 may be incorporated in or on first board segment 1712 of electronics board 1710. To allow for adequate detection of a user's skin, touch sensor 1410 and first board segment 1712 may be located close to tissue-engaging surface 4 of housing 3. Tissue-engaging surface 4 of housing 3, or a portion thereof adjacent to touch sensor 1410, may be sufficiently thin such that an electric field of detectable magnitude may form between touch sensor 1410 and a user's skin. In some embodiments, the portion of tissue-engaging surface 4 adjacent touch sensor 1410 may be less than about 2 mm, about 1 mm, or less than about 1 mm. Further, the portion of tissue-engaging surface 4 adjacent touch sensor 1410 may be made from a solid material, such as plastic. By forming the portion of tissue-engaging surface 4 adjacent touch sensor 1410 from a solid material, as opposed to a ribbed, cored, or hollow material, a dielectric constant between the user's skin and touch sensor 1410 may optimize a responsiveness of touch sensor 1410.

Additionally, touch sensor 1410 may be positioned in or on electronics board 1710 so as to be adjacent to or near opening 6 through which the needle may be deployed. By positioning touch sensor 1410 adjacent to or near opening 6, a likelihood that touch sensor 1410 may detect a user's skin when auto-injector is positioned appropriately is increased. Furthermore, a curvature of tissue-engaging surface 4 may decrease the likelihood that touch sensor 1410 may falsely interpret a flat surface such as a tabletop to be a user's skin by creating a space between touch sensor 1410 and the flat surface.

By incorporating touch sensor 1410 in or on electronics board 1710, a need for one or more wires and/or other circuitry connecting touch sensor 1410 to a separate electronics board may be eliminated. Assembly of the auto-injector 2 may thereby be simplified and a cost of the auto-injector may be reduced.

As electronics board 1710 may be located adjacent to tissue-engaging surface 4, electronics board 1710 may include a cutout to allow the needle to be deployed through electronics board 1710 and subsequently through opening 6. Further, electronics board 1710 may be positioned such that touch sensor 1410 is directly adjacent opening 6 and no gap exists between an edge of touch sensor 1410 and opening 6. Alternatively, electronics board 1710 may be positioned such that a gap exists between an edge of touch sensor 1410 and opening 6 and the gap has a maximum of width of 5 mm, 2 mm, or 1 mm, for example.

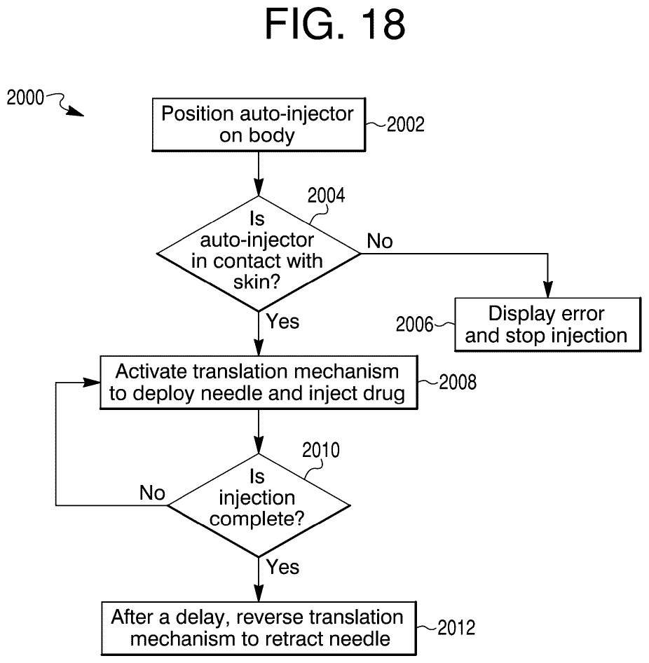

Controller 1408 may be configured to accept information from the system and system components described above, and process the information according to various algorithms to produce control signals for controlling internal mechanisms of auto-injector 2, including translation mechanism 1366. Examples of such algorithms are described hereinafter with reference to FIGS. 18 and 20-23. The processor may accept information from the system and system components, process the information according to various algorithms, and produce information signals that may be directed to audio module 1418, visual module 1420, haptic module 1422, or other indicators of, e.g., second electronics board 1404, in order to inform a user of the system status, component status, procedure status or any other useful information that is being monitored by the system. The processor may be a digital IC processor, analog processor or any other suitable logic or control system that carries out the control algorithms.

As discussed above with respect to FIGS. 3A and 3B, activating switch 1409 may be a mechanical plunger-type switch that extends away from tissue-engaging surface 4 of auto-injector 2. Activating switch 1409 may include an electrical circuit that is complete unless activating switch 1409 is depressed. For example, when auto-injector 2 is attached to a user's skin, switch 1409 may be depressed, breaking the electrical circuit, and indicating to controller 1408 that auto-injector 2 should be activated. In order to conserve power, the components of auto-injector 2 may be in an idle or sleep mode until switch 1409 is activated. In yet another example, auto-injector 2 may not be powered at all until switch 1409 is activated, and deactivation of switch 1409 may cut off power to auto-injector 2 entirely. While a mechanical plunger-type switch is disclosed, any other suitable mechanism for activating auto-injector 2 may be utilized, including, e.g., a button depressed by the user, voice signals, and a wireless signal from another electronic device, among others.

Touch sensor 1410 may be configured to help controller 1408 determine whether auto-injector 2 is properly deployed on the skin of a user. In one example, touch sensor 1410 may be a capacitive sensing electrode or any other device configured to differentiate contact with skin versus other materials, such as, e.g., wood, plastic, metal, or another material. When skin is in the proximity of the capacitive sensing electrode, a signal indicative of such contact may be sent to controller 1408. Thus, touch sensor 1410 may serve to verify that auto-injector 2 is properly placed on a user's skin, even if switch 1409 is depressed. Touch sensor 1410 may include a capacitive sensing electrode coupled to first electronics board 1402 and also to an interior of housing 3. Housing 3 and adhesive patch 12 may act as an overlay (insulator) that acts as a dielectric between the skin of the user and the capacitive sensing electrode. Alternatively, touch sensor 1410 may be incorporated in or on electronics board 1710, as described herein previously, such that the capacitive sensing electrode is also incorporated in or on electronics board 1710, Contact of portions of housing 3 and/or adhesive patch 12 near the capacitive sensing electrode may cause the capacitance of the electrode to increase, for example, by about 1 to about 10 pF, indicating placement of auto-injector 2 on a skin surface.

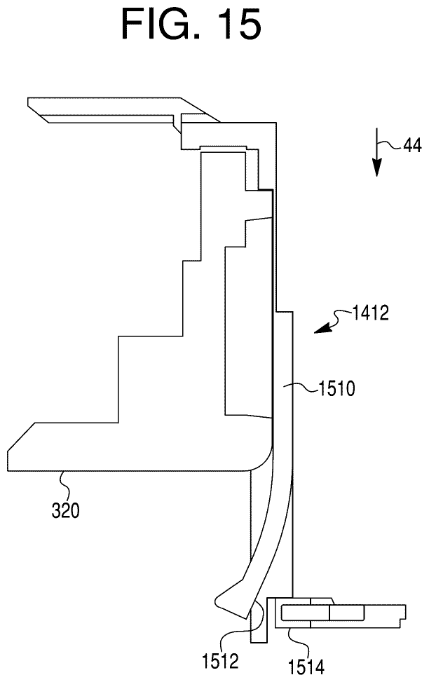

Needle insert switch 1412 may be configured to send a signal to controller 1408 that needle 306 is deployed within a user. For example, referring to FIG. 15, needle insert switch 1412 may include a curved cantilever 1510 including a first contact 1512. Needle insert switch 1412 also may include a second contact 1514. First contact 1512 may be placed into electrical contact with second contact 1514 when needle 306 is deployed into the user. During deployment of needle 306, driver 320 may move downward along axis 44 and deflect curved cantilever 1510 and first contact 1512 toward second contact 1514. When first contact 1512 and second contact 1514 connect to one another, a signal may be sent to controller 1408 indicating that needle 306 has been successfully deployed into the user. The separation of first contact 1512 and second contact 1514 may indicate that needle 306 has been retracted from the user.

Emitter 1414 and detector 1416 may operate as an optical interruption sensor, or photo-interrupter in order to allow controller 1408 to determine a state of auto-injector 2. Emitter 1414 may be a light emitting diode (LED) or other suitable light emitter, and detector 1416 may be, e.g., a phototransistor configured to receive light emitted by emitter 1414. In one example, emitter 1414 may emit infrared light, although other suitable wavelengths of light also may be used. The use of infrared light may help reduce interference from external light.

As shown in FIG. 13B, emitter 1414 and detector 1416 may be arranged across from one another within housing 3 to enable a beam of light 1430 to pass from emitter 1414, through cartridge 1302, to detector 1416. Cartridge 1302, and any fluid contained therein may be at least partially transparent to beam 1430 so that beam 1430 may pass through cartridge 1302 and its contents. As piston 1316 is moved toward second end 1306 during drug delivery (referring to FIGS. 13 and 14), piston 1316, and in particular a shoulder of piston 1316, may interrupt beam 1430. When detector 1416 fails to sense beam 1430, a signal may be sent to controller 1408, which may interpret the signal to indicate an end of an injection (e.g., that all of the drug contained within cartridge 1302 has been expelled). In some examples, the refraction path of beam 1430 may be considered when positioning emitter 1414 and detector 1416 relative to one another. For example, beam 1430 may be refracted as it passes through cartridge 1302 and any liquid contained therein, and emitter 1414 and detector 1416 may be offset from one another accordingly. Additionally, emitter 1414 and detector 1416 may be offset from a center of housing 3 so that the shoulder of piston 1316 may block beam 1430. In at least some examples, an optical interruption sensor or similar mechanism may help avoid false positives in the event of a drive train failure. That is, the optical switch may help controller 1408 determine that an injection was not completed with greater accuracy than other mechanisms.

Audio module 1418 may include a speaker or the like to provide audio feedback to the user. Openings in housing 3 may facilitate the travel of sound from audio module 1418 to the user. Audio module 1418 may generate a tone or other sound at the start and at the end of injection, and/or to indicate any other benchmark during the injection, such as an error, for example. Visual module 1420 may include one or more LEDs or similar devices to provide visual feedback to the user. Visual module 1420 may include different colored LEDs to provide various messages to the user. For example, a plurality of blue LEDs arranged in a ring could be used to display progress of the injection over time, one or more green LEDs could be used to display completion of the injection, and a red LED could be used to display an error to the user. Any other suitable colors, combinations, and/or numbers of LEDs may be used in various examples. For example, a combination of red, blue, and purple LEDs may be utilized. In one arrangement, eight LEDs may be arranged in a circle having a diameter of about 26.5 mm, or a diameter from about 10.0 mm to about 40.0 mm. It is to be understood that this exemplary quantity and positioning of LEDs is not intended to be limiting and any quantity and/or positioning of LEDs may be used. The LEDs may be activated sequentially around the circle to indicate progress of an injection (e.g., in a progress ring arranged in a similar manner as a clock-see, for example, LEDs 52 on FIG. 4B). Controller 1408 also may be configured to receive feedback from various sensors, and rescale a speed that various LEDs are activated based on feedback from the sensors. For example, the LEDs in the progress ring may be activated in three or more operation phases including, e.g., an injection sequence activation phase, an injection phase, and a retraction phase. Those of ordinary skill in the art will recognize that auto-injector 2 may have more or less than the above-described three operation phases. There may be an expected time for completing each phase, but there also may be some variability in the actual times experienced during any of the aforementioned operation phases of auto-injector 2. An algorithm may be utilized to help avoid the premature activation of LEDs, for example, when a certain phase finishes earlier than expected, or to have progress along the ring stopped when a certain phase takes longer than expected. At any given point, the algorithm may divide the remaining estimated time for completion of drug delivery by the number of unactivated LEDs in the progress ring, to determine a rate at which the remaining LEDs in the progress ring should be activated.

For example, before the injection sequence activation phase, the LEDs may be activated at a rate equal to the estimated time of the entire drug delivery process (e.g., the estimated time to complete all of injection sequence activation phase, the injection phase, and the retraction phase) divided by the total number of unactivated LEDs in the progress ring. Stated differently, the estimated time of the entire drug delivery process may be divided by a number that is the total number of LEDs in the progress ring less any already-activated LEDs. Thus, if, for example, one LED is already activated, the estimated time of the entire drug delivery process may be divided by one less than the total number of LEDs in the progress ring.

After completion of the injection sequence activation phase, the LEDs may be activated at a rate equal to the sum of estimated times for completing the remaining phases (e.g., the injection phase and the retraction phase) divided by the number of unlit LEDs in the progress ring. After completion of the injection phase, the LEDs may be activated at a rate equal to the estimated time to complete the retraction phase, divided by the number of unlit LEDs.

In some embodiments, subsets of LEDs may be used to indicate progress of injection phases. For example, in embodiments having eight LEDs positioned on a housing of auto-injector 2, a first LED may be illuminated to indicate needle insertion. The second through seventh LEDs may then be illuminated sequentially to indicate a progress of the injection phase. Lastly, the eighth LED may be illuminated to indicate needle retraction. While an exemplary configuration of the LEDs and corresponding logic has been described, it should be understood that the quantities of LEDs for each phase of an injection process may be varied as desired.

Visual module 1420 also may include a display screen, touch screen, or other suitable device to provide one-way or two-way communication with the user. Visual module 1420 may be visible by the user from outside of housing 3 via a window in housing 3. Haptic module 1422 may include, e.g., a haptic motor configured to generate vibrations that can be felt by the user. Vibrations may signal the start and the end of an injection, and/or may help provide additional information to a user.

Controller 1408 may be coupled to a wireless communication module and an antenna. The wireless communication module may be configured to transmit data from controller 1408 to, e.g., a mobile device, computer, cell phone, or the like. The wireless communication module may be configured to transmit information over one or more wireless modalities, such as, e.g., Bluetooth, Bluetooth low energy (BLE), near-field communication (NFC), infrared, cellular networks, and wireless networks, among others. The antenna may be any suitable device configured to assist the wireless communication module in data transmission and/or amplification. Thus, controller 1408 may be configured to transmit diagnostic information of the user and/or auto-injector 2, information pertaining to completion of an injection, and/or information pertaining to an error state of auto-injector 2 to a device of the user, or to the cloud. Signals indicative of needle insertion and/or early device removal also could be transmitted via the wireless communication module. Controller 1408 may also be configured to transmit temperature information for auto-injector 2. For example, a user may be able to monitor, via a mobile device and/or application, for example, a temperature of auto-injector 2 when auto-injector 2 is removed from refrigeration. Controller 1408 also may receive activation and/or delay commands via the wireless communication module. Controller 1408 may further receive operation adjustment commands such as commands relating to adjustment of preferred operation speed, for example. In some embodiments, controller 1408 may receive a command to pause an injection.

In some embodiments, controller 1408 may communicate with a mobile application of a user's mobile device via the wireless communication module. The mobile application may be configured to facilitate use of auto-injector 2 and improve user experience. In some embodiments, the mobile application may be used to automatically check an expiration date of a medicament contained within auto-injector 2. Such functionality may relieve a user from having to manually check the expiration date and may improve user safety. Based on an expiration date, the mobile device may be configured to alert the user and/or disable use of the auto-injector 2. In some embodiments, the mobile application may be used to alert a user as to product recalls and/or may disable the device in the event of product recalls. For example, the mobile application may access a database via the internet to determine whether particular devices, lots of devices, medicaments, and/or lots of medicaments have been recalled. In some embodiments, the mobile application may be configured to confirm whether the auto-injector 2 and/or medicament is authentic as opposed to counterfeit. The mobile application may do so by, for example, cross-referencing a product serial number or a digital signature against a database of authenticated products. In some embodiments, a portion or portions of auto-injector 2 may be disposable and the mobile application may be configured to confirm the authenticity of such portion or portions prior to use.

In some embodiments, the mobile application may be used to facilitate an injection sequence. For example, the mobile application may sync with the events of an injection sequence and provide contemporaneous instructions to the user as to which tasks (e.g., depress switch 1409, hold auto-injector 2 against skin, remove auto-injector) to perform at which times. In some embodiments, the instructions may be narrated audibly. In some embodiments, the instructions may be provided visually via a display on the mobile device. In some embodiments, the mobile application may be configured to provide a detailed indication of a progress of an injection sequence. For example, the mobile application may provide text, visual, and/or audible indications of progress with greater granularity than shown by LEDs, for example, as described herein previously.

In some embodiments, the mobile application may be configured to record and store a date and/or time of an injection. Based on the date and/or time of the injection, and a user's prescription information, the mobile application may be configured to automatically create a reminder for a subsequent injection. In some embodiments, upon completion of an injection, the mobile application may be configured to provide a notification to the user with positive feedback for adherence to a prescription regimen. In some embodiments, the mobile application may provide points and/or rewards for continued adherence.

In some embodiments, the mobile application may be configured to authenticate a user of the auto-injector 2 prior to use. For example, the mobile application, in connection with the user's mobile device, may use biometric identification, two-factor authentication, or any other suitable authentication protocol to confirm the identity of the user prior to an injection. Upon authentication of the user, the mobile application may cause the auto-injector to become activated or otherwise be unlocked. Such user authentication may inhibit misuse and/or waste of costly medicaments by persons other than an intended user.

In some embodiments, the mobile application may be configured to detect operating conditions of auto-injector 2. For example, the mobile application may be configured to detect a battery level of the device and in case of a low battery indication, the mobile application may be configured to provide a notification to the user indicative of a need to charge the device. In some embodiments, the mobile application may be configured to detect mechanical and/or electrical malfunctions of auto-injector 2 and convey such information to the user.

FIG. 18 shows an exemplary method 2000 according to the disclosure. Method 2000 may start at step 2002, where a user may position auto-injector 2 on her body so that tissue-engaging surface 4 contacts a skin surface. The user may position auto-injector 2 on her skin after removing locking component 1610, as described herein previously, thereby allowing touch sensor 1410 to detect proximity of the skin. Auto-injector 2 may be mounted in any suitable location, such as, e.g., the thigh, abdomen, shoulder, forearm, upper arm, leg, buttocks, or another suitable location. Auto-injector 2 may be secured to the skin by adhesive patch 12. The securement of auto-injector 2 at step 2002 may cause activating switch 1409, which extends outward from tissue-engaging surface 4, to be depressed and break a circuit. The breaking of the circuit may cause a signal to be sent to controller 1408 indicative that activating switch 1409 has been depressed. Alternatively, any other suitable mechanism may power on or otherwise activate auto-injector 2 before or after step 2002. Upon depression of activating switch 1409, auto-injector 2 may emit an audio tone and/or illuminate one or more LEDs (e.g., one or more LEDs of a first color, e.g., blue) to indicate depression of activating switch 1409.

Once auto-injector 2 is activated at step 2002, method 2000 may proceed to step 2004, where controller 1408 may determine whether tissue-engaging surface 4 is positioned on a skin surface. At step 2004, controller 1408 may receive a measurement from touch sensor 1410 indicating whether auto-injector 2 is positioned on skin or another surface. If controller 1408 determines that touch sensor 1410 is in contact with skin, for example, when a capacitance value received from touch sensor 1410 is within a predetermined range, method 2000 may proceed to step 2008. If controller 1408 determines that touch sensor is not in contact with skin, for example, if the capacitance measurement received from touch sensor 1410 indicates that auto-injector 2 is in contact with a non-skin surface like wood or metal, method 2000 may proceed to step 2006. At step 2006, auto-injector 2 may be placed into an error condition. In the error condition, an LED may be activated (e.g., a red LED) to indicate to the user that an error has occurred, or a message may be displayed on a display screen. In some examples, auto-injector 2 may need to be manually reset before an injection can be completed. In other examples, auto-injector 2 may loop back to step 2004, wherein controller 1408 continuously attempts to determine whether touch sensor 1410 is in contact with skin. Method 2000 also may require that touch sensor 1410 be in contact with skin during the entire injection. Thus, if at any point during the injection, controller 1408 determines that touch sensor 1410 is no longer in contact with skin, controller 1408 may stop the injection (e.g., by stopping further movement of translation mechanism 1366), may generate an error signal or message, and may retract needle 306 if it had been extended. By stopping the injection and retracting needle 306, a risk of dispensing the drug outside of the body (i.e. a wet injection) and/or needle stick injuries may be mitigated. Upon the determination of step 2004, auto-injector 2 may emit an audio tone and/or illuminate one or more LEDs to indicate that the auto-injector 2 is positioned on the skin surface. In one example, one or more additional LEDs of the first color may be illuminated at this stage to indicate further progress of the injection.

At step 2008, controller 1408 may send a signal to activate translation mechanism 1366. Once activated, translation mechanism 1366 may move toward second end 1306 of cartridge 1302 (referring to FIGS. 13 and 14), causing cartridge 1302 itself to move in the same direction. This may cause needle 308 to move in the opposing direction to access cartridge 1302 as set forth above. The movement of driver 1398 and needle 308 causes carrier 202 to move in the same direction, which sets forth the chain of events that ultimately deploys needle 306 into the user by the mechanisms set forth in FIGS. 5-11. Translation mechanism 1366 will continue to move toward second end 1306 until a desired amount of the drug contained within cartridge 1302 is dispensed into the user. Upon activation of the translation mechanism, auto-injector 2 may emit an audio tone and/or illuminate one or more LEDs to indicate that the injection is in progress. For example, yet additional LEDs of the first color may be illuminated as the injection progresses to give the user a visual indication of the progression.

Method 2000 may proceed to step 2010, where controller 1408 may determine whether the injection is complete. This determination may be based on interruption of beam 1430 by piston 1316 (as described with reference to FIGS. 4A, 13, and 14). That is, when beam 1430 is broken (not received by detector 1416), controller 1408 may determine that injection is complete. Once controller 1408 determines that the injection is complete, controller 1408 may send a signal to translation mechanism 1366 to reverse the direction of rotation of the lead screw, which may cause ramp 1500 to push against ramp 243 of stop 240, enabling retraction of needle 306 as discussed above with reference to FIG. 11. In one example, controller 1408 may institute a delay after receiving an indication that beam 1430 has been interrupted. The delay may be from, e.g., 0.1 to 60 seconds.

An additional end detection mechanism may be used instead of or in combination with the interruption-type sensor described above. For example, a current of the motor of translation mechanism 1366 may be utilized to determine whether an injection has been completed. That is, when piston 1316 reaches second end 1306 of cartridge 1302, the current on the motor will increase (e.g., as a result of piston 1316 engaging the end of cartridge 1302), signaling the expulsion of all or substantially all of the contents of cartridge 1302. One exemplary combination could include the use of beam 1430, where interruption of beam 1430 indicates that, e.g., 90 to 98 percent of the injection has been completed. Then, the current of the motor of translation mechanism 1366 could be analyzed to determine whether the remaining 2 to 10 percent of the injection has been completed. In another example, instead of using an optical switch, a delay from the initiation of the translation mechanism 1366 may be used by controller 1408 to determine when to reverse translation mechanism 1366. In one example, this delay may be from, e.g., about 1 to about 120 seconds, although other suitable times are also contemplated. In any event, the delay from initiation may be long enough to permit emptying of cartridge 1302. In still another example, beam 1430 may be used in combination with an encoder. The encoder may be configured to detect a position of piston 1316. If the encoder were used to detect the position of piston 1316 alone, a drive train issue could inhibit accurate detection. For example, piston 1316 may rotate when pushed by the lead screw. Such rotation may cause uncertainty as to actual position of piston 1316. When used in conjunction with beam 1430, however, controller 1408 may be configured to recalibrate the encoder in response to interruption of beam 1430. Such recalibration may allow controller 1408 to update the actual position of the encoder and resume accurate detection of the position of piston 1316 using the encoder.

Upon determination that the injection is complete, auto-injector 2 may emit an audio tone and/or illuminate one or more LEDs to indicate completion of the injection. In some examples, one or more LEDs of a second color (e.g., green) that is different from the first color may be illuminated to signal to the user that the injection is complete. In some examples, all of the LEDs of the device may be illuminated with the second color, and other indications also may be used. For example, all of the LEDs may be illuminated with the second color and may flash intermittently at the end of the injection.

In some examples, a timing of an injection procedure, measured from the initial activation of activating switch 1409 to retraction of needle 306 from the user after drug delivery, may be from about 20 seconds to about 90 seconds, or from about 25 seconds to about 60 seconds, from about 30 seconds to about 45 seconds, or less than or equal to about 120 seconds, or less than or equal to about 90 seconds, or less than or equal to about 60 seconds, or less than or equal to about 45 seconds, or less than or equal to about 30 seconds. Such timing represents a significant improvement over existing devices, for which the timing of an injection may be much longer and, in some cases, as long as about 9 minutes or even longer.

Method 2000 also may include additional steps. For example, method 2000 may include determining whether a drug within cartridge 1302 is too cold for delivery into the user, whether power source 1406 has enough energy to complete an injection, whether needle 306 has been prematurely deployed and/or retracted, whether the current of the motor of translation mechanism 1366 is in an appropriate range, and whether an injection procedure has extended beyond a maximum acceptable procedure time. When controller 1408 senses any of the above errors, it may communicate such errors to the user, and may end an ongoing injection by, e.g., halting or reversing translation mechanism 1366 and retracting needle 306 from the user. Auto-injector 2 may emit an audio tone and/or illuminate one or more LEDs indicative of any of the foregoing additional steps. For example, one or more LEDs of a third color (e.g., red) that is different than the first and second colors may be illuminated.

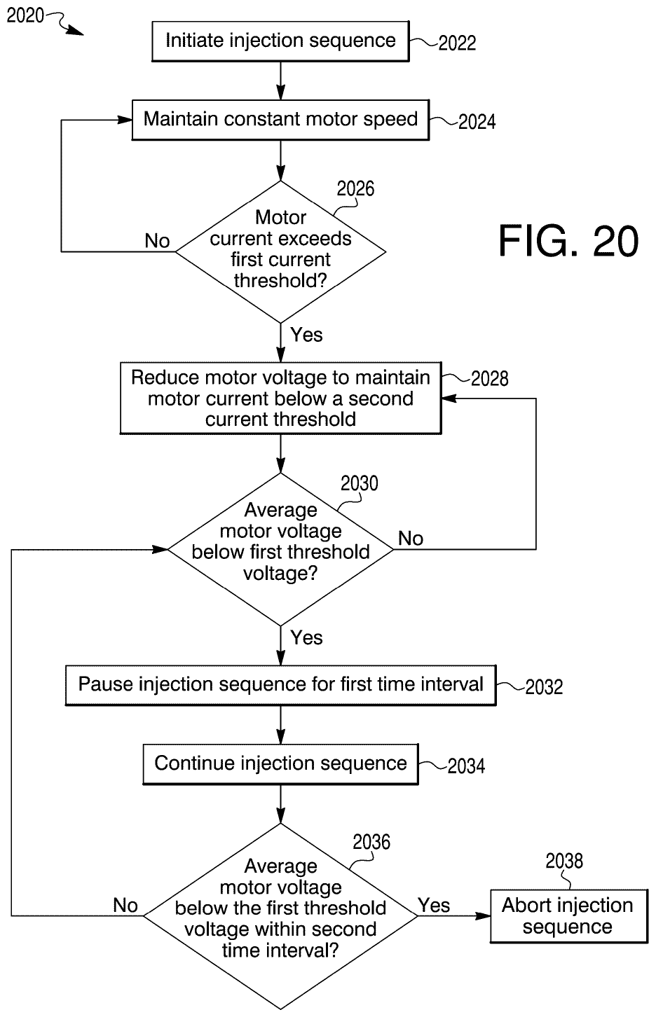

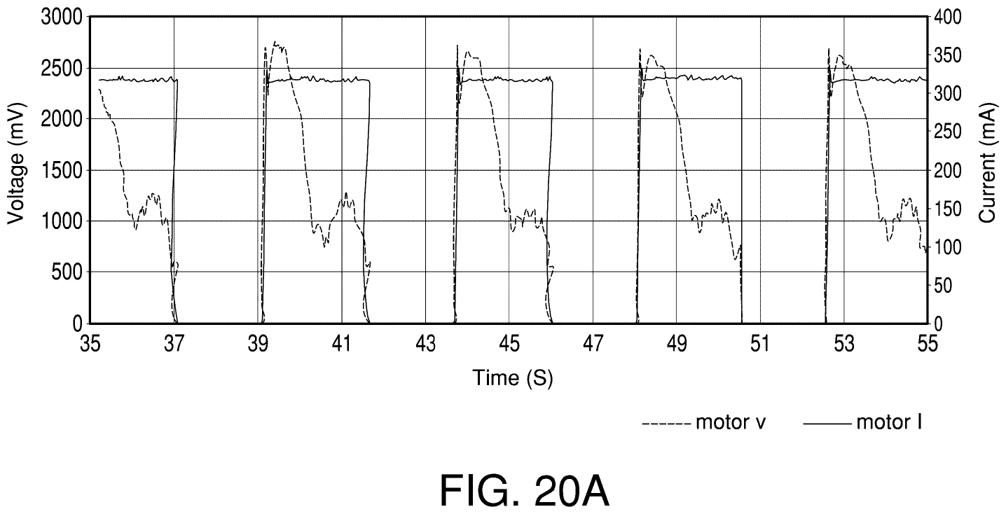

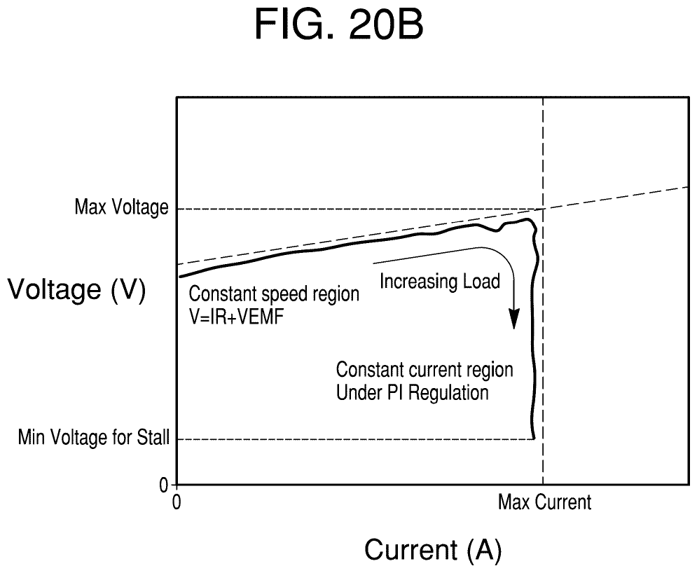

FIG. 20 shows an exemplary method 2020 of controlling a torque of the motor of translation mechanism 1366 and detecting when the motor stalls. At step 2022, controller 1408 may initiate an injection sequence. As described herein previously, an injection sequence may be initiated upon depressing activating switch 1409 against a user's skin and/or detecting the user's skin by touch sensor 1410. During the injection sequence, a voltage may be applied to the motor of translation mechanism 1366 to drive the motor.

At step 2024, as the injection sequence progresses, controller 1408 may maintain the motor of translation mechanism 1366 at a constant speed. The constant speed may be, for example, a rotational speed measured in revolutions per minute (RPM). Controller 1408 may maintain the motor at a constant speed by varying the voltage applied to the motor. For example, when a higher load is applied to the motor due to an obstruction, increased fluid pressure, increased component friction, or any other cause, controller 1408 may compensate for the increased load by increasing the voltage applied to the motor. Conversely, when a load applied to the motor is reduced, controller 1408 may compensate for the reduction in load by decreasing the voltage applied to the motor. Maintaining the motor at a constant speed may reduce a likelihood that the user experiences injection site pain. For example, maintaining the motor at a constant speed may prevent the bolus from become excessively large, thereby mitigating the risk of pain.

During the injection sequence, controller 1408 may monitor a current supplied to the motor. The motor current may be indicative of a torque generated by the motor. For example, a higher motor current may indicate a higher torque being generated by the motor. At step 2026, controller 1408 may determine whether the motor current exceeds a first current threshold. The first current threshold may be determined and/or set based on a maximum torque that may be safely generated by the motor. The maximum torque may be reached, for example, when the injection sequence is obstructed in some way. If controller 1408 determines that the motor current does not exceed the first current threshold, the method 2020 may revert to step 2024 and controller 1408 may continue to maintain the motor at a constant speed. If, on the other hand, controller 1408 determines that the motor current exceeds the first current threshold, method 2020 may proceed to step 2028.

At step 2028, controller 1408 may reduce the motor voltage to maintain the motor current below a second current threshold. In some embodiments, the second current threshold may be greater than the first current threshold and may more closely correlate to the maximum torque that may be safely generated by the motor. In some embodiments, the second current threshold may be less than, or the same as, the first current threshold. In the event that the injection sequence is obstructed, the motor speed may slow and the motor impedance may decrease. As the motor impedance decreases, a lower voltage may be required to maintain the motor current below the second current threshold. Controller 1408 may monitor an average motor voltage applied to the motor. The average motor voltage may be, for example, a time average.

Steps 2024 through 2028 of method 2020 may generally be illustrated by the graph depicted in FIG. 20B, in which a curve representing a relationship between the voltage applied to the motor of translation mechanism 1366 and the current consumed by the motor is plotted. The curve may be characterized by the following equation:

$$V = iR + {V}_{emf}$$

In the equation above, V is the voltage applied to the motor, i is a current consumed by the motor, R is a coil resistance of the motor, and V_{emf} is a back electromotive force that acts against the applied voltage at a given speed. As shown in FIG. 20B, the curve may include a constant speed region, in which the motor may be maintained a constant speed (step 2024). In the constant speed region, V_{emf} may remain approximately constant and the curve may be approximately linear.

As shown in FIG. 20B, as a load (i.e. a torque) acting on the motor of translation mechanism 1366 increases, the current consumed by the motor may increase. As the current approaches a Max Current, a voltage applied to the motor may be reduced, thereby maintaining the current below the Max Current (steps 2026 and 2028). The current consumed by the motor may be maintained below the Max Current using proportional integral (PI) regulation. As shown, there may be a minimum voltage for the motor below which the motor may stall.

Steps 2030 through 2038 of method 2020 may correspond to a control sequence for preventing stalling of the motor. FIG. 20A depicts a graph which may represent the voltage applied to the motor and the current consumed by the motor over time and in accordance with steps 2030 through 2038.

At step 2030, controller 1408 may determine whether the average motor voltage has decreased below a first threshold voltage. The average motor voltage decreasing below the first threshold voltage may indicate that the injection sequence is obstructed. If controller 1408 determines that the average motor voltage has not decreased below a first threshold voltage, method 2020 may revert to step 2028, at which controller 1408 may continue to maintain the motor current below the second current threshold. FIG. 20A illustrates five intervals during which controller 1408 may maintain the motor current at a constant value (e.g. below the second current threshold): between about 35 seconds and about 37 seconds, between about 39 seconds and about 41.5 seconds, between about 43.5 seconds and about 46 seconds, between about 48 seconds and about 50.5 seconds, and between about 52.5 seconds and about 55 seconds. As shown in FIG. 20A, the voltage applied to the motor during each interval may decrease, albeit with some fluctuations, to maintain the motor current below the second current threshold. Though the voltage during each interval in FIG. 20A is shown as decreasing, the voltage need not necessarily decrease to maintain the motor current below the second current threshold, but instead may stay flat in certain situations.

If, on the other hand, controller 1408 determines that the average motor voltage has decreased below the first threshold voltage, controller 1408 may cause the injection sequence to be paused for a first time interval. When causing the injection sequence to be paused, controller 1408 may cease applying voltage to the motor. In some embodiments, the first time interval may be 2 seconds, for example. FIG. 20A illustrates four such pauses: between about 37 seconds and about 39 seconds, between about 41.5 seconds and about 43.5 seconds, between about 46 seconds and about 48 seconds, and between about 50.5 seconds and about 52.5 seconds.

The first time interval may be sufficiently long to allow fluid pressure within auto-injector 2 to dissipate. The first time interval may also be sufficiently short such that the user may not be prompted to remove auto-injector 2 from the user's skin (e.g., the first time interval is set to be less than a typical reaction time of the user to falsely identify the end of the injection). The first time interval may further be indicated by illumination of one or more of the LEDs of the progress ring or another light within auto-injector 2 and visible by a user. The LEDs may be illuminated, for example, in a particular pattern or according to a particular color scheme to indicate the first time interval and that the injection sequence is paused rather than stopped.

After pausing the injection sequence, controller 1408 may continue the injection sequence at step 2034. To continue the injection sequence, controller 1408 may resume supplying voltage to the motor of translation mechanism 1366. At step 2036, controller 1408 may determine whether the average motor voltage has decreased below the first threshold voltage within a second time interval. The second time interval may be shorter than the first time interval and may be set and/or determined to be indicative of a confirmation that the injection sequence is obstructed. The second time interval may be, for example, about 0.9 seconds. If the motor voltage has not decreased below the first threshold voltage within the second time interval, method 2020 may revert to step 2030. If, on the other hand, controller 1408 determines that the motor voltage has decreased below the first threshold voltage within the second time interval, method 2020 may proceed to step 2038 at which controller 1408 may cause the injection sequence to be aborted.

In some embodiments, controller 1408 may perform step 2026 continuously as it performs steps 2028 to 2036. For example, controller 1408 may continue to determine whether the motor current exceeds the first current threshold as steps 2028 to 2036 are performed. If the motor current continues to exceed the first current threshold, method 2020 may proceed through steps 2028 to 2036 as described herein previously. In the event the motor current falls below the first current threshold, on the other hand, method 2020 may revert to step 2024 and controller 1408 may maintain the motor at a constant speed. In other words, if a high load on the motor, due to obstruction, high fluid pressure, or the like, dissipates during performance of steps 2028 to 2036, controller 1408 may simply revert to maintaining a constant motor speed rather than proceeding through any remaining steps unnecessarily.

Accordingly, method 2020 may allow controller 1408 to effectively distinguish between situations in which the needle may be partially blocked or a high friction force may be acting against the injection sequence, and situations in which the injection sequence is insurmountably obstructed. In the former situations, auto-injector 2 may have the ability to complete the injection sequence and the injection sequence may not be prematurely terminated. In the latter situations, auto-injector 2 may not have the ability to complete the injection sequence and the injection sequence may be appropriately terminated. In such situations, auto-injector 2 may emit an audio tone and/or illuminate one or more LEDs to indicate that the injection was terminated before completion. Method 2020 may further appropriately terminate an injection sequence in which the piston 1316 extends completely, indicating that the cartridge 1302 is empty. Method 2020 may further allow the auto-injector 2 to be used on an emergency basis if, for example, a user performs an injection without first warming up auto-injector 2 to decrease a viscosity of the medicament. Method 2020 may further allow an injection of a viscous medicament to proceed at a slower rate than the motor and gear reduction ratio may otherwise allow.

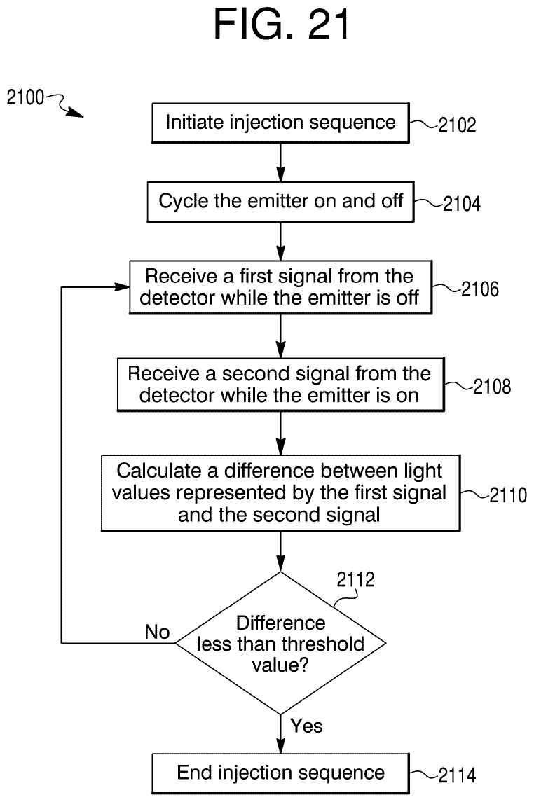

FIG. 21 shows an exemplary method 2100 of detecting an end of a dose of medicament using emitter 1414 and detector 1416. Method 2100 may be used, for example, to detect a time at which a full dose of medicament has been dispensed to a user and end the corresponding injection sequence.

At step 2102, controller 1408 may initiate an injection sequence. As described herein previously, an injection sequence may be initiated upon depressing activating switch 1409 against a user's skin and/or detecting the user's skin by touch sensor 1410. At step 2104, controller 1408 may cycle emitter 1414 on and off periodically. Emitter 1414 may be cycled on and off rapidly in a square wave pattern, such that emitter 1414 is turned off and on several times per second. Cycling emitter 1414 on and off may allow detector 1416 to be exposed to light produced by emitter 1414 in combination with ambient light, and also to ambient light alone.

At step 2106, controller 1408 may receive a first signal from detector 1416 corresponding to a time when emitter 1414 is off. The first signal may correspond to, and/or be indicative of, ambient light detected by the detector 1416. At step 2108, controller 1408 may receive a second signal from detector 1416 corresponding to a time when emitter 1414 is on. The second signal may correspond to, and/or be indicative of, light emitted by emitter 1414 in combination with ambient light as detected by the detector 1416.

At step 2110, controller 1408 may calculate a difference between a first light value represented by the first signal and a second light value represented by the second signal. The difference may be indicative of how much light detected by detector 1416 is attributable to light emitted by emitter 1414 as opposed to ambient light. At step 2112, controller 1408 may determine whether the difference is less than a threshold value. If controller 1408 determines that the difference is not less than a threshold value, method 2100 may revert to step 2106. If, on the other hand, controller 1408 determines that the difference is less than the threshold value, controller 1408 may end the injection sequence at step 2114.

Accordingly, method 2100 may be used to reduce the impact of ambient light when detecting an end of a dose of medicament. Specifically, method 2100 may address a situation in which light from emitter 1414 is blocked from reaching the detector 1416 indicating an end of a dose, yet ambient light is able to reach detector 1416 and create a false negative reading indicating that an end of dose has not been reached.

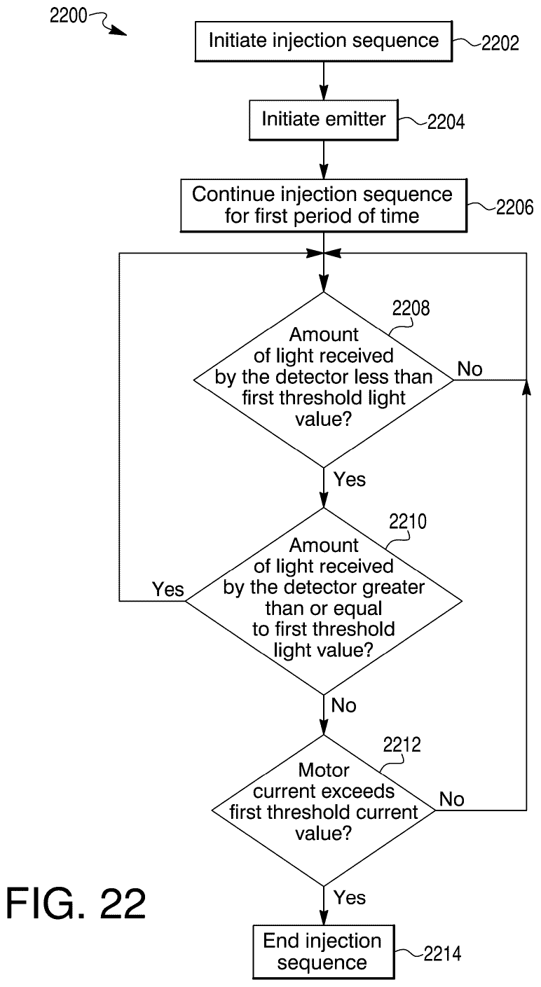

FIG. 22 shows another exemplary method 2200 of detecting an end of a dose of medicament using emitter 1414 and detector 1416. Method 2200 may be used, for example, to detect a time at which a full dose of medicament has been dispensed to a user and end the corresponding injection sequence.

At step 2202, controller 1408 may initiate an injection sequence. As described herein previously, an injection sequence may be initiated upon depressing activating switch 1409 against a user's skin and/or detecting the user's skin by touch sensor 1410. At step 2204, controller 1408 may initiate emitter 1414 or otherwise cause emitter 1414 to emit light.

At step 2206, controller 1408 may cause the injection sequence to continue for a first period of time. The first period of time may be a predetermined period of time corresponding to a duration in which a full dose cannot possibly be, or is unlikely to be, dispensed. For example, the first period of time may be between about 20% and 50% of the total injection time. During the first period of time, controller 1408 is not able to interrupt the injection sequence in response to a signal received from detector 1416 (but could still interrupt the injection sequence due to obstructions or stalling as discussed with reference to FIG. 20).

At step 2208, after the end of the first period of time, controller 1408 may determine whether an amount of light received by detector 1416 is less than a first threshold light value. Controller 1408 may make the determination based on a signal received from detector 1416 indicative of light received by detector 1416. The first threshold light value may correspond to an amount of light received by detector 1416 at the end of a dose. If controller 1408 determines that the amount of light received by detector 1416 is not less than the first threshold light value, controller 1408 may continue the injection sequence and method 2200 may otherwise remain at step 2208. If, on the other hand, controller 1408 determines that the amount of light received by detector 1416 is less than the first threshold light value, the method may proceed to step 2210.

At step 2210, controller 1408 may determine whether the amount of light received by detector 1416 is greater than or equal to the first threshold light value. If controller 1408 determines that the amount of light received by detector 1416 has risen to or above the first threshold light value, controller 1408 may continue the injection sequence and method 2200 may revert to step 2208. If, on the other hand, controller 1408 determines that the amount of light received by detector 1416 has remained less than the first threshold light value, the method may proceed to step 2212. Step 2210 may in effect enable controller 1408 to “clear” the injection sequence of anomalous interruptions of the light received by the detector, which may be caused by an air bubble within cartridge 1302 that blocks the path of light between emitter 1414 and detector 1416, for example, provided the amount of light subsequently meets or exceeds the first threshold light value.

At step 2212, controller 1408 may determine whether the motor current exceeds a first threshold current value. The first threshold current value may be determined and/or set based on a current indicative of an end of the injection sequence. The first current threshold value may be set, for example, based on a current indicative of piston 1316 reaching second end 1306 of cartridge 1302. If controller 1408 determines that the motor current does not exceed the first threshold current value, controller 1408 may continue the injection sequence and method 2200 may revert to step 2208. If, on the other hand, controller 1408 determines that the motor current exceeds the first threshold current value, method 2200 may proceed to step 2214 at which controller 1408 may cause the injection sequence to end.

Method 2200 may accordingly allow for accurate identification of the end of an injection sequence by identifying an instant in which both the light received by detector 1416 and the motor current are indicative of an end of the dose. By performing steps 2208, 2210, and 2212 sequentially, false identifications of the end of the dose due to either anomalous interruptions of light or anomalous high current events alone may be mitigated. Method 2200 may specifically reduce the impact of bubbles within cartridge 1302 on detection of the end of a dose of medicament.

FIG. 23 shows an exemplary method 2300 of operating activating switch 1409 of auto-injector 2 according to the disclosure. In particular, FIG. 23 depicts an exemplary sequence of positions of activating switch 1409 and corresponding functions of auto-injector 2.

Initially, at step 2302, auto-injector 2 may be disposed within a packaging such that plunger 1450 is in a depressed state and auto-injector 2 is in a low-power sleep mode. In some embodiments, during manufacturing auto-injector 2 may be programmed in an awake or active state. In some embodiments, if plunger 1450 is depressed for a predetermined period of time following programming, such as when auto-injector is placed in the packaging, auto-injector 2 may be configured to transition to the low-power sleep mode. The predetermined period of time may be any suitable period of time, such as 60 minutes, 30 minutes, 15 minutes, 10 minutes, 5 minutes, 2 minutes, or any other suitable period. Auto-injector 2 may be sealed in the packaging such that the packaging indicates that the auto-injector 2 has not been previously used. The packaging may be made from any suitable material, including paper, cardboard, plastic, cellophane, and the like. The packaging may press against plunger 1450 such that plunger 1450 is flush or nearly flush with housing 3 of auto-injector 2 and plunger 1450 is blocked from extending outwardly from auto-injector 2. With plunger 1450 in the depressed state, the circuit associated with activating switch 1409 may be open, thereby maintaining the auto-injector 2 in the low-power sleep mode.

At step 2304, auto-injector 2 may be removed from the packaging such that plunger 1450 is no longer depressed by the packaging and plunger 1450 may extend outwardly from the auto-injector 2. As plunger 1450 transitions from the depressed state to the free or extended state, plunger flange 1454 may contact or otherwise depress plunger switch 1448, thereby completing the circuit associated with activating switch 1409.

At step 2306, in response to the circuit associated with activating switch 1409 being completed, auto-injector may transition from the low-power sleep mode to an active mode. In the active mode, auto-injector 2 may calibrate touch sensor 1410. Auto-injector 2 may calibrate touch sensor 1410 by detecting a value or measurement of the touch sensor 1410 in ambient air, i.e. not against a user's skin. Auto-injector 2 may perform such calibration during a predetermined time period after auto-injector 2 is removed from the packaging (in some cases immediately after removal) so that such calibration occurs before a user may expose their skin to touch sensor 1410. In the active mode, auto-injector 2 may further detect whether emitter 1414 and/or detector 1416 are functioning properly, detect whether a needle is positioned properly, detect whether the motor of translation mechanism 1366 is responsive and/or operational, and/or perform any other suitable status tests. Auto-injector 2 may detect the positioning of the needle, for example, using a switch or detector configured to report the position of the needle to the controller 1408. In the active mode, auto-injector 2 may further illuminate one or more backlights to allow a user to inspect a vial and/or a drug contained in the vial through transparent window 50. In the active mode, auto-injector 2 may further display any other indication that auto-injector 2 is ready to be used.

At step 2308, auto-injector 2 may be placed against a user's skin such that plunger 1450 is depressed into auto-injector 2. Upon plunger 1450 being depressed, the circuit associated with activating switch 1409 may transition to an open state. As described above with reference to FIG. 18 and method 2000, auto-injector 2 may further detect contact with skin using touch sensor 1410. In response to depression of plunger 1450 and detection of contact with skin, auto-injector 2 may initiate an injection sequence at step 2310. The injection sequence may be a sequence resulting in injection of the user with a medicament, as described herein previously.

At step 2312, auto-injector 2 may be removed from the user's skin and plunger 1450 may again extend outwardly from auto-injector 2. Upon plunger 1450 extending outwardly, the circuit associated with activating switch 1409 may transition from the open state to the closed state. In response, auto-injector 2 may end the injection sequence at step 2310 and, for example, initiate retraction of the patient needle by reversing the motor. Auto-injector 2 may initiate retraction of the needle if the injection sequence has proceeded to completion or if the auto-injector has prematurely or accidentally been removed from the skin to prevent wet injection. Alternatively, in some embodiments, controller 1408 may determine whether a value received from touch sensor 1410 is indicative of the auto-injector 2 remaining in contact with the user's skin. If the value received by controller 1408 is indicative of the auto-injector 2 remaining in contact with the user's skin, auto-injector 2 may pause the injection sequence, thereby preventing wet injection. If the plunger 1450 is again depressed, thereby placing the circuit associated with activating switch 1409 in the open state, auto-injector 2 may resume the injection sequence.

According to the foregoing method 2300, activating switch 1409 may serve to keep auto-injector 2 in a lower-power sleep mode when in the packaging, transition auto-injector 2 to an active mode upon removal from the packaging, indicate when auto-injector 2 has been placed against a user's skin for an injection sequence, and indicate when auto-injector 2 has been removed from the user's skin at the end of an injection sequence. Moreover, a signal from activating switch 1409 may be cross-checked against a signal from touch sensor 1410 to more accurately determine whether auto-injector 2 has been removed from the user's skin, or whether, for example, an inadvertent or minor movement of auto-injector occurred.

It should be understood that steps of one or more of the various methods described herein may be combined in certain embodiments. Furthermore, in certain embodiments, fewer than all of the steps of a method described herein may be performed and/or additional steps not described herein may be performed. Moreover, the steps described herein need not necessarily be performed in the exact order presented.

### Medicaments and Drugs

The embodiments described herein may be used in connection with the administration of various medicaments, drugs, and/or pharmaceutical formulations to patients. Exemplary medicaments, drugs, and/or pharmaceutical formulations with which the embodiments described herein may be used are described in U.S. Pat. Nos. 8,945,559 B2, 9,987,500 B2, and 11,603,407 B2, the entireties of which are incorporated herein by reference. Medicaments, drugs, and/or pharmaceutical formulations that may be used with the embodiments of the present disclosure are described in further detail hereinafter.

First, exemplary medicaments, drugs, and/or pharmaceutical formulations consistent with U.S. Pat. No. 8,945,559 B2 are described. As used herein, the expression “pharmaceutical formulation” means a combination of at least one active ingredient (e.g., a small molecule, macromolecule, compound, etc. which is capable of exerting a biological effect in a human or non-human animal), and at least one inactive ingredient which, when combined with the active ingredient or one or more additional inactive ingredients, is suitable for therapeutic administration to a human or non-human animal. The term “formulation”, as used herein, means “pharmaceutical formulation” unless specifically indicated otherwise.

The present disclosure provides pharmaceutical formulations comprising at least one therapeutic polypeptide. According to certain embodiments of the present disclosure, the therapeutic polypeptide is an antibody, or an antigen-binding fragment thereof, which binds specifically to human interleukin-4 receptor alpha (hIL-4Rα). More specifically, the present disclosure includes pharmaceutical formulations that comprise: (i) a human antibody that specifically binds to hIL-4Rα; (ii) an acetate/histidine buffer system; (iii) an organic cosolvent that is a non-ionic surfactant; (iv) thermal stabilizer that is a carbohydrate; and (v) a viscosity reducer. Specific exemplary components and formulations included within the present disclosure are described in detail below.

The pharmaceutical formulations of the present disclosure may comprise a human antibody, or an antigen-binding fragment thereof, that binds specifically to hIL-4Rα. As used herein, the term “hIL-4Rα” means a human cytokine receptor that specifically binds interleukin-4 (IL-4). In certain embodiments, the antibody contained within the pharmaceutical formulations of the present disclosure binds specifically to the extracellular domain of hIL-4Rα. An exemplary human IL-4 receptor alpha (hIL-4Rα) amino acid sequence is described in SEQ ID NO:25. Antibodies to hIL-4Rα are described in U.S. Pat. Nos. 7,605,237 and 7,608,693. The extracellular domain of hIL-4Rα is represented by the amino acid sequence of SEQ ID NO: 26.

The term “antibody”, as used herein, is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody”. Each heavy chain comprises a heavy chain variable region (abbreviated herein as HCVR or V_{H}) and a heavy chain constant region. The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementary determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

Unless specifically indicated otherwise, the term “antibody”, as used herein, shall be understood to encompass complete antibody molecules as well as antigen-binding fragments thereof. The term “antigen-binding portion” or “antigen-binding fragment” of an antibody (or simply “antibody portion” or “antibody fragment”), as used herein, refers to one or more fragments of an antibody that retain the ability to specifically bind to hIL-4Rα or an epitope thereof.

An “isolated antibody”, as used herein, is intended to refer to an antibody that is substantially free of other antibodies having different antigenic specificities (e.g., an isolated antibody that specifically binds hIL-4Rα is substantially free of antibodies that specifically bind antigens other than hIL-4Rα).

The term “specifically binds”, or the like, means that an antibody or antigen-binding fragment thereof forms a complex with an antigen that is relatively stable under physiologic conditions. Specific binding can be characterized by a dissociation constant of at least about 1×10⁻⁶M or greater. Methods for determining whether two molecules specifically bind are well known in the art and include, for example, equilibrium dialysis, surface plasmon resonance, and the like. An isolated antibody that specifically binds hIL-4Rα may, however, have cross-reactivity to other antigens, such as IL-4R molecules from other species (orthologs). In the context of the present disclosure, multispecific (e.g., bispecific) antibodies that bind to hIL-4Rα as well as one or more additional antigens are deemed to “specifically bind” hIL-4Rα. Moreover, an isolated antibody may be substantially free of other cellular material or chemicals.

Exemplary anti-hIL-4Rα antibodies that may be included in the pharmaceutical formulations of the present disclosure are set forth in U.S. Pat. Nos. 7,605,237 and 7,608,693, the disclosures of which are incorporated by reference in their entirety.

According to certain embodiments of the present disclosure, the anti-hIL-4Rα antibody is a human IgG1 comprising a heavy chain variable region that is of the IGHV3-9 subtype and a light chain variable region that is of the IGKV2-28 subtype (see Barbie and Lefranc, The Human Immunoglobulin Kappa Variable (IGKV) Genes and Joining (IGKJ) Segments, Exp. Clin. Immunogenet. 1998; 15:171-183; and Scaviner, D. et al., Protein Displays of the Human Immunoglobulin Heavy, Kappa and Lambda Variable and Joining Regions, Exp. Clin. Immunogenet., 1999; 16:234-240).

In some embodiments, the anti-hIL-4Rα comprises at least one amino acid substitution, which results in a charge change at an exposed surface of the antibody relative to the germline IGHV3-9 sequence or the germline IGKV2-28 sequence. The germline IGHV3-9 and IGKV2-28 sequences, and the amino acid position assignment numbers presented herein comport with the international Immunogenetics (IMGT) information system, as described in Lefranc, M.-P., et al., IMGT®, the international ImMunoGeneTics Information System®, Nucl. Acids Res, 37, D1006-D1012 (2009). In some embodiments, the exposed surface comprises a complementarity determining region (CDR). In some embodiments, the amino acid substitution or substitutions are selected from the group consisting of (a) a basic amino acid substituted for a neutral amino acid within CDR2 (e.g., at position 58) of IGHV3-9, (b) a neutral amino acid substituted for an acidic amino acid within CDR3 (e.g., at position 107) of IGHV3-9, and (c) a neutral amino acid substituted for a basic amino acid within CDR1 (e.g., at position 33) of IGKV2-28. Unique permutations in the charge distribution of an antibody, especially at an environmental interface (such as, e.g., in a CDR) would be expected to create unpredictable conditions for antibody stability in solution.

In some embodiments, the anti-hIL-4Rα antibody comprises at least one amino acid substitution, which creates a change in the torsional strain within a framework region of a variable region of the antibody relative to the germline IGHV3-9 sequence or the germline IGKV2-28 sequence. In some embodiments, the amino acid substitution or substitutions are selected from the group consisting of (a) a proline substituted for a non-proline amino acid in framework region 3 (FR3) (e.g., at position 96) of IGHV3-9, and (b) a non-proline amino acid substituted for a proline in framework region 2 (FR2) (e.g., at position 46) of IGKV2-28. Changes in the ability of the peptide chain to rotate, especially within a framework region, which affects the CDR interface with the solvent, would be expected to create unpredictable conditions for antibody stability in solution.

According to certain embodiments of the present disclosure, the anti-hIL-4Rα antibody, or antigen-binding fragment thereof, comprises a heavy chain complementary determining region (HCDR) 1 of SEQ ID NO: 2, an HCDR2 of SEQ ID NO: 3, and an HCDR3 of SEQ ID NO: 4. In certain embodiments, the anti-hIL-4Rα antibody, or antigen-binding fragment thereof, comprises an HCVD of SEQ ID NO:1.

According to certain embodiments of the present disclosure, the anti-hIL-4Rα, or antigen-binding fragment thereof, comprises a light (kappa) chain complementary determining region (LCDR) 1 of SEQ ID NO: 6, an LCDR2 of SEQ ID NO: 7, and an LCDR3 of SEQ ID NO: 8. In certain embodiments, the anti-hIL-4Rα antibody, or antigen-binding fragment thereof, comprises an LCVD of SEQ ID NO:5.

According to certain other embodiments of the present disclosure, the anti-hIL-4Rα antibody, or antigen-binding fragment thereof, comprises an HCDR1 of SEQ ID NO: 10, an HCDR2 of SEQ ID NO:11, an HCDR3 of SEQ ID NO: 12, an LCDR1 of SEQ ID NO: 14, an LCDR2 of SEQ ID NO: 15, and an LCDR3 of SEQ ID NO: 16. In certain embodiments, the anti-hIL-4Rα antibody, or antigen-binding fragment thereof, comprises an HCVD of SEQ ID NO:9 and an LCVD of SEQ ID NO: 13.

According to certain other embodiments of the present disclosure, the anti-hIL-4Rα antibody, or antigen-binding fragment thereof, comprises an HCDR1 of SEQ ID NO: 18, an HCDR2 of SEQ ID NO:19, an HCDR3 of SEQ ID NO: 20, an LCDR1 of SEQ ID NO: 22, an LCDR2 of SEQ ID NO:23, and an LCDR3 of SEQ ID NO: 24. In certain embodiments, the anti-hIL-4Rα antibody, or antigen-binding fragment thereof, comprises an HCVD of SEQ ID NO:17 and an LCVD of SEQ ID NO: 21.

Another non-limiting, exemplary antibody which may be used in the practice of this disclosure is referred to as “mAb2”. This antibody is also referred to in U.S. Pat. No. 7,608,693 as H4H083P. mAb2 (H4H083P) comprises an HCVR/LCVR amino acid sequence pair having SEQ ID NOs: 9/13, and HCDR1-HCDR2-HCDR3/LCDR1-LCDR2-LCDR3 domains represented by SEQ ID NOs: Oct. 11, 2012/SEQ ID NOs: 14-15-16.

Yet another non-limiting, exemplary antibody which may be used in the practice of this disclosure is referred to as “mAb3”. This antibody is also referred to in U.S. Pat. No. 7,608,693 as H4H095P. mAb3 (H4H095P) comprises an HCVR/LCVR amino acid sequence pair having SEQ ID NOs: 17/21, and HCDR1-HCDR2-HCDR3/LCDR1-LCDR2-LCDR3 domains represented by SEQ ID NOs: 18-19-20/SEQ ID NOs: 22-23-24.

The amount of antibody, or antigen-binding fragment thereof, contained within the pharmaceutical formulations of the present disclosure may vary depending on the specific properties desired of the formulations, as well as the particular circumstances and purposes for which the formulations are intended to be used. In certain embodiments, the pharmaceutical formulations are liquid formulations that may contain about 100±10 mg/mL to about 200±20 mg/ml of antibody; about 110±11 mg/mL to about 190±19 mg/mL of antibody; about 120±12 mg/mL to about 180±18 mg/mL of antibody; about 130±13 mg/mL to about 170±17 mg/ml of antibody; about 140±14 mg/mL to about 160±16 mg/mL of antibody; or about 150±15 mg/ml of antibody. For example, the formulations of the present disclosure may comprise about 90 mg/mL; about 95 mg/ml; about 100 mg/ml; about 105 mg/mL; about 110 mg/mL; about 115 mg/ml; about 120 mg/mL; about 125 mg/mL; about 130 mg/mL; about 131 mg/mL; about 132 mg/mL; about 133 mg/mL; about 134 mg/ml; about 135 mg/ml; about 140 mg/mL; about 145 mg/mL; about 150 mg/mL; about 155 mg/mL; about 160 mg/mL; about 165 mg/mL; about 170 mg/mL; about 175 mg/mL; about 180 mg/mL; about 185 mg/ml; about 190 mg/mL; about 195 mg/mL; or about 200 mg/mL of an antibody or an antigen-binding fragment thereof, that binds specifically to hIL-4Rα.

The pharmaceutical formulations of the present disclosure comprise one or more excipients. The term “excipient”, as used herein, means any non-therapeutic agent added to the formulation to provide a desired consistency, viscosity or stabilizing effect.

In certain embodiments, the pharmaceutical formulation of the disclosure comprises at least one organic cosolvent in a type and in an amount that stabilizes the hIL-4Rα antibody under conditions of rough handling, such as, e.g., vortexing. In some embodiments, what is meant by “stabilizes” is the prevention of the formation of more than 2% aggregated antibody of the total amount of antibody (on a molar basis) over the course of rough handling. In some embodiments, rough handling is vortexing a solution containing the antibody and the organic cosolvent for about 120 minutes.

In certain embodiments, the organic cosolvent is a non-ionic surfactant, such as an alkyl poly(ethylene oxide). Specific non-ionic surfactants that can be included in the formulations of the present disclosure include, e.g., polysorbates such as polysorbate 20, polysorbate 28, polysorbate 40, polysorbate 60, polysorbate 65, polysorbate 80, polysorbate 81, and polysorbate 85; poloxamers such as poloxamer 181, poloxamer 188, poloxamer 407; or polyethylene glycol (PEG). Polysorbate 20 is also known as TWEEN 20, sorbitan monolaurate and polyoxyethylenesorbitan monolaurate. Poloxamer 181 is also known as PLURONIC F68.

The amount of organic cosolvent contained within the pharmaceutical formulations of the present disclosure may vary depending on the specific properties desired of the formulations, as well as the particular circumstances and purposes for which the formulations are intended to be used. In certain embodiments, the formulations may contain about 0.1%±0.01% to about 2%±0.2% surfactant. For example, the formulations of the present disclosure may comprise about 0.09%; about 0.10%; about 0.11%; about 0.12%; about 0.13%; about 0.14%; about 0.15%; about 0.16%; about 0.17%; about 0.18%; about 0.19%; about 0.20%; about 0.21%; about 0.22%; about 0.23%; about 0.24%; about 0.25%; about 0.26%; about 0.27%; about 0.28%; about 0.29%; or about 0.30% polysorbate 20 or poloxamer 181. For example, the formulations of the present disclosure may comprise about 0.5%; about 0.6%; about 0.7%; about 0.8%; about 0.9%; about 1%; about 1.1%; about 1.2%; about 1.3%; about 1.4%; about 1.5%; about 1.6%; about 1.7%; about 1.8%; about 1.9%; or about 2.0% PEG 3350.

Exemplary organic cosolvents that stabilize the hIL-4Rα antibody include 0.2%±0.02% polysorbate 20, 0.2%±0.02% poloxamer 181, or 1%±0.1% PEG 3350.

The pharmaceutical formulations of the present disclosure may also comprise one or more thermal stabilizers in a type and in an amount that stabilizes the hIL-4Rα antibody under conditions of thermal stress. In some embodiments, what is meant by “stabilizes” is maintaining greater than about 92% of the antibody in a native conformation when the solution containing the antibody and the thermal stabilizer is kept at about 45° C. for up to about 28 days. In some embodiments, what is meant by “stabilizes” is wherein less than about 5% of the antibody is aggregated when the solution containing the antibody and the thermal stabilizer is kept at about 45° C. for up to about 28 days.

In certain embodiments, the thermal stabilizer is a sugar or sugar alcohol selected from sucrose, trehalose and mannitol, or any combination thereof, the amount of which contained within the formulation can vary depending on the specific circumstances and intended purposes for which the formulation is used. In certain embodiments, the formulations may contain about 2.5% to about 10% sugar or sugar alcohol; about 3% to about 9.5% sugar or sugar alcohol; about 3.5% to about 9% sugar or sugar alcohol; about 4% to about 8.5% sugar or sugar alcohol; about 4.5% to about 8% sugar or sugar alcohol; about 5% to about 7.5% sugar or sugar alcohol; about 5.5% to about 7% sugar or sugar alcohol; or about 6.0% to about 6.5% sugar or sugar alcohol. For example, the pharmaceutical formulations of the present disclosure may comprise about 2.5%±0.375%; about 3%±0.45%; about 3.5%±0.525%; about 4.0%±0.6%; about 4.5%±0.675%; about 5.0%±0.75%; about 5.5%±0.825%; about 6.0%±0.9%; about 6.5%±0.975%; about 7.0%±1.05%; about 7.5%±1.125%; about 8.0%±1.2%; 8.5%±1.275%; about 9.0%±1.35%; or about 10.0%±1.5% sugar or sugar alcohol (e.g., sucrose, trehalose or mannitol).

The pharmaceutical formulations of the present disclosure may also comprise a buffer or buffer system, which serves to maintain a stable pH and to help stabilize the hIL-4Rα antibody. In some embodiments, what is meant by “stabilizes” is wherein less than 3.0%±0.5% of the antibody is aggregated when the solution containing the antibody and the buffer is kept at about 45° C. for up to about 14 days. In some embodiments, what is meant by “stabilizes” is wherein less than 3.7%±0.5% of the antibody is aggregated when the solution containing the antibody and the buffer is kept at about 25° C. for up to about 6 months. In some embodiments, what is meant by “stabilizes” is wherein at least 95%±0.5% of the antibody is in its native conformation as determined by size exclusion chromatography when the solution containing the antibody and the buffer is kept at about 45° C. for up to about 14 days. In some embodiments, what is meant by “stabilizes” is wherein at least 96%±0.5% of the antibody is in its native conformation as determined by size exclusion chromatography when the solution containing the antibody and the buffer is kept at about 25° C. for up to about 6 months. In some embodiments, what is meant by “stabilizes” is wherein at least 62%±0.5% of the antibody is in its neutral conformation as determined by cation exchange chromatography when the solution containing the antibody and the buffer is kept at about 45° C. for up to about 14 days. In some embodiments, what is meant by “stabilizes” is wherein at least 54%±0.5% of the antibody is in its neutral conformation as determined by cation exchange chromatography when the solution containing the antibody and the buffer is kept at about 25° C. for up to about 6 months. By “neutral conformation”, what is meant is the faction of antibody that elutes from an ion exchange resin in the main peak, which is generally flanked by more “basic” peaks on one side and more “acidic” peaks on the other side.

The pharmaceutical formulations of the present disclosure may have a pH of from about 5.2 to about 6.4. For example, the formulations of the present disclosure may have a pH of about 5.2; about 5.3; about 5.4; about 5.5; about 5.6; about 5.7; about 5.8; about 5.9; about 6.0; about 6.1; about 6.2; about 6.3; or about 6.4. In some embodiments, the pH is about 5.3±0.2; about 5.9±0.2; or about 6.0±0.2.

In some embodiments, the buffer or buffer system comprises at least one buffer that has a buffering range that overlaps fully or in part the range of pH 5.2-6.4. In one embodiment, the buffer or buffer system comprises two buffers, the first of which has an effective pH range within 3.6-5.6 and the second of which has an effective pH range within 5.5-7.4. In one embodiment, the first buffer has a pKa of about 4.8±0.3 and the second buffer has a pKa of about 6.0±0.3. In certain embodiments, the buffer system comprises an acetate buffer and a histidine buffer. In certain embodiments, the histidine is present at about 1.3-1.9 parts per 1 part of acetate by mole. In certain embodiments, the histidine is present at about 1.6±0.25 parts to 1 part of acetate by mole. In certain embodiments, the acetate is present at a concentration of about 2.5 mM to about 22.5 mM; about 3.0 mM to about 22 mM; about 3.5 mM to about 21.5 mM; about 4.0 mM to about 21.0 mM; about 4.5 mM to about 20.5 mM; about 5.0 mM to about 20 mM; about 5.5 mM to about 19.5 mM; about 6.0 mM to about 19.0 mM; about 6.5 mM to about 18.5 mM; about 7.0 mM to about 18.0 mM; about 7.5 mM to about 17.5 mM; about 8.0 mM to about 17 mM; about 8.5 mM to about 16.5 mM; about 9.0 mM to about 16.0 mM; about 9.5 mM to about 15.5 mM; about 10.0 mM to about 15.0 mM; about 10.5 mM to about 14.5 mM; about 12.5 mM±1.875 mM; about 11.0 mM to about 14.0 mM; about 11.5 mM to about 13.5 mM; or about 12.0 mM to about 13.0 mM. In certain embodiments, the histidine is present at a concentration of about 10 mM to about 30 mM; about 11 mM to about 29 mM; about 12 mM to about 28 mM; about 13 mM to about 27 mM; about 14 mM to about 26 mM; about 15 mM to about 25 mM; about 16 mM to about 24 mM; about 17 mM to about 23 mM; about 18 mM to about 22 mM; or about 19 mM to about 21 mM. In certain embodiments, the buffer system comprises acetate at about 12.5 mM and histidine at about 20 mM, at a pH of about 5.9.

The pharmaceutical formulations of the present disclosure may also comprise one or more excipients, which serve to maintain a reduced viscosity or to lower the viscosity of formulations containing a high concentration of protein (e.g., generally >100 mg/ml of protein). In some embodiments, the formulation comprises arginine in an amount sufficient to maintain the viscosity of the liquid formulation at less than about 35 cPoise, less than about 30 cPoise, less than about 25 cPoise, less than about 20 cPoise, less than about 15 cPoise, less than about 14 cPoise, less than about 13 cPoise, less than about 12 cPoise, less than about 10 cPoise, or less than about 9 cPoise.

In certain embodiments, the pharmaceutical formulation of the present disclosure contains arginine, preferably as L-arginine hydrochloride, at a concentration of about 25 mM±3.75 mM, about 50 mM±7.5 mM, or about 100 mM±15 mM. In certain embodiments, the arginine is at about 20 mM to about 30 mM, about 21 mM to about 29 mM, about 21.25 mM to about 28.75 mM, about 22 mM to about 28 mM, about 23 mM to about 27 mM or about 24 mM to about 26 mM.

According to one aspect of the present disclosure, the pharmaceutical formulation is a low viscosity, generally physiologically isotonic liquid formulation, which comprises: (i) a human antibody that specifically binds to hIL-4Rα (e.g., mAb1, mAb2 or mAb3 [supra]), at a concentration of about 100 mg/ml or greater; (ii) a buffer system that provides sufficient buffering at about 5.9±0.6; (iii) a sugar which serves inter alia as a thermal stabilizer; (iv) an organic cosolvent, which protects the structural integrity if the antibody; and (v) an amino acid, which serves to keep the viscosity manageable for subcutaneous injection.

According to one embodiment, the pharmaceutical formulation comprises: (i) a human IgG1 antibody that specifically binds to hIL-4Rα and which comprises a substituted IGHV3-9 type heavy chain variable region and a substituted IGLV2-28 type light chain variable region (e.g., mAb1) at a concentration from about 100 mg/ml to about 200 mg/ml; (ii) a buffer system comprising acetate and histidine, which buffers effectively at about pH 5.9±0.6; (iii) sucrose as a thermal stabilizer; (iv) a polysorbate as an organic cosolvent; and (v) arginine as a viscosity reducer.

According to one embodiment, the pharmaceutical formulation comprises: (i) a human IgG1 antibody that specifically binds to hIL-4Rα, and which comprises an HCDR1 of SEQ ID NO:2, an HCDR2 of SEQ ID NO:3, an HCDR3 of SEQ ID NO:4, an LCDR1 of SEQ ID NO:6, an LCDR2 of SEQ ID NO:7, and an LCDR3 of SEQ ID NO:8, at a concentration of about 150 mg/ml±25 mg/ml; (ii) acetate at about 12.5 mM±1.9 mM and histidine at about 20 mM±3 mM, which buffers effectively at about pH 5.9±0.3; (iii) sucrose at about 5% w/v±0.75% w/v; (iv) polysorbate 20 at about 0.2% w/v±0.03% w/v; and (v) arginine as L-arginine hydrochloride at about 25 mM±3.75 mM.

Additional non-limiting examples of pharmaceutical formulations encompassed by the present disclosure are set forth elsewhere herein, including the working Examples presented below.

The pharmaceutical formulations of the present disclosure typically exhibit high levels of stability. The term “stable”, as used herein in reference to the pharmaceutical formulations, means that the antibodies within the pharmaceutical formulations retain an acceptable degree of chemical structure or biological function after storage under defined conditions. A formulation may be stable even though the antibody contained therein does not maintain 100% of its chemical structure or biological function after storage for a defined amount of time. Under certain circumstances, maintenance of about 90%, about 95%, about 96%, about 97%, about 98% or about 99% of an antibody's structure or function after storage for a defined amount of time may be regarded as “stable”.

Stability can be measured, inter alia, by determining the percentage of native antibody that remains in the formulation after storage for a defined amount of time at a defined temperature. The percentage of native antibody can be determined by, inter alia, size exclusion chromatography (e.g., size exclusion high performance liquid chromatography [SE-HPLC]). An “acceptable degree of stability”, as that phrase is used herein, means that at least 90% of the native form of the antibody can be detected in the formulation after storage for a defined amount of time at a given temperature. In certain embodiments, at least about 90%, 91%, 92%, 93%, 94%, 95%, 96%, 97%, 98%, 99% or 100% of the native form of the antibody can be detected in the formulation after storage for a defined amount of time at a defined temperature. The defined amount of time after which stability is measured can be at least 2 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The defined temperature at which the pharmaceutical formulation may be stored when assessing stability can be any temperature from about −80° C. to about 45° C., e.g., storage at about −30° C., about −20° C., about 0° C., about 4°−8° C., about 5° C., about 25° C., or about 45° C. For example, a pharmaceutical formulation may be deemed stable if after 3 months of storage at 5° C., greater than about 90%, 95%, 96%, 97% or 98% of native antibody is detected by SE-HPLC. A pharmaceutical formulation may also be deemed stable if after 6 months of storage at 5° C., greater than about 90%, 95%, 96%, 97% or 98% of native antibody is detected by SE-HPLC. A pharmaceutical formulation may also be deemed stable if after 9 months of storage at 5° C., greater than about 90%, 95%, 96%, 97% or 98% of native antibody is detected by SE-HPLC. A pharmaceutical formulation may also be deemed stable if after 3 months of storage at 25° C., greater than about 90%, 95%, 96% or 97% of native antibody is detected by SE-HPLC. A pharmaceutical formulation may also be deemed stable if after 6 months of storage at 25° C., greater than about 90%, 95%, 96% or 97% of native antibody is detected by SE-HPLC. A pharmaceutical formulation may also be deemed stable if after 9 months of storage at 25° C., greater than about 90%, 95%, 96% or 97% of native antibody is detected by SE-HPLC.

Stability can be measured, inter alia, by determining the percentage of antibody that forms in an aggregate within the formulation after storage for a defined amount of time at a defined temperature, wherein stability is inversely proportional to the percent aggregate that is formed. The percentage of aggregated antibody can be determined by, inter alia, size exclusion chromatography (e.g., size exclusion high performance liquid chromatography [SE-HPLC]). An “acceptable degree of stability”, as that phrase is used herein, means that at most 5% of the antibody is in an aggregated form detected in the formulation after storage for a defined amount of time at a given temperature. In certain embodiments an acceptable degree of stability means that at most about 5%, 4%, 3%, 2%, 1%, 0.5%, or 0.1% of the antibody can be detected in an aggregate in the formulation after storage for a defined amount of time at a given temperature. The defined amount of time after which stability is measured can be at least 2 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 45° C., e.g., storage at about −30° C., about −20° C., about 0° C., about 4°-8° C., about 5° C., about 25° C., or about 45° C. For example, a pharmaceutical formulation may be deemed stable if after 3 months of storage at 5° C., less than about 5%, 4%, 3%, 2%, 1%, 0.5%, or 0.1% of the antibody is detected in an aggregated form. A pharmaceutical formulation may also be deemed stable if after 6 months of storage at 5° C., less than about 5%, 4%, 3%, 2%, 1%, 0.5%, or 0.1% of the antibody is detected in an aggregated form. A pharmaceutical formulation may also be deemed stable if after 9 months of storage at 5° C., less than about 5%, 4%, 3%, 2%, 1%, 0.5%, or 0.1% of the antibody is detected in an aggregated form. A pharmaceutical formulation may also be deemed stable if after 3 months of storage at 25° C., less than about 5%, 4%, 3%, 2%, 1%, 0.5%, or 0.1% of the antibody is detected in an aggregated form. A pharmaceutical formulation may also be deemed stable if after 6 months of storage at 25° C., less than about 5%, 4%, 3%, 2%, 1%, 0.5%, or 0.1% of the antibody is detected in an aggregated form. A pharmaceutical formulation may also be deemed stable if after 9 months of storage at 25° C., less than about 5%, 4%, 3%, 2%, 1%, 0.5%, or 0.1% of the antibody is detected in an aggregated form.

Stability can be measured, inter alia, by determining the percentage of antibody that migrates in a more acidic fraction during ion exchange (“acidic form”) than in the main fraction of antibody (“neutral conformation”), wherein stability is inversely proportional to the fraction of antibody in the acidic form. While not wishing to be bound by theory, deamidation of the antibody may cause the antibody to become more negatively charged and thus more acidic relative to the non-deamidated antibody (see, e.g., Robinson, N., Protein Deamidation, PNAS, Apr. 16, 2002, 99 (8): 5283-5288). The percentage of “acidified” or “deamidated” antibody can be determined by, inter alia, ion exchange chromatography (e.g., cation exchange high performance liquid chromatography [CEX-HPLC]). An “acceptable degree of stability”, as that phrase is used herein, means that at most 45% of the antibody is in a more acidic form detected in the formulation after storage for a defined amount of time at a defined temperature. In certain embodiments an acceptable degree of stability means that at most about 45%, 40%, 35%, 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. The defined amount of time after which stability is measured can be at least 2 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 45° C., e.g., storage at about −30° C., about −20° C., about 0° C., about 4°-8° C., about 5° C., about 25° C., or about 45° C. For example, a pharmaceutical formulation may be deemed stable if after 3 months of storage at 5° C., less than about 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 3 months of storage at 25° C., less than about 18%, 17%, 16%, 15%, 14%, 13%, 12%, 10%, 9%, 8%, 7%, 6%, 5%, 4%, 3%, 2%, 1%, 0.5% or 0.1 of the antibody is in a more acidic form. A pharmaceutical formulation may also be deemed stable if after 8 weeks of storage at 45° C., less than about 45%, 40%, 35%, 30%, 25%, 20%, 15%, 10%, 5%, 4%, 3%, 2%, 1%, 0.5%, or 0.1% of the antibody is in a more acidic form. A pharmaceutical formulation may also be deemed stable if after 2 weeks of storage at 40° C., less than about 20%, 19%, 18%, 17%, 16%, 15%, 14%, 13%, 12%, 11%, 10%, 9%, 8%, 7%, 6%, 5%, 4%, 3%, 2%, 1%, 0.5%, or 0.1% of the antibody can be detected in a more acidic form.

Other methods may be used to assess the stability of the formulations of the present disclosure such as, e.g., differential scanning calorimetry (DSC) to determine thermal stability, controlled agitation to determine mechanical stability, and absorbance at about 350 nm or about 405 nm to determine solution turbidities. For example, a formulation of the present disclosure may be considered stable if, after 6 or more months of storage at about 5° C. to about 25° C., the change in OD405 of the formulation is less than about 0.05 (e.g., 0.04, 0.03, 0.02, 0.01, or less) from the OD405 of the formulation at time zero.

Stability may also be assessed by measuring the biological activity or binding affinity of the antibody to its target. For example, a formulation of the present disclosure may be regarded as stable if, after storage at e.g., 5° C., 25° C., 45° C., etc. for a defined amount of time (e.g., 1 to 12 months), the anti-IL-4Rα antibody contained within the formulation binds to IL-4Rα with an affinity that is at least 90%, 95%, or more of the binding affinity of the antibody prior to said storage. Binding affinity may be determined by e.g., ELISA or plasmon resonance. Biological activity may be determined by an IL-4Rα activity assay, such as e.g., contacting a cell that expresses IL-4Rα with the formulation comprising the anti IL-4Rα antibody. The binding of the antibody to such a cell may be measured directly, such as e.g., via FACS analysis. Alternatively, the downstream activity of the IL-4Rα system may be measured in the presence of the antibody and an IL-4Rα agonist, and compared to the activity of the IL-4Rα system in the absence of antibody. In some embodiments, the IL-4Rα may be endogenous to the cell. In other embodiments, the IL-4Rα may be ectopically expressed in the cell.

Additional methods for assessing the stability of an antibody in formulation are demonstrated in the Examples presented below.

The liquid pharmaceutical formulations of the present disclosure may, in certain embodiments, exhibit low to moderate levels of viscosity. “Viscosity” as used herein may be “kinematic viscosity” or “absolute viscosity”. “Kinematic viscosity” is a measure of the resistive flow of a fluid under the influence of gravity. When two fluids of equal volume are placed in identical capillary viscometers and allowed to flow by gravity, a viscous fluid takes longer than a less viscous fluid to flow through the capillary. For example, if one fluid takes 200 seconds to complete its flow and another fluid takes 400 seconds, the second fluid is twice as viscous as the first on a kinematic viscosity scale. “Absolute viscosity”, sometimes called dynamic or simple viscosity, is the product of kinematic viscosity and fluid density (Absolute Viscosity=Kinematic Viscosity×Density). The dimension of kinematic viscosity is L2/T where L is a length and T is a time. Commonly, kinematic viscosity is expressed in centistokes (cSt). The SI unit of kinematic viscosity is mm2/s, which is 1 cSt. Absolute viscosity is expressed in units of centipoise (cP). The SI unit of absolute viscosity is the milliPascal-second (mPa-s), where 1 cP=1 mPa-s.

As used herein, a low level of viscosity, in reference to a fluid formulation of the present disclosure, will exhibit an absolute viscosity of less than about 15 cPoise (cP). For example, a fluid formulation of the disclosure will be deemed to have “low viscosity”, if, when measured using standard viscosity measurement techniques, the formulation exhibits an absolute viscosity of about 15 CP, about 14 cP, about 13 cP, about 12 cP, about 11 cP, about 10 cP, about 9 cP, about 8 cP, or less. As used herein, a moderate level of viscosity, in reference to a fluid formulation of the present disclosure, will exhibit an absolute viscosity of between about 35 cP and about 15 cP. For example, a fluid formulation of the disclosure will be deemed to have “moderate viscosity”, if when measured using standard viscosity measurement techniques, the formulation exhibits an absolute viscosity of about 34 cP, about 33 cP, about 32 cP, about 31 cP, about 30 cP, about 29 cP, about 28 cP, about 27 cP, about 26 cP, about 25 cP, about 24 cP, about 23 CP, about 22 cP, about 21 cP, about 20 cP, about 19 cP, 18 cP, about 17 cP, about 16 cP, or about 15.1 cP.

As illustrated in the examples below, the present inventors have made the surprising discovery that low to moderate viscosity liquid formulations comprising high concentrations of an anti-hIL-4Rα antibody (e.g., from about 100 mg/ml up to at least 200 mg/mL) can be obtained by formulating the antibody with arginine from about 25 mM to about 100 mM. In addition, it was further discovered that the viscosity of the formulation could be decreased to an even greater extent by adjusting the sucrose content to less than about 10%.

The pharmaceutical formulations of the present disclosure may be contained within any container suitable for storage of medicines and other therapeutic compositions. For example, the pharmaceutical formulations may be contained within a sealed and sterilized plastic or glass container having a defined volume such as a vial, ampule, syringe, cartridge, or bottle. Different types of vials can be used to contain the formulations of the present disclosure including, e.g., clear and opaque (e.g., amber) glass or plastic vials. Likewise, any type of syringe can be used to contain or administer the pharmaceutical formulations of the present disclosure.

The pharmaceutical formulations of the present disclosure may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than 500 parts per billion (ppb) of tungsten. The term “low tungsten” means that the syringe contains less than 500 ppb of tungsten. For example, a low tungsten syringe, according to the present disclosure, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial, or to preserve their stability. Thus, pharmaceutical formulations of the present disclosure, according to certain embodiments, may be contained within a syringe that comprises a coated plunger, or within a vial that is sealed with a coated rubber stopper. For example, the plunger or stopper may be coated with a fluorocarbon film. Examples of coated stoppers or plungers suitable for use with vials and syringes containing the pharmaceutical formulations of the present disclosure are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present disclosure are commercially available under the tradename “FluoroTec®”, available from West Pharmaceutical Services, Inc. (Lionville, Pa.).

According to certain embodiments of the present disclosure, the pharmaceutical formulations may be contained within a low tungsten syringe that comprises a fluorocarbon-coated plunger.

In one embodiment, the liquid pharmaceutical formulation containing about 150 mg/ml±15 mg/ml anti-IL-4Rα antibody is administered subcutaneously in a volume of approximately 1 ml±0.15 ml from a prefilled syringe in an autoinjector.

The pharmaceutical formulations of the present disclosure are useful, inter alia, for the treatment, prevention or amelioration of any disease or disorder associated with IL-4 activity, including diseases or disorders mediated by activation of IL-4Rα. Exemplary, non-limiting diseases and disorders that can be treated or prevented by the administration of the pharmaceutical formulations of the present disclosure include various atopic diseases such as, e.g., atopic dermatitis, allergic conjunctivitis, allergic rhinitis, asthma and other IgE/Th2 mediated diseases.

Thus, the present disclosure includes methods of treating, preventing, or ameliorating any disease or disorder associated with IL-4 activity or IL-4Rα activation (including any of the above mentioned exemplary diseases, disorders and conditions). The therapeutic methods of the present disclosure comprise administering to a subject any formulation comprising an anti-hIL-4Rα antibody as disclosed herein. The subject to which the pharmaceutical formulation is administered can be, e.g., any human or non-human animal that is in need of such treatment, prevention or amelioration, or who would otherwise benefit from the inhibition or attenuation of IL-4 or IL-4Rα-mediated activity. For example, the subject can be an individual that is diagnosed with, or who is deemed to be at risk of being afflicted by any of the aforementioned diseases or disorders. The present disclosure further includes the use of any of the pharmaceutical formulations disclosed herein in the manufacture of a medicament for the treatment, prevention or amelioration of any disease or disorder associated with IL-4 activity or IL-4Rα activation (including any of the above mentioned exemplary diseases, disorders and conditions).

### EXAMPLES

The following examples are presented so as to provide those of ordinary skill in the art with a complete disclosure and description of how to make and use the methods and compositions of the disclosure, and are not intended to limit the scope of the disclosure. Efforts have been made to ensure accuracy with respect to numbers used (e.g., amounts, temperature, etc.) but some experimental errors and deviations should be accounted for. Unless indicated otherwise, parts are parts by mole, molecular weight is average molecular weight, temperature is in degrees Centigrade, and pressure is at or near atmospheric pressure.

Initial formulation development activities involved screening organic cosolvents, thermal stabilizers, and buffers in liquid formulations of mAb1 (anti-IL-4Rα antibody of the disclosure) to identify excipients that are compatible with the protein and enhance its stability, while maintaining osmolality and viscosity for subcutaneous injection. Buffer conditions were also examined to determine the optimal pH for maximum protein stability.

### Example 1: Organic Cosolvents

It was observed that mAb1 is unstable when subjected to agitation stress. Analysis by reverse phase high performance liquid chromatography (RP-HPLC) and size exclusion high performance liquid chromatography (SE-HPLC) demonstrated a loss of protein and an increase of protein aggregates when mAb1 was vortexed at room temperature (Table 1, see “No Cosolvent” data). The addition of organic cosolvents to the mAb1 solution prevented the protein from degradation, as measured by SE-HPLC and RP-HPLC (Table 1). However, the additions of some of the organic cosolvents were observed to decrease the thermal stability of mAb1 (Table 2). A loss of protein recovery was observed in formulations containing PEG 3350 (3%) and PEG 300 (10% and 20%) as determined by RP-HPLC following thermal stress (Table 2). In addition, there was more aggregate formation in the formulations containing PLURONIC F68 (poloxamer 181) (0.2%), PEG 300 (10% and 20%), and Propylene Glycol (20%) than in the formulation without cosolvent as determined by SE-HPLC. Polysorbate 20 (0.2%) and polysorbate 80 (0.2%) provided comparable stability to agitation and thermal stress.

According to Table 1, 0.3 ml of 15 mg/ml of mAb1 in 10 mM phosphate, pH 6.0, and various organic cosolvents in a 2 ml glass vial were subjected to vortexing for about 120 minutes. Turbidity was assessed via optical density (OD) at 405 nm and reported as the relative change in OD at 405 nm as compared to the starting material. The percent of total mAb1 recovered was determined via reverse phase HPLC (RP-HPLC). The percent native and aggregated mAb1 was determined via size exclusion HPLC (SE-HPLC). The SE-HPLC results presented in the “Starting Material” results are the average of the values of each of the formulations in the absence of vortexing.

TABLE 1

| | Visual | | | % Total | % Native | % mAb1 |
|---|---|---|---|---|---|---|
| | Appear- | | | mAB1 | mAb1 | Aggregate |
| Organic Cosolvent | ance | Turbidity | pH | (RP-HPLC) | (SE-HPLC) | (SE-HPLC) |
| Starting Material² | Pass | 0.00 | 6.0 | 100 | 96.8 | 1.8 |
| (no vortexing) | | | | | | |
| No Cosolvent | Fall | 0.87 | 6.0 | 86 | 95.6 | 3.5 |
| 0.2% Polysorbate 20 | Pass | 0.01 | 5.9 | 98 | 97.0 | 1.7 |
| 0.2% Polysorbate 80 | Pass | 0.00 | 5.9 | 100 | 96.6 | 2.0 |
| 0.2% Pluronic F68 | Pass | 0.00 | 5.9 | 99 | 96.9 | 1.7 |
| 3% PEG 3350 | Pass | 0.00 | 6.0 | 102 | 96.7 | 2.0 |
| 1% PEG 3350 | Pass | 0.01 | 6.0 | 99 | 96.8 | 1.8 |
| 20% PEG 300 | Pass | 0.01 | 5.9 | 101 | 96.1 | 2.6 |
| 10% PEG 300 | Pass | 0.01 | 6.0 | 100 | 96.7 | 2.0 |
| 20% Propylene | Pass | 0.00 | 6.0 | 101 | 96.7 | 2.0 |
| Glycol | | | | | | |

According to Table 2, 0.3 ml of 15 mg/ml of mAb1 in 10 mM phosphate, pH 6.0, and various organic cosolvents in a 2 ml glass vial were kept at about 45° C. for about 28 days. Turbidity was assessed via optical density (OD) at 405 nm and reported as the relative change in OD at 405 nm as compared to the starting material. The percent of total mAb1 recovered was determined via reverse phase HPLC (RP-HPLC). The percent native and aggregated mAb1 was determined via size exclusion HPLC (SE-HPLC). The SE-HPLC results presented in the “Starting Material” results are the average of the values of each of the formulations in the absence of thermal stress.

TABLE 2

| | Visual | | | % Total | % Native | % mAb1 |
|---|---|---|---|---|---|---|
| | Appear- | | | mAb1 | mAb1 | Aggregate |
| Organic Cosolvent | ance | Turbidity | pH | (RP-HPLC) | (SE-HPLC) | (SE-HPLC) |
| Starting Material | Pass | 0.00 | 6.0 | 100 | 96.8 | 1.8 |
| (no acceleration) | | | | | | |
| No Cosolvent | Pass | 0.00 | 6.2 | 98 | 94.9 | 3.5 |
| 0.2% Polysorbate 20 | Pass | 0.00 | 6.3 | 98 | 94.6 | 3.6 |
| 0.2% Polysorbate 80 | Pass | 0.00 | 6.2 | 97 | 94.3 | 3.8 |
| 0.2% Pluronic F68 | Pass | 0.00 | 6.2 | 96 | 93.0 | 5.1 |
| 3% PEG 3350 | Pass | 0.00 | 6.2 | 73 | 96.5 | 1.4 |
| 1% PEG 3350 | Pass | 0.01 | 6.0 | 97 | 94.6 | 3.8 |
| 20% PEG 300 | Pass | 0.04 | 4.5 | 74 | 8.5 | 87.5 |
| 10% PEG 300 | Pass | 0.02 | 4.8 | 93 | 57.7 | 38.1 |
| 20% Propylene | Pass | 0.00 | 6.3 | 97 | 93.6 | 4.7 |
| Glycol | | | | | | |

### Example 2: Thermal Stabilizers

Various thermal stabilizers, such as sugars, amino acids, and inorganic salts, were examined for their ability to inhibit the degradation of mAb1 when kept at about 45° C. A summary of the thermal stabilizers studied is presented in Table 3. Formulations containing either sucrose or trehalose had the greatest stabilizing effect for mAb1 in solution when incubated at elevated temperature (as determined by SE-HPLC). Sucrose was selected as the stabilizer since it has a safe history of use in monoclonal antibody formulations.

According to Table 3, 0.3 ml of 25 mg/ml of mAb1 in 10 mM acetate, pH 5.3, and various thermal stabilizers in a 2 ml glass vial were kept at about 45° C. for about 28 days. Turbidity was assessed via optical density (OD) at 405 nm and reported as the relative change in OD at 405 nm as compared to the starting material. Turbidity was negligible for all samples. The percent of total mAb1 recovered was determined via reverse phase HPLC (RP-HPLC). The percent native and aggregated mAb1 was determined via size exclusion HPLC (SE-HPLC). Acidic or basic species are defined as the sum of the mAb1 peaks that elute from the cation exchange (CEX-HPLC) column with earlier or later retention times than the main peak, respectively. The SE-HPLC results presented in the “Starting Material” results are the average of the values of each of the formulations in the absence of thermal stress.

TABLE 3

| | | | | | % mAb1 | | |
|---|---|---|---|---|---|---|---|
| | | % Total | % Native | % mAb1 | (CEX- HPLC) | | |
| | | mAb1 | mAb1 | Aggregate | Acidic | Main | Basic |
| Buffer and pH | pH | (RP-HPLC) | (SE-HPLC) | (SE-HPLC) | Peak | Peak | Peak |
| Starting Material | 5.3 | 100 | 97.8 | 1.2 | 17.6 | 68.2 | 13.2 |
| (no 45° C. incubation) | | | | | | | |
| No Thermal Stabilizer | 5.4 | 106 | 91.9 | 5.8 | 28.1 | 56.5 | 15.4 |
| 8.5% Sucrose | 5.4 | 105 | 93.3 | 4.6 | 29.5 | 54.7 | 15.8 |
| 4.5% Sorbitol | 5.3 | 105 | 91.2 | 6.6 | 34.4 | 51.5 | 14.1 |
| 4.5% Mannitol | 5.3 | 104 | 92.6 | 5.2 | 28.4 | 66.0 | 15.6 |
| 9.4% Trehalose | 5.4 | 103 | 93.4 | 4.5 | 29.1 | 55.6 | 15.3 |
| dihydrate | | | | | | | |
| 2.2% Glycine | 5.4 | 104 | 86.6 | 10.6 | 33.5 | 50.7 | 15.8 |
| 0.9% NaCl | 5.4 | 98 | 85.0 | 8.7 | 25.2 | 56.0 | 18.7 |
| 2.5% Glycerol | 5.4 | 104 | 91.9 | 6.0 | 29.7 | 56.1 | 14.3 |
| 5% Arginine | 5.4 | 97 | 83.2 | 11.4 | 25.3 | 57.1 | 17.6 |

### Example 3: Buffers and pH

The effect of pH and buffer species on mAb1 stability was also examined. 15 mg/ml of mAb1 was incubated in different buffers at different pH values ranging from pH 4.5 to 7.0. Protein stability was monitored by SE-HPLC and cation exchange HPLC (CEX-HPLC). Maximum protein stability was observed, as determined by both SE-HPLC and CEX-HPLC, when mAb1 was formulated at pH 6.0 in histidine buffer or at pH 5.3 in acetate buffer (Table 4 and Table 5). The acetate buffer system provided a broader pH stability range and lower rate of charge variant formation relative to the formulation containing histidine buffer (Table 5). Therefore, acetate buffer, at pH 5.3, was selected in part for the formulation of the mAb1 drug substance.

According to Table 4, 0.3 ml of 15 mg/ml of mAb1, 0.2% polysorbate 20, combined with 10 mM of various buffers in a 2 ml glass vial were kept at about 45° C. for about 14 days. Turbidity was assessed via optical density (OD) at 405 nm and reported as the relative change in OD at 405 nm as compared to the starting material. Turbidity was negligible for all samples. The percent of total mAb1 recovered was determined via reverse phase HPLC (RP-HPLC). The percent native and aggregated mAb1 was determined via size exclusion HPLC (SE-HPLC). Acidic or basic species are defined as the sum of the mAb1 peaks that elute from the cation exchange (CEX-HPLC) column with earlier or later retention times than the main peak, respectively. The SE-HPLC results presented in the “Starting Material” results are the average of the values of each of the formulations in the absence of thermal stress.

TABLE 4

| | | % Native | % mAb1 | | | |
|---|---|---|---|---|---|---|
| | % Total | mAb1 | Aggregate | % mAbl Recovered² | | |
| | mAb1 | Recovered | Recovered | (CEX-HPLC) | | |
| | Recovered | (SE- | (SE- | Acidic | Main | Basic |
| Buffer and pH | (RP-HPLC) | HPLC) | HPLC) | Peaks | Peak | Peaks |
| Starting Material³ | 100 | 96.8 | 1.7 | 19.1 | 66.4 | 14.5 |
| (no 45° C. incubation) | | | | | | |
| pH 7.0, Phosphate | 97 | 93.9 | 4.5 | 39.1 | 50.1 | 10.8 |
| pH 6.5, Phosphate | 96 | 94.4 | 4.0 | 31.7 | 55.9 | 12.5 |
| pH 6.0, Phosphate | 99 | 95.2 | 3.1 | 23.8 | 62.2 | 14.0 |
| pH 6.0, Histidine | 97 | 95.5 | 2.8 | 23.9 | 63.8 | 14.3 |
| pH 6.0, Succinate | 99 | 94.8 | 3.5 | 26.7 | 59.6 | 13.7 |
| pH 6.0, Citrate | 98 | 95.5 | 2.9 | 26.1 | 59.8 | 14.1 |
| pH 5.5, Citrate | 96 | 94.7 | 3.4 | 25.0 | 60.9 | 14.2 |
| pH 5.0, Citrate | 97 | 89.5 | 7.4 | 23.6 | 61.5 | 15.0 |
| pH 5.0, Acetate | 94 | 94.7 | 3.6 | 18.1 | 66.3 | 15.5 |
| pH 4.5, Acciate | 94 | 89.9 | 8.3 | 20.8 | 62.8 | 16.4 |

According to Table 5, 0.3 ml of 15 mg/ml of mAb1, 0.2% polysorbate 20, combined with 10 mM of various buffers in a 2 ml glass vial were stored at about 45° C. for about 14 days. Turbidity was assessed via optical density (OD) at 405 nm and reported as the relative change in OD at 405 nm as compared to the starting material. Turbidity was negligible for all samples. The percent of total mAb1 recovered was determined via reverse phase HPLC (RP-HPLC). The percent native and aggregated mAb1 was determined via size exclusion HPLC (SE-HPLC). Acidic or basic species are defined as the sum of the mAb1 peaks that elute from the cation exchange (CEX-HPLC) column with earlier or later retention times than the main peak, respectively. The SE-HPLC results presented in the “Starting Material” results are the average of the values of each of the formulations in the absence of thermal stress.

Formulation development studies indicated that under basic conditions (pH 6.5), mAb1 in solution may deamidate. Conversely, below pH 5.0, the rate of formation of molecular weight variants of mAb1 was observed to increase. Based on these data, the pH of the mAb1 formulation was maintained between pH 5.6 and pH 6.2. mAb1 was observed to be stable over this pH range.

TABLE 5

| | % Total | % Native | % mAb1 | % Native mAb1 Recovered | | |
|---|---|---|---|---|---|---|
| | mAb1 | mAb1 | Aggregate | (CEX-HPLC) | | |
| | Recovered | Recovered | Recovered | Acidic | Main | Basic |
| Buffer and pH | [RP-HPLC) | (SE-HPLC) | (SE-HPLC) | Peaks | Peak | Peaks |
| Starting Material | 100 | 96.5 | 2.1 | 18.7 | 66.7 | 14.6 |
| (no 45° C. incubation) | | | | | | |
| pH 5.5, Histidine | 94 | 87.5 | 9.1 | 22.7 | 58.7 | 18.6 |
| pH 6.0, Histidine | 100 | 96.6 | 2.4 | 22.7 | 63.0 | 14.2 |
| pH 6.5, Histidine | 97 | 89.8 | 7.7 | 32.1 | 43.8 | 24.0 |
| pH 4.7, Acetate | 90 | 90.1 | 6.4 | 18.4 | 66.1 | 15.5 |
| pH 5.0, Acetate | 100 | 93.7 | 4.3 | 18.0 | 67.0 | 15.0 |
| pH 5.3. Acetate | 99 | 95.2 | 3.0 | 18.1 | 67.5 | 14.5 |
| pH 5.6, Acetate | 100 | 93.6 | 5.3 | 22.1 | 61.7 | 14.3 |

The effect of pH and buffer species on the stability of mAb1 was further evaluated in formulations containing either 20 mM histidine pH 6, 12.5 mM acetate pH 5.3, or a combination of 20 mM histidine and 12.5 acetate pH 5.9 (Table 6). Compared to the individual buffer system, mAb1 was most stable in a formulation containing both histidine and acetate at approximately pH 5.9. The slowest rate of aggregation was detected when mAb1 was formulated in this combined buffer system (SE-HPLC) (Table 6).

According to Table 6, 0.4 ml of 150 mg/ml of mAb1, 10% sucrose, 0.2% polysorbate 20, combined with various buffers in a 2 ml glass vial were kept at about 45° C. for about 14 days. Turbidity was assessed via optical density (OD) at 405 nm and reported as the relative change in OD at 405 nm as compared to the starting material. Turbidity was negligible for all samples. The percent of total mAb1 recovered was determined via reverse phase HPLC (RP-HPLC). The percent native and aggregated mAb1 was determined via size exclusion HPLC (SE-HPLC). Acidic or basic species are defined as the sum of the mAb1 peaks that elute from the cation exchange (CEX-HPLC) column with earlier or later retention times than the main peak, respectively. The SE-HPLC results presented in the “Starting Material” results are the average of the values of each of the formulations in the absence of thermal stress.

TABLE 6

| | % Total | % Native | % mAb1 | % mAb1 Recovered | | |
|---|---|---|---|---|---|---|
| | mAb1 | mAb1 | Aggregate | (CEX-HPLC) | | |
| | Recovered | Recovered | Recovered | Acidic | Main | Basic |
| Buffer and pH | (RP-HPLC) | (SE-HPLC) | (SE-HPLC) | Peaks | Peak | Peaks |
| Starting Material³ | 100 | 97.0 | 2.6 | 27.4 | 62.1 | 10.5 |
| (no 45° C. incubation) | | | | | | |
| 20 mM Histidine, pH 5.9 | 100 | 95.2 | 4.3 | 34.8 | 53.9 | 11.4 |
| 12.5 mM Acetate, pH 5.3. | 103 | 94.8 | 4.8 | 30.9 | 56.0 | 13.1 |
| Combined 20 mM Histidine | 104 | 95.9 | 3.7 | 33.7 | 54.1 | 12.1 |
| & 12.5 mM Acetate, pH 5.9 | | | | | | |

### Example 4: Management of Viscosity and Tonicity

Combinations of various excipients with high concentrations of mAb1 (i.e., 150 mg/ml, 175 mg/ml and 200 mg/ml) were assessed for viscosity and tonicity (as expressed in osmolality). The levels of sucrose, sodium chloride and L-arginine hydrochloride were adjusted to develop a formulation containing a high concentration of mAb1 at a low viscosity and at a physiological tonicity to enable the easy, comfortable and fast subcutaneous delivery of a high amount of mAb1 (Table 7). The liquid formulation containing 25 mM arginine, 20 mM histidine, 12.5 mM acetate, 5% (w/v) sucrose, 0.2% (w/v) Polysorbate 20, and 150 mg/ml mAb1, at pH 5.9 (Formulation A) represents an optimized formulation having a low viscosity (about 8.5 cPoise) and being physiologically isotonic (about 293 mOsm/kg), while maintaining the stability of mAb1.

### Example 5: Characterization of Formulation A

The main degradation pathways identified during the development of the mAb1 liquid formulation were the formation of aggregates, cleavage products, and charge variants. The formation of these degradation products was minimized by formulating mAb1 in a formulation containing 20 mM histidine, 12.5 mM acetate, 0.2% polysorbate 20, 5% sucrose and 25 mM L-arginine hydrochloride at pH 5.9.

The formulated 150 mg/ml mAb1 was observed to be clear to slightly opalescent liquid solution, essentially free from visible particles.

The formulated mAb1 was physically and chemically stable when subjected to various stress (25° C. and 45° C. incubation) and real-time storage condition (5° C.) (Table 8). The appearance was unaffected when the mAb1 was incubated at 25° C. (3 months) or stored at 5° C. for 6 months. In addition, no affect on solution pH, turbidity, or on the amount of recovered mAb1 was observed. Following incubation of formulated mAb1 for 3 months at 25° C., the antibody was not significantly degraded as determined by SE-HPLC and there was 3.3% more degraded as determined by CEX-HPLC. There was increased degradation observed following incubation at 45° C. for 8 weeks as determined by SE-HPLC and CEX-HPLC indicating that aggregate and charge variant formation are the main degradation routes for the mAb1 antibody molecule. No degradation was observed when the formulated mAb1 antibody was stored for 6 months at 5° C.

TABLE 7

| | mAb1 | Histidine | Acetate | Arginine | NaCl | Sucrose | | Viscosity | Osmolality |
|---|---|---|---|---|---|---|---|---|---|
| | (mg/ml) | (mM) | (mM) | (mM) | (mM) | (% w/v) | pH | (cPols.) | (mOsm/kg) |
| A | 150 | 20 | 12.5 | 25 | 0 | 5 | 5.9 | 8.5 | 293 |
| B | 150 | 20 | 12.5 | 0 | 0 | 10 | 5.9 | 11 | 448 |
| C | 175 | 20 | 12.5 | 100 | 0 | 1 | 5.9 | ~8.0 | ~290 |
| D | 175 | 20 | 12.5 | 50 | 0 | 5 | 5.9 | ~9.5 | ~370 |
| E | 175 | 20 | 12.5 | 0 | 0 | 10 | 5.9 | ~20 | ~440 |
| F | 200 | 20 | 12.5 | 100 | 0 | 1 | 5.9 | ~15 | ~290 |
| G | 200 | 20 | 12.5 | 0 | 100 | 5 | 5.9 | ~19.2 | ~430 |
| H | 200 | 20 | 12.5 | 100 | 0 | 5 | 5.9 | ~17 | ~430 |
| I | 200 | 20 | 12.5 | 50 | 0 | 5 | 5.8 | ~18 | ~330 |
| J | 200 | 20 | 12.5 | 25 | 0 | 5 | 5.9 | ~23 | ~290 |
| K | 200 | 20 | 12.5 | 0 | 0 | 10 | 5.9 | ~35 | ~440 |

According to Table 8, OD=Optical density; RP-HPLC=Reversed phase high performance liquid chromatography; SE-HPLC=Size exclusion high performance liquid chromatography; and CEX-HPLC=Cation exchange high performance liquid chromatography. Acidic or basic species are defined as the sum of mAb1 peaks that elute from the CEX-HPLC column with earlier or later retention times than the main peak, respectively.

### Example 6: Containers

Formulations containing mAb1 have been determined to be stable when filter sterilized. A Millipore MILLIPAK filtration unit was used in the manufacturing of the clinical supplies while a filter of identical composition was used in the research studies (Millipore MILLEX DURAPORE).

A 5-mL glass vial was filled with a minimum of 2.5 mL 150 mg/ml mAb1, 5% (w/v) sucrose, 25 mM L-arginine hydrochloride, 0.2% (w/v) polysorbate 20, 12.5 mM acetate, 20 mM histidine, pH 5.9. An overage of 0.5 mL of formulation was applied in the 5-mL vial to ensure that 2.0 mL of the formulation could be withdrawn. This overage was not designed to compensate for losses during manufacture of the mAb1 or formulation containing the mAb1, degradation during manufacture, degradation during storage (shelf life), or to extend the expiration dating period.

Compared to storage in glass vials, the stability of the formulated mAb1 (Formulation A) was not affected when stored in a either a polypropylene tube, a polystyrene tube, a polycarbonate tube, or in a glass vial containing pieces of stainless steel (Table 9).

TABLE 8

| | | No | | | | | |
|---|---|---|---|---|---|---|---|
| Stress Test | | Storage | 5° C. | | | 25° C. | |
| Length of Storage | | — | 2 mo. | 3 mo. | 6 mo. | 1 mo. | 3 mo. |
| Visual Appearance | | Pass | Pass | Pass | Pass | Pass | Pass |
| Turbidity (OD 405 nm) | | 0.00 | 0.00 | 0.00 | 0.00 | 0.01 | 0.01 |
| pH | | 6.0 | 6.0 | 5.9 | 5.9 | 6.0 | 6.0 |
| % mAb1 (RP-HPLC) | | 100 | 97 | 104 | | 97 | 102 |
| % Native mAb1 (SE-MPLC) | | 98.1 | 98.2 | 98.2 | | 98.1 | 97.8 |
| % mAb1 | Acidic | 14.6 | 14.7 | 14.7 | | 16.0 | 17.6 |
| (Peaks from | Main Peak | 70.7 | 70.5 | 70.4 | | 69.8 | 67.4 |
| CEX-HPLC) | Basic | 14.7 | 14.8 | 14.9 | | 14.3 | 15.0 |

| | | No | | | |
|---|---|---|---|---|---|
| Stress Test | | Storage | 45° C. | | |
| Length of Storage | | | 2 wk | 4 wk | 8 wk |
| Visual Appearance | | Pass | Pass | Pass | Pass |
| Turbidity (OD 405 nm) | | 0.00 | 0.02 | 0.03 | 0.05 |
| pH | | 6.0 | 6.0 | 6.0 | 6.0 |
| % mAb1 (RP-HPLC) | | 100 | 102 | 98 | 100 |
| % Native mAb1 (SE-HPLC) | | 98.1 | 95.9 | 94.2 | 90.5 |
| % mAb1 | Acidic | 14.6 | 20.8 | 29.9 | 44.0 |
| (Peaks from | Main Peak | 70.7 | 64.5 | 56.7 | 45.1 |
| CEX-HPLC) | Basic | 14.7 | 14.7 | 13.4 | 10.9 |

According to Table 9, 150 mg/ml mAb1, 5% Sucrose, 25 mM Arginine Hydrochloride, 0.2% PS-20, 20 mM Histidine, 12.5 mM Acetate, pH 5.9 was incubated with/in various materials at 40° C. for 14 days. OD=Optical density; RP-HPLC=Reversed phase high performance liquid chromatography; SE-HPLC=Size exclusion high performance liquid chromatography; and CEX-HPLC=Cation exchange high performance liquid chromatography. Turbidity is reported as the relative change in OD at 405 nm as compared to the starting material. Acidic or basic species are defined as the sum of mAb1 peaks that elute from the CEX-HPLC column with earlier or later retention times than the main peak, respectively.

TABLE 9

| Storage | | No | | | | | |
|---|---|---|---|---|---|---|---|
| Temperature | | Storage | 40 ° C. for 14 days | | | | |
| | | | | Poly- | Poly- | Poly- | Stainless |
| Storage Container | | Glass | Glass | propylene | styrene | carbonate | Steel |
| Visual Appearance | | Pass | Pass | Pass | Pass | Pass | Pass |
| Turbidity (OD at 405 am) | | 0.00 | 0.01 | 0.01 | 0.02 | 0.02 | 0.01 |
| pH | | 5.9 | 5.9 | 5.7 | 5.8 | 5.8 | 5.9 |
| % mAb1 (RP-HPLC) | | 100 | 102 | 103 | 107 | 106 | 102 |
| % Native mAb1 (SE- | | 98.4 | 97.6 | 97.4 | 97.5 | 97.5 | 96.1 |
| HPLC) | | | | | | | |
| % Peak | Acidic | 14.8 | 18.4 | 19.1 | 18.4 | 18.4 | 20.3 |
| mAb1 | Main Peak | 70.7 | 65.8 | 65.5 | 66.0 | 66.5 | 65.0 |
| (CEX-HPLC) | Basic | 14.5 | 15.8 | 15.3 | 15.6 | 15.1 | 14.7 |

Next, exemplary medicaments, drugs, and/or pharmaceutical formulations consistent with U.S. Pat. No. 9,987,500 B2 are described. The term “PD-1” refers to the programmed death-1 protein, a T-cell co-inhibitor, also known as CD279. The amino acid sequence of full-length PD-1 is provided in GenBank as accession number NP_005009.2 and is also referred to herein as SEQ ID NO: 327. The term “PD-1” also includes protein variants of PD-1 having the amino acid sequence of SEQ ID NOs: 321, 322, 323, or 324. The term “PD-1” includes recombinant PD-1 or a fragment thereof. The term also encompasses PD-1 or a fragment thereof coupled to, for example, histidine tag, mouse or human Fc, or a signal sequence such as ROR1. For example, the term includes sequences exemplified by SEQ ID NOs: 323 or 324, comprising a mouse Fc (mIgG2a) or human Fc (hIgG1) at the C-terminal, coupled to amino acid residues 25-170 of full-length PD-1 with a C93S change. Protein variants as exemplified by SEQ ID NO: 321 comprise a histidine tag at the C-terminal, coupled to amino acid residues 25-170 of full length PD-1. Unless specified as being from a non-human species, the term “PD-1” means human PD-1.

PD-1 is a member of the CD28/CTLA-4/ICOS family of T-cell co-inhibitors. PD-1 is a 288-amino acid protein with an extracellular N-terminal domain which is IgV-like, a transmembrane domain and an intracellular domain containing an immunoreceptor tyrosine-based inhibitory (ITIM) motif and an immunoreceptor tyrosine-based switch (ITSM) motif (Chattopadhyay et al 2009, Immunol. Rev.). The PD-1 receptor has two ligands, PD-ligand-1 (PD-L1) and PD-L2.

The term “PD-L1” refers to the ligand of the PD-1 receptor also known as CD274 and B7H1. The amino acid sequence of full-length PD-L1 is provided in GenBank as accession number NP_054862.1 and is also referred to herein as SEQ ID NO: 328. The term also encompasses PD-L1 or a fragment thereof coupled to, for example, histidine tag, mouse or human Fc, or a signal sequence such as ROR1. For example, the term includes sequences exemplified by SEQ ID NOs: 325 or 326, comprising a mouse Fc (mIgG2a) or human Fc (hIgG1) at the C-terminal, coupled to amino acid residues 19-239 of full-length PD-L1. PD-L1 is a 290 amino acid protein with an extracellular IgV-like domain, a transmembrane domain and a highly conserved intracellular domain of approximately 30 amino acids. PD-L1 is constitutively expressed on many cells such as antigen presenting cells (e.g., dendritic cells, macrophages, and B-cells) and on hematopoietic and non-hematopoietic cells (e.g., vascular endothelial cells, pancreatic islets, and sites of immune privilege). PD-L1 is also expressed on a wide variety of tumors, virally-infected cells and autoimmune tissue, and is a component of the immunosuppressive milieu (Ribas 2012, NEJM 366:2517-2519).

As used herein, the term “T-cell co-inhibitor” refers to a ligand and/or receptor which modulates the immune response via T-cell activation or suppression. The term “T-cell co-inhibitor”, also known as T-cell co-signaling molecule, includes, but is not limited to, lymphocyte activation gene 3 protein (LAG-3, also known as CD223), cytotoxic T-lymphocyte antigen-4 (CTLA-4), B and T lymphocyte attenuator (BTLA), CD-28, 2B4, LY108, T-cell immunoglobulin and mucin 3 (TIM3), T-cell immunoreceptor with immunoglobulin and ITIM (TIGIT; also known as VSIG9), leucocyte associated immunoglobulin-like receptor 1 (LAIR1; also known as CD305), inducible T-cell costimulator (ICOS; also known as CD278), V-domain Ig suppressor of T-cell activation (VISTA) and CD160.

As used herein, the term “Fc receptor” refers to the surface receptor protein found on immune cells including B lymphocytes, natural killer cells, macrophages, basophils, neutrophils, and mast cells, which has a binding specificity for the Fc region of an antibody. The term “Fc receptor” includes, but is not limited to, a Fcγ receptor [e.g., FcγRI (CD64), FcγRIIA (CD32), FcγRIIB (CD32), FcγRIIIA (CD16a), and FcγRIIIB (CD16b)], Fcα receptor (e.g., FcαRI or CD89) and Fcε receptor [e.g., FcεRI, and FcεRII (CD23)].

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “VH”) and a heavy chain constant region (comprised of domains C H1, CH2 and CH3). Each light chain is comprised of a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In certain embodiments of the disclosure, the FRs of the antibody (or antigen binding fragment thereof) may be identical to the human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The fully human anti-PD-1 monoclonal antibodies disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes fully human anti-PD-1 monoclonal antibodies comprising variants of any of the HCVR, LCVR, and/or CDR amino acid sequences disclosed herein having one or more conservative substitutions. For example, the present disclosure includes anti-PD-1 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody”, as used herein, is intended to include antibodies having variable and constant regions derived from human germline immunoglobulin sequences. The human mAbs of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “recombinant”, as used herein, refers to antibodies or antigen-binding fragments thereof of the disclosure created, expressed, isolated or obtained by technologies or methods known in the art as recombinant DNA technology which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

The term “multi-specific antigen-binding molecules”, as used herein refers to bispecific, tri-specific or multi-specific antigen-binding molecules, and antigen-binding fragments thereof. Multi-specific antigen-binding molecules may be specific for different epitopes of one target polypeptide or may contain antigen-binding domains specific for epitopes of more than one target polypeptide. A multi-specific antigen-binding molecule can be a single multifunctional polypeptide, or it can be a multimeric complex of two or more polypeptides that are covalently or non-covalently associated with one another. The term “multi-specific antigen-binding molecules” includes antibodies of the present disclosure that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multi-specific antigen-binding molecules” also includes bi-specific, tri-specific or multi-specific antibodies or antigen-binding fragments thereof. In certain embodiments, an antibody of the present disclosure is functionally linked to another antibody or antigen-binding fragment thereof to produce a bispecific antibody with a second binding specificity. Bispecific and multi-specific antibodies of the present disclosure are described elsewhere herein.

The term “specifically binds,” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10-8 M or less (e.g., a smaller KD denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to PD-1. Moreover, multi-specific antibodies that bind to one domain in PD-1 and one or more additional antigens or a bi-specific that binds to two different regions of PD-1 are nonetheless considered antibodies that “specifically bind”, as used herein.

The term “high affinity” antibody refers to those mAbs having a binding affinity to PD-1, expressed as KD, of at least 10-7 M; preferably 10-8 M; more preferably 10-9M, even more preferably 10-10 M, even more preferably 10-11 M, as measured by surface plasmon resonance, e.g., BIACORE™ or solution-affinity ELISA.

By the term “slow off rate”, “Koff” or “kd” is meant an antibody that dissociates from PD-1, with a rate constant of 1×10-3 s-1 or less, preferably 1×10-4 s-1 or less, as determined by surface plasmon resonance, e.g., BIACORE™.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to bind to PD-1.

In specific embodiments, antibody or antibody fragments of the disclosure may be conjugated to a moiety such a ligand or a therapeutic moiety (“immunoconjugate”), such as an antibiotic, a second anti-PD-1 antibody, or an antibody to another antigen such a tumor-specific antigen, an autoimmune tissue antigen, a virally-infected cell antigen, a Fc receptor, a T-cell receptor, or a T-cell co-inhibitor, or an immunotoxin, or any other therapeutic moiety useful for treating a disease or condition including cancer, autoimmune disease or chronic viral infection.

An “isolated antibody”, as used herein, is intended to refer to an antibody that is substantially free of other antibodies (Abs) having different antigenic specificities (e.g., an isolated antibody that specifically binds PD-1, or a fragment thereof, is substantially free of Abs that specifically bind antigens other than PD-1.

A “blocking antibody” or a “neutralizing antibody”, as used herein (or an “antibody that neutralizes PD-1 activity” or “antagonist antibody”), is intended to refer to an antibody whose binding to PD-1 results in inhibition of at least one biological activity of PD-1. For example, an antibody of the disclosure may prevent or block PD-1 binding to PD-L1.

An “activating antibody” or an “enhancing antibody”, as used herein (or an “agonist antibody”), is intended to refer to an antibody whose binding to PD-1 results in increasing or stimulating at least one biological activity of PD-1. For example, an antibody of the disclosure may increase PD-1 binding to PD-L1.

The term “surface plasmon resonance”, as used herein, refers to an optical phenomenon that allows for the analysis of real-time biomolecular interactions by detection of alterations in protein concentrations within a biosensor matrix, for example using the BIACORE™ system (Pharmacia Biosensor AB, Uppsala, Sweden and Piscataway, N.J.).

The term “KD”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 90%, and more preferably at least about 95%, 96%, 97%, 98% or 99% of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24:307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410 and (1997) Nucleic Acids Res. 25:3389-3402, each of which is herein incorporated by reference.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

As used herein, the term “subject” refers to an animal, preferably a mammal, in need of amelioration, prevention and/or treatment of a disease or disorder such as chronic viral infection, cancer or autoimmune disease.

As used herein, “anti-cancer drug” means any agent useful to treat cancer including, but not limited to, cytotoxins and agents such as antimetabolites, alkylating agents, anthracyclines, antibiotics, antimitotic agents, procarbazine, hydroxyurea, asparaginase, corticosteroids, mytotane (O,P′-(DDD)), biologics (e.g., antibodies and interferons) and radioactive agents. As used herein, “a cytotoxin or cytotoxic agent”, also refers to a chemotherapeutic agent and means any agent that is detrimental to cells. Examples include Taxol® (paclitaxel), temozolamide, cytochalasin B, gramicidin D, ethidium bromide, emetine, cisplatin, mitomycin, etoposide, tenoposide, vincristine, vinbiastine, coichicin, doxorubicin, daunorubicin, dihydroxy anthracin dione, mitoxantrone, mithramycin, actinomycin D,1-dehydrotestosterone, glucocorticoids, procaine, tetracaine, lidocaine, propranolol, and puromycin and analogs or homologs thereof.

As used herein, the term “anti-viral drug” refers to any drug or therapy used to treat, prevent, or ameliorate a viral infection in a host subject. The term “anti-viral drug” includes, but is not limited to zidovudine, lamivudine, abacavir, ribavirin, lopinavir, efavirenz, cobicistat, tenofovir, rilpivirine, analgesics and corticosteroids. In the context of the present disclosure, the viral infections include long-term or chronic infections caused by viruses including, but not limited to, human immunodeficiency virus (HIV), hepatitis B virus (HBV), hepatitis C virus (HCV), human papilloma virus (HPV), lymphocytic choriomeningitis virus (LCMV), and simian immunodeficiency virus (SIV).

The antibodies and antigen-binding fragments of the present disclosure specifically bind to PD-1 and modulate the interaction of PD-1 with PD-L1. The anti-PD-1 antibodies may bind to PD-1 with high affinity or with low affinity. In certain embodiments, the antibodies of the present disclosure may be blocking antibodies wherein the antibodies may bind to PD-1 and block the interaction of PD-1 with PD-L1. In some embodiments, the blocking antibodies of the disclosure may block the binding of PD-1 to PD-L1 and/or stimulate or enhance T-cell activation. In some embodiments, the blocking antibodies may be useful for stimulating or enhancing the immune response and/or for treating a subject suffering from cancer, or a chronic viral infection. The antibodies when administered to a subject in need thereof may reduce the chronic infection by a virus such as HIV, LCMV or HBV in the subject. They may be used to inhibit the growth of tumor cells in a subject. They may be used alone or as adjunct therapy with other therapeutic moieties or modalities known in the art for treating cancer, or viral infection.

In other embodiments, the antibodies of the present disclosure may be activating antibodies, wherein the antibodies may bind to PD-1 and enhance the interaction of PD-1 and PD-L1. In some embodiments, the activating antibodies may enhance binding of PD-1 to PD-L1 and/or inhibit or suppress T-cell activation. The activating antibodies of the present disclosure may be useful for inhibiting the immune response in a subject and/or for treating autoimmune disease.

In certain embodiments, the anti-PD-1 antibodies may be multi-specific antigen-binding molecules, wherein they comprise a first binding specificity to PD-1 and a second binding specificity to an antigen selected from the group consisting of another T-cell co-inhibitor, an autoimmune tissue antigen, T-cell receptor, Fc receptor, T-cell receptor, PD-L1, and a different epitope of PD-1.

In certain embodiments, the antibodies of the disclosure are obtained from mice immunized with a primary immunogen, such as a full length PD-1 [See GenBank accession number NP_005009.2 (SEQ ID NO: 327)] or with a recombinant form of PD-1 or modified human PD-1 fragments (SEQ ID NOs: 321, 323, or 324) or with modified cynomolgus PD-1 fragments (SEQ ID NO: 322), followed by immunization with a secondary immunogen, or with an immunogenically active fragment of PD-1.

The immunogen may be a biologically active and/or immunogenic fragment of PD-1 or DNA encoding the active fragment thereof. The fragment may be derived from the N-terminal or C-terminal domain of PD-1. In certain embodiments of the disclosure, the immunogen is a fragment of PD-1 that ranges from amino acid residues 25-170 of SEQ ID NO: 327 with a C93S change.

The peptides may be modified to include addition or substitution of certain residues for tagging or for purposes of conjugation to carrier molecules, such as, KLH. For example, a cysteine may be added at either the N terminal or C terminal end of a peptide, or a linker sequence may be added to prepare the peptide for conjugation to, for example, KLH for immunization.

The full-length amino acid sequence of full length human PD-1 is shown as SEQ ID NO: 327.

In certain embodiments, antibodies that bind specifically to PD-1 may be prepared using fragments of the above-noted regions, or peptides that extend beyond the designated regions by about 5 to about 20 amino acid residues from either, or both, the N or C terminal ends of the regions described herein. In certain embodiments, any combination of the above-noted regions or fragments thereof may be used in the preparation of PD-1 specific antibodies. In certain embodiments, any one or more of the above-noted regions of PD-1, or fragments thereof may be used for preparing monospecific, bispecific, or multispecific antibodies.

Certain anti-PD-1 antibodies of the present disclosure are able to bind to and neutralize the activity of PD-1, as determined by in vitro or in vivo assays. The ability of the antibodies of the disclosure to bind to and neutralize the activity of PD-1 may be measured using any standard method known to those skilled in the art, including binding assays, or activity assays, as described herein.

Non-limiting, exemplary in vitro assays for measuring binding activity are illustrated in Examples herein. In Example 3, the binding affinities and kinetic constants of human anti-PD-1 antibodies for human PD-1 and cynomolgus PD-1 were determined by surface plasmon resonance and the measurements were conducted on a Biacore 4000 or T200 instrument. In Examples 4 and 5, blocking assays were used to determine the ability of the anti-PD-1 antibodies to block PD-L1-binding ability of PD-1 in vitro. In Example 6, blocking assays were used to determine cross-competition between anti-PD-1 antibodies. Example 7 describes the binding of the antibodies to cells overexpressing PD-1. In Example 8, a luciferase assay was used to determine the ability of anti-PD-1 antibodies to antagonize PD-1/PD-L1 signaling in T-cells.

In certain embodiments, the antibodies of the present disclosure are able to enhance or stimulate T-cell activation in vitro and in a subject with cancer or in a subject infected with a virus such as LCMV. In certain embodiments, the antibodies of the present disclosure are used in combination with a second therapeutic agent, such as an antibody to a second T-cell co-inhibitor, to enhance the immune response and inhibit tumor growth in a subject.

The antibodies specific for PD-1 may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface. In one embodiment, the label may be a radionuclide, a fluorescent dye or a MRI-detectable label. In certain embodiments, such labeled antibodies may be used in diagnostic assays including imaging assays.

### Antigen-Binding Fragments of Antibodies

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen-binding fragments thereof. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. The terms “antigen-binding fragment” of an antibody, or “antibody fragment”, as used herein, refers to one or more fragments of an antibody that retain the ability to specifically bind to PD-1. An antibody fragment may include a Fab fragment, a F(ab′)2 fragment, a Fv fragment, a dAb fragment, a fragment containing a CDR, or an isolated CDR. In certain embodiments, the term “antigen-binding fragment” refers to a polypeptide fragment of a multi-specific antigen-binding molecule. In such embodiments, the term “antigen-binding fragment” includes, e.g., an extracellular domain of PD-L1 which binds specifically to PD-1. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-C H1; (ix) VL-CH2; (X) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present disclosure using routine techniques available in the art.

### Preparation of Human Antibodies

Methods for generating human antibodies in transgenic mice are known in the art. Any such known methods can be used in the context of the present disclosure to make human antibodies that specifically bind to PD-1.

An immunogen comprising any one of the following can be used to generate antibodies to PD-1. In certain embodiments, the antibodies of the disclosure are obtained from mice immunized with a full length, native PD-1 (See GenBank accession number NP_005009.2) (SEQ ID NO: 327), or with a recombinant PD-1 peptide. Alternatively, PD-1 or a fragment thereof may be produced using standard biochemical techniques and modified (SEQ ID NOS: 321-324) and used as immunogen.

In certain embodiments, the immunogen may be a peptide from the N terminal or C terminal end of PD-1. In one embodiment, the immunogen is the extracellular domain or the IgV-like domain of PD-1. In certain embodiments of the disclosure, the immunogen is a fragment of PD-1 that ranges from about amino acid residues 25-170 of SEQ ID NO: 327 with a C93S change.

In some embodiments, the immunogen may be a recombinant PD-1 peptide expressed in E. coli or in any other eukaryotic or mammalian cells such as Chinese hamster ovary (CHO) cells.

In certain embodiments, antibodies that bind specifically to PD-1 may be prepared using fragments of the above-noted regions, or peptides that extend beyond the designated regions by about 5 to about 20 amino acid residues from either, or both, the N or C terminal ends of the regions described herein. In certain embodiments, any combination of the above-noted regions or fragments thereof may be used in the preparation of PD-1 specific antibodies.

Using VELOCIMMUNE® technology (see, for example, U.S. Pat. No. 6,596,541, Regeneron Pharmaceuticals, VELOCIMMUNE®) or any other known method for generating monoclonal antibodies, high affinity chimeric antibodies to PD-1 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

### Bioequivalents

The anti-PD-1 antibodies and antibody fragments of the present disclosure encompass proteins having amino acid sequences that vary from those of the described antibodies, but that retain the ability to bind PD-1. Such variant antibodies and antibody fragments comprise one or more additions, deletions, or substitutions of amino acids when compared to parent sequence, but exhibit biological activity that is essentially equivalent to that of the described antibodies. Likewise, the antibody-encoding DNA sequences of the present disclosure encompass sequences that comprise one or more additions, deletions, or substitutions of nucleotides when compared to the disclosed sequence, but that encode an antibody or antibody fragment that is essentially bioequivalent to an antibody or antibody fragment of the disclosure.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, or potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antibodies of the disclosure may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include antibody variants comprising amino acid changes, which modify the glycosylation characteristics of the antibodies, e.g., mutations that eliminate or remove glycosylation.

### Anti-PD-1 Antibodies Comprising Fc Variants

According to certain embodiments of the present disclosure, anti-PD-1 antibodies are provided comprising an Fc domain comprising one or more mutations which enhance or diminish antibody binding to the FcRn receptor, e.g., at acidic pH as compared to neutral pH. For example, the present disclosure includes anti-PD-1 antibodies comprising a mutation in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434 W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present disclosure includes anti-PD-1 antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 257I and 311I (e.g., P257I and Q311I); 257I and 434H (e.g., P257I and N434H); 376V and 434H (e.g., D376V and N434H); 307A, 380A and 434A (e.g., T307A, E380A and N434A); and 433K and 434F (e.g., H433K and N434F). In one embodiment, the present disclosure includes anti-PD-1 antibodies comprising an Fc domain comprising a S108P mutation in the hinge region of IgG4 to promote dimer stabilization. All possible combinations of the foregoing Fc domain mutations, and other mutations within the antibody variable domains disclosed herein, are contemplated within the scope of the present disclosure.

The present disclosure also includes anti-PD-1 antibodies comprising a chimeric heavy chain constant (CH) region, wherein the chimeric CH region comprises segments derived from the CH regions of more than one immunoglobulin isotype. For example, the antibodies of the disclosure may comprise a chimeric CH region comprising part or all of a CH2 domain derived from a human IgG1, human IgG2 or human IgG4 molecule, combined with part or all of a CH3 domain derived from a human IgG1, human IgG2 or human IgG4 molecule. According to certain embodiments, the antibodies of the disclosure comprise a chimeric CH region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric CH region as described herein may, in certain embodiments, exhibit modified Fc effector functions without adversely affecting the therapeutic or pharmacokinetic properties of the antibody. (See, e.g., U.S. Ser. No. 14/170,166, filed Jan. 31, 2014, the disclosure of which is hereby incorporated by reference in its entirety).

### Biological Characteristics of the Antibodies

In general, the antibodies of the present disclosure function by binding to PD-1. The present disclosure includes anti-PD-1 antibodies and antigen-binding fragments thereof that bind soluble monomeric or dimeric PD-1 molecules with high affinity. For example, the present disclosure includes antibodies and antigen-binding fragments of antibodies that bind monomeric PD-1 (e.g., at 25° C. or at 37° C.) with a KD of less than about 50 nM as measured by surface plasmon resonance, e.g., using the assay format as defined in Example 3 herein. In certain embodiments, the antibodies or antigen-binding fragments thereof bind monomeric PD-1 with a KD of less than about 40 nM, less than about 30 nM, less than about 20 nM, less than about 10 nM less than about 5 nM, less than about 2 nM or less than about 1 nM, as measured by surface plasmon resonance, e.g., using the assay format as defined in Example 3 herein, or a substantially similar assay.

The present disclosure also includes antibodies and antigen-binding fragments thereof that bind dimeric PD-1 (e.g., at 25° C. or at 37° C.) with a KD of less than about 400 pM as measured by surface plasmon resonance, e.g., using the assay format as defined in Example 3 herein. In certain embodiments, the antibodies or antigen-binding fragments thereof bind dimeric PD-1 with a KD of less than about 300 pM, less than about 250 pM, less than about 200 pM, less than about 100 pM, or less than about 50 pM, as measured by surface plasmon resonance, e.g., using the assay format as defined in Example 3 herein, or a substantially similar assay.

The present disclosure also includes antibodies or antigen-binding fragments thereof that bind cynomolgus (Macaca fascicularis) PD-1 (e.g., at 25° C. or at 37° C.) with a KD of less than about 35 nM as measured by surface plasmon resonance, e.g., using the assay format as defined in Example 3 herein. In certain embodiments, the antibodies or antigen-binding fragments thereof bind cynomolgus PD-1 with a KD of less than about 30 nM, less than about 20 nM, less than about 15 nM, less than about 10 nM, or less than about 5 nM, as measured by surface plasmon resonance, e.g., using the assay format as defined in Example 3 herein, or a substantially similar assay.

The present disclosure also includes antibodies and antigen-binding fragments thereof that bind PD-1 with a dissociative half-life (t½) of greater than about 1.1 minutes as measured by surface plasmon resonance at 25° C. or 37° C., e.g., using an assay format as defined in Example 3 herein, or a substantially similar assay. In certain embodiments, the antibodies or antigen-binding fragments of the present disclosure bind PD-1 with a t½ of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, or greater than about 1200 minutes, as measured by surface plasmon resonance at 25° C. or 37° C., e.g., using an assay format as defined in Example 3 herein (e.g., mAb-capture or antigen-capture format), or a substantially similar assay.

The present disclosure also includes antibodies or antigen-binding fragments thereof that block PD-1 binding to PD-L1 with an IC50 of less than about 3 nM as determined using a ELISA-based immunoassay assay, e.g., as shown in Example 4, or a substantially similar assay. The present disclosure also includes antibodies and antigen-binding fragments thereof that bind to PD-1 and enhance the binding of PD-1 to PD-L1.

In some embodiments, the antibodies of the present disclosure may bind to the extracellular domain of PD-1 or to a fragment of the domain. In some embodiments, the antibodies of the present disclosure may bind to more than one domain (cross-reactive antibodies). In certain embodiments, the antibodies of the present disclosure may bind to an epitope located in the extracellular domain comprising amino acid residues 21-171 of PD-1 (SEQ ID NO: 327). In one embodiment, the antibodies may bind to an epitope comprising one or more amino acids selected from the group consisting of amino acid residues 1-146 of SEQ ID NOs: 321-324.

In certain embodiments, the antibodies of the present disclosure may function by blocking or inhibiting the PD-L1-binding activity associated with PD-1 by binding to any other region or fragment of the full length protein, the amino acid sequence of which is shown in SEQ ID NO: 327. In certain embodiments, the antibodies may attenuate or modulate the interaction between PD-1 and PD-L1.

In certain embodiments, the antibodies of the present disclosure may be bi-specific antibodies. The bi-specific antibodies of the disclosure may bind one epitope in one domain and may also bind a second epitope in a different domain of PD-1. In certain embodiments, the bi-specific antibodies of the disclosure may bind two different epitopes in the same domain. In one embodiment, the multi-specific antigen-binding molecule comprises a first binding specificity wherein the first binding specificity comprises the extracellular domain or fragment thereof of PD-L1; and a second binding specificity to another epitope of PD-1.

In one embodiment, the disclosure provides an isolated fully human monoclonal antibody or antigen-binding fragment thereof that binds to PD-1, wherein the antibody or fragment thereof exhibits one or more of the following characteristics: (i) comprises a HCVR having an amino acid sequence selected from the group consisting of SEQ ID NO: 2, 18, 34, 50, 66, 82, 98, 114, 130, 146, 162, 178, 194, 210, 218, 226, 234, 242, 250, 258, 266, 274, 282, 290, 298, 306, and 314, or a substantially similar sequence thereof having at least 90%, at least 95%, at least 98% or at least 99% sequence identity; (ii) comprises a LCVR having an amino acid sequence selected from the group consisting of SEQ ID NO: 10, 26, 42, 58, 74, 90, 106, 122, 138, 154, 170, 186, and 202, or a substantially similar sequence thereof having at least 90%, at least 95%, at least 98% or at least 99% sequence identity; (iii) comprises a HCDR3 domain having an amino acid sequence selected from the group consisting of SEQ ID NO: 8, 24, 40, 56, 72, 88, 104, 120, 136, 152, 168, 184, 200, 216, 224, 232, 240, 248, 256, 264, 272, 280, 288, 296, 304, 312, and 320, or a substantially similar sequence thereof having at least 90%, at least 95%, at least 98% or at least 99% sequence identity; and a LCDR3 domain having an amino acid sequence selected from the group consisting of SEQ ID NO: 16, 32, 48, 64, 80, 96, 112, 128, 144, 160, 176, 192, and 208, or a substantially similar sequence thereof having at least 90%, at least 95%, at least 98% or at least 99% sequence identity; (iv) comprises a HCDR1 domain having an amino acid sequence selected from the group consisting of SEQ ID NO: 4, 20, 36, 52, 68, 84, 100, 116, 132, 148, 164, 180, 196, 212, 220, 228, 236, 244, 252, 260, 268, 276, 284, 292, 300, 308, and 316, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 70, 86, 102, 118, 134, 150, 166, 182, 198, 214, 222, 230, 238, 246, 254, 262, 270, 278, 286, 294, 302, 310, and 318, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 76, 92, 108, 124, 140, 156, 172, 188, and 204, or a substantially similar sequence thereof having at least 90%, at least 95%, at least 98% or at least 99% sequence identity; and a LCDR2 domain having an amino acid sequence selected from the group consisting of SEQ ID NO: 14, 30, 46, 62, 78, 94, 110, 126, 142, 158, 174, 190, and 206, or a substantially similar sequence thereof having at least 90%, at least 95%, at least 98% or at least 99% sequence identity; (v) is a multi-specific antigen-binding molecule comprising a first binding specificity to PD-1 and a second binding specificity to an antigen selected from the group consisting of PD-1, a tumor specific antigen, an autoimmune tissue specific antigen, a virally infected cell antigen, a different T-cell co-inhibitor, T-cell receptor, and a Fc receptor; (vi) binds to human PD-1 with a KD of about 28 pM to about 1.5 μM; (vii) binds to cynomolgus PD-1 with a KD of about 3 nM to about 7.5 μM; (viii) blocks or enhances the binding of PD-1 to PD-L1 with an IC50≤about 3.3 nM; (ix) blocks PD-1-induced T-cell down regulation and/or rescues T-cell signaling in a T-cell/APC luciferase reporter assay; (x) stimulates T-cell proliferation and activity in a mixed lymphocyte reaction (MLR) assay; (xi) induces IL-2 and/or IFNγ production in a MLR assay; and (xii) suppresses tumor growth and increases survival in subjects with cancer.

In one embodiment, the disclosure provides an isolated fully human monoclonal antibody or antigen-binding fragment thereof that blocks PD-1 binding to PD-L1, wherein the antibody or fragment thereof exhibits one or more of the following characteristics: (i) comprises a HCVR having an amino acid sequence selected from the group consisting of SEQ ID NO: 130, 162, 234 and 314, or a substantially similar sequence thereof having at least 90%, at least 95%, at least 98% or at least 99% sequence identity; (ii) comprises a LCVR having an amino acid sequence selected from the group consisting of SEQ ID NO: 138, 170, 186, and 202, or a substantially similar sequence thereof having at least 90%, at least 95%, at least 98% or at least 99% sequence identity; (iii) comprises a HCDR3 domain having an amino acid sequence selected from the group consisting of SEQ ID NO: 136, 168, 240, and 320, or a substantially similar sequence thereof having at least 90%, at least 95%, at least 98% or at least 99% sequence identity; and a LCDR3 domain having an amino acid sequence selected from the group consisting of SEQ ID NO: 144, 176, 192, and 208, or a substantially similar sequence thereof having at least 90%, at least 95%, at least 98% or at least 99% sequence identity; (iv) comprises a HCDR1 domain having an amino acid sequence selected from the group consisting of SEQ ID NO: 132, 164, 236, and 316, or a substantially similar sequence thereof having at least 90%, at least 95%, at least 98% or at least 99% sequence identity; a HCDR2 domain having an amino acid sequence selected from the group consisting of SEQ ID NO: 134, 166, 238, and 318, or a substantially similar sequence thereof having at least 90%, at least 95%, at least 98% or at least 99% sequence identity; a LCDR1 domain having an amino acid sequence selected from the group consisting of SEQ ID NO: 140, 172, 188, and 204, or a substantially similar sequence thereof having at least 90%, at least 95%, at least 98% or at least 99% sequence identity; and a LCDR2 domain having an amino acid sequence selected from the group consisting of SEQ ID NO: 142, 174, 190, and 206, or a substantially similar sequence thereof having at least 90%, at least 95%, at least 98% or at least 99% sequence identity; (v) is a multi-specific antigen-binding molecule comprising a first binding specificity to PD-1 and a second binding specificity to an antigen selected from the group consisting of a different epitope of PD-1, a tumor specific antigen, an autoimmune tissue specific antigen, a virally infected cell antigen, a different T-cell co-inhibitor, T-cell receptor, and a Fc receptor; (vi) binds to human PD-1 with a KD≤10-9M; (vii) binds to cynomolgus PD-1 with a KD-10-8M; (viii) blocks the binding of PD-1 to PD-L1 with an IC50≤10-10M; (ix) blocks PD-1-induced T-cell down regulation and/or rescues T-cell signaling in a T-cell/APC luciferase reporter assay; (x) stimulates T-cell proliferation and activity in a mixed lymphocyte reaction (MLR) assay; (xi) induces IL-2 and/or IFNγ production in a MLR assay; and (xii) suppresses tumor growth and increases survival in subjects with cancer.

The antibodies of the present disclosure may possess one or more of the aforementioned biological characteristics, or any combinations thereof. Other biological characteristics of the antibodies of the present disclosure will be evident to a person of ordinary skill in the art from a review of the present disclosure including the working Examples herein.

### Species Selectivity and Species Cross-Reactivity

According to certain embodiments of the disclosure, the anti-PD-1 antibodies bind to human PD-1 but not to PD-1 from other species. Alternatively, the anti-PD-1 antibodies of the disclosure, in certain embodiments, bind to human PD-1 and to PD-1 from one or more non-human species. For example, the anti-PD-1 antibodies of the disclosure may bind to human PD-1 and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee PD-1. In certain embodiments, the anti-PD-1 antibodies of the disclosure may bind to human and cynomolgus PD-1 with the same affinities or with different affinities, but do not bind to rat and mouse PD-1.

### Epitope Mapping and Related Technologies

The present disclosure includes anti-PD-1 antibodies which interact with one or more amino acids found within one or more domains of the PD-1 molecule including, e.g., extracellular (IgV-like) domain, a transmembrane domain, and an intracellular domain containing the immunoreceptor tyrosine-based inhibition motif (ITIM) and immunoreceptor tyrosine-based switch motif (ITSM). The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within any of the aforementioned domains of the PD-1 molecule (e.g. a linear epitope in a domain). Alternatively, the epitope may consist of a plurality of non-contiguous amino acids (or amino acid sequences) located within either or both of the aforementioned domains of the PD-1 molecule (e.g. a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Other methods include alanine scanning mutational analysis, peptide blot analysis (Reineke (2004) Methods Mol. Biol. 248:443-63), peptide cleavage analysis crystallographic studies and NMR analysis. In addition, methods such as epitope excision, epitope extraction and chemical modification of antigens can be employed (Tomer (2000) Prot. Sci. 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267:252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

In certain embodiments, the anti-PD-1 antibodies or antigen-binding fragments thereof bind an epitope within any one or more of the regions exemplified in PD-1, either in natural form, as exemplified in SEQ ID NO: 327, or recombinantly produced, as exemplified in SEQ ID NOS: 321-324, or to a fragment thereof. In some embodiments, the antibodies of the disclosure bind to an extracellular region comprising one or more amino acids selected from the group consisting of amino acid residues 21-171 of PD-1. In some embodiments, the antibodies of the disclosure bind to an extracellular region comprising one or more amino acids selected from the group consisting of amino acid residues 1-146 of cynomolgus PD-1, as exemplified by SEQ ID NO: 322.

In certain embodiments, the antibodies of the disclosure, as shown in Table 1, interact with at least one amino acid sequence selected from the group consisting of amino acid residues ranging from about position 21 to about position 136 of SEQ ID NO: 327; or amino acid residues ranging from about position 136 to about position 171 of SEQ ID NO: 327. These regions are partially exemplified in SEQ ID NOs: 321-324.

The present disclosure includes anti-PD-1 antibodies that bind to the same epitope, or a portion of the epitope, as any of the specific exemplary antibodies described herein in Table 1, or an antibody having the CDR sequences of any of the exemplary antibodies described in Table 1. Likewise, the present disclosure also includes anti-PD-1 antibodies that compete for binding to PD-1 or a PD-1 fragment with any of the specific exemplary antibodies described herein in Table 1, or an antibody having the CDR sequences of any of the exemplary antibodies described in Table 1. For example, the present disclosure includes anti-PD-1 antibodies that cross-compete for binding to PD-1 with one or more antibodies as defined in Example 6 herein (e.g., H2aM7788N, H4×H8992P, H4×H8999P, H1M7799N, H2aM7780N, H1M7800N, H2aM7794N, H2aM7798N, H4×H9145P2, H4H9057P2, H4×H9120P2, H4×H9128P2, H4H9019P, H4×H9119P2, H4×H9135P2, H4×H9034P, H2aM7790N, H4×H9035P, H4×H9037P, H4×H9045P and H2aM7795N).

One can easily determine whether an antibody binds to the same epitope as, or competes for binding with, a reference anti-PD-1 antibody by using routine methods known in the art. For example, to determine if a test antibody binds to the same epitope as a reference anti-PD-1 antibody of the disclosure, the reference antibody is allowed to bind to a PD-1 protein or peptide under saturating conditions. Next, the ability of a test antibody to bind to the PD-1 molecule is assessed. If the test antibody is able to bind to PD-1 following saturation binding with the reference anti-PD-1 antibody, it can be concluded that the test antibody binds to a different epitope than the reference anti-PD-1 antibody. On the other hand, if the test antibody is not able to bind to the PD-1 protein following saturation binding with the reference anti-PD-1 antibody, then the test antibody may bind to the same epitope as the epitope bound by the reference anti-PD-1 antibody of the disclosure.

To determine if an antibody competes for binding with a reference anti-PD-1 antibody, the above-described binding methodology is performed in two orientations: In a first orientation, the reference antibody is allowed to bind to a PD-1 protein under saturating conditions followed by assessment of binding of the test antibody to the PD-1 molecule. In a second orientation, the test antibody is allowed to bind to a PD-1 molecule under saturating conditions followed by assessment of binding of the reference antibody to the PD-1 molecule. If, in both orientations, only the first (saturating) antibody is capable of binding to the PD-1 molecule, then it is concluded that the test antibody and the reference antibody compete for binding to PD-1. As will be appreciated by a person of ordinary skill in the art, an antibody that competes for binding with a reference antibody may not necessarily bind to the identical epitope as the reference antibody, but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art.

### Immunoconjugates

The disclosure encompasses a human anti-PD-1 monoclonal antibody conjugated to a therapeutic moiety (“immunoconjugate”), such as a cytotoxin or a chemotherapeutic agent to treat cancer. As used herein, the term “immunoconjugate” refers to an antibody which is chemically or biologically linked to a cytotoxin, a radioactive agent, a cytokine, an interferon, a target or reporter moiety, an enzyme, a toxin, a peptide or protein or a therapeutic agent. The antibody may be linked to the cytotoxin, radioactive agent, cytokine, interferon, target or reporter moiety, enzyme, toxin, peptide or therapeutic agent at any location along the molecule so long as it is able to bind its target. Examples of immunoconjugates include antibody drug conjugates and antibody-toxin fusion proteins. In one embodiment, the agent may be a second different antibody to PD-1. In certain embodiments, the antibody may be conjugated to an agent specific for a tumor cell or a virally infected cell. The type of therapeutic moiety that may be conjugated to the anti-PD-1 antibody and will take into account the condition to be treated and the desired therapeutic effect to be achieved. Examples of suitable agents for forming immunoconjugates are known in the art; see for example, WO 05/103081.

### Multi-Specific Antibodies

The antibodies of the present disclosure may be mono-specific, bi-specific, or multi-specific. Multi-specific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244.

In one aspect, the present disclosure includes multi-specific antigen-binding molecules or antigen-binding fragments thereof wherein one specificity of an immunoglobulin is specific for the extracellular domain of PD-1, or a fragment thereof, and the other specificity of the immunoglobulin is specific for binding outside the extracellular domain of PD-1, or a second therapeutic target, or is conjugated to a therapeutic moiety. In certain embodiments, the first antigen-binding specificity may comprise PD-L1 or PD-L2, or a fragment thereof. In certain embodiments of the disclosure, one specificity of an immunoglobulin is specific for an epitope comprising amino acid residues 21-171 of PD-1 (SEQ ID NO: 327) or a fragment thereof, and the other specificity of the immunoglobulin is specific for a second target antigen. The second target antigen may be on the same cell as PD-1 or on a different cell. In one embodiment, the second target cell is on an immune cell other than a T-cell such as a B-cell, antigen-presenting cell, monocyte, macrophage, or dendritic cell. In some embodiments, the second target antigen may be present on a tumor cell or an autoimmune tissue cell or on a virally infected cell.

In another aspect, the disclosure provides multi-specific antigen-binding molecules or antigen-binding fragments thereof comprising a first antigen-binding specificity that binds to PD-1 and a second antigen-binding specificity that binds to a T-cell receptor, a B-cell receptor or a Fc receptor. In a related aspect, the disclosure provides multi-specific antigen-binding molecules or antigen-binding fragments thereof comprising a first antigen-binding specificity that binds to PD-1 and a second antigen-binding specificity that binds to a different T-cell co-inhibitor such as LAG-3, CTLA-4, BTLA, CD-28, 2B4, LY108, TIGIT, TIM3, LAIR1, ICOS and CD160.

In another aspect, the disclosure provides multi-specific antigen-binding molecules or antigen-binding fragments thereof comprising a first antigen-binding specificity that binds to PD-1 and a second antigen-binding specificity that binds to an autoimmune tissue-specific antigen. In certain embodiments, the antibodies may be activating or agonist antibodies.

Any of the multi-specific antigen-binding molecules of the disclosure, or variants thereof, may be constructed using standard molecular biological techniques (e.g., recombinant DNA and protein expression technology), as will be known to a person of ordinary skill in the art.

In some embodiments, PD-1-specific antibodies are generated in a bi-specific format (a “bi-specific”) in which variable regions binding to distinct domains of PD-1 are linked together to confer dual-domain specificity within a single binding molecule. Appropriately designed bi-specifics may enhance overall PD-1 inhibitory efficacy through increasing both specificity and binding avidity. Variable regions with specificity for individual domains, (e.g., segments of the N-terminal domain), or that can bind to different regions within one domain, are paired on a structural scaffold that allows each region to bind simultaneously to the separate epitopes, or to different regions within one domain. In one example for a bi-specific, heavy chain variable regions (VH) from a binder with specificity for one domain are recombined with light chain variable regions (VL) from a series of binders with specificity for a second domain to identify non-cognate VL partners that can be paired with an original VH without disrupting the original specificity for that VH. In this way, a single VL segment (e.g., VL1) can be combined with two different VH domains (e.g., V H1 and VH2) to generate a bi-specific comprised of two binding “arms” (VH1-VL1 and VH2-VL1). Use of a single VL segment reduces the complexity of the system and thereby simplifies and increases efficiency in cloning, expression, and purification processes used to generate the bi-specific (See, for example, U.S. Ser. No. 13/022,759 and US2010/0331527).

Alternatively, antibodies that bind more than one domains and a second target, such as, but not limited to, for example, a second different anti-PD-1 antibody, may be prepared in a bi-specific format using techniques described herein, or other techniques known to those skilled in the art. Antibody variable regions binding to distinct regions may be linked together with variable regions that bind to relevant sites on, for example, the extracellular domain of PD-1, to confer dual-antigen specificity within a single binding molecule. Appropriately designed bi-specifics of this nature serve a dual function. Variable regions with specificity for the extracellular domain are combined with a variable region with specificity for outside the extracellular domain and are paired on a structural scaffold that allows each variable region to bind to the separate antigens.

An exemplary bi-specific antibody format that can be used in the context of the present disclosure involves the use of a first immunoglobulin (Ig) CH3 domain and a second Ig CH3 domain, wherein the first and second Ig CH3 domains differ from one another by at least one amino acid, and wherein at least one amino acid difference reduces binding of the bi-specific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise a Y96F modification (by IMGT; Y436F by EU). Further modifications that may be found within the second CH3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of the present disclosure.

Other exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab2 bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

### Therapeutic Administration and Formulations

The disclosure provides therapeutic compositions comprising the anti-PD-1 antibodies or antigen-binding fragments thereof of the present disclosure. Therapeutic compositions in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody may vary depending upon the age and the size of a subject to be administered, target disease, conditions, route of administration, and the like. When an antibody of the present disclosure is used for treating a disease or disorder in an adult patient, or for preventing such a disease, it is advantageous to administer the antibody of the present disclosure normally at a single dose of about 0.1 to about 60 mg/kg body weight, more preferably about 5 to about 60, about 10 to about 50, or about 20 to about 50 mg/kg body weight. Depending on the severity of the condition, the frequency and the duration of the treatment can be adjusted. In certain embodiments, the antibody or antigen-binding fragment thereof of the disclosure can be administered as an initial dose of at least about 0.1 mg to about 800 mg, about 1 to about 500 mg, about 5 to about 300 mg, or about 10 to about 200 mg, to about 100 mg, or to about 50 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, transdermal, intramuscular, intraperitoneal, intravenous, subcutaneous, intranasal, epidural and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The pharmaceutical composition can be also delivered in a vesicle, in particular a liposome (see, for example, Langer (1990) Science 249:1527-1533).

The use of nanoparticles to deliver the antibodies of the present disclosure is also contemplated herein. Antibody-conjugated nanoparticles may be used both for therapeutic and diagnostic applications. Antibody-conjugated nanoparticles and methods of preparation and use are described in detail by Arruebo, M., et al. 2009 (“Antibody-conjugated nanoparticles for biomedical applications” in J. Nanomat. Volume 2009, Article ID 439389, 24 pages, doi: 10.1155/2009/439389), incorporated herein by reference. Nanoparticles may be developed and conjugated to antibodies contained in pharmaceutical compositions to target tumor cells or autoimmune tissue cells or virally infected cells. Nanoparticles for drug delivery have also been described in, for example, U.S. Pat. No. 8,257,740, or U.S. Pat. No. 8,246,995, each incorporated herein in its entirety.

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous, intracranial, intraperitoneal and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition of the present disclosure can be delivered subcutaneously or intravenously with an auto-injector, as described herein previously. Such auto-injector can be reusable or disposable. A reusable auto-injector generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The auto-injector can then be reused. In a disposable auto-injector, there is no replaceable cartridge. Rather, the auto-injector comes prefilled with the pharmaceutical composition held in a reservoir within the device. Once the reservoir is emptied of the pharmaceutical composition, the entire device is discarded.

The amount of the antibody contained is generally about 5 to about 500 mg per dosage form in a unit dose; especially in the form of injection, it is preferred that the antibody is contained in about 5 to about 100 mg and in about 10 to about 250 mg for the other dosage forms.

### Therapeutic Uses of the Antibodies

The antibodies of the disclosure are useful, inter alia, for the treatment, prevention and/or amelioration of any disease or disorder associated with or mediated by PD-1 expression, signaling, or activity, or treatable by blocking the interaction between PD-1 and a PD-1 ligand (e.g., PD-L1, or PD-L2) or otherwise inhibiting PD-1 activity and/or signaling. For example, the present disclosure provides methods for treating cancer (tumor growth inhibition), chronic viral infections and/or autoimmune disease by administering an anti-PD-1 antibody (or pharmaceutical composition comprising an anti-PD-1 antibody) as described herein to a patient in need of such treatment. The antibodies of the present disclosure are useful for the treatment, prevention, and/or amelioration of disease or disorder or condition such as cancer, autoimmune disease or a viral infection and/or for ameliorating at least one symptom associated with such disease, disorder or condition. In the context of the methods of treatment described herein, the anti-PD-1 antibody may be administered as a monotherapy (i.e., as the only therapeutic agent) or in combination with one or more additional therapeutic agents (examples of which are described elsewhere herein).

In some embodiments of the disclosure, the antibodies described herein are useful for treating subjects suffering from primary or recurrent cancer, including, but not limited to, renal cell carcinoma, colorectal cancer, non-small-cell lung cancer, brain cancer (e.g., glioblastoma multiforme), squamous cell carcinoma of head and neck, gastric cancer, prostate cancer, ovarian cancer, kidney cancer, breast cancer, multiple myeloma, and melanoma.

The antibodies may be used to treat early stage or late-stage symptoms of cancer. In one embodiment, an antibody or fragment thereof of the disclosure may be used to treat metastatic cancer. The antibodies are useful in reducing or inhibiting or shrinking tumor growth of both solid tumors and blood cancers. In certain embodiments, treatment with an antibody or antigen-binding fragment thereof of the disclosure leads to more than 50% regression, more than 60% regression, more than 70% regression, more than 80% regression or more than 90% regression of a tumor in a subject. In certain embodiments, the antibodies may be used to prevent relapse of a tumor. In certain embodiments, the antibodies are useful in extending overall survival in a subject with cancer. In some embodiments, the antibodies are useful in reducing toxicity due to chemotherapy or radiotherapy while maintaining long-term survival in a patient suffering from cancer.

In certain embodiments, the antibodies of the disclosure are useful to treat subjects suffering from a chronic viral infection. In some embodiments, the antibodies of the disclosure are useful in decreasing viral titers in the host and/or rescuing exhausted T-cells. In certain embodiments, an antibody or fragment thereof of the disclosure may be used to treat chronic viral infection by lymphocytic choriomeningitis virus (LCMV). In some embodiments, an antibody or antigen-binding fragment thereof the disclosure may be administered at a therapeutic dose to a patient with an infection by human immunodeficiency virus (HIV) or human papilloma virus (HPV) or hepatitis B/C virus (HBV/HCV). In a related embodiment, an antibody or antigen-binding fragment thereof of the disclosure may be used to treat an infection by simian immunodeficiency virus (SIV) in a simian subject such as cynomolgus.

In certain embodiments, a blocking antibody of the present disclosure may be administered in a therapeutically effective amount to a subject suffering from a cancer or a viral infection.

In certain embodiments, the antibodies of the disclosure are useful for treating an autoimmune disease, including but not limited to, alopecia areata, autoimmune hepatitis, celiac disease, Graves' disease, Guillain-Barre syndrome, Hashimoto's disease, hemolytic anemia, inflammatory bowel disease, inflammatory myopathies, multiple sclerosis, primary biliary cirrhosis, psoriasis, rheumatoid arthritis, scleroderma, Sjögren's syndrome, systemic lupus erthyematosus, vitiligo, autoimmune pancreatitis, autoimmune urticaria, autoimmune thrombocytopeni purpura, Crohn's disease, diabetes type I, eosinophilic fasciitis, eosinophilic enterogastritis, Goodpasture's syndrome, myasthenia gravis, psoriatic arthritis, rheumatic fever, ulcerative colitis, vasculitis and Wegener's granulomatosis. In certain embodiments, an activating antibody of the disclosure may be used to treat a subject suffering from autoimmune disease.

One or more antibodies of the present disclosure may be administered to relieve or prevent or decrease the severity of one or more of the symptoms or conditions of the disease or disorder.

It is also contemplated herein to use one or more antibodies of the present disclosure prophylactically to patients at risk for developing a disease or disorder such as cancer, autoimmune disease and chronic viral infection.

In a further embodiment of the disclosure the present antibodies are used for the preparation of a pharmaceutical composition for treating patients suffering from cancer, autoimmune disease or viral infection. In another embodiment of the disclosure, the present antibodies are used as adjunct therapy with any other agent or any other therapy known to those skilled in the art useful for treating cancer, autoimmune disease or viral infection.

### Combination Therapies and Formulations

Combination therapies may include an anti-PD-1 antibody of the disclosure and any additional therapeutic agent that may be advantageously combined with an antibody of the disclosure, or with a biologically active fragment of an antibody of the disclosure.

The antibodies of the present disclosure may be combined synergistically with one or more anti-cancer drugs or therapy used to treat cancer, including, for example, renal cell carcinoma, colorectal cancer, glioblastoma multiforme, squamous cell carcinoma of head and neck, non-small-cell lung cancer, colon cancer, ovarian cancer, adenocarcinoma, prostate cancer, glioma, and melanoma. It is contemplated herein to use anti-PD-1 antibodies of the disclosure in combination with immunostimulatory and/or immunosupportive therapies to inhibit tumor growth, and/or enhance survival of cancer patients. The immunostimulatory therapies include direct immunostimulatory therapies to augment immune cell activity by either “releasing the brake” on suppressed immune cells or “stepping on the gas” to activate an immune response. Examples include targeting other checkpoint receptors, vaccination and adjuvants. The immunosupportive modalities may increase antigenicity of the tumor by promoting immunogenic cell death, inflammation or have other indirect effects that promote an anti-tumor immune response. Examples include radiation, chemotherapy, anti-angiogenic agents, and surgery.

In various embodiments, one or more antibodies of the present disclosure may be used in combination with an antibody to PD-L1, a second antibody to PD-1 (e.g., nivolumab), a LAG-3 inhibitor, a CTLA-4 inhibitor (e.g., ipilimumab), a TIM3 inhibitor, a BTLA inhibitor, a TIGIT inhibitor, a CD47 inhibitor, an antagonist of another T-cell co-inhibitor or ligand (e.g., an antibody to CD-28, 2B4, LY108, LAIR1, ICOS, CD160 or VISTA), an indoleamine-2,3-dioxygenase (IDO) inhibitor, a vascular endothelial growth factor (VEGF) antagonist [e.g., a “VEGF-Trap” such as aflibercept or other VEGF-inhibiting fusion protein as set forth in U.S. Pat. No. 7,087,411, or an anti-VEGF antibody or antigen binding fragment thereof (e.g., bevacizumab, or ranibizumab) or a small molecule kinase inhibitor of VEGF receptor (e.g., sunitinib, sorafenib, or pazopanib)], an Ang2 inhibitor (e.g., nesvacumab), a transforming growth factor beta (TGFβ) inhibitor, an epidermal growth factor receptor (EGFR) inhibitor (e.g., erlotinib, cetuximab), an agonist to a co-stimulatory receptor (e.g., an agonist to glucocorticoid-induced TNFR-related protein), an antibody to a tumor-specific antigen (e.g., CA9, CA125, melanoma-associated antigen 3 (MAGE3), carcinoembryonic antigen (CEA), vimentin, tumor-M2-PK, prostate-specific antigen (PSA), mucin-1, MART-1, and CA19-9), a vaccine (e.g., Bacillus Calmette-Guerin, a cancer vaccine), an adjuvant to increase antigen presentation (e.g., granulocyte-macrophage colony-stimulating factor), a bispecific antibody (e.g., CD3×CD20 bispecific antibody, PSMA×CD3 bispecific antibody), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), cyclophosphamide, radiotherapy, an IL-6R inhibitor (e.g., sarilumab), an IL-4R inhibitor (e.g., dupilumab), an IL-10 inhibitor, a cytokine such as IL-2, IL-7, IL-21, and IL-15, an antibody-drug conjugate (ADC) (e.g., anti-CD19-DM4 ADC, and anti-DS6-DM4 ADC), an anti-inflammatory drug (e.g., corticosteroids, and non-steroidal anti-inflammatory drugs), a dietary supplement such as anti-oxidants or any palliative care to treat cancer. In certain embodiments, the anti-PD-1 antibodies of the present disclosure may be used in combination with cancer vaccines including dendritic cell vaccines, oncolytic viruses, tumor cell vaccines, etc. to augment the anti-tumor response. Examples of cancer vaccines that can be used in combination with anti-PD-1 antibodies of the present disclosure include MAGE3 vaccine for melanoma and bladder cancer, MUC1 vaccine for breast cancer, EGFRv3 (e.g., Rindopepimut) for brain cancer (including glioblastoma multiforme), or ALVAC-CEA (for CEA+ cancers).

In certain embodiments, the anti-PD-1 antibodies of the disclosure may be administered in combination with radiation therapy in methods to generate long-term durable anti-tumor responses and/or enhance survival of patients with cancer. In some embodiments, the anti-PD-1 antibodies of the disclosure may be administered prior to, concomitantly or after administering radiation therapy to a cancer patient. For example, radiation therapy may be administered in one or more doses to tumor lesions followed by administration of one or more doses of anti-PD-1 antibodies of the disclosure. In some embodiments, radiation therapy may be administered locally to a tumor lesion to enhance the local immunogenicity of a patient's tumor (adjuvinating radiation) and/or to kill tumor cells (ablative radiation) followed by systemic administration of an anti-PD-1 antibody of the disclosure. For example, intracranial radiation may be administered to a patient with brain cancer (e.g., glioblastoma multiforme) in combination with systemic administration of an anti-PD-1 antibody of the disclosure. In certain embodiments, the anti-PD-1 antibodies of the disclosure may be administered in combination with radiation therapy and a chemotherapeutic agent (e.g., temozolomide) or a VEGF antagonist (e.g., aflibercept).

In certain embodiments, the anti-PD-1 antibodies of the disclosure may be administered in combination with one or more anti-viral drugs to treat chronic viral infection caused by LCMV, HIV, HPV, HBV or HCV. Examples of anti-viral drugs include, but are not limited to, zidovudine, lamivudine, abacavir, ribavirin, lopinavir, efavirenz, cobicistat, tenofovir, rilpivirine and corticosteroids. In some embodiments, the anti-PD-1 antibodies of the disclosure may be administered in combination with a LAG3 inhibitor, a CTLA-4 inhibitor or any antagonist of another T-cell co-inhibitor to treat chronic viral infection.

In certain embodiments, the anti-PD-1 antibodies of the disclosure may be combined with an antibody to a Fc receptor on immune cells for the treatment of an autoimmune disease. In one embodiment, an antibody or fragment thereof of the disclosure is administered in combination with an antibody or antigen-binding protein targeted to an antigen specific to autoimmune tissue. In certain embodiments, an antibody or antigen-binding fragment thereof of the disclosure is administered in combination with an antibody or antigen-binding protein targeted to a T-cell receptor or a B-cell receptor, including but not limited to, Fcα (e.g., CD89), Fcγ (e.g., CD64, CD32, CD16a, and CD16b), CD19, etc. The antibodies of fragments thereof of the disclosure may be used in combination with any drug or therapy known in the art (e.g., corticosteroids and other immunosuppressants) to treat an autoimmune disease or disorder including, but not limited to alopecia areata, autoimmune hepatitis, celiac disease, Graves' disease, Guillain-Barre syndrome, Hashimoto's disease, hemolytic anemia, inflammatory bowel disease, inflammatory myopathies, multiple sclerosis, primary biliary cirrhosis, psoriasis, rheumatoid arthritis, scleroderma, Sjögren's syndrome, systemic lupus erthyematosus, vitiligo, autoimmune pancreatitis, autoimmune urticaria, autoimmune thrombocytopeni purpura, Crohn's disease, diabetes type I, eosinophilic fasciitis, eosinophilic enterogastritis, Goodpasture's syndrome, myasthenia gravis, psoriatic arthritis, rheumatic fever, ulcerative colitis, vasculitis and Wegener's granulomatosis.

The additional therapeutically active agent(s)/component(s) may be administered prior to, concurrent with, or after the administration of the anti-PD-1 antibody of the present disclosure. For purposes of the present disclosure, such administration regimens are considered the administration of an anti-PD-1 antibody “in combination with” a second therapeutically active component.

The additional therapeutically active component(s) may be administered to a subject prior to administration of an anti-PD-1 antibody of the present disclosure. For example, a first component may be deemed to be administered “prior to” a second component if the first component is administered 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration of the second component. In other embodiments, the additional therapeutically active component(s) may be administered to a subject after administration of an anti-PD-1 antibody of the present disclosure. For example, a first component may be deemed to be administered “after” a second component if the first component is administered 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration of the second component. In yet other embodiments, the additional therapeutically active component(s) may be administered to a subject concurrent with administration of an anti-PD-1 antibody of the present disclosure. “Concurrent” administration, for purposes of the present disclosure, includes, e.g., administration of an anti-PD-1 antibody and an additional therapeutically active component to a subject in a single dosage form (e.g., co-formulated), or in separate dosage forms administered to the subject within about 30 minutes or less of each other. If administered in separate dosage forms, each dosage form may be administered via the same route (e.g., both the anti-PD-1 antibody and the additional therapeutically active component may be administered intravenously, subcutaneously, etc.); alternatively, each dosage form may be administered via a different route (e.g., the anti-PD-1 antibody may be administered intravenously, and the additional therapeutically active component may be administered subcutaneously). In any event, administering the components in a single dosage from, in separate dosage forms by the same route, or in separate dosage forms by different routes are all considered “concurrent administration,” for purposes of the present disclosure. For purposes of the present disclosure, administration of an anti-PD-1 antibody “prior to”, “concurrent with,” or “after” (as those terms are defined herein above) administration of an additional therapeutically active component is considered administration of an anti-PD-1 antibody “in combination with” an additional therapeutically active component).

The present disclosure includes pharmaceutical compositions in which an anti-PD-1 antibody of the present disclosure is co-formulated with one or more of the additional therapeutically active component(s) as described elsewhere herein using a variety of dosage combinations.

In exemplary embodiments in which an anti-PD-1 antibody of the disclosure is administered in combination with a VEGF antagonist (e.g., a VEGF trap such as aflibercept), including administration of co-formulations comprising an anti-PD-1 antibody and a VEGF antagonist, the individual components may be administered to a subject and/or co-formulated using a variety of dosage combinations. For example, the anti-PD-1 antibody may be administered to a subject and/or contained in a co-formulation in an amount selected from the group consisting of 0.01 mg, 0.02 mg, 0.03 mg, 0.04 mg, 0.05 mg, 0.1 mg, 0.2 mg, 0.3 mg, 0.4 mg, 0.5 mg, 0.6 mg, 0.7 mg, 0.8 mg, 0.9 mg, 1.0 mg, 1.5 mg, 2.0 mg, 2.5 mg, 3.0 mg, 3.5 mg, 4.0 mg, 4.5 mg, 5.0 mg, 6.0 mg, 7.0 mg, 8.0 mg, 9.0 mg, and 10.0 mg; and the VEGF antagonist (e.g., a VEGF trap such as aflibercept) may be administered to the subject and/or contained in a co-formulation in an amount selected from the group consisting of 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg and 3.0 mg. The combinations/co-formulations may be administered to a subject according to any of the administration regimens disclosed elsewhere herein, including, e.g., twice a week, once every week, once every 2 weeks, once every 3 weeks, once every month, once every 2 months, once every 3 months, once every 4 months, once every 5 months, once every 6 months, etc.

### Administrative Regimens

According to certain embodiments of the present disclosure, multiple doses of an anti-PD-1 antibody (or a pharmaceutical composition comprising a combination of an anti-PD-1 antibody and any of the additional therapeutically active agents mentioned herein) may be administered to a subject over a defined time course. The methods according to this aspect of the disclosure comprise sequentially administering to a subject multiple doses of an anti-PD-1 antibody of the disclosure. As used herein, “sequentially administering” means that each dose of anti-PD-1 antibody is administered to the subject at a different point in time, e.g., on different days separated by a predetermined interval (e.g., hours, days, weeks or months). The present disclosure includes methods which comprise sequentially administering to the patient a single initial dose of an anti-PD-1 antibody, followed by one or more secondary doses of the anti-PD-1 antibody, and optionally followed by one or more tertiary doses of the anti-PD-1 antibody. The anti-PD-1 antibody may be administered at a dose between 0.1 mg/kg to 100 mg/kg.

The terms “initial dose,” “secondary doses,” and “tertiary doses,” refer to the temporal sequence of administration of the anti-PD-1 antibody of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-PD-1 antibody, but generally may differ from one another in terms of frequency of administration. In certain embodiments, however, the amount of anti-PD-1 antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of anti-PD-1 antibody which is administered to a patient prior to the administration of the very next dose in the sequence with no intervening doses.

The methods according to this aspect of the disclosure may comprise administering to a patient any number of secondary and/or tertiary doses of an anti-PD-1 antibody. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

The present disclosure includes administration regimens in which 2 to 6 loading doses are administered to a patient at a first frequency (e.g., once a week, once every two weeks, once every three weeks, once a month, once every two months, etc.), followed by administration of two or more maintenance doses to the patient on a less frequent basis. For example, according to this aspect of the disclosure, if the loading doses are administered at a frequency of, e.g., once a month (e.g., two, three, four, or more loading doses administered once a month), then the maintenance doses may be administered to the patient once every five weeks, once every six weeks, once every seven weeks, once every eight weeks, once every ten weeks, once every twelve weeks, etc.).

### Diagnostic Uses of the Antibodies

The anti-PD-1 antibodies of the present disclosure may be used to detect and/or measure PD-1 in a sample, e.g., for diagnostic purposes. Some embodiments contemplate the use of one or more antibodies of the present disclosure in assays to detect a disease or disorder such as cancer, autoimmune disease or chronic viral infection. Exemplary diagnostic assays for PD-1 may comprise, e.g., contacting a sample, obtained from a patient, with an anti-PD-1 antibody of the disclosure, wherein the anti-PD-1 antibody is labeled with a detectable label or reporter molecule or used as a capture ligand to selectively isolate PD-1 from patient samples. Alternatively, an unlabeled anti-PD-1 antibody can be used in diagnostic applications in combination with a secondary antibody which is itself detectably labeled. The detectable label or reporter molecule can be a radioisotope, such as 3H, 14C, 32P, 35S, or 125I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure PD-1 in a sample include enzyme-linked immunosorbent assay (ELISA), radioimmunoassay (RIA), and fluorescence-activated cell sorting (FACS).

Samples that can be used in PD-1 diagnostic assays according to the present disclosure include any tissue or fluid sample obtainable from a patient, which contains detectable quantities of either PD-1 protein, or fragments thereof, under normal or pathological conditions. Generally, levels of PD-1 in a particular sample obtained from a healthy patient (e.g., a patient not afflicted with cancer or an autoimmune disease) will be measured to initially establish a baseline, or standard, level of PD-1. This baseline level of PD-1 can then be compared against the levels of PD-1 measured in samples obtained from individuals suspected of having a cancer-related condition, or symptoms associated with such condition.

The antibodies specific for PD-1 may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface.

Aspects of the disclosure relate to use of the disclosed antibodies as markers for predicting prognosis of cancer or an autoimmune disorder in patients. Antibodies of the present disclosure may be used in diagnostic assays to evaluate prognosis of cancer in a patient and to predict survival.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Generation of Human Antibodies to PD-1

Human antibodies to PD-1 were generated using a fragment of PD-1 that ranges from about amino acids 25-170 of GenBank Accession NP_005009.2 (SEQ ID NO: 327) with a C93S change. The immunogen was administered directly, with an adjuvant to stimulate the immune response, to a VELOCIMMUNE® mouse comprising DNA encoding human Immunoglobulin heavy and kappa light chain variable regions. The antibody immune response was monitored by a PD-1-specific immunoassay. When a desired immune response was achieved splenocytes were harvested and fused with mouse myeloma cells to preserve their viability and form hybridoma cell lines. The hybridoma cell lines were screened and selected to identify cell lines that produce PD-1-specific antibodies. Using this technique, and the immunogen described above, several anti-PD-1 chimeric antibodies (i.e., antibodies possessing human variable domains and mouse constant domains) were obtained; exemplary antibodies generated in this manner were designated as H1M7789N, H1M7799N, H1M7800N, H2M7780N, H2M7788N, H2M7790N, H2M7791N, H2M7794N, H2M7795N, H2M7796N, and H2M7798N.

Anti-PD-1 antibodies were also isolated directly from antigen-positive B cells without fusion to myeloma cells, as described in U.S. 2007/0280945A1, herein specifically incorporated by reference in its entirety. Using this method, several fully human anti-PD-1 antibodies (i.e., antibodies possessing human variable domains and human constant domains) were obtained; exemplary antibodies generated in this manner were designated as follows: H4H9019P, H4×H9034P2, H4×H9035P2, H4×H9037P2, H4×H9045P2, H4×H9048P2, H4H9057P2, H4H9068P2, H4×H9119P2, H4×H9120P2, H4×H9128P2, H4×H9135P2, H4×H9145P2, H4×H8992P, H4×H8999P, and H4×H9008P.

The biological properties of the exemplary antibodies generated in accordance with the methods of this Example are described in detail in the Examples set forth below.

### Example 2: Heavy and Light Chain Variable Region Amino Acid and Nucleotide Sequences

Table 1 sets forth the amino acid sequence identifiers of the heavy and light chain variable regions and CDRs of selected anti-PD-1 antibodies of the disclosure. The corresponding nucleic acid sequence identifiers are set forth in Table 2.

TABLE 1
Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H1M7789N | 2 | 4 | 6 | 8 | 10 | 12 | 14 | 16 |
| H1M7799N | 18 | 20 | 22 | 24 | 26 | 28 | 30 | 32 |
| H1M7800N | 34 | 36 | 38 | 40 | 42 | 44 | 46 | 48 |
| H2M7780N | 50 | 52 | 54 | 56 | 58 | 60 | 62 | 64 |
| H2M7788N | 66 | 68 | 70 | 72 | 74 | 76 | 78 | 80 |
| H2M7700N | 82 | 84 | 86 | 88 | 90 | 92 | 94 | 96 |
| H2M7791N | 98 | 100 | 102 | 104 | 106 | 108 | 110 | 112 |
| H2M7794N | 114 | 116 | 118 | 120 | 122 | 124 | 126 | 128 |
| H2M7795N | 130 | 132 | 134 | 136 | 138 | 140 | 142 | 144 |
| H2M7796N | 146 | 148 | 150 | 152 | 154 | 156 | 168 | 160 |
| H2M7798N | 162 | 164 | 166 | 168 | 170 | 172 | 174 | 176 |
| H4xH9019P | 178 | 180 | 182 | 184 | 186 | 188 | 190 | 192 |
| H4xH8034P2 | 194 | 196 | 188 | 200 | 202 | 204 | 206 | 208 |
| H4xH9035P2 | 210 | 212 | 214 | 216 | 202 | 204 | 206 | 208 |
| H4xH9037P2 | 218 | 220 | 222 | 224 | 202 | 204 | 206 | 208 |
| H4xH9045P2 | 226 | 228 | 230 | 232 | 202 | 204 | 206 | 208 |
| H4xH9048P2 | 234 | 236 | 238 | 240 | 202 | 204 | 206 | 208 |
| H4H9057P2 | 242 | 244 | 246 | 248 | 202 | 204 | 206 | 208 |
| H4H9068P2 | 250 | 262 | 254 | 258 | 202 | 204 | 206 | 208 |
| H4xH9119P2 | 258 | 260 | 262 | 264 | 202 | 204 | 206 | 208 |
| H4xH9120P2 | 266 | 268 | 270 | 272 | 202 | 204 | 206 | 208 |
| H4xH9128P2 | 274 | 276 | 278 | 280 | 202 | 204 | 206 | 208 |
| H4xH9135P2 | 282 | 284 | 286 | 288 | 202 | 204 | 206 | 208 |
| H4xH8145P2 | 290 | 292 | 294 | 296 | 202 | 204 | 206 | 208 |
| H4xH8992P | 298 | 300 | 302 | 304 | 186 | 188 | 190 | 192 |
| H4xH8999P | 306 | 308 | 310 | 312 | 186 | 188 | 190 | 192 |
| H4xH9008P | 314 | 316 | 318 | 320 | 186 | 188 | 190 | 192 |

TABLE 2
Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H1M7789N | 1 | 3 | 5 | 7 | 9 | 11 | 13 | 15 |
| H1M7799N | 17 | 19 | 21 | 23 | 25 | 27 | 29 | 31 |
| H1M7800N | 33 | 35 | 37 | 39 | 41 | 43 | 45 | 47 |
| H2M7780N | 49 | 51 | 53 | 55 | 57 | 59 | 61 | 63 |
| H2M7788N | 65 | 67 | 69 | 71 | 73 | 75 | 77 | 79 |
| H2M7790N | 81 | 83 | 85 | 87 | 89 | 91 | 93 | 95 |
| H2M7791N | 97 | 99 | 101 | 103 | 105 | 107 | 109 | 111 |
| H2M7794N | 113 | 115 | 117 | 119 | 121 | 123 | 125 | 127 |
| H2M7795N | 129 | 131 | 133 | 135 | 137 | 139 | 141 | 143 |
| H2M7796N | 145 | 147 | 149 | 151 | 153 | 155 | 157 | 159 |
| H2M7798N | 161 | 163 | 165 | 167 | 169 | 171 | 173 | 175 |
| H4H9019P | 177 | 179 | 181 | 183 | 185 | 187 | 189 | 191 |
| H4xH9034P2 | 193 | 195 | 197 | 198 | 201 | 203 | 205 | 207 |
| H4xH9035P2 | 209 | 211 | 213 | 215 | 201 | 203 | 205 | 207 |
| H4xH9037P2 | 217 | 219 | 221 | 223 | 201 | 203 | 205 | 207 |
| HAxH9045P2 | 225 | 227 | 229 | 231 | 201 | 203 | 205 | 207 |
| H4xH9048P2 | 233 | 235 | 237 | 239 | 201 | 203 | 205 | 207 |
| H4H9067P2 | 241 | 243 | 245 | 247 | 201 | 203 | 205 | 207 |
| H4H9068P2 | 249 | 251 | 253 | 255 | 201 | 203 | 205 | 207 |
| H4xH9119P2 | 257 | 259 | 261 | 263 | 201 | 203 | 205 | 207 |
| H4xH9120P2 | 265 | 267 | 269 | 271 | 201 | 202 | 205 | 207 |
| H4xH9128P2 | 273 | 275 | 277 | 279 | 201 | 203 | 205 | 207 |
| H4xH9136P2 | 281 | 283 | 285 | 287 | 201 | 203 | 205 | 207 |
| H4xH9145P2 | 289 | 291 | 293 | 295 | 201 | 203 | 205 | 207 |
| H4xH8992P | 297 | 299 | 301 | 303 | 185 | 187 | 189 | 191 |
| H4xH8999P | 305 | 307 | 309 | 311 | 185 | 187 | 189 | 191 |
| H4xH9008P | 313 | 315 | 317 | 319 | 185 | 187 | 189 | 191 |

Antibodies are typically referred to herein according to the following nomenclature: Fc prefix (e.g. “H4×H,” “H1M,” “H2M,” etc.), followed by a numerical identifier (e.g. “7789,” “7799,” etc., as shown in Table 1), followed by a “P,” “P2,” “N,” or “B” suffix. Thus, according to this nomenclature, an antibody may be referred to herein as, e.g., “H1H7789N,” “H1M7799N,” “H2M7780N,” etc. The H4×H, H1M, H2M and H2aM prefixes on the antibody designations used herein indicate the particular Fc region isotype of the antibody. For example, an “H4×H” antibody has a human IgG4 Fc with 2 or more amino acid changes as disclosed in US20100331527, an “H1M” antibody has a mouse IgG1 Fc, and an “H2M” antibody has a mouse IgG2 Fc (a or b isotype) (all variable regions are fully human as denoted by the first ‘H’ in the antibody designation). As will be appreciated by a person of ordinary skill in the art, an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs)—which are indicated by the numerical identifiers shown in Table 1—will remain the same, and the binding properties to antigen are expected to be identical or substantially similar regardless of the nature of the Fc domain.

In certain embodiments, selected antibodies with a mouse IgG1 Fc were converted to antibodies with human IgG4 Fc. In one embodiment, the IgG4 Fc domain comprises a serine to proline mutation in the hinge region (S108P) to promote dimer stabilization. Table 3 sets forth the amino acid sequence identifiers of heavy chain and light chain sequences of selected anti-PD-1 antibodies with human IgG4 Fc.

TABLE 3

| Antibody | SEQ ID NOs: | |
|---|---|---|
| Designation | Heavy Chain | Light Chain |
| H4H7798N | 330 | 331 |
| H4H7795N2 | 332 | 333 |
| H4H9008P | 334 | 335 |
| H4H9048P2 | 336 | 337 |

Each heavy chain sequence in Table 3 comprised a variable region (VH or HCVR; comprising HCDR1, HCDR2 and HCDR3) and a constant region (comprising CH1, CH2 and CH3 domains). Each light chain sequence in Table 3 comprised a variable region (VL or LCVR; comprising LCDR1, LCDR2 and LCDR3) and a constant region (CO. SEQ ID NO: 330 comprised a HCVR comprising amino acids 1-117 and a constant region comprising amino acids 118-444. SEQ ID NO: 331 comprised a LCVR comprising amino acids 1-107 and a constant region comprising amino acids 108-214. SEQ ID NO: 332 comprised a HCVR comprising amino acids 1-122 and a constant region comprising amino acids 123-449. SEQ ID NO: 333 comprised a LCVR comprising amino acids 1-107 and a constant region comprising amino acids 108-214. SEQ ID NO: 334 comprised a HCVR comprising amino acids 1-119 and a constant region comprising amino acids 120-446. SEQ ID NO: 335 comprised a LCVR comprising amino acids 1-108 and a constant region comprising amino acids 109-215. SEQ ID NO: 336 comprised a HCVR comprising amino acids 1-121 and a constant region comprising amino acids 122-448. SEQ ID NO: 337 comprised a LCVR comprising amino acids 1-108 and a constant region comprising amino acids 109-215.

### Example 3: Antibody Binding to PD-1 as Determined by Surface Plasmon Resonance

Binding association and dissociation rate constants (ka and kd, respectively), equilibrium dissociation constants and dissociation half-lives (KD and t½, respectively) for antigen binding to purified anti-PD1 antibodies were determined using a real-time surface plasmon resonance biosensor assay on a Biacore 4000 or Biacore T200 instrument. The Biacore sensor surface was derivatized with either a polyclonal rabbit anti-mouse antibody (GE, #BR-1008-38) or with a monoclonal mouse anti-human Fc antibody (GE, #BR-1008-39) to capture approximately 100-900 RUs of anti-PD-1 monoclonal antibodies, expressed with either a mouse Fc or a human Fc, respectively. The PD-1 reagents tested for binding to the anti-PD-1 antibodies included recombinant human PD-1 expressed with a C-terminal myc-myc-hexahistidine tag (hPD-1-MMH; SEQ ID NO: 321), recombinant cynomolgus monkey PD-1 expressed with a C-terminal myc-myc-hexahistidine tag (MfPD-1-MMH; SEQ ID NO: 322), recombinant human PD-1 dimer expressed with either a C-terminal mouse IgG2a Fc tag (hPD-1-mFc; SEQ ID NO: 323) or with a C-terminal human IgG1 Fc (hPD1-hFc; SEQ ID NO: 324), and monkey PD-1 with mFc (SEQ ID NO: 329). Different concentrations of PD-1 reagents ranging from 200 nM to 3.7 nM were injected over the anti-PD-1 monoclonal antibody captured surface at a flow rate of 30 μL/min on Biacore 4000 or at 50 L/min on Biacore T200. The binding of the PD-1 reagents to captured monoclonal antibodies was monitored for 3 to 5 minutes while their dissociation from the antibodies was monitored for 7 to 10 minutes in HBST running buffer (0.01 M HEPES pH 7.4, 0.15 M NaCl, 3 mM EDTA, 0.05% v/v Surfactant P20). Experiments were performed at 25° C. and 37° C. Kinetic association (ka) and dissociation (kd) rate constants were determined by processing and fitting the data to a 1:1 binding model using Scrubber 2.0c curve fitting software. Binding dissociation equilibrium constants (KD) and dissociative half-lives (t½) were then calculated from the kinetic rate constants as: KD (M)=kd/ka and t½ (min)=[ln 2/(60*kd)]. Binding kinetics parameters for different anti-PD-1 monoclonal antibodies binding to different PD-1 reagents at 25° C. and 37° C. are tabulated in Tables 4-11.

TABLE 4
Binding Kinetics parameters of anti-PD-1 monoclonal antibodies
binding to human PD-1-MMH at 25° C.

| | kₐ | k_{d} | K_{D} | t½ |
|---|---|---|---|---|
| Antibody | (1/Ms) | (1/s) | (M) | (min) |
| H2aM7780N | 9.32E+03 | 3.59E−04 | 3.85E−08 | 32 |
| H2aM7788N | 1.97E+04 | 3.88E−04 | 1.96E−08 | 30 |
| H1M7789N | 2.53E+04 | 5.31E−05 | 2.10E−09 | 218 |
| H2aM7790N | 4.63E+04 | 8.23E−04 | 1.78E−08 | 14 |
| H2aM7791N | 3.01E+04 | 7.06E−04 | 2.34E−08 | 16 |
| H2aM7794N | 5.50E+04 | 2.12E−03 | 3.80E−08 | 5.4 |
| H2aM7795N | 4.91E+04 | 1.15E−03 | 2.35E−08 | 10 |
| H2aM7796N | 6.73E+03 | 1.93E−03 | 2.86E−07 | 6.0 |
| H2AM7798N | 1.32E+05 | 3.06E−04 | 2.31E−09 | 38 |
| H1M7799N | 5.04E+04 | 1.23E−02 | 2.44E−07 | 0.9 |
| H1M7800N | 5.88E+04 | 9.47E−03 | 1.61E−07 | 1.2 |
| M4H9019P | 2.05E+04 | 8.08E−04 | 3.94E−08 | 14 |
| H4xH9034P | 1.02E+05 | 1.49E−03 | 1.45E−08 | 7.8 |
| H4xH9035P | 1.03E+05 | 4.75E−04 | 4.62E−09 | 24 |
| H4xH9037P | 7.32E+04 | 7.95E−04 | 1.09E−08 | 15 |
| H4xH9045P | 5.40E+04 | 4.03E−03 | 7.46E−08 | 2.9 |
| H4xH9048P2 | 1.37E+05 | 1.23E−03 | 8.95E−09 | 9.4 |
| H4H905792 | 4.50E+04 | 1.34E−02 | 2.91E−07 | 0.9 |
| H4H9068P2 | NB* | NB* | NB* | NB* |
| H4xH9119P2 | 7.84E+04 | 1.22E−03 | 1.56E−08 | 9.5 |
| H4xH9120P2 | 3.32E+04 | 9.98E−04 | 3.01E−08 | 12 |
| H4xH9128P2 | 4.95E+04 | 7.19E−04 | 1.45E−08 | 16 |
| H4xH9135P2 | 1.17E+05 | 1.20E−03 | 1.02E−08 | 10 |
| H4xH9145P2 | 3.47E+04 | 1.34E−03 | 3.85E−08 | 8.6 |
| H4xH8992P | 1.50E+05 | 2.13E−02 | 1.41E−07 | 0.5 |
| HxXH8999P | 2.83E+05 | 1.23E−03 | 4.33E−09 | 9.4 |
| H4xH9008P | 4.29E+04 | 1.33E−03 | 3.10E−08 | 8.7 |
| H4H7795N2 | 5.35E+04 | 1.48E−03 | 2.33E−08 | 8 |
| H4H7798N | 1.47E+05 | 4.43E−04 | 3.01E−09 | 26 |

*NB indicates that under the experimental conditions, PD-1 reagent did not bind to the captured anti-PD-1 monoclonal antibody

TABLE 5
Binding Kinetics parameters of anti-PD-1 monocional antibodies
binding to human PD-1-MMH at 37° C.

| | kₐ | k_{d} | K_{D} | t½ |
|---|---|---|---|---|
| Antibody | (1/Ms) | (1/s) | (M) | (min) |
| H2aM7780N | 2.72E+04 | 1.52E−03 | 5.58E−08 | 7.6 |
| H2aM7788M | 2.88E+04 | 1.49E−03 | 5.19E−08 | 7.7 |
| H1M7789N | 4.53E+04 | 2.95E−04 | 6.52E−09 | 39 |
| H2aM7790N | 6.13E+04 | 5.20E−03 | 8.49E−08 | 2.2 |
| H2aM7791N | 4.18E+04 | 2.24E−03 | 5.35E−08 | 5.2 |
| H2aM7794N | 1.20E+05 | 7.92E−03 | 6.61E−08 | 1.5 |
| H2aM7795N | 6.75E+04 | 4.58E−03 | 6.78E−08 | 2.5 |
| H2aM7798N | 1.09E+04 | 1.65E−02 | 1.51E−06 | 0.7 |
| H2aM7798N | 1.73E+05 | 6.56E−04 | 3.79E−09 | 18 |
| H1M7799N | 7.94E+04 | 4.25E−02 | 5.36E−07 | 0.3 |
| H1M7800N | 7.83E+04 | 3.99E−02 | 5.10E−07 | 0.3 |
| H4H9019P | 1.20E+04 | 5.44E−03 | 4.53E−07 | 2.1 |
| H4xH9034P | 2.79E+05 | 1.12E−02 | 4.02E−08 | 1.0 |
| H4xH9035P | 2.98E+05 | 4.26E−03 | 1.43E−08 | 2.7 |
| H4xH9037P | 2.26E+05 | 6.68E−03 | 2.95E−08 | 1.7 |
| H4xH9045P | 8.04E+04 | 5.32E−02 | 6.62E−07 | 0.2 |
| H4xM9048P2 | 3.70E+05 | 8.60E−03 | 2.32E−08 | 1.3 |
| H4H9057P2 | NB* | NB* | NB* | NB* |
| H4H9068P2 | NB* | NB* | NB* | NB* |
| H4xH9119P2 | 2.40E+05 | 1.04E−02 | 4.35E−08 | 1.1 |
| H4xH9120P2 | 6.86E+04 | 7.01E−03 | 1.02E−07 | 1.6 |
| H4xH9128P2 | 1.04E+05 | 4.36E−03 | 4.20E−08 | 2.6 |
| H4xH9135P2 | 4.18E+05 | 1.11E−02 | 2.66E−08 | 1.0 |
| H4xH9145P2 | 1.31E+05 | 1.23E−02 | 9.40E−08 | 0.9 |
| H4xH8992P | IC* | IC* | IC* | IC* |
| H4xH8999P | 5.99E+05 | 9.42E−03 | 1.57E−08 | 1.2 |
| H4xH9008P | 1.29E+05 | 8.09E−03 | 6.26E−08 | 1.4 |
| H4H7795N2 | 6.41E+04 | 6.64E−03 | 1.04E−07 | 1.7 |
| H4H7798N | 2.27E+05 | 1.70E−03 | 7.48E−09 | 7 |

*NB indicates that under the experimental conditions, PD-1 reagent did not bind to the captured anti-PD-1 monoclonal antibody. IC indicates that under the experimental conditions, PD-1 binding is inconclusive.

TABLE 6
Binding Kinetics parameters of anti-PD-1 monoclonal
antibodies binding to human PD-1 dimer (human PD-1-mFc
or human PD-1-hFc) at 25° C.

| | kₐ | k_{d} | K_{D} | t½ |
|---|---|---|---|---|
| Antibody | (1/Ms) | (1/s) | (M) | (min) |
| H2aM7780N | 4.21E+04 | 9.94E−06 | 2.36E−10 | 1162 |
| H2aM7788N | 8.94E+04 | 2.82E−05 | 3.15E−10 | 410 |
| H1M7789N | 3.91E+04 | 4.31E−05 | 1.10E−09 | 268 |
| H2aM7790N | 1.86E+05 | 3.02E−05 | 1.62E−10 | 383 |
| H2aM7791N | 4.05E+04 | 1.01E−04 | 2.49E−09 | 114 |
| H2aM7794N | 1.79E+05 | 1.06E−04 | 5.93E−10 | 109 |
| H2aM7795N | 1.38E+05 | 3.14E−05 | 2.27E−10 | 368 |
| H2aM7796N | 2.61E+04 | 8.67E−05 | 3.32E−09 | 133 |
| H2aM7798N | 3.50E+05 | 2.29E−06 | 6.55E−11 | 505 |
| H1M7799N | 2.38E+05 | 8.55E−05 | 3.60E−10 | 135 |
| H1M7800N | 1.52E+05 | 7.72E−06 | 5.09E−10 | 150 |
| H4M9019P | 4.38E+04 | 8.61E−05 | 1.97E−09 | 134 |
| H4xH8034P | 2.15E+05 | 1.51E−04 | 7.01E−10 | 77 |
| H4xH9036P | 2.01E+05 | 1.03E−04 | 5.13E−10 | 112 |
| H4xH9037F | 1.50E+05 | 1.29E−04 | 8.62E−10 | 89 |
| H4xH9045P | 9.13E+04 | 1.60E−04 | 1.75E−09 | 72 |
| H4xH0048P2 | 2.36E+05 | 1.88E−04 | 7.98E−10 | 61 |
| H4H9057P2 | 1.01E+05 | 1.77E−04 | 1.75E−09 | 65 |
| H4H9068P2 | 4.72E+04 | 2.80E−03 | 5.94E−08 | 4 |
| H4xH9119P2 | 1.63E+05 | 1.52E−04 | 9.92E−10 | 71 |
| H4xH9120P2 | 6.52E+04 | 1.19E−04 | 1.82E−09 | 97 |
| H4xH9128P2 | 8.37E+04 | 1.33E−04 | 1.59E−09 | 87 |
| H4xH9135P2 | 2.12E+05 | 1.38E−04 | 6.51E−10 | 84 |
| H4xH9145P2 | 6.58E+04 | 1.58E−04 | 2.40E−09 | 73 |
| H4xH8992P | 2.35E+05 | 1.60E−04 | 6.80E−10 | 72 |
| H4xH8999P | 5.55E+05 | 1.20E−04 | 2.17E−10 | 96 |
| H4xH9008P | 3.52E+04 | 2.80E−05 | 7.86E−10 | 412 |
| H4H7795N2 | 1.50E+05 | 9.25E−05 | 6.15E−10 | 125 |
| H4H7798N | 4.41E+05 | 5.40E−05 | 1.22E−10 | 214 |

TABLE 7
Binding Kinetics parameters of anti-PD-1 monoclonal
antibodies binding to human PD-1
dimer (human PD-1-mFc or human PD-1-hFc) at 37° C.

| | kₐ | k_{d} | K_{D} | t½ |
|---|---|---|---|---|
| Antibody | (1/Ms) | (1/s) | (M) | (min) |
| H2aM7780N | 9.94E+04 | 2.29E−05 | 2.30E−10 | 505 |
| H2aM7788N | 1.31E+05 | 2.13E−05 | 1.63E−10 | 542 |
| H1M7789N | 1.09E+05 | ≤1.0E−05 | ≤9.17E−11 | ≥1155 |
| H2aM7790N | 2.01E+05 | 8.49E−05 | 4.22E−10 | 136 |
| H2aM7791N | 4.98E+04 | 1.79E−04 | 3.59E−09 | 65 |
| H2aM7794N | 4.68E+05 | 2.11E−04 | 4.52E−10 | 65 |
| H2aM7795N | 1.65E+05 | 6.13E−05 | 3.71E−10 | 188 |
| H2aM7796N | 2.21E+04 | 4.34E−04 | 1.96E−08 | 27 |
| H2aM7798N | 4.90E+05 | 1.40E−05 | 2.80E−11 | 825 |
| H1M7798N | 4.41E+05 | 1.81E−04 | 4.11E−10 | 64 |
| H1M7800N | 4.00E+05 | 1.81E−04 | 4.50E−10 | 64 |
| H4H9019P | 7.17E+04 | 1.95E−04 | 2.71E−09 | 59 |
| H4xH9034P | 3.02E+05 | 6.30E−04 | 2.09E−09 | 18 |
| H4xH9036P | 3.16E+05 | 5.54E−04 | 1.75E−09 | 21 |
| H4xH9037P | 2.63E+05 | 9.21E−04 | 3.50E−09 | 13 |
| H4xH9045P | 2.14E+05 | 1.10E−03 | 5.13E−09 | 11 |
| H4xH9048P2 | 3.61E+05 | 1.10E−03 | 3.05E−09 | 10 |
| H4H9057P2 | 2.33E+05 | 2.11E−03 | 9.07E−09 | 5 |
| H4H9068P2 | 9.69E+04 | 1.20E−02 | 1.24E−07 | 1 |
| H4xH9119P2 | 2.40E+05 | 9.09E−04 | 3.80E−09 | 13 |
| H4xH9120P2 | 8.08E+04 | 4.82E−04 | 5.96E−09 | 24 |
| H4xH9128P2 | 1.86E+05 | 6.86E−04 | 3.68E−09 | 17 |
| H4xH9135P2 | 3.10E+05 | 7.02E−04 | 2.27E−09 | 16 |
| H4xHB145P2 | 1.60E+05 | 5.71E−04 | 3.58E−09 | 20 |
| H4xH8992P | 3.49E+05 | 1.02E−03 | 2.91E−09 | 11 |
| H4xH8999P | 7.57E+05 | 4.51E−04 | 5.96E−10 | 26 |
| H4x19008P | 5.52E+04 | ≤1.0E−05 | ≤1.81E−10 | ≥1155 |
| H4H7795N2 | 1.60E+05 | 2.64E−04 | 1.65E−09 | 44 |
| M4H7798N | 6.60E+05 | 1.15E−04 | 1.75E−10 | 100 |

TABLE 8
Binding Kinetics parameters of ant-PD-1 monoclonal
antibodies binding to MfPD-1-MMH 25° C.

| | kₐ | k_{d} | K_{D} | t½ |
|---|---|---|---|---|
| Antibody | (1/Ms) | (1/s) | (M) | (min) |
| H2aM7780N | 1.00E+04 | 3.15E−04 | 3.15E−08 | 37 |
| H2aM7788N | 8.53E+03 | 6.62E−04 | 7.66E−08 | 17 |
| H1M7789N | 1.55E+04 | 1.23E−04 | 7.89E−09 | 94 |
| H2aM7790N | 3.11E+04 | 9.37E−04 | 3.01E−08 | 12 |
| H2aM7791N | 1.61E+04 | 5.53E−04 | 3.44E−08 | 21 |
| H2aM7794N | 3.60E+04 | 5.99E−03 | 1.66E−07 | 1.9 |
| H2aM7795N | 4.44E+04 | 8.89E−04 | 2.01E−08 | 13 |
| H2aM7796N | NB* | NB* | NB* | NB* |
| H2aM7798N | 8.72E+04 | 3.93E−04 | 4.50E−09 | 29 |
| H1M7799N | 5.78E+04 | 1.30E−02 | 2.24E−07 | 0.9 |
| H1M7800N | 5.89E+04 | 1.04E−02 | 1.76E−07 | 1.1 |
| H4H9019P | 1.94E+04 | 8.33E−04 | 4.29E−08 | 14 |
| H4xH8034P | 9.61E+04 | 2.69E−03 | 2.80E−08 | 4.3 |
| H4xH9035P | 9.36E+04 | 4.34E−04 | 4.64E−09 | 27 |
| H4xH9037P | 6.99E+04 | 9.15E−04 | 1.31E−08 | 13 |
| H4xH9045P | 6.25E+04 | 7.05E−03 | 1.13E−07 | 1.6 |
| H4xH9048P2 | 1.28E+05 | 8.97E−04 | 7.00E−09 | 13 |
| H4H9057P2 | 3.46E+04 | 1.91E−02 | 5.51E−07 | 0.6 |
| H4H9068P2 | NB* | NB* | NB* | NB* |
| H4xH9119P2 | 7.50E+04 | 1.66E−03 | 2.22E−08 | 6.9 |
| H4xH9120P2 | 3.17E+04 | 1.08E−03 | 3.41E−08 | 11 |
| H4xH9128P2 | 3.68E+04 | 6.49E−04 | 1.77E−08 | 18 |
| H4xH9135P2 | 1.24E+05 | 1.31E−03 | 1.06E−08 | 8.8 |
| H4xH9145P2 | 2.86E+04 | 1.24E−03 | 4.31E−08 | 9.3 |
| H4xH8992P | 1.88E+05 | 3.76E−02 | 2.00E−07 | 0.3 |
| H4xH8999P | 4.29E+05 | 1.33E−03 | 3.09E−09 | 8.7 |
| H4xH9008P | 1.05E+05 | 2.49E−03 | 2.38E−08 | 4.6 |
| H4H7795N2 | 6.59E+04 | 1.48E−03 | 2.24E−08 | 8 |
| HAH7798N | 1.43E+05 | 5.51E−04 | 3.80E−09 | 21 |

*NB indicates that under the experimental conditions, PD-1 reagent did not bind to the captured anti-PD-1 monodonal antibody

TABLE 9
Binding Kinetics parameters of anti-PD-1 monoclonal
antibodies binding to MIPD-1-MMH at 37°C.

| | kₐ | k_{d} | K_{D} | t½ |
|---|---|---|---|---|
| Antibody | (1/Ms) | (1/s) | (M) | (min) |
| H2aM7780N | 2.29E+04 | 1.38E−03 | 6.05E−08 | 8.3 |
| H2aM7788N | 1.88E+04 | 3.28E−03 | 1.74E−07 | 3.6 |
| M1M7789N | 4.79E+04 | 4.08E−04 | 8.50E−09 | 28 |
| H2aM7790N | 2.55E+04 | 6.93E−03 | 2.71E−07 | 1.7 |
| H2aM7791N | 3.79E+04 | 1.91E−03 | 5.05E−08 | 6.0 |
| H2aM7794N | 6.66E+04 | 2.01E−02 | 3.02E−07 | 0.6 |
| H2aM7795N | 6.47E+04 | 3.89E−03 | 6.02E−08 | 3.0 |
| H2aM7798N | NB* | NB* | NB* | NB* |
| H2aM7798N | 1.42E+05 | 9.93E−04 | 7.00E−09 | 12 |
| H1M7799N | 8.80E+04 | 4.67E−02 | 5.30E−07 | 0.2 |
| H1M7800N | 8.40E+04 | 4.43E−02 | 5.27E−07 | 0.3 |
| H4H9019P | 2.14E+04 | 7.63E−03 | 3.56E−07 | 1.5 |
| H4xH9034P | 2.83E+05 | 2.47E−02 | 8.73E−08 | 0.6 |
| H4xH9035P | 3.06E+05 | 4.29E−03 | 1.40E−08 | 2.7 |
| H4xH9037P | 2.22E+05 | 8.80E−03 | 3.97E−08 | 1.3 |
| H4xH9045P | 1.40E+04 | 1.05E−01 | 7.54E−06 | 0.1 |
| H4xH9048P2 | 4.15E+05 | 6.97E−03 | 1.68E−08 | 1.7 |
| H4H9057P2 | NB* | NB* | NB* | NB* |
| H4H9068P2 | NB* | NB* | NB* | NB* |
| H4xH9119P2 | 2.40E+05 | 1.23E−02 | 5.14E−08 | 0.9 |
| H4xH9120P2 | 6.98E+04 | 7.48E−03 | 1.07E−07 | 1.5 |
| H4xH9128P2 | 9.06E+04 | 4.18E−03 | 4.61E−08 | 2.8 |
| H4xH9135P2 | 4.62E+05 | 1.34E−02 | 2.89E−08 | 0.9 |
| H4xH9145P2 | 1.71E+05 | 1.43E−02 | 8.37E−08 | 0.8 |
| H4xH8992P | IC* | IC* | IC* | IC* |
| H4xH8999P | 9.83E+05 | 9.26E−03 | 9.41E−09 | 1.2 |
| H4xH9008P | 5.86E+05 | 1.38E−02 | 2.35E−08 | 0.8 |
| H4H7795N2 | 7.80E+04 | 6.89E−03 | 8.83E−08 | 1.7 |
| H4H7798N | 2.13E+05 | 2.23E−3 | 1.05E−08 | 5 |

*NB indicates that under the experimental conditions, PD-1 reagent did not bind to the captured anti-PD-1 monoclonal antibody. IC Indicates that under the experimental conditions, PD-1 binding is inconclusive.

TABLE 10
Binding Kinetics parameters of anti-PO-1 monoclonal antibodies
binding to monkey PD-1 dimer (monkey PD-1-mFc) at 25° C.

| | | 100 nM | | | | |
|---|---|---|---|---|---|---|
| | Amount | Monkey | | | | |
| | of mAb | PD-1-mFc | | | | |
| | Captured | Bound | kₐ | k_{d} | K_{D} | t½ |
| Antibody | (RU) | (RU) | (1/Ms) | (1/s) | (M) | (min) |
| H4H9019P | 116 | 31 | 4.55E+04 | 8.06E−05 | 1.97E−09 | 129 |
| H4xH9034P | 215 | 95 | 2.03E+05 | 1.66E−04 | 8.18E−10 | 70 |
| H4xH9036P | 153 | 78 | 2.16E+05 | 9.96E−06 | 4.60E−10 | 116 |
| H4xH9037P | 137 | 58 | 1.50E+05 | 1.37E−04 | 9.12E−10 | 84 |
| H4xH9045P | 202 | 78 | 9.78E+04 | 1.68E−04 | 1.72E−09 | 69 |
| H4xH9048P2 | 227 | 115 | 2.43E+05 | 1.84E−04 | 7.54E−10 | 63 |
| H4H9057P2 | 196 | 75 | 1.02E+05 | 3.03E−04 | 2.96E−09 | 38 |
| H4H0068P2 | 178 | 17 | 5.70E+04 | 3.09E−03 | 5.42E−08 | 4 |
| H4xH9119P2 | 209 | 83 | 1.63E+06 | 1.72E−04 | 1.05E−09 | 67 |
| H4xH9120P2 | 195 | 52 | 5.84E+04 | 1.12E−04 | 1.91E−09 | 104 |
| H4xH9128P2 | 175 | 64 | 7.87E+04 | 1.24E−04 | 1.57E−09 | 94 |
| H4xH9135P2 | 150 | 74 | 2.38E+05 | 1.43E−04 | 6.02E−10 | 81 |
| H4xH9145P2 | 304 | 84 | 7.24E+04 | 1.50E−04 | 2.08E−09 | 77 |
| H4xH8992P | 260 | 122 | 2.03E+06 | 2.51E−04 | 1.24E−09 | 46 |
| H4xH8999P | 217 | 128 | 5.50E+05 | 1.15E−04 | 2.10E−10 | 100 |
| H4xH9008P | 248 | 93 | 1.20E+05 | 5.77E−05 | 4.80E−10 | 200 |
| H4H7795N2 | 204 | 60 | 1.60E+05 | 9.92E−05 | 6.21E−10 | 116 |
| H4H7798N | 223 | 93 | 4.49E+05 | 6.14E−06 | 1.37E−10 | 188 |

TABLE 11
Binding Kinetics parameters of anti-PD-1 monoclonal antibodies
binding to monkey PD-1 dimer (monkey PD-1-mFc) at 37° C.

| | | 100 nM | | | | |
|---|---|---|---|---|---|---|
| | Amount | Monkey | | | | |
| | of mAb | PD-1-mFc | | | | |
| | Captured | Bound | kₐ | k_{d} | K_{D} | t½ |
| Antibody | (RU) | (RU) | (1/Ms) | (1/s) | (M) | (min) |
| H4H9019P | 89 | 36 | 8.16E+04 | 2.59E−04 | 3.17E−09 | 45 |
| H4xH9034P | 184 | 81 | 3.07E+05 | 7.49E−04 | 2.44E−09 | 15 |
| H4xH9035P | 88 | 40 | 3.67E+05 | 6.23E−04 | 1.70E−09 | 19 |
| H4xH9037P | 55 | 24 | 2.80E+05 | 8.97E−04 | 3.21E−09 | 13 |
| H4xH9045P | 161 | 65 | 2.41E+05 | 1.36E−03 | 5.66E−09 | 8 |
| H4xH8048P2 | 184 | 84 | 4.94E+05 | 1.13E−03 | 2.29E−09 | 10 |
| H4H9057P2 | 105 | 28 | 1.61E+05 | 4.77E−03 | 2.96E−08 | 2.4 |
| H4H9068P2 | 90 | 6 | 1.21E+05 | 1.05E−02 | 8.63E−08 | 1.1 |
| H4xH9119P2 | 98 | 40 | 2.79E+05 | 8.85E−04 | 3.17E−09 | 13 |
| H4xH9120P2 | 141 | 46 | 8.29E+04 | 5.02E−04 | 6.06E−09 | 23 |
| H4xH9128P2 | 148 | 60 | 1.87E+05 | 8.16E−04 | 4.36E−09 | 14 |
| H4xH913592 | 106 | 52 | 3.42E+05 | 7.94E−04 | 2.32E−09 | 15 |
| H4xH9145P2 | 284 | 94 | 1.51E+05 | 6.09E−04 | 4.04E−09 | 19 |
| H4xH8992P | 206 | 86 | 3.50E+05 | 1.53E−03 | 4.38E−09 | 8 |
| H4xH8999P | 160 | 83 | 7.30E+05 | 5.10E−04 | 7.00E−10 | 23 |
| H4xH9008P | 216 | 98 | 2.04E+05 | 1.00E−05* | 4.90E−11* | 1155* |
| H4H7795N2 | 164 | 47 | 1.70E+06 | 2.90E−04 | 1.71E−09 | 40 |
| H4H7798N | 203 | 88 | 6.30E+05 | 1.27E−04 | 2.02E−10 | 91 |

*indicates that under the current experimental conditions, no dissociation of PD-1 reagent was observed and the value of kd was manually fixed at 1.00E−05

As shown in Table 4, at 25° C., 28 of the 29 anti-PD-1 antibodies of the disclosure bound to hPD-1-MMH with KD values ranging from 2.1 nM to 291 nM. One antibody, H4H9068P2, did not demonstrate any measurable binding to hPD-1-MMH at 25° C. As shown in Table 5, at 37° C., 26 of the 29 anti-PD-1 antibodies of the disclosure bound to hPD-1-MMH with KD values ranging from 3.79 nM to 1.51 UM. Three antibodies of the disclosure did not demonstrate any conclusive binding to hPD-1-MMH at 37° C. As shown in Table 6, at 25° C., all 29 anti-PD-1 antibodies of the disclosure bound to hPD-1 dimer proteins with KD values ranging from 65.5 pM to 59.4 nM. As shown in Table 7, at 37° C., all 27 anti-PD-1 antibodies of the disclosure bound to hPD-1 dimer proteins with KD values ranging from 3.09 pM to 551 nM. As shown in Table 8, at 25° C., 27 of the 29 anti-PD-1 antibodies of the disclosure bound to MfPD-1-MMH with KD values ranging from 3.09 nM to 551 nM. Two antibodies of the disclosure did not demonstrate any conclusive binding to MfPD-1-MMH at 25° C. As shown in Table 9, at 37° C., 25 of the 29 anti-PD-1 antibodies of the disclosure bound to MfPD-1-MMH with KD values ranging from 7.00 nM to 7.54 M. Four antibodies of the disclosure did not demonstrate any conclusive binding to MfPD-1-MMH at 37° C. As shown in Table 10, at 25° C., all 18 of the tested anti-PD-1 antibodies of the disclosure bound to MfPD-1 dimer with KD values ranging from 137 pM to 54.2 nM. As shown in Table 11, at 37° C., all 18 of the tested anti-PD-1 antibodies of the disclosure bound to MfPD-1 dimer with KD values ranging from less than 49 pM to 86.3 nM.

### Example 4: Blocking of PD-1 Binding to PD-L1 as Determined by ELISA

The ability of anti-PD-1 antibodies to block human PD-1 binding to its ligand, the PD-L1 receptor, was measured using three competition sandwich ELISA formats. Dimeric human PD-L1 proteins, comprised of a portion of the human PD-L1 extracellular domain expressed with either a C-terminal human Fc tag (hPD-L1-hFc; SEQ ID: 325) or a C-terminal mouse Fc tag (hPD-L1-mFc; SEQ ID: 326), or dimeric human PD-L2, comprised of the human PD-L2 extracellular region produced with a C-terminal human Fc tag (hPD-L2-hFc; R&D Systems, #1224-PL) were separately coated at a concentration of 2 μg/mL in PBS on a 96-well microtiter plate overnight at 4° C. Nonspecific binding sites were subsequently blocked using a 0.5% (w/V) solution of BSA in PBS. In a first competition format, a constant concentration of 1.5 nM of a dimeric human PD-1 protein, comprised of the human PD-1 extracellular domain expressed with a C-terminal mouse Fc tag (hPD-1-mFc; SEQ ID: 323) was added to serial dilutions of anti-PD-1 antibodies or isotype control antibodies so that the final concentrations of antibodies ranged from 0 to 200 nM. In a second competition format, a constant concentration of 200 pM of dimeric biotinylated human PD-1 protein, comprised of the human PD-1 extracellular domain that was expressed with a C-terminal human Fc tag (biot-hPD-1-hFc; SEQ ID: 323), was similarly added to serial dilutions of anti-PD-1 antibodies or an isotype control at final antibody concentrations ranging from 0 to 50 nM. In a third competition format, a constant concentration of 100 pM of dimeric hPD-1-mFc protein was similarly added to serial dilutions of anti-PD-1 antibodies or an isotype control at final antibody concentrations ranging from 0 to 100 nM. These antibody-protein complexes were then incubated for 1 hour at room temperature (RT). Antibody-protein complexes with 1.5 nM constant hPD-1-mFc were transferred to microtiter plates coated with hPD-L1-hFc, antibody-protein complexes with 200 pM constant biot-hPD-1-hFc were transferred to hPD-L1-mFc coated plates, and antibody-protein complexes with 100 pM constant hPD-1-mFc were transferred to microtiter plates coated with hPD-L2-hFc. After incubating for 1 hour at RT, the wells were washed, and plate-bound hPD-1-mFc was detected with an anti-mFc polyclonal antibody conjugated with horseradish peroxidase (HRP) (Jackson ImmunoResearch Inc., #115-035-164), and plate-bound biot-hPD-1-hFc was detected with streptavidin conjugated with HRP (Thermo Scientific, #N200). Samples were developed with a TMB solution (BD Biosciences, #51-2606KC and #51-2607KC) to produce a colorimetric reaction and then color development was stabilized by addition of 1M sulfuric acid before measuring absorbance at 450 nm on a Victor X5 plate reader. Data analysis was performed using a sigmoidal dose-response model within Prism™ software (GraphPad). The calculated IC50 value, defined as the concentration of antibody required to reduce 50% of human PD-1 binding to human PD-L1 or PD-L2, was used as an indicator of blocking potency. Percent maximum blockade was calculated as a measure of the ability of the antibodies to completely block binding of human PD-1 to human PD-L1 or PD-L2 on the plate as determined from the dose curve. This percent maximum blockade was calculated by subtracting from 100% the ratio of the reduction in signal observed in the presence of the highest tested concentration for each antibody relative to the difference between the signal observed for a sample of human PD-1 containing no anti-PD-1 antibody (0% blocking) and the background signal from HRP-conjugated secondary antibody alone (100% blocking).

Percent maximum blockade and the calculated IC50 values for antibodies blocking greater than 35% of the hPD-1 binding signal are shown in Tables 12-14. Antibodies that showed a decrease in the hPD-1 binding signal of 35% or less were defined as non-blockers. Antibodies that showed an increase of 35% or more in the binding signal of human PD-1 were characterized as non-blocker/enhancers. The theoretical assay bottom, defined as the minimum antibody concentration theoretically needed to occupy 50% binding sites of human PD-1 in the assay, is 0.75 nM for the format using 1.5 nM constant hPD-1-mFc, 100 pM for the format using 200 pM constant biot-hPD-1-hFc, and 50 pM for the format using 100 pM constant hPD-1-mFc, indicating that lower calculated IC50 values may not represent quantitative protein-antibody site binding. For this reason, antibodies with calculated IC50 values less than 0.75 nM in the assay with hPD-1-mFc constant and hPD-L1 coat, less than 100 pM in the assay with biot-hPD-1-hFc constant and hPD-L1 coat, and less than 50 pM in the assay with hPD-1-mFc constant and hPD-L2 coat are reported in Tables 12-14 as <7.5E-10M, <1.0E-10M and <5.0E-11M, respectively.

TABLE 12
ELISA blocking of human PD-1 binding lo human
PD-L1 by anti-PD-1 antibodies

| | Blocking 1.5 nM | 200 nM Antibody |
|---|---|---|
| | of hPD-1-mFc | blocking 1.5 nM |
| | binding to hPD- | hPD-1-mFc binding |
| Antibody | L1-hFc, IC₅₀ (M) | hPD-L1-hFc, % blocking |
| H4H9019P | 1.3E−09 | 96 |
| H4xH9034P | 5.1E−10* | 98 |
| H4xH9045P | 2.8E−10* | 98 |
| H4xH9048P2 | 3.3E−09 | 67 |
| H4xH9120P2 | 1.0E−09 | 98 |
| H4xH9128P2 | 6.4E−10* | 98 |
| H4xH9035P | 6.2E−10* | 99 |
| H4xH9135P2 | 1.1E−09 | 97 |
| H4xH9145P2 | 9.3E−10 | 90 |
| H4xH9119P2 | 2.0E−10* | 78 |
| H4H9657P2 | 1.9E−10* | 98 |
| H4H9068P2 | NBI/ | −142 |
| | Enchancer | |
| H4xH9037P | 8.9E−10 | 100 |
| H2aM7780N | 6.9E−10* | 94 |
| H2aM7788N | 2.2E−10* | 74 |
| H1M7799N | NBI/ | −170 |
| | Enchancer | |
| H2aM7790N | 1.5E−09 | 74 |
| H2aM47791N | NBI/ | −154 |
| | Enchancer | |
| H2aM7794N | 1.1E−09 | 95 |
| H2aM7795N2 | 8.6E−10 | 92 |
| H2aM7796N | NBI | −20 |
| H2aM7798N | 6.8E−10* | 93 |
| H1M7799N | 2.2E−10* | 82 |
| H1M7800N | 6.0E−10* | 83 |
| H4xH8992P | 1.3E−09 | 93 |
| H4xH8999P | 1.3E−09 | 88 |
| H4xH9008P | 2.4E−09 | 88 |
| Isotype control 1 | NBI | −3 |
| Isotype control 2 | NBI | −34 |
| Isotype control 2 | NBI | −7 |
| Isotype control 2 | NBI | −18 |

Assay theoretical bottom: for blocking ELISA with hPD-1-mFc constant and hPD-L1 coat is 7.5E−10M
*Below theoretical bottom of the assay:
NT—not tested;
NBI—non-blocker;
NBI/Enhancer—non-blocker/enhancer,
IC—inconclusive

TABLE 13
ELISA blocking of biotinylated human PD-1 binding
to human PD-L1 by anti-PD-1 antibodies

| | Blocking 200 pM biot- | 50 nM Antibody blocking |
|---|---|---|
| | hPD-1-hFc binding | 200 pM biot-hPD-1-hFc |
| | to hPD-L1-mFc, | binding to hPD-L1-mFc, |
| Antibody | IC₅₀ (M) | % blocking |
| H4H9019P | 6.4E−10 | 97 |
| H4xH9034P | 6.6E−11* | 96 |
| H4xH9045P | 1.3E−10 | 95 |
| H4xH9048P2 | IC | 76 |
| H4xH9120P2 | 3.9E−10 | 96 |
| H4xH9128P2 | 1.9E−10 | 97 |
| H4xH9035P | 8.0E−11* | 95 |
| H4xH9135P2 | 1.5E−10 | 96 |
| H4xH9119P2 | 3.5E−10 | 97 |
| H4xH9057P2 | 8.2E−11* | 96 |
| H4H9068P2 | NBI/Enhancer | −57 |
| H4H9068P2 | NBI/Enhancer | −43 |
| H4xH9037P | 7.8E−11* | 95 |
| H2aM7780N | 9.1E−11* | 100 |
| H2aM7788N | 6.5E−11* | 100 |
| H1M7789N | NBI | 9 |
| H2aM7790N | 1.9E−10 | 99 |
| H2aM7791N | NBI/Enhancer | -45 |
| H2aM7794N | 2.3E−10 | 99 |
| H2aM7795N2 | 6.9E−11* | 99 |
| H2aM7798N | 1.3E−09 | 60 |
| H2aM7798N | 7.3E−11* | 100 |
| H1M7799N | 5.9E−11* | 100 |
| H1M7800N | 6.5E−11* | 99 |
| H4xH8992P | 1.8E−10 | 97 |
| H4xH8999P | 1.8E−10 | 92 |
| H4xH9008P | 1.3E−09 | 93 |
| Isotype control 1 | NBI | 19 |
| Isotype control 2 | NBI | 35 |
| Isotype control 2 | NBI | −18 |
| Isotype control 2 | NBI | −11 |

Assay theoretical bottom: for blocking ELISA with biot-hPD-1-mFc constant and hPD-L1 coat is 1.0E−10M
*Below theoretical bottom of the assay;
NT—not tasted;
NBI—non-blocker;
NBI/Enhancer—non-blocker/enhancer;
IC—inconclusive

TABLE 14
ELISA blocking of human PD-1 binding to human
PD-L2 by anti-PD-1 antibodies

| | Blocking 100 pM of | 100 nM Antibody blocking |
|---|---|---|
| | hPD-1-mFc binding | 100 pM hPD-1-mFc binding |
| | to hPD-L2-hFc, IC₅₀ | to hPD-L2-hFc. |
| Antibody | (M) | % blocking |
| H4xH9048P2 | 1.4E−10 | 98 |
| H2aM7795N2 | 2.6E−10 | 100 |
| H2aM7798N | 1.3E−10 | 100 |
| H4xH9008P | 1.3E−09 | 94 |
| Isotype control 2 | NBI | −27 |

Assay theoretical bottom: blocking ELISA with hPD-1-mfc constant and hPD-L2 coat is 5.0E−11M
NBI—non-blocker

As indicated in Table 12, in the first assay format, 23 of the 27 anti-PD-1 antibodies blocked 1.5 nM of hPD-1-mFc from binding to hPD-L1-hFc with IC50 values ranging from 190 pM to 3.3 nM with the percent maximum blockage ranging from 67% to 100%. One antibody, H2aM7796N, was identified as a non-blocker. Three anti-PD-1 antibodies (H4H9068P2, H1M7789N, and H2aM7791N) were identified as non-blockers/enhancers.

As shown in Table 13, in the second assay format, 23 of the 27 anti-PD-1 antibodies blocked 200 pM of biot-hPD-1-hFc from binding to hPD-L1-mFc with IC50 values ranging from 59 pM to 1.3 nM with maximum percent blockade ranging from 60% to 101%. One antibody, H1M7789N, was identified as a non-blocker. Three anti-PD-1 antibodies (H4H9057P2, H4H9068P2, and H2aM7791N) were identified as non-blockers/enhancers.

In the third assay format as shown in Table 14, four anti-PD-1 antibodies of the disclosure, and an Isotype control were tested. All 4 anti-PD-1 antibodies of the disclosure blocked 100 pM (fixed concentration) of hPD-1-mFc from binding to plate-coated hPD-L2-hFc with IC50 values ranging from 0.13 nM to 1.3 nM and with maximum percent blockade ranging from 94% to 100%.

### Example 5: Blocking of PD-1 Binding to PD-L1 as Determined by Biosensor Assay and by Surface Plasmon Resonance

Inhibition of human PD-1 from binding to human PD-L1 by different anti-PD-1 monoclonal antibodies was studied either using real time bio-layer interferometry assay on an Octet Red96 biosensor instrument (Fortebio Inc.) or using a real-time surface plasmon resonance biosensor assay on a Biacore 3000 instrument.

Inhibition studies for anti-PD-1 monoclonal antibodies expressed with a mouse Fc were performed on an Octet Red 96 instrument. First, 100 nM of a recombinant human PD-1 expressed with a C-terminal mouse IgG2a Fc tag (hPD-1-mFc; SEQ ID NO: 323) was incubated with 500 nM of each anti-PD-1 monoclonal antibody for at least 1 hour before running the inhibition assay. Around 0.8 nm to 1.2 nm of recombinant human PD-L1 expressed with a C-terminal human IgG1 Fc tag (hPD-L1-hFc; SEQ ID NO: 325) was captured using anti-human IgG Fc capture Octet biosensor. The Octet biosensors coated with hPD-L1-hFc were then dipped into wells containing the mixture of hPD-1-mFc and different anti-PD-1 monoclonal antibodies. The entire experiment was performed at 25° C. in Octet HBST buffer (0.01 M HEPES PH7.4, 0.15M NaCl, 3 mM EDTA, 0.05% v/v Surfactant P20, 0.1 mg/mL BSA) with the plate shaking at a speed of 1000 rpm. The biosensors were washed in Octet HBST buffer in between each step of the experiment. The real-time binding responses were monitored during the entire course of the experiment and the binding response at the end of every step was recorded. Binding of hPD-1-mFc to the captured hPD-L1-hFc was compared in the presence and absence of different anti-PD-1 monoclonal antibodies and was used to determine the blocking behavior of the tested antibodies as shown in Table 15.

TABLE 15
Inhibition of human PD-L1 binding to PD-1
by anti-PD-1 monoclonal antibodies expressed with
mouse Fc as measured on an Octet Red 96 instrument

| | | Binding of the mixture of | |
|---|---|---|---|
| | Amount of | 100 nM hPD-1-mFc | |
| | hPD-L1-hFc | and 500 nM | |
| Anti-PD-1 | Captured | anti-PD-1 monoclonal | % |
| antibody | (nm) | antibody (nm) | Blocking |
| No Antibody | 0.77 | 0.07 | 0 |
| H2aM7780N | 1.07 | −0.01 | 114 |
| H2aM7788N | 0.74 | 0.00 | 100 |
| H1M7789N | 0.80 | 0.05 | 29 |
| H2aM7790N | 0.90 | −0.01 | 114 |
| H2aM7791N | 1.17 | 0.23 | −229 |
| H2aM7794N | 0.87 | −0.01 | 114 |
| H2aM7795N | 0.28 | −0.01 | 114 |
| H2aM7700N | 0.82 | −0.02 | 129 |
| H2aM7798N | 0.85 | 0.01 | 86 |
| H1M7799N | 0.79 | 0.00 | 100 |
| H1M7800N | 0.96 | 0.00 | 100 |

As shown in Table 15, 9 of the 11 anti-PD-1 antibodies tested on the Octet Red 96 instrument demonstrated strong blocking of hPD-1-mFc from binding to hPD-L1-hFc ranging from 86% to complete blockade of binding. One anti-PD-1 antibody (H1M7789N) tested showed weaker blocking of hPD-1-mFc binding to hPD-L1-hFc with 29% blockade. One antibody (H2aM7791N) tested demonstrated the ability to enhance the binding of hPD-1-mFc to hPD-L1-hFc.

Next, inhibition studies for anti-PD-1 monoclonal antibodies expressed with human Fc were performed on a Biacore 3000 instrument. First, 100 nM of a recombinant human PD-1 expressed with a C-terminal human IgG1 Fc tag (hPD-1-hFc; SEQ ID: 324) was incubated with 500 nM of each anti-PD-1 monoclonal antibody for at least 2 hours before running the inhibition assay. A CM5 Biacore sensor surface was first derivatized with polyclonal rabbit anti-mouse antibody (GE Catalog #BR-1008-38) using standard EDC-NHS chemistry. Around 730 RUs of recombinant human PD-L1 expressed with a C-terminal mouse IgG2a Fc tag (hPD-L1mFc; SEQ ID: 326) was then captured followed by the injection of 100 nM of hPD-1hFc in the presence and absence of different anti-PD-1 monoclonal antibodies at a flow rate of 25 μL/min for 3 minutes. The entire experiment was performed at 25° C. in running buffer comprised of 0.01 M HEPES PH7.4, 0.15M NaCl, 3 mM EDTA, 0.05% v/v Surfactant Tween-20 (HBS-ET running buffer). The real-time binding responses were monitored during the entire course of the experiment and the binding response at the end of every step was recorded. Binding of hPD-1-hFc to the captured hPD-L1-mFc was compared in the presence and absence of different anti-PD-1 monoclonal antibodies and was used to determine the blocking behavior of the tested antibodies as shown in Table 16.

TABLE 16
Inhibition of human PD-L1 binding to PD-1 by anti-
PD-1 monoclonal antibodies expressed with human Fc
as measured on a Biacore 3000 instrument

| | | Binding of the mixture of | |
|---|---|---|---|
| Anti-PD-1 | 500 nM of anti- | 100 nM hPD-1.hFc and | |
| monoclonal | PD-1 monoclonal | 500 nM anti-PD-1 | % |
| antibody | antibody (RU) | monocional antibody (RU) | Blocking |
| No Antibody | N/A | 100 ± 1.78 | N/A |
| H4H9019P | −2 | −1 | 101 |
| H4xH9034P | −4 | −5 | 105 |
| H4xH9035P | −3 | −4 | 104 |
| H4xH9037P | −4 | −4 | 104 |
| H4xH9046P | −4 | −5 | 105 |
| H4H9048P2 | −7 | 9 | 91 |
| H4H9057P2 | 58 | 57 | 43 |
| H4H9068P2 | −2 | 365 | −265 |
| H4xH9119P2 | −5 | −5 | 105 |
| H4xH9120P2 | 1 | 0 | 100 |
| H4xH9128P2 | −5 | −5 | 105 |
| H4xH9135P2 | −3 | −3 | 102 |
| H4xH9145P2 | −8 | −6 | 106 |

As shown in Table 16, 18 out of 20 anti-PD-1 antibodies of the disclosure tested on the Biacore 3000 instrument demonstrated strong blocking of hPD-1-hFc from binding to hPD-L1-mFc with the blockade ranging from 96% to 100%. One antibody demonstrated the ability to enhance the binding of hPD-1-hFc binding to hPD-L1-mFc. In this study, one of the tested antibodies of the disclosure (H4H9057P2) demonstrated non-specific background binding to the anti-mouse Fc capture surface.

### Example 6: Octet Cross-Competition Between Anti-PD-1 Antibodies

Binding competition between anti-PD-1 monoclonal antibodies was determined using a real time, label-free bio-layer interferometry assay on an Octet RED384 biosensor (Pall ForteBio Corp.). The entire experiment was performed at 25° C. in 0.01 M HEPES pH7.4, 0.15M NaCl, 3 mM EDTA, 0.05% v/v Surfactant Tween-20, 0.1 mg/mL BSA (Octet HBS-ET buffer) with the plate shaking at the speed of 1000 rpm. To assess whether 2 antibodies were able to compete with one another for binding to their respective epitopes on a recombinantly expressed human PD-1 with a C-terminal myc-myc-hexahistidine tag (hPD-1-MMH; SEQ ID: 321), around 0.1 nM of hPD-1-MMH was first captured onto anti-Penta-His antibody coated Octet biosensor tips (Pall ForteBio Corp., #18-5079) by submerging the tips for 5 minutes into wells containing a 50 μg/mL solution of hPD-1-MMH. The antigen captured biosensor tips were then saturated with the first anti-PD-1 monoclonal antibody (subsequently referred to as mAb-1) by dipping into wells containing 50 μg/mL solution of mAb-1 for 5 minutes. The biosensor tips were then subsequently dipped into wells containing a 50 μg/mL solution of a second anti-PD-1 monoclonal antibody (subsequently referred to as mAb-2). The biosensor tips were washed in Octet HBS-ET buffer in between every step of the experiment. The real-time binding response was monitored during the course of the experiment and the binding response at the end of every step was recorded. The response of mAb-2 binding to hPD-1-MMH pre-complexed with mAb-1 was compared and competitive/non-competitive behavior of different anti-PD-1 monoclonal antibodies was determined. Results are summarized in Table 17 (*Self-competing mAb2s are not listed).

TABLE 17
Cross-competition between pairs of selected anti-PD-1 antibodies

| First antibody | |
|---|---|
| applied | |
| (“mAb1”) | mAb2 Antibodies shown to compete with mAb1* |
| H4xH8992P | H4xH8999P, H1M7799N, H2aM7780N, |
| | H1M7800N, H2aM7788N, |
| | H2aM7794N, H2aM7798N, H4xH9146P2, |
| | H4H9057P2, H4xH9120P2, |
| | H4xH9128P2, H4H8019P H4xH9119P2, |
| | H4xH9135P2, H4xH9034P, |
| | H2aM7790N, H4xH9035P, H4xH9037P, |
| | H4xH9045P, H2aM7795N |
| H4xH8999P | H4xH6992P, H1M7799N, H2aM7780N, |
| | H1M7800N, H2aM7788N, |
| | H2aMY794N, H2aM7798N, H4xH9145P2, |
| | H4M9057P2, H4xH9120P2, |
| | H4xH9128P2, H4H9019P, H4xH9119P2, |
| | H4xH9136P2, H4xH9034P, |
| | H2aM7790N, H4xH9035P, H4xH9037P, |
| | H4xH9045P, H2aM7795N, |
| | H4xH9008P |
| H1M7799N | H4xH8992P, H4xH8999P, H2aM7780N, |
| | H1M7800N, H2aM7788N, |
| | H2aM7794N, H2aM7798N, H4xH9145P2, |
| | H4H9057P2, H4xH [symbol] 120P2, |
| | H4xH9128P2, H4H9019P, H4xH9119P2, |
| | H4xH9135P2, H4xH9034P, |
| | H2aM7790N, M4xH9035P, H4xH9037P, |
| | H4xH9045P, H2aM7795N |
| H2aM7780N | H4xH8982P, H4xH89999, H1M7799N, |
| | H1M7800N, H2aM7788N, |
| | H2aM7794N, H2AM7798N, H4xH9145P2, |
| | H4H9057P2, H4xH9120P2, |
| | H4xH9128P2, H4M8019P, H4xH9119P2, |
| | H4xH9135P2, H4xH9034P, |
| | H2AM7790N, H4xH9035P, H4xH8037P, |
| | H4xH9045P, H2aM7795N, |
| | H4xH9008P |
| H1M7800N | H4xH8992P, H4xH8999P, H1M7799N, |
| | H2aM7780N, H2aM7788N, |
| | H2aM7794N, H2aM7798N, H4xH9145P2, |
| | H4H9057P2, H4xH9120P2, |
| | H4xH9128P2, H4H9019P, H4xH9119P2, |
| | H4xH9135P2, H4xH93034P, |
| | H2aM7790N, H4xH9035P, H4xH9037P, |
| | H4xH9045P, H2aM7795N, |
| | H4xH9008P |
| H2aM7788N | H4xH8992P, H4xH8999P, H1M7799N, |
| | H2aM7780N, H1M7800N, |
| | H2aM7794N, H2aM7798N, H4xH9145P2, |
| | H4H9057P2, H4xH9120P2, |
| | H4xH9128P2, H4H9019P, H4xH9119P2, |
| | H4xH9135P2, H4xH9034P, |
| | H2aM7790N, H4xH9035P, H4xH9037P, |
| | H4xH9045P, H2aM7795N, |
| | H2aM7791N |
| H2aM7794N | H4xH8992P, H4xH8999P, H1M7790N, |
| | H2aM7780N, H1M7800N, |
| | H2aM7788N, H2aM7798N, H4xH9145P2, |
| | H4H9057P2, H4xH9120P2, |
| | H4xH9128P2, H4H9019P, H4xH9119P2, |
| | H4xH9135P2, H4xH9034P, |
| | H2AM7790N, H4xH9035P, H4xH9037P, |
| | H4xH9045P, H2aM7795N, |
| | H4xH9008P |
| H2aM7798N | H4xH8992P, H4xH8999P, H1M7799N, |
| | H2aM7780N, H1M7800N, |
| | H2aM7788N, H2aM7794N, H4xH9145P2, |
| | H4H9067P2, H4xH9120P2, |
| | H4xH9128P2, H4H8019P, H4xH9119P2, |
| | H4xH9135P2, H4xH9034P, |
| | H23M7780N, H4xH9035P, H4xH9037P, |
| | H4xH8045P, H2aM7795M, |
| | H4xH9008P |
| H4xH9145P2 | H4xH8992P, H4xH8999P, H1M7799N, |
| | H2aM7780N, H1M7800N, |
| | H2aM7788N, H2aM7794N, H2aM7798N, |
| | H4H9057P2, H4xH9120P2, |
| | H4xH9128P2, H4H8019P, N4xH9119P2, |
| | H4xH9135P2, H4xH9034P, |
| | H2aM7790N, H4xH9035P, H4xH9037P, |
| | H4xH9045P, H4xH9008P |
| H4H9057P2 | H4xH8992P, H4xH8999P, H1M799N, |
| | H2aM7780N, H1M7800N, |
| | H2aM7788N, H2aM7794N, H2aM7798N, |
| | H4xH9145P2, H4xH9120P2, |
| | H4xH9128P2, H4H9019P, H4xH9119P2, |
| | H4xH9135P2, H4xH9034P, |
| | H2aM7790N, H4xH8035P, H4xH9037P, |
| | H4xH8045P, H2aM7795N, |
| | H2aM7791N, H4xH9048P2 |
| H4xH9120P2 | H4xH8992P, H4xH8999P H1M7799N, |
| | H2aM7780N, H1M7800N, |
| | H2aM7788N, H2aM7794N, H2aM7798N, |
| | H4xH9145P2, H4H9067P2, |
| | H4xH9128P2, H4H9019P, H4xH9119P2, |
| | H4xH9135P2, H4xH9034P, |
| | H2aM7790N, H4xH9035P, H4xH9037P, |
| | H4xH9045P, H4xH9048P2 |
| H4xH9128P2 | H4xM8992P, H4xM8999P, H1M7799N, |
| | H2aM7780N, H1M7800N, |
| | H2aM7788N, H2aM7794N, H2aM7798N, |
| | H4xH9145P2, H4H9067P2, |
| | H4xH8120P2, H4H9019P, H4xH9119P2, |
| | H4xH9135P2, H4xH8034P |
| | H2aM7790N, H4xH9035P, H4xH9037P, |
| | H4xH9045P, H4xH9008P, |
| | H4H9066P2, H4xH9048P2 |
| H4H9019P | H4xH8992P, H4xH8999P, H1M7799N, |
| | H2aM7780N, H1M7800N, |
| | H2aM7794N, H2aM7798N, H4xH9145P2, |
| | H4H9057P2, H4xH9120P2, |
| | H4xH9128P2, H2aM7788N, |
| | H4xH9119P2, H4xH9135P2, |
| | H4xH9034P, H4xH9036P, H4xH9037P, |
| | H4xH9046P, H2aM7796N, |
| | H2aM7791N |
| H4xH9119P2 | H4xH8992P, H4xH8999P, H1M7798N, |
| | H2AM7780N, H1M7800N, |
| | H2aM7794N, H2aM7798N, H4xH0146P2, |
| | H4H9057P2, H4xH9120P2, |
| | H4xH9128P2, H2aM7788N, H4H9019P, |
| | H4xH9136P2, H4xH9034P, |
| | H4xH9035P, H4xH9037P, H4xH9045P, |
| | H2aM7795N, H2aM7791N |
| H4xH9136P2 | H4xH8992P, H4xH9999P, H1M7799N, |
| | H2aM7780N, H1M7800N, |
| | H2aM7794N, H2aM7798N, H4xH9146P2, |
| | H4H9057P2, H4xH9120P2, |
| | H4xH9128P2, H2aM7788N, H4H9019P, |
| | H4xH9119P2, H4xH9034P, |
| | H4xH9035P, H4xH9037P, H4xH9045P, |
| | H2aM7795N, H2aM7791N |
| H4xH9034P | H4xH8992P, H4xH8999P, H1M7799N, |
| | H2aM77800N, H1M7800N, |
| | H2aM7794N, H2aM7798N, H4xH9145P2, |
| | H4H9057P2, H4xH9120P2, |
| | H4xH9128P2, H4H9019P, H4xH9119P2, |
| | H4xH9135P2, H2aM7788N, |
| | H2aM17790N, H4xH9035P, H4xH9037P, |
| | H4xH9045P, M2aM17795N, |
| | H2aM7791N |
| H2aM7790N | H4xH8992P, H1M7799N, H2aM7780N, |
| | H1M7800N, H2aM7788N, |
| | H2aM7704N, H2aM7798N, H4xH9145P2, |
| | H4H9057P2, H4xH9120P2, |
| | H4xH9128P2, H4xH9034P, |
| | H4xH8999P, H4xH9008P |
| H4xH9036P | H4xH8992P, H4xH8999P, H1M7799N, |
| | H2aM7780N, H1M7800N, |
| | H2aM7794N, H2aM7798N, H4xH9145P2, |
| | H4H9057P2, H4xH9120P2, |
| | H4xH9128P2, H2aM7788N, H4H9019P, |
| | H4XH9119P2, H4xH9034P, |
| | H4xH9135P2, H4xH9037P, H4xH9045P, |
| | H2aM7795N, H2aM7791N |
| H4xH9037P | H4xH8992P, H4xH8999P, H1M7799N, |
| | H2aM7780N, H1M7800N, |
| | H2aM7794N H2aM7798N, H4xH9145P2, |
| | H4H9057P2, H4xH9120P2, |
| | H4xH9128P2, H2aM7788N, H4H9019P, |
| | H4xH9119P2, H4xH9034P, |
| | H4xH9135P2, H4xH9035P, H4xH9045P, |
| | H2aM7795N, H2aM7791N |
| H4xH9045P | H4xH8982P, H4xH8999P, H1M7799N, |
| | H2aM7780N, H1M7800N, |
| | H2aM7784N, H2aM7798N, H4xH9145P2, |
| | H4H9057P2, H4xH9120P2, |
| | H4xH9128P2, H2aM7788N, H4H9019P, |
| | H4xH9119P2, H4xH9034P, |
| | H4xH9135P2, H4xH9035P, H4xH9037P, |
| | H2aM7795N, H2aM7791N |
| H2aM7795N | H4xH8992P, H4xH8999P, H1M7798N, |
| | H2aM7780N, H1M7800N, |
| | H2aM7794N, H2aM7798N, H4H9057P2, |
| | H2aM7788N, H4H9019P |
| | H4xH9119P2, H4xH9034P, H4xH9135P2, |
| | H4xH9035P, H4xH9037P, |
| | H4xH9045P, H2AMY791N |
| H4xH9008P | H4xH8989P, H2aM7780N, H2aM7794N, |
| | H2aM7798N, H4xH9145P2, |
| | H4xH9128P2 H2aM7790N, H4H9068P2, |
| | H1M7799N, H4xH9048P2 |
| H2aM7791N | H2aM7788N, H4H9057P2, H4H9019P, |
| | H4xH9119P2, H4xH9135P2, |
| | H4xH9034P, H4xH9035P, H4xH9037P, |
| | H4xH9046P, H2aM7796N |
| H4H9068P2 | H4xH9128P2, H4xH8008P, |
| | H1M7789N, H4xH9048P2 |
| H1M7789N | H4xH9008P, H4H9068P2, H4xH9048P2 |
| H4xH9048P2 | H4H9057P2, H4xH9120P2, H4xH9128P2, |
| | H4H9019P, H4xH9008P, |
| | H4H9068P2, H1M7799N |

[symbol]

indicates data missing or illegible when filed

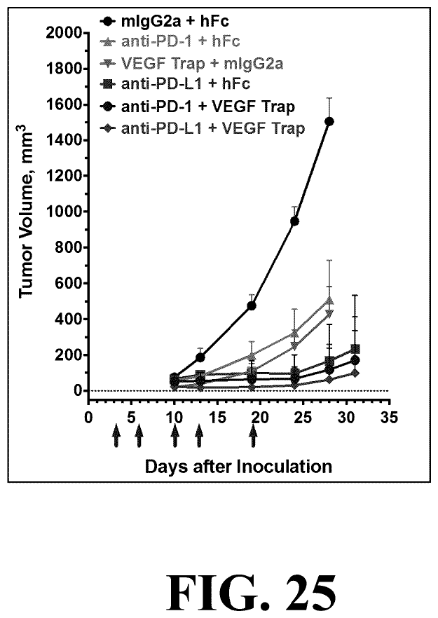

A second binding competition between a panel of selected anti-PD-1 monoclonal antibodies was determined using a real time, label-free bio-layer interferometry assay on an Octet HTX biosensor (Pall ForteBio Corp.). The entire experiment was performed at 25° C. in 0.01 M HEPES PH7.4, 0.15M NaCl, 3 mM EDTA, 0.05% v/v Surfactant Tween-20, 0.1 mg/mL BSA (Octet HBS-ET buffer) with the plate shaking at the speed of 1000 rpm. To assess whether 2 antibodies were able to compete with one another for binding to their respective epitopes on the hPD-1-MMH, around 0.25 nm of hPD-1-MMH was first captured onto anti-Penta-His antibody coated Octet biosensor tips (Fortebio Inc, #18-5079) by submerging the tips for 150 seconds into wells containing a 10 μg/mL solution of hPD-1-MMH. The antigen-captured biosensor tips were then saturated with a first anti-PD-1 monoclonal antibody (subsequently referred to as mAb-1) by dipping into wells containing 100 μg/mL solution of mAb-1 for 5 minutes. The biosensor tips were then subsequently dipped into wells containing a 100 μg/mL solution of second anti-PD-1 monoclonal antibody (subsequently referred to as mAb-2) for 4 minutes. All the biosensors were washed in Octet HBS-ET buffer in between every step of the experiment. The real-time binding response was monitored during the course of the experiment and the binding response at the end of every step was recorded as shown in FIG. 25. The response of mAb-2 binding to hPD-1-MMH pre-complexed with mAb-1 was compared and competitive/non-competitive behavior of different anti-PD-1 monoclonal antibodies was determined. Results are summarized in Table 18 (*Self-competing mAb2s are not listed).

TABLE 18
Cross-competition between pairs of selected anti-PD-1 antibodies

| First Antibody applied | mAb2 Antibodies Shown to |
|---|---|
| (“mAb1”) | Compete with mAb1* |
| H4H7795N2 | H4H7798N |
| H4H7798N | H4H7795N2; H4H9008P |
| H4H9008P | H4H7798N; H4H9068P2 |
| H4H9068P2 | H4H9008P; H4H9048P2 |
| H4H9048P2 | H4H9068P2 |

Under the experimental conditions disclosed in this Example, H4H7795N2 cross-competed with H4H7798N; H4H7798N cross-competed with H4H7795N2 and H4H9008P; H4H9008P cross-competed with H4H7798N and H4H9068P2; H4H9068P2 cross-competed with H4H9008P and H4H9048P2.

### Example 7: Antibody Binding to Cells Overexpressing PD-1

The binding of anti-PD-1 antibodies to a human embryonic kidney cell line (HEK293; ATCC, #CRL-1573) stably transfected with full length human PD-1 (amino acids 1 to 289 of accession number NP_005009.2) (HEK293/hPD-1) was determined by FACS.

For the assay, adherent cells were detached using trypsin or enzyme-free dissociation buffer and blocked with complete medium. Cells were centrifuged and resuspended at a concentration of 2.5-6×10\hat{ }6 cells/mL in cold PBS containing 2% FBS. HEK293 parental and HEK293/hPD-1 cells were then incubated for 15-30 min on ice with 100 nM of each anti-PD-1 antibody. Unbound antibodies were removed by washing with D-PBS containing 2% FBS, and cells were subsequently incubated with an allophycocyanin-conjugated secondary F(ab′)2 recognizing either human Fc (Jackson ImmunoResearch, #109-136-170) or mouse Fc (Jackson ImmunoResearch, #115-136-146) for 15-30 minutes on ice. Cells were washed with D-PBS containing 2% FBS to remove unbound secondary F(ab′)2 and fluorescence measurements were acquired using either a HyperCyte (IntelliCyt, Inc.) flow cytometer or an Accuri flow cytometer (BD Biosciences). Data was analyzed using FlowJo software (Tree Star).

TABLE 19
FACS binding of anti-PD-1 antibodies to HEK293/hPD-1 cElls
and parental HEK283 cells

| | | FACS on | Ratio of |
|---|---|---|---|
| | | HEK293/ | HEK293/hPD-1 |
| | FACS on HEK293 | hPD-1 | to HEX293 |
| Antibody | parental cells [MFI] | cells [MFI] | parental cells |
| H1M7789N | 262 | 24168 | 92.3 |
| H1M7799N | 255 | 6855 | 26.9 |
| H1M7800N | 275 | 6812 | 24.7 |
| H2aM7780N | 320 | 23656 | 73.8 |
| H2aM7788N | 305 | 23112 | 75.7 |
| H2aM7790N | 270 | 47310 | 175.5 |
| H2aM7791N | 274 | 4948 | 18.0 |
| H2aM7794N | 270 | 19127 | 71.0 |
| H2aM7795N | 288 | 817 | 2.8 |
| H2aM7796N | 297 | 49765 | 167.8 |
| H2aM7798N | 300 | 23443 | 78.1 |
| H4H9019P | 111 | 8610 | 77.2 |
| H4H9057P2 | 141 | 6501 | 46.1 |
| H4H9068P2 | 285 | 1940 | 6.8 |
| H4xH8992P | 358 | 17502 | 48.9 |
| H4xH8999P | 809 | 28875 | 35.7 |
| H4xH8008P | 509 | 26233 | 51.5 |
| H4xH9034P | 147 | 10116 | 69.0 |
| H4xH9036P | 108 | 9916 | 91.7 |
| H4xH9037P | 108 | 8787 | 81.4 |
| H4xH9046P | 95 | 8884 | 93.7 |
| H4xH9048P2 | 102 | 7196 | 70.8 |
| H4xH9119P2 | 109 | 9142 | 84.0 |
| H4xH9120P2 | 109 | 9975 | 91.9 |
| H4xH9128P2 | 135 | 9081 | 67.5 |
| H4xH9135P2 | 114 | 9380 | 82.2 |
| H4xH9145P2 | 226 | 11552 | 51.2 |

As shown in Table 19, 25 of the 27 anti-PD-1 antibodies of the disclosure showed strong binding to the HEK293/hPD-1 cells compared to binding on the parental HEK293 line. Two antibodies of the disclosure (H2aM7795N and H4H9068P2) bound weaker to human PD-1 expressing cells compared to the other antibodies tested.

To further characterize anti-PD1 antibodies of the disclosure, dose-dependent binding to a human embryonic kidney cell line (HEK293; ATCC, #CRL-1573) stably transfected with full length human PD-1 (amino acids 1 to 289 of accession number NP_005009.2) (HEK293/hPD-1) was determined by FACS.

For the assay, adherent cells were detached using trypsin and blocked with complete medium. Cells were centrifuged and resuspended at a concentration of 6×10\hat{ }6 cells/mL in staining buffer (1% FBS in PBS). To determine the EC50 and Emax of the anti-PD1 antibodies, 90 μL of cell suspension was incubated for 30 minutes on ice with a serial dilution of anti-PD-1 antibodies and controls diluted to a final concentration ranging from 5 pM to 100 nM (no mAb sample was included as negative control) in staining buffer. Cells were then centrifuged and pellets were washed once with staining buffer to remove unbound antibodies. Cells were subsequently incubated for 30 minutes on ice either with an allophycocyanin-conjugated secondary F(ab′)2 recognizing human Fc (Jackson ImmunoResearch, #109-136-170) or mouse Fc (Jackson ImmunoResearch, #115-136-071). Cells were centrifuged and pellets were washed once with staining buffer to remove unbound secondary F(ab′)2 and then fixed overnight with a 1:1 dilution of Cytofix (BD Biosciences, #554655) and staining buffer. The following day, cells were centrifuged and pellets were washed once with staining buffer, resuspended and filtered. Fluorescence measurements were acquired on Hypercyt® cytometer and analyzed in ForeCyt™ (IntelliCyt; Albuquerque, N. Mex.) to determine the mean fluorescence intensities (MFI). The EC50 values were calculated from a four-parameter logistic equation over an 11-point response curve using GraphPad Prism. Emax for each antibody was defined as the binding at the highest antibody dose (100 nM) tested.

TABLE 20
Dose dependent FACS binding of anti-PD-1 antibodies
to HEK293/PD-1 cells

| | | Max Geom. Mean |
|---|---|---|
| Antibody | EC₅₀ [M] | [MFI] @ 100 nM |
| H2aM7779N | 2.59E−09 | 16832 |
| H2aM7780N | 1.69E−09 | 18415 |
| H2aM7781N | 5.67E−10 | 13740 |
| H2aM7782N | 1.26E−09 | 17302 |
| H2aM7787N | 2.40E−09 | 15744 |
| H2aM7788N | 3.21E−40 | 14827 |
| H2aM7790N | 1.71E−10 | 19196 |
| H2aM7791N | No EC₅₀ determined | 1397 |
| H2aM7794N | 1.37E−09 | 16406 |
| H2aM7796N | No EC₅₀ determined | 624 |
| H2aM7798N | 6.985E−11 | 20900 |
| H1M7799N | 3.318E−11 | 24405 |
| H1M7800N | 4.80E−11 | 20763 |
| H4xH8992P | 5.45E−11 | 11368 |
| H4xH8899P | 5.27E−11 | 28341 |
| H4H9019P | 1.40E−09 | 29201 |
| H4xH9034P | 2.09E−10 | 32388 |
| H4xH9035P | 1.15E−10 | 28708 |
| H4xH9037P | 6.74E−10 | 36441 |
| H4xH9045P | 9.17E−11 | 24662 |
| H4xH9048P2 | 6.68E−10 | 33687 |
| H4H9057P2 | 2.363E−10 | 19963 |
| H4H9068P2 | No EC₅₀ determined | 639 |
| H4xH9119P2 | 3.476E−10 | 37789 |
| H4xH9120P2 | 4.797E−10 | 34057 |
| H4xH9128P2 | 1.551E−09 | 37167 |
| H4xH9135P2 | 1.048E−18 | 32793 |
| H4xH9145P2 | 2.321E−10 | 30613 |
| mlgG1 isotype | N/A | 200 |
| mlgG2a isotype | N/A | 239 |
| hlgG4 isotype | N/A | 459 |

TABLE 21
Dose dependent FACS binding of anti-PD-1 antibodies
to HEK293/hPD-1 cells

| | | Max Geom. Mean |
|---|---|---|
| Antibody | EC₅₀ [M] | [MFI] @ 100 nM |
| H4H7795N2 | Inconclusive | 15188 |
| H4H7798N | 5.09E−10 | 20305 |
| H4H9008P | Inconclusive | 32230 |
| H4H9048P2 | 1.60E−09 | 39774 |
| H1M7789N | Inconclusive | 35574 |
| H2aM7796N | 4.81E−09 | 14111 |
| mlgG1 isotype | N/A | 858 |
| mlgG2a isotype | N/A | 352 |
| hlgG4 isotype | N/A | 809 |

As shown in Table 20, 25 of 28 anti-PD1 antibodies of the disclosure showed dose dependent binding to HEK293/hPD-1 cells with EC50 values ranging from 33.18 pM to 2.59 nM and Emax values ranging from 37,789 to 11,368 MFI. Three anti-PD1 antibodies of the disclosure did not demonstrate strong binding to HEK293/hPD-1 cells and therefore an EC50 value could not be determined. None of the isotype controls demonstrated any measurable binding in this assay.

As shown in Table 21, 3 of 6 anti-PD1 antibodies of the disclosure showed dose dependent binding to HEK293/hPD-1 cells with EC50 values ranging from 509 pM to 4.81 nM and Emax values ranging from 39,774 to 14, 111 MFI. Three antibodies of the disclosure tested bound to HEK293/hPD-1 cells, but did not reach a plateau. Therefore their precise EC50 values could not be determined and their EC50 values are referred to as inconclusive. None of the isotype controls demonstrated any measurable binding in this assay.

### Example 8: Blocking of PD-1-Induced T-Cell Down-Regulation in a T-Cell/APC Luciferase Reporter Assay

T-cell activation is achieved by stimulating T-cell receptors (TcR) that recognize specific peptides presented by major histocompatibility complex class I or II proteins on antigen-presenting cells (APC). Activated TcRs in turn initiate a cascade of signaling events that can be monitored by reporter genes driven by transcription factors such as activator-protein 1 (AP-1), Nuclear Factor of Activated T-cells (NFAT) or Nuclear factor kappa-light-chain-enhancer of activated B cells (NFκb). T-cell response is modulated via engagement of co-receptors expressed either constitutively or inducibly on T-cells. One such receptor is PD-1, a negative regulator of T-cell activity. PD-1 interacts with its ligand, PD-L1, which is expressed on target cells including APCs or cancer cells, and acts to deliver inhibitory signals by recruiting phosphatases to the TcR signalosome, resulting in the suppression of positive signaling.

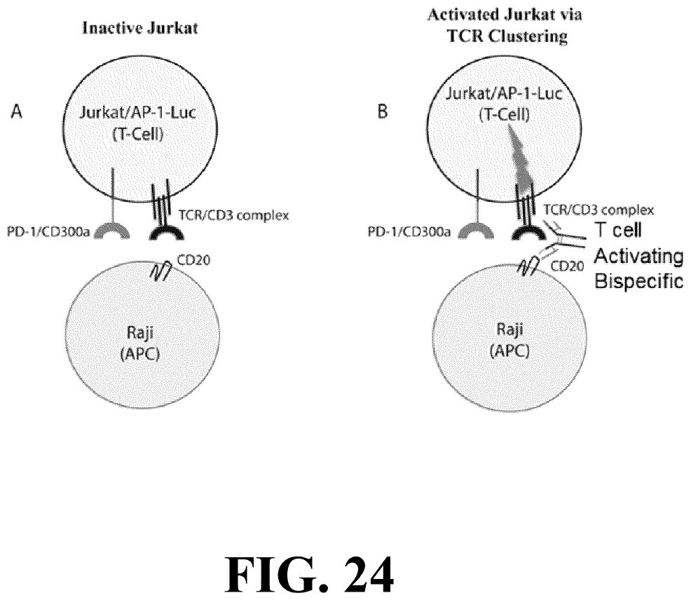

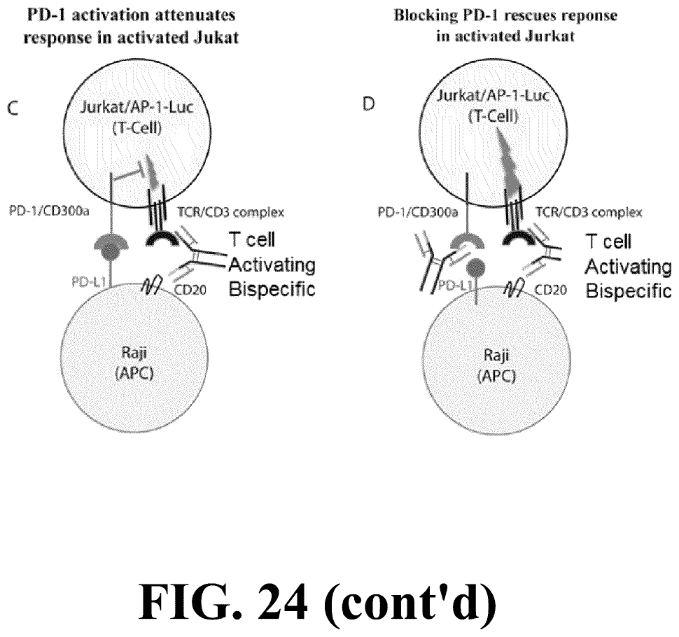

The ability of anti-PD-1 antibodies to antagonize PD-1/PD-L1-mediated signaling through the PD-1 receptor in human T cell lines was assessed using an in vitro cell based assay shown in FIG. 24. The bioassay was developed to measure T cell signaling induced by interaction between APC and T cells by utilizing a mixed culture derived from two mammalian cell lines: Jurkat cells (an immortalized T cell line) and Raji cells (a B cell line). For the first component of the bioassay, Jurkat Clone E6-1 cells (ATCC, #TIB-152) were transduced with the Cignal Lenti AP-1 Luc Reporter (Qiagen-Sabiosciences, #CLS-011L) as per the manufacturer's instructions. The lentivirus encodes the firefly luciferase gene under the control of a minimal CMV promoter, tandem repeats of the TPA-inducible transcriptional response element (TRE) and a puromycin resistance gene. The engineered Jurkat cell line was subsequently transduced with a PD-1 chimera comprising the extracellular domain of human PD-1 (amino acids from 1 to 170 of human PD1; accession number NP_005009.2) and the trans-membrane and cytoplasmic domains of human CD300a (amino acids from 181 to 299 of human CD300a; accession number NP_009192.2). The resulting stable cell line (Jurkat/AP1-Luc/hPD1-hCD300a) was selected and maintained in RPMI/10% FBS/penicillin/streptomycin/glutamine supplemented with 500 μg/mL G418+1 μg/mL puromycin.

For the second component of the bioassay, Raji cells (ATCC, #CCL-86) were transduced with human PD-L1 gene (amino acids 1-290 of accession number NP_054862.1) that had been cloned into a lentiviral (pLEX) vector system (Thermo Scientific Biosystems, #OHS4735). Raji cells, positive for PD-L1 (Raji/hPD-L1) were isolated by FACS using a PD-L1 antibody and maintained in Iscove/10% FBS/penicillin/streptomycin/glutamine supplemented with 1 μg/mL puromycin.

To simulate the APC/T cell interaction, a bispecific antibody composed of one Fab arm that binds to CD3 on T cells and the other one Fab arm binding that binds to CD20 on Raji cells (CD3×CD20 bispecific antibody; e.g., as disclosed in US20140088295) was utilized. The presence of the bispecific molecule in the assay results in the activation of the T cell and APC by bridging the CD3 subunits on T-cells to CD20 endogenously expressed on Raji cells. Ligation of CD3 with anti-CD3 antibodies has been demonstrated to lead to activation of T cells. In this bioassay, antibodies blocking the PD1/PD-L1 interaction rescue T-cell activity by disabling the inhibitory signaling and subsequently leading to increased AP1-Luc activation.

In the luciferase-based bioassay, RPMI1640 supplemented with 10% FBS and penicillin/streptomycin/glutamine was used as assay medium to prepare cell suspensions and antibody dilutions to carry out the screening of anti-PD1 monoclonal antibodies (mAbs). On the day of the screening, EC50 values of anti-PD1 mAbs, in the presence of a fixed concentration of CD3×CD20 bispecific antibody (30 pM), as well as the EC50 of the bispecific antibody alone, were determined. In the following order, cells and reagents were added to 96 well white, flat-bottom plates. For the anti-PD1 mAb EC50 determinations, first a fixed concentration of CD3×CD20 bispecific antibody (final 30 pM) was prepared and added to the microtiter plate wells. Then 12-point serial dilutions of anti-PD1 mAbs and controls were added (final concentrations ranging from 1.7 pM to 100 nM; plus wells with assay medium alone). For the bispecific antibody (alone) EC50 determination, the bispecific antibody, at final concentrations ranging from 0.17 pM to 10 nM (plus wells with assay medium alone), was added to the microtiter plate wells. Subsequently, a 2.5×10\hat{ }6/mL Raji/hPD-L1 cell suspension was prepared and 20 μL per well was added (final cell number/well 5×10\hat{ }4 cells). Plates were left at room temperature (15-20 minutes), while a suspension of 2.5×10\hat{ }6/mL of Jurkat/AP1-Luc/hPD1 (ecto)-hCD300a (TM-Cyto) was prepared. 20 μL of the Jurkat suspension (final cell number/well 5×10\hat{ }4 cells) was added per well. Plates containing the co-culture were incubated for 5 to 6 hours at 37° C./5% CO2. Samples were tested in duplicates and luciferase activity was then detected after the addition of ONE-Glo™ (Promega, #E6051) reagent and relative light units (RLUs) were measured on a Victor luminometer.

RLU values for each screened antibody were normalized by setting the assay condition with fixed (30 pM) concentration of the CD3/CD20 bispecific antibody, but without anti-PD-1 antibody to 100%. This condition corresponds to the maximal AP1-Luc response elicited by the bispecific molecule in the presence of the PD-1/PD-L1 inhibitory signal. Upon addition of the anti-PD-1 antibody, the inhibitory signal is suppressed, and the increased stimulation is shown here as Emax, the percentage increase in the signal in the presence of the highest antibody dose tested (100 nM). To compare potency of the anti-PD1 antibodies tested, the concentration of antibody at which the normalized RLU value reached 150% activation was determined from a four-parameter logistic equation over a 12-point response curve using GraphPad Prism. The results are summarized in Table 22 and Table 23, respectively.

TABLE 22
Anti-PD1 antibody blocking PD-1/PD-L1 dependent inhibition
of AP1-Luc signaling in Experiment 1

| | Antagonistic assay | Antagonistic assay |
|---|---|---|
| | Concentration (M) | Eₘₐₓ mean [%] @ |
| | of Antibody at 150% | 100 nM |
| Antibody | activation Experiment 1 | Experiment 1 |
| H1M7789W | N/A | 135 |
| H1M7799N | 2.97E−08 | 183 |
| H1M7800N | 1.65E−08 | 182 |
| H2aM7779N | 8.92E−09 | 214 |
| H2aM7780N | 6.62E−09 | 228 |
| H2aM7781N | 6.70E−09 | 230 |
| H2aM7782N | 9.96E−08 | 215 |
| H2aM7787N | 1.38E−08 | 215 |
| H2aM7788N | 4.72E−09 | 189 |
| H2aM7790N | 5.24E−09 | 234 |
| H2aM7791N | N/A | 103 |
| H2aM7794N | 4.09E−08 | 170 |
| H2aM7786N | N/A | 109 |
| H2aM7796N | N/A | 121 |
| H2aM7798N | 7.99E−10 | 239 |
| H4H9019P | 1.79E−08 | 180 |
| H4xH9034P | 2.62E−09 | 202 |
| H4xH9035P | 1.20E−09 | 227 |
| H4xH9037P | 2.82E−09 | 195 |
| H4xH9045P | 2.23E−08 | 176 |
| H4xH9048P2 | N/A | 138 |
| H4H9057P2 | 2.88E−08 | 212 |
| H4H9068P2 | N/A | 102 |
| H4xH9119P2 | 1.11E−08 | 163 |
| H4xH9120P2 | 1.10E−08 | 166 |
| H4xH9128P2 | 3.99E−09 | 187 |
| H4xH9135P2 | 1.55E−09 | 193 |
| H4xH9145P2 | 2.40E−09 | 185 |
| H4xH8902P | 5.32E−09 | 178 |
| H4xH8999P | 8.63E−10 | 217 |
| H4H7798N | 1.54E−09 | 202 |
| mlgG1 isotype control | N/A | 92 |
| mlgG2a isotype control | N/A | 91 |
| hlgG4 isotype control | N/A | 94 |

N/A = not applicable because at the concentrations tested these antibodies did not activate 150%

TABLE 23
Anti-PD1 antibody blocking PD-1/PD-L1 dependent inhibition
of AP1-Luc signaling in Experiment 2

| | Antagonistic assay | |
|---|---|---|
| | Concentration (M) of | |
| | Antibody at 150% | Antagonistic assay Eₘₐₓ |
| | activation | mean [%] @ 100 nM |
| Antibody | Experiment 2 | Experiment 2 |
| H4H7795N2 | N/A | 110 |
| H4H7708N | 1.59E−10 | 343 |
| H4H9008P | 9.84E−08 | 150 |
| H4H9048P | N/A | 134 |
| hlgG4 isotype control | N/A | 98 |

N/A = not applicable because at the concentrations tested these antibodies did not activate 150%

As shown in Table 23, 2 out of the 4 anti-PD-1 antibodies of the disclosure tested blocked PD-1/PD-L1 inhibition with Emax values of 150 and 343%, respectively. 2 out of the 4 anti-PD-1 antibodies of the disclosure did not demonstrate substantial blockade of PD1/PD-L1 interaction when tested in this assay.

### Example 9: In Vivo Efficacy of Anti-PD-1 Antibodies

To determine the effect of a select number of anti-PD-1 antibodies of the disclosure in a relevant in vivo model, three MC38.ova tumor growth studies, involving subcutaneous injection of tumor cells and started on different days, were conducted in mice that were homozygous for the expression of the extracellular domain of human PD-1 in place of extracellular domain of mouse PD-1 (PD-1 Humin mice) on a 75% C57/B16/25% 129 strain background.

For the studies, mice were divided evenly according to body weight into 5 treatment or control groups for Study 1 (5 mice per group), 8 treatment or control groups for Study 2 (5 mice per group), and 5 treatment or control groups for Study 3 (7 mice per group). At day 0, mice were anesthetized by isoflurane inhalation and then injected subcutaneously into the right flank with 5×105 MC38.ova cells in suspension of 100 μL of DMEM for Study 1 or 1×106 MC38.ova cells in suspension of 100 μL of DMEM for Study 2 and Study 3. For Study 1, treatment groups were intraperitoneally injected with 200 μg of either one of three anti-PD-1 antibodies of the disclosure, or an isotype control antibody with irrelevant specificity on days 3, 7, 10, 14, and 17 of the experiment, while one group of mice was left untreated. For Study 2, treatment groups were intraperitoneally injected with either one of three anti-PD-1 antibodies of the disclosure at 10 mg/kg or 5 mg/kg per/dose, one antibody of the disclosure (H4H7795N2) at 10 mg/kg per dose, or an isotype control antibody with irrelevant specificity at 10 mg/kg on days 3, 7, 10, 14, and 17 of the experiment. For Study 3, treatment groups were intraperitoneally injected with either one of two anti-PD-1 antibodies of the disclosure at 5 mg/kg or 2.5 mg/kg per/dose, or an isotype control antibody with irrelevant specificity at 5 mg/kg on days 3, 7, 10, 14, and 17 of the experiment. Experimental dosing and treatment protocol for groups of mice are shown in Table 24.

TABLE 24
Experimental dosing and treatment protocol for groups of mice

| | | Dosage amount | |
|---|---|---|---|
| | | at each dosage | |
| Study # | Samples Tested | time poial | Dosing interval |
| 1 | Isotype Control | 200 ug | Days 3, 7, 10, 14, 17 |
| | No treatment | N/A | N/A |
| | H4H7798N | 200 ug | Days 3, 7, 10, 14, 17 |
| | M4H7795N2 | 200 ug | Days 3, 7, 10, 14, 17 |
| | H4H9008P | 200 ug | Days 3, 7, 10, 14, 17 |
| 2 | Isotype Control | 10 mg/kg | Days 3, 7, 10, 14, 17 |
| | H4H7795N2 | 10 mg/kg | Days 3, 7, 10, 14, 17 |
| | H4H7798N | 10 mg/kg | Days 3, 7, 10, 14, 17 |
| | H4H7798N | 5 mg/kg | Days 3, 7, 10, 14, 17 |
| | H4H9048P2 | 10 mg/kg | Days 3, 7, 10, 14, 17 |
| | H4H9048P2 | 5 mg/kg | Days 3, 7, 10, 14, 17 |
| | H4H8008P | 10 mg/kg | Days 3, 7, 10, 14, 17 |
| | H4H9008P | 5 mg/kg | Days 3, 7, 10, 14, 17 |
| 3 | Isotype Control | 6 mg/kg | Days 3, 7, 10, 14, 17 |
| | H4H7798N | 5 mg/kg | Days 3, 7, 10, 14, 17 |
| | H4H7798N | 2.5 mg/kg | Gays 3, 7, 10, 14, 17 |
| | H419008P | 5 mg/kg | Days 3, 7, 10, 14, 17 |
| | H419008P | 2.5 mg/kg | Days 3, 7, 10, 14, 17 |

For the studies, average tumor volumes determined by caliper measurements and percent survival at Day 14 or 17 and Day 23 or 24 of each experiment for each treatment group were recorded. In addition, the number of tumor-free mice were also assessed at the end of the study (Day 42 for Study 1 and Day 31 for Study 2 and Study 3). Results, expressed as mean tumor volume (mm3) (+SD), percent survival, and number of tumor-free mice are shown in Table 23 for Study 1, Table 3 for Study 2, and Table 4 for Study 3.

TABLE 25
Mean tumor volume, percent survival and numbers of tumor free
mice in each treatment group from in vivo tumor Study 1

| | Tumor Volume, mm³ | | | | Tumor- |
|---|---|---|---|---|---|
| | | | | | Free |
| | moan (±SD) | | Survival, % | | Mice |
| Treatment | Day 17 | Day 23 | Day 17 | Day 23 | Day 42 |
| group | 200 | 200 | 200 | 200 | 200 |
| (n = 5) | ug/mouse | ug/mouse | ug/mouse | ug/mouse | ug/mouse |
| No treatment | 189 (±110) | 554 (±317) | 100% | 100% | 1/5 (20%) |
| Isotype | 86 (±114) | 515 (±859) | 100% | 60% | 2/5 (40%) |
| control | | | | | |
| H4H7798N | 0 (0) | 0 (0) | 100% | 100% | 5/5 (100%) |
| H4R9008P | 14 (±19) | 205 (±312) | 100% | 100% | 3/5 (60%) |
| H4H7795N2 | 89 (±176) | 446 (±889) | 100% | 80% | 3/6 (60%) |

As shown in Table 25 for Study 1, mice treated with one antibody of the disclosure, H4H7798N did not develop any detectable tumors during the course of the study. Mice treated with H4H9008P exhibited a sustained reduced tumor volume as compared to controls at days 17 and 24 of the study with 3 out of 5 mice or 4 out of 5 mice being tumor free by the end of the experiment, respectively. In contrast, treatment with one of the anti-PD1 antibodies, H4H7795N2, did not demonstrate significant efficacy in reducing tumor volume in this study as compared to controls. By day 23 of the study, 1 out of 5 mice died in the H4H7795N2 group, and 2 out of 5 mice died in the isotype control treatment group. In non-treatment group and isotype control group some mice exhibited spontaneous regression of tumors (1 out of 5 mice and 2 out of 5 mice, respectively).

TABLE 26
Mean tumor volume, percent survival and numbers of tumor free mice in each
treatment group from in vivo tumor Study 2

| Treatment | Tumor Volume, mm³ mean (±SD) | | | | Survival, % | | | | Tumor-Free Mice | |
|---|---|---|---|---|---|---|---|---|---|---|
| group | Days 17 | | Day 24 | | Day 17 | | Day 24 | | Day 31 | |
| (n = 5) | 5 mg/kg | 10 mg/kg | 5 mg/kg | 10 mg/kg | 5 mg/kg | 10 mg/kg | 5 mg/kg | 10 mg/kg | 5 mg/kg | 10 mg/kg |
| Isotype | N/A | 449 | N/A | [symbol] | N/A | 100% | N/A | [symbol] | N/A | [symbol] |
| control | | (±434) | | | | | | | | [symbol] |
| H4H7798N | 17 | 0 (0) | 104 | 0 (0) | 100% | 100% | 100% | 100% | [symbol] | [symbol] |
| | (±28) | | (±233) | | | | | | [symbol] | [symbol] |
| H4H9008P | 91 | 12 | 104 | 96 | 100% | 100% | 80% | 100% | [symbol] | [symbol] |
| | [symbol] | (±28) | [symbol] | (±215) | | | | | [symbol] | [symbol] |
| H4H9048P2 | 94 | 10 | [symbol] | 67 | 100% | 100% | 80% | 100% | [symbol] | [symbol] |
| | (±160) | (±21) | [symbol] | [symbol] | | | | | [symbol] | [symbol] |
| H4H7795N2 | N/A | 124 | N/A | 359 | N/A | 100% | N/A | 80% | N/A | [symbol] |
| | | (±209) | | (±657) | | | | | | (40%) |

[symbol]

indicates data missing or illegible when filed

As shown in Table 26 for Study 2, mice treated with one antibody of the disclosure, H4H7798N at 10 mg/kg did not develop detectable tumors during the course of the study. Groups of mice treated with 10 mg/kg of either H4H9008P or H4H9048P2 exhibited substantially reduced tumor volume as compared to controls at days 17 and 24 of the study. Four out of 5 mice in each group treated with 10 mg/kg of either H4H9008P or H4H9048P2 were tumor free at Day 31, whereas in the isotype control treatment group only 1 out of 5 animals was tumor free as a result of spontaneous tumor regression. One antibody tested at 10 mg/kg, H4H7795N2, demonstrated substantially reduced tumor volume as compared to controls at days 17 and 24 of the study, but this antibody was the least efficacious anti-PD1 antibody with only 2 out of 5 mice surviving at the end of the experiment.

A dose-dependent response in tumor suppression at the tested doses (5 mg/kg and 10 mg/kg) was observed in groups treated with H4H7798N, H4H9008P, and H4H9048P2. H4H7798N or H4H9008P therapy at 5 mg/kg was less efficacious, with 4 out of 5 tumor-free mice at the end of experiment on day 21, whereas 5 out of 5 mice remained tumor-free in both 10 mg/kg dose groups of H4H7798N, and H4H9008P.

Dunett's test in 2 way ANOVA multiple comparisons revealed that the differences in tumor growth between the group treated with isotype control antibody at 10 mg/kg as reference and the groups treated at 10 mg/kg with either H4H7798N, H4H9008P, or H4H9048P2 were statistically significant with p value<0.005. The differences in tumor growth between the group treated with isotype control antibody at 10 mg/kg as reference and the groups treated at 5 mg/kg with either H4H7798N, H4H9008P, or H4H9048P2 were also statistically significant with a p value<0.05.

TABLE 27
Mean tumor volume, percent survival and numbers of tumor free mice in each
treatment group from in vivo tumor Study 3

| Treatment | Tumor Volume, mm³ mean (±SD) | | | | Survival, % | | | | Tumor-Free Mice | |
|---|---|---|---|---|---|---|---|---|---|---|
| group | Days 14 | | Day 21 | | Day 14 | | Day 21 | | Day 31 | |
| (n = 7) | 2.5 mg/kg | 5 mg/kg | 2.5 mg/kg | 5 mg/kg | 2.5 mg/kg | 5 mg/kg | 2.5 mg/kg | 5 mg/kg | 2.5 mg/kg | 5 mg/kg |
| Isotype | N/A | 94 (±44) | N/A | 405 | N/A | 100% | N/A | 86% | N/A | 0/7 |
| control | | | | (±326) | | | | | | (0%) |
| H4H7798N | 0 (0) | 0 (0) | 19 (±51) | 13 (±35) | 100% | 100% | 100% | 100% | 6/7 | 6/7 |
| | | | | | | | | | (86%) | (86%) |
| H4H9008P | 41 (±68) | 7 (±20) | 87 (±123) | 16 (±42) | 100% | 100% | 100% | 100% | 4/7 | 6/7 |
| | | | | | | | | | (57%) | (86%) |

As shown in Table 27 for Study 3, 6 out or 7 mice treated with one antibody of the disclosure, H4H7798N, or another antibody of the disclosure, H4H9008P, at 5 mg/kg were tumor free at the end of the experiment, whereas there were no tumor free animals in the isotype control group. One tumor-bearing mouse in the IgG4 control group died on post-implantation day 17. Only 4 out of 7 mice treated with H4H9008P at 2.5 mg/kg dose remained tumor free at the end of the experiment. The difference in tumor volumes at day 21 between anti-PD-1 antibodies tested and an isotype control group was statistically significant as determined by one-way ANOVA with Dunnett's multiple comparison post-test with p<0.01. All four anti-PD-1 antibodies were equally more efficacious at the 5 mg/kg dose than at the 2.5 mg/kg dose.

### Example 10: Anti-Tumor Effects of a Combination of an Anti-PD-1 Antibody and a VEGF Antagonist in a Mouse Early-Treatment Tumor Model

An early-treatment tumor model was developed to test the efficacy of a combination of an anti-PD-1 antibody and a VEGF antagonist. In this model, the combination therapy is administered shortly after tumor implantation. The experiment also used an anti-PD-L1 antibody alone and in combination with the VEGF antagonist. The anti-PD-1 antibody used in this experiment was anti-mouse PD-1 clone “RPMI-14” with rat IgG2b (Bio X Cell, West Lebanon, N.H.). The VEGF antagonist used in this experiment was aflibercept (a VEGF receptor-based chimeric molecule, also known as “VEGF-trap” or “VEGFR1R2-FcΔC1 (a),” a full description of which is provided elsewhere herein). The anti-PD-L1 antibody used in this experiment was an anti-PD-L1 monoclonal antibody with VH/VL sequences of antibody “YW243.55S70” according to U.S. Pat. No. 20100203056A1 (Genentech, Inc.), with mouse IgG2a and which was cross-reactive with mouse PD-L1.

For this experimental model, 1.0×106 Colon-26 tumor cells were implanted sub-cutaneously into BALB/c mice at Day 0. Starting on Day 3, prior to the establishment of measurable tumors, mice were treated with one of the mono- or combination therapies, or control combination, as set forth in Table 28.

TABLE 28
Experimental dosing and treatment groups

| Treatment Group | First Agent | Second Agent |
|---|---|---|
| Control Combination | IgG2a isotype control | hFc control |
| | (250 μg, IP) | (250 μg, SC) |
| VEGF Trap only | IgG2a isotype control | Aflibercept |
| | (250 μg, IP) | (10 mg/kg, SC) |
| anti-PD-1 only | anti-PD-1 mAb RPMI-14 | hFc control |
| | (250 μg, IP) | (250 μg, SC) |
| anti-PD-L1 only | anti-PD-L1 mAb | HFc control |
| | (250 μg, IP) | (250 μg, SC) |
| VEGF Trap + anti-PD-1 | anti-PD-1 mAb | Aflibercept |
| | RPMI-14 (250 μg, IP) | (10 mg/kg, SC) |
| VEGF Trap + anti-PD-L1 | anti-PD-L1 mAb | Aflibercept |
| | (250 μg, IP) | (10 mg/kg, SC) |

The various therapies were administered at five different time points over a two week period (i.e., injections at Day 3, Day 6, Day 10, Day 13 and Day 19).

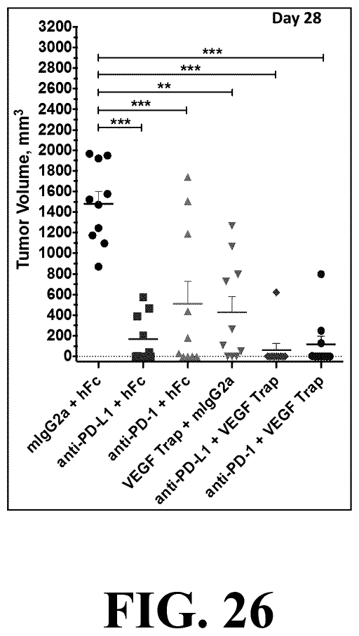

Animals in each therapy group were evaluated in terms of tumor incidence, tumor volume, median survival time, and number of tumor-free animals at Day 50. The extent of tumor growth is summarized in FIG. 25 (tumor growth curves) and FIG. 26 (tumor volume at Day 28). Results are also summarized in Table 29.

TABLE 29
Tumor-free mice in treatment groups

| | No. of Tumor-Free |
|---|---|
| Treatment Group | Animals by Day 50 |
| Control Combination | 0/10 |
| VEGF Trap only | 3/10 |
| anti-PD-1 only | 4/10 |
| anti-PD-L1 only | 5/10 |
| VEGF Trap + anti-PD-1 | 7/10 |
| VEGF Trap + anti-PD-L1 | 9/10 |

Tumor growth was substantially reduced in animals treated with the combination of VEGF Trap+anti-PD-1 antibody as compared with treatment regimens involving either therapeutic agent alone (see FIGS. 25 and 26). Furthermore, survival was substantially increased in the VEGF Trap+anti-PD-1 antibody group, with 70% of animals surviving to at least day 50 after tumor implantation. By contrast, for the anti-PD-1 and VEGF Trap monotherapy groups, survival to Day 50 was only 40% and 30% respectively (see FIG. 26 and Table 29).

### Example 11: Clinical Trial Study of Repeat Dosing with Anti-PD-1 Antibody as Single Therapy and in Combination with Other Anti-Cancer Therapies in Patients with Advanced Malignancies

This is a dose-escalation study of anti-PD-1 antibody, alone or in combination with radiation therapy, cyclophosphamide, or both in patients with advanced malignancies. The exemplary anti-PD-1 antibody (“mAb”) used in this Example comprises HCVR of SEQ ID NO: 162 and LCVR of SEQ ID NO: 170.

### Study Objectives

The primary objective of the study is to characterize the safety, tolerability, DLTs of mAb administered IV as monotherapy, or in combination with targeted radiation (with the intent to have this serve as an immuno-stimulatory, rather than primarily tumor-ablative therapy), low-dose cyclophosphamide (a therapy shown to inhibit regulatory T-cell responses), or both in patients with advanced malignancies.

The secondary objectives of the study are: (1) to determine a recommended phase 2 dose (RP2D) of mAb as monotherapy and in combination with other anti-cancer therapies (targeted radiation, low-dose cyclophosphamide, or both); (2) to describe preliminary antitumor activity of mAb, alone and with each combination partner(s); (3) to characterize the PK of mAb as monotherapy and in combination with other anti-cancer therapies (targeted radiation, low-dose cyclophosphamide, or both); and (4) to assess immunogenicity of mAb.

### Study Design

Safety will be assessed in separate, standard 3+3 dose escalation cohorts (in monotherapy, combination with radiation therapy, combination with cyclophosphamide, and combination with radiation therapy plus cyclophosphamide). The choice of combination therapy with radiation, cyclophosphamide, or both will be based on investigator assessment of the best choice of therapy for an individual patient in consultation with the sponsor. To be enrolled in a radiotherapy cohort, a patient must have a lesion that can be safely irradiated and for which radiation at the limited, palliative doses contemplated would be considered medically appropriate, and at least one other lesion suitable for response evaluation. A patient will be allowed to enroll only if a slot is available in the cohort for the chosen treatment.

Patients will undergo screening procedures to determine eligibility within 28 days prior to the initial administration of mAb. Following enrollment of patients into a mAb monotherapy cohort, enrollment of subsequent cohorts will be determined by occurrence of DLTs in prior cohorts (i.e., no DLT in a cohort of 3 patients, or no more than 1 DLT in an expanded cohort of 6 patients), and the availability of patient slots. The planned monotherapy dose levels are 1, 3, or 10 mg/kg administered IV every 14 days (2 weeks).

Once one or both of the 1 mg/kg or 3 mg/kg mAb monotherapy cohort DLT observation periods are completed without a DLT in a cohort of 3 patients or with no more than 1 DLT in an expanded cohort of 6 patients, patients can be enrolled into a cohort combining cyclophosphamide or radiotherapy with mAb at that monotherapy dose level. Patients can be enrolled into a combination mAb+cyclophosphamide/radiotherapy cohort once the DLT observation periods for both the cohort for that mAb dose level+cyclophosphamide and the cohort for that mAb dose level+the same radiotherapy regimen are completed with no DLT in a cohort of 3 patients, or no more than 1 DLT in an expanded cohort of 6 patients.

Once the 3 mg/kg mAb monotherapy cohort DLT observation period is completed with no DLT in a cohort of 3 patients, or no more than 1 DLT in an expanded cohort of 6 patients, a 10 mg/kg mAb monotherapy cohort may also enroll.

mAb 3 mg/kg and 10 mg/kg monotherapy cohorts will enroll only after the requisite number of patients in the prior monotherapy dose cohort (ie, 1 mg/kg and 3 mg/kg, respectively) have cleared the 28 day DLT observation period without a maximum tolerated dose (MTD) being demonstrated for that dose level. A mAb 1 mg/kg combination treatment cohort will enroll only after completion of the DLT observation period for the 1 mg/kg monotherapy cohort. Combination cohorts receiving 3 mg/kg mAb will enroll only when the requisite number of patients in the respective 1 mg/kg mAb combination cohorts has cleared the DLT observation period without demonstrating a MTD. Triple combination cohorts combining mAb with cyclophosphamide and a radiation regimen will enroll only when the requisite number of patients in both corresponding double combination cohorts at that dosage level have cleared the DLT observation period without a MTD being demonstrated.

Table 30 summarizes the dose-escalation cohorts in which patients will be enrolled.

TABLE 30
Possible Dose-escalation Cohorts

| n | Possible Assigned Treatment Cohort |
|---|---|
| 3-6 | 0.3 mg/kg mAb monotherapy (to be enrolled |
| | only if MTD <1 mg/kg mAb) |
| 3-6 | 1 mg/kg [symbol] mAb monotherapy |
| 3-6 | 3 mg/kg [symbol] mAb monotherapy |
| 3-6 | 10 mg/kg [symbol] mAb monotherapy |
| 3-6 | 1 mg/kg [symbol] mAb + radiotherapy (6 Gy × 5) |
| 3-6 | 1 mg/kg [symbol] mAb + radiotherapy (9 Gy × 3) |
| 3-8 | 3 mg/kg [symbol] (or MTD) mAb + cyclophosphamide |
| 3-6 | 3 mg/kg [symbol] (or MTD) mAb + radiotherapy (6 Gy x 6) |
| 3-6 | 3 mg/kg [symbol] (or MTD) mAb + radiotherapy (9 Gy = 3) |
| 3-6 | 3 mg/kg [symbol] (or MTD) mAb + radiotherapy (6 Gy × 5) + |
| | cyclophosphamide |
| 3-6 | 3 mg/kg [symbol] (or MTD) mAb + radiotherapy (9 Gy × 3) + |
| | cyclophosphamide |

[symbol]

indicates data missing or illegible when filed

A DLT is defined as any of the following: a non-hematologic toxicity (e.g., uveitis, or any other irAE), or a hematologic toxicity (e.g., neutropenia, thrombocytopenia, febrile neutropenia).

The maximum tolerated dose (MTD) is defined as the highest dose at which fewer than a third of an expanded cohort of 6 patients experience a DLT during the first cycle of treatment. Thus, the MTD is defined as the dose level immediately below the level at which dosing is stopped due to the occurrence of 2 or more DLTs in an expanded cohort of 6 patients. If dose escalation is not stopped due to the occurrence of DLTs, it will be considered that the MTD has not been determined. It is possible that an MTD may not be defined in this study, either for a monotherapy group or for individual combination groups. Additionally, it is possible that mAb MTDs may differ between monotherapy and each combination treatment regimen.

### Study Duration

Patients will receive up to 48 weeks of treatment, after which there will be a 24 week follow-up period. A patient will receive treatment until the 48 week treatment period is complete, or until disease progression, unacceptable toxicity, withdrawal of consent, or meeting of another study withdrawal criterion. After a minimum of 24 weeks of treatment, patients with confirmed complete responses (CR) may elect to discontinue treatment and continue with all relevant study assessments (eg, efficacy assessments). After a minimum of 24 weeks of treatment, patients with tumor burden assessments of stable disease (SD) or partial response (PR) that have been unchanged for 3 successive tumor evaluations may also elect to discontinue treatment and continue with all relevant study assessments (e.g., efficacy assessments).

### Study Population

The target population for this study comprises patients with advanced malignancies who are not candidates for standard therapy, unwilling to undergo standard therapy, or for whom no available therapy is expected to convey clinical benefit; and patients with malignancies that are incurable and have failed to respond to or showed tumor progression despite standard therapy.

### Inclusion Criteria:

A patient must meet with the following criteria to be eligible for inclusion in the study: (1) demonstrated progression of a solid tumor with no alternative standard-of-care therapeutic option available; (2) at least 1 lesion for response assessment. Patients assigned to radiotherapy require at least one additional lesion that can be safely irradiated while sparing the index lesions and for which radiation at the limited, palliative doses contemplated would be considered medically appropriate; (3) Eastern Cooperative Oncology Group (ECOG) performance status ≤1; (4) more than 18 years old; (5) hepatic function: a. total bilirubin≤1.5× upper limit of normal (ULN; if liver metastases≤3×ULN), b. transaminases≤3×ULN (or ≤5.0×ULN, if liver metastases), c. alkaline phosphatase (ALP)≤2.5×ULN (or 5.0×ULN, if liver metastases); (6) renal function: serum creatinine≤1.5×ULN; (7) neutrophil count (ANC) ≥1.5×109/L, c. platelet count ≥75×109/L; (8) ability to provide signed informed consent; and (9) ability and willingness to comply with scheduled visits, treatment plans, laboratory tests, and other study-related procedures.

### Exclusion Criteria:

A patient who meets any of the following criteria will be excluded from the study: (1) Ongoing or recent (within 5 years) evidence of significant autoimmune disease that required treatment with systemic immunosuppressive treatments, which may suggest risk for irAEs; (2) Prior treatment with an agent that blocks the PD-1/PD-L1 pathway; (3) Prior treatment with other immune modulating agents within fewer than 4 weeks or 4 half-lives, whichever is greater, prior to the first dose of mAb; (4) Examples of immune modulating agents include blockers of CTLA-4, 4-1 BB (CD137), OX-40, therapeutic vaccines, or cytokine treatments; (5) Untreated brain metastasis(es) that may be considered active. Patients with previously treated brain metastases may participate provided they are stable (ie, without evidence of progression by imaging for at least 4 weeks prior to the first dose of study treatment, and any neurologic symptoms have returned to baseline), and there is no evidence of new or enlarging brain metastases; (6) Immunosuppressive corticosteroid doses (>10 mg prednisone daily or equivalent) within 4 weeks prior to the first dose of mAb; (7) Deep vein thrombosis, pulmonary embolism (including asymptomatic pulmonary embolism identified on imaging), or other thromboembolic event within the 6 months preceding the first dose of mAb; (8) Active infection requiring therapy, including known infection with human immunodeficiency virus, or active infection with hepatitis B or hepatitis C virus; (9) History of pneumonitis within the last 5 years; (10) Any investigational or antitumor treatment within 30 days prior to the initial administration of mAb; (11) History of documented allergic reactions or acute hypersensitivity reaction attributed to treatment with antibody therapies in general, or to agents specifically used in the study; (12) Known allergy to doxycycline or tetracycline (precaution due to presence of trace components in mAb); (13) Breast-feeding; (14) Positive serum pregnancy test; (15) History within the last 5 years of an invasive malignancy other than the one treated in this study, with the exception of resected/ablated basal or squamous-cell carcinoma of the skin or carcinoma in situ of the cervix, or other local tumors considered cured by local treatment; (16) Acute or chronic psychiatric problems that, under the evaluation of the investigator, make the patient ineligible for participation; and (17) Continued sexual activity in men or women of childbearing potential who are unwilling to practice adequate contraception during the study.

### Study Treatments

mAb will be supplied as a liquid in sterile, single-use vials. Each vial will contain a volume sufficient to withdraw 10 mL of mAb at a concentration of 25 mg/mL. Instructions on dose preparation are provided in the study reference manuals. mAb will be administered in an outpatient setting as a 30 minute IV infusion. Each patient's dose will depend on individual body weight. The dose of mAb must be adjusted each cycle for changes in body weight of ≥10%. mAb will be administered alone and in combination with radiation and or cyclophosphamide.

### Monotherapy

mAb will be administered in an outpatient setting by IV infusion over 30 minutes every 14 days for 48 weeks (ie, Days 1, 15±3, 29±3, and 43±3 of a 56 day cycle). Planned monotherapy regimens to be assigned may include: (i) 1 mg/kg IV infusion over 30 minutes every 14 days for 48 weeks; (ii) 3 mg/kg infusion over 30 minutes every 14 days for 48 weeks; (iii) 10 mg/kg infusion over 30 minutes every 14 days for 48 weeks; and (iv) 0.3 mg/kg infusion over 30 minutes every 14 days for 48 weeks (if MTD is determined to be below 1 mg/kg).

### Combination Therapy

Concomitant radiation therapy and cyclophosphamide will be supplied through a prescription and their usage, dose, dose modifications, reductions, or delays, as well as any potential AEs resulting from their use, will be tracked along with that of mAb.

### Co-Administration of mAb and Radiation:

mAb will be administered by IV infusion over 30 minutes every 14 days for 48 weeks in combination with radiation treatment from day 8 to day 12. Planned combination mAb and radiation therapy regimens may include:
1 mg/kg mAb infusion over 30 minutes every 14 days for 48 weeks plus 30 Gy radiotherapy (6 Gy×5 times/week; given 1 week after the first dose of mAb, preferably on consecutive days)
1 mg/kg mAb infusion over 30 minutes every 14 days for 48 weeks plus 27 Gy radiotherapy (9 Gy×3 times/week; given 1 week after the first dose of mAb, preferably not on consecutive days)
3 mg/kg mAb infusion over 30 minutes every 14 days for 48 weeks plus 30 Gy radiotherapy (6 Gy×5 times/week; given 1 week after the first dose of mAb, preferably on consecutive days)
3 mg/kg mAb infusion over 30 minutes every 14 days for 48 weeks plus 27 Gy radiotherapy (9 Gy×3 times/week; given 1 week after the first dose of mAb, preferably not on consecutive days)

Patients will receive either 30 Gy given as 5 fractions of 6 Gy administered daily starting 1 week after the first dose of mAb, or 27 Gy given as 3 fractions of 9 Gy administered every other day starting 1 week after the first dose of mAb. The lesion selected for radiation should be a lesion that can be safely irradiated with focal irradiation while sparing the index lesion(s), and for which radiation at the limited, palliative doses contemplated would be considered medically appropriate. The target dose for a patient will be based on cohort assignment and should conform to the normal tissue requirements, in accord with standard radiation oncology practice. Treatment at the protocol-specified dosing regimen is permitted only if the normal tissue criteria are met. If the normal tissue criteria cannot be met at either of the radiation therapy regiments specified in the protocol, the patient is not eligible for enrollment in a combination radiation treatment cohort in this study.

### Co-Administration of mAb and Cyclophosphamide:

mAb will be administered by IV infusion over 30 minutes every 14 days (2 weeks) for 48 weeks in combination with cyclophosphamide 200 mg/m2 every 14 days for 4 doses. Each of the 4 cyclophosphamide doses will be administered 1 day before each of the first 4 mAb doses (days—1, 14, 28, and 42 of the first 56 day cycle).

Though cyclophosphamide has been used successfully concurrently with other drugs, the rate of metabolism and the leukopenic activity of cyclophosphamide reportedly are increased by chronic administration of high doses of phenobarbital. Cyclophosphamide treatment causes a marked and persistent inhibition of cholinesterase activity, thus potentiating the effect of succinylcholine chloride. The planned combination mAb and cyclophosphamide regimen to be assigned is:
Cyclophosphamide 200 mg/m2 every 14 days (days—1, 14, 28, and 42 of the first 56 day cycle) for a total of 4 doses plus 3 mg/kg mAb infusion over 30 minutes every 14 days for 48 weeks (provided monotherapy dose of 3 mg/kg<MTD; if 3 mg/kg>MTD, dose will be 1 mg/kg).
   Co-Administration of mAb, Radiation and Cyclophosphamide:

The planned combination mAb, radiation, and cyclophosphamide regimen includes:
Cyclophosphamide 200 mg/m2 every 14 days (days—1, 14, 28, and 42 of the first 56 day cycle) for a total of 4 doses plus
27 Gy radiotherapy (9 Gy×3 times/week; given 1 week after the first dose of mAb, preferably not on consecutive days) OR
30 Gy radiotherapy (6 Gy×5 times/week; given 1 week after the first dose of mAb, preferably on consecutive days)
plus
3 mg/kg mAb infusion over 30 minutes every 14 days for 48 weeks (provided monotherapy dose of 3 mg/kg<MTD; if 3 mg/kg>MTD, dose will be 1 mg/kg)

### Study Variables

Primary Variables: Primary safety variables include incidence of DLTs, incidence and severity of treatment-emergent adverse events (TEAEs), and abnormal laboratory findings through 48 weeks of treatment.

### Secondary Variables: Key Secondary Variables Include the Following:

Serum concentration and pharmacokinetics (PK) of mAb.
Antitumor activities assessed using the appropriate criteria for the indication:
   i. Response Evaluation Criteria in Solid Tumors (RECIST) criteria measured by computed tomography (CT) or magnetic resonance imaging (MRI)
   ii. Other assessment criteria should also be used for specific tumors in which RECIST measurements are not the standard.
   iii. Immune-Related Response Criteria (irRC) applied to RECIST measurements.
In all cases, irRC will be the governing tool to determine progression of disease (PD), SD, CR, or PR. Standard RECIST data will also be collected for information purposes.
Anti-mAb antibodies

### Study Procedures

The following procedures will be performed at screening for the purpose of determining study eligibility or characterizing the baseline population: (i) serum β-HCG (result must be ≤72 hours before first dose); (ii) Collection of archived tumor material: After a patient has given informed consent, the patient will be asked to arrange to provide any available previously collected tumor samples; (iii) Brain MRI: Brain MRI is required at screening if not performed in the prior 60 days; and (iv) Chest x-ray: Chest is x-ray required at screening if not performed in the prior 60 days.

### Efficacy Procedures:

A CT or MRI for tumor assessment will be performed at the screening visit (within 28 days prior to infusion) and during every cycle (approximately every 8 weeks) on day 56±3, and when disease progression is suspected. Additionally, for patients who have not progressed on study, tumor assessment will be performed for follow-up visits 3, 5, and 7. Once the choice has been made to use CT scan or MRI, subsequent assessments will be made using the same modality.

Tumor response evaluation will be performed according to immune-related response criteria (irRC; Nishino 2013). Assessments according to Response Evaluation Criteria in Solid Tumors (RECIST) version 1.1 (Eisenhauer 2009) will also be performed as a supportive exploration; however, the primary determination of disease progression for an individual patient will be made according to irRC. Measurable lesions selected as target lesions for RECIST assessments will also be included as index lesions for irRC assessments.

### Safety Procedures:

Vital signs, including temperature, resting blood pressure, pulse, and respiration, will be collected. When scheduled at the same visit as other procedures, vital signs should be measured prior to clinical laboratory assessments, PK, or exploratory sample collection. During cycle 1, vital signs will be recorded on treatment days prior to treatment, at the end of the infusion, every 30 minutes for the first 4 hours post-infusion, and at 6 and 8 hours post study drug administration. On subsequent cycles, vital signs on treatment days will be assessed and documented prior to the infusion, every 30 minutes for the first 2 hours, and then hourly until 4 hours following study drug administration.

A thorough complete or limited physical examination will be performed at visits. Complete physical examination will include examination of skin, head, eyes, nose, throat, neck, joints, lungs, heart, pulse, abdomen (including liver and spleen), lymph nodes, and extremities, as well as a brief neurologic examination. Limited physical examination will include lungs, heart, abdomen, and skin.

A standard 12-lead ECG will be performed. Any ECG finding that is judged by the investigator as a clinically significant change (worsening) compared to the baseline value will be considered an AE, recorded, and monitored.

Immune safety assays consist of rheumatoid factor (RF), thyroid stimulating hormone (TSH), C-reactive protein (CRP), and antinuclear antibody (ANA) titer and pattern. If, during the course of the study, a 4-fold or greater increase from baseline in RF or ANA or abnormal levels of TSH or CRP are observed, the following tests may also be performed: anti-DNA antibody, anti-Sjögren's syndrome A antigen (SSA) antibody (Ro), anti-Sjögren's syndrome B antigen (SSB) antibody (La), antithyroglobulin antibody, anti-LKM antibody, antiphospholipid antibody, anti-islet cell antibody, antineutrophil cytoplasm antibody, C3, C4, CHSO. Activated partial thromboplastin time (aPTT) and International Normalized Ratio (INR) will be analyzed by the site's local laboratory.

### Safety

An adverse event (AE) is any untoward medical occurrence in a patient administered a study drug which may or may not have a causal relationship with the study drug. Therefore, an AE is any unfavorable and unintended sign (including abnormal laboratory finding), symptom, or disease which is temporally associated with the use of a study drug, whether or not considered related to the study drug. An AE also includes any worsening (ie, any clinically significant change in frequency and/or intensity) of a pre-existing condition that is temporally associated with the use of the study drug. Progression of underlying malignancy will not be considered an AE if it is clearly consistent with the typical progression pattern of the underlying cancer (including time course, affected organs, etc.). Clinical symptoms of progression may be reported as AEs if the symptom cannot be determined as exclusively due to the progression of the underlying malignancy, or does not fit the expected pattern of progression for the disease under study.

An serious adverse event (SAE) is any untoward medical occurrence that at any dose results in death, is life-threatening, requires in-patient hospitalization or prolongation of existing hospitalization, results in persistent or significant disability/incapacity (substantial disruption of one's ability to conduct normal life functions), is a congenital anomaly/birth defect.

Patient information on all AEs and SAEs will be recorded.

### Statistical Plan

The study dose escalation is based on a traditional 3+3 design with 3 to 6 patients assigned per dose level. The exact number of patients enrolled in the study will depend on the number of protocol-defined DLTs observed, and the need to expand currently defined dose levels, or open additional cohorts at lower dose levels. After the required initial enrollment to the next cohort in the dose escalation has occurred, enrollment to each of the previous cohorts below the MTD for that treatment will be expanded (if not previously expanded during escalation) to a total of 6 patients.

Data will be summarized using descriptive statistics only. In general, data will be summarized by dose levels and combinations. The safety summaries and analyses will be performed on the safety analysis set (SAF). The primary analysis of safety will be based on treatment-emergent AEs (TEAEs).

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

Next, exemplary medicaments, drugs, and/or pharmaceutical formulations consistent with U.S. Pat. No. 11,603,407 B2 are described. The present disclosure may also provide pharmaceutical formulations comprising at least one therapeutic polypeptide. According to certain embodiments of the present disclosure, the therapeutic polypeptide is an antibody, or an antigen-binding fragment thereof, which binds specifically to human programmed death-1 (PD-1) protein. More specifically, the present disclosure includes pharmaceutical formulations that comprise: (i) a human antibody that specifically binds to human PD-1 (ii) a histidine buffer; (iii) an organic cosolvent that is a non-ionic surfactant; (iv) a stabilizer that is a carbohydrate; and, optionally, (v) a viscosity modifier that is an amino acid. Specific exemplary components and formulations included within the present disclosure are described in detail below.

Antibodies that Bind Specifically to PD-1

The pharmaceutical formulations of the present disclosure may comprise a human antibody, or an antigen-binding fragment thereof, that binds specifically to human PD-1. As used herein, the term “PD-1” means human programmed death-1 protein. Antibodies to human PD-1 are described in, for example, U.S. Pat. Nos. 8,008,449, and 8,168,757, 20110008369, 20130017199, 20130022595, 20150203579, and in WO2006121168, WO20091154335, WO2012145493, WO2013014668, WO2009101611, WO2015112800, EP2262837, and EP2504028.

The term “specifically binds”, or the like, means that an antibody or antigen-binding fragment thereof forms a complex with an antigen that is relatively stable under physiologic conditions. Specific binding can be characterized by a dissociation constant of at least about 1×10-8M or greater. Methods for determining whether two molecules specifically bind are well known in the art and include, for example, equilibrium dialysis, surface plasmon resonance, and the like. An isolated antibody that specifically binds human PD-1 may, however, have cross-reactivity to other antigens, such as PD-1 molecules from other species (orthologs). In the context of the present disclosure, multispecific (e.g., bispecific) antibodies that bind to human PD-1 as well as one or more additional antigens are deemed to “specifically bind” human PD-1. Moreover, an isolated antibody may be substantially free of other cellular material or chemicals.

Exemplary anti-human PD-1 antibodies that may be included in the pharmaceutical formulations of the present disclosure are set forth in patent application publications US20150203579, and WO2015112800, the disclosures of which are incorporated by reference in their entirety.

According to certain embodiments of the present disclosure, the anti-human PD-1 antibody, or antigen-binding fragment thereof, comprises a heavy chain complementary determining region (HCDR) 1 of SEQ ID NO: 3, an HCDR2 of SEQ ID NO: 4, and an HCDR3 of SEQ ID NO: 5. In certain embodiments, the anti-human PD-1 antibody, or antigen-binding fragment thereof, comprises an HCVR of SEQ ID NO: 1.

According to certain embodiments of the present disclosure, the anti-human PD-1, or antigen-binding fragment thereof, comprises a light chain complementary determining region (LCDR) 1 of SEQ ID NO: 6, an LCDR2 of SEQ ID NO: 7, and an LCDR3 of SEQ ID NO: 8. In certain embodiments, the anti-human PD-1 antibody, or antigen-binding fragment thereof, comprises an LCVR of SEQ ID NO: 2.

According to certain embodiments of the present disclosure, the anti-human PD-1, or antigen-binding fragment thereof, comprises a HCVR having 90%, 95%, 98% or 99% sequence identity to SEQ ID NO: 1.

According to certain embodiments of the present disclosure, the anti-human PD-1, or antigen-binding fragment thereof, comprises a LCVR having 90%, 95%, 98% or 99% sequence identity to SEQ ID NO: 2.

According to certain embodiments of the present disclosure, the anti-human PD-1, or antigen-binding fragment thereof, comprises a HCVR comprising an amino acid sequence of SEQ ID NO: 1 having no more than 5 amino acid substitutions.

According to certain embodiments of the present disclosure, the anti-human PD-1, or antigen-binding fragment thereof, comprises a LCVR comprising an amino acid sequence of SEQ ID NO: 2 having no more than 2 amino acid substitutions.

Sequence identity may be measured by any method known in the art (e.g., GAP, BESTFIT, and BLAST).

The present disclosure also includes formulations comprising anti-PD-1 antibodies, wherein the anti-PD-1 antibodies comprise variants of any of the HCVR, LCVR and/or CDR amino acid sequences disclosed herein having one or more conservative amino acid substitutions. For example, the present disclosure includes formulations comprising anti-PD-1 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein.

In certain embodiments, the anti-PD1 antibody comprises a Fc region elected from the group consisting of human IgG1, IgG2, IgG3, and IgG4 isotypes.

The non-limiting, exemplary antibody used in the Examples herein is referred to as “mAb1”. This antibody is also referred to in US 20150203579 as H2M7798N or H4H7798N, and is also known as “REGN2810” or “cemiplimab”. mAb1 (H4H7798N) comprises an HCVR/LCVR amino acid sequence pair having SEQ ID NOs: 1/2, and HCDR1-HCDR2-HCDR3/LCDR1-LCDR2-LCDR3 domains represented by SEQ ID NOs: 3-4-5/SEQ ID NOs: 6-7-8.

According to certain embodiments of the present disclosure, the anti-human PD-1, or antigen-binding fragment thereof, comprises a heavy chain of SEQ ID NO: 9 and a light chain of SEQ ID NO: 10.

It is well known in the art that terminal cleavage of amino acids can occur during production of antibodies (see, for example, Wang et al 2007, J. Pharma. Sci. 96:1-26). Accordingly, in certain embodiments, the anti-PD-1 antibody comprises a heavy chain and a light chain, wherein the heavy chain comprises the amino acid sequence of SEQ ID NO: 11. SEQ ID NO: 11 comprises the heavy chain amino acid sequence wherein the C-terminal lysine is absent from the amino acid sequence of SEQ ID NO: 9. In certain embodiments, formulations of the present disclosure contain about 50%, about 60%, about 70%, about 80%, about 90%, about 95%, about 98% or more of the anti-PD-1 antibody wherein the C-terminal lysine is absent.

The amount of antibody, or antigen-binding fragment thereof, contained within the pharmaceutical formulations of the present disclosure may vary depending on the specific properties desired of the formulations, as well as the particular circumstances and purposes for which the formulations are intended to be used. In certain embodiments, the pharmaceutical formulations are liquid formulations that may contain 5±0.75 mg/mL to 250±37.5 mg/ml of antibody; 10±1.5 mg/mL to 240±36 mg/ml of antibody; 20±3.0 mg/mL to 230±34.5 mg/ml of antibody; 25±3.75 mg/mL to 240±36 mg/mL of antibody; 50±7.5 mg/mL to 230±34.5 mg/mL of antibody; 60±9 mg/mL to 240±36 mg/mL of antibody; 70±10.5 mg/mL to 230±34.5 mg/ml of antibody; 80±12 mg/mL to 220±33 mg/mL of antibody; 90±13.5 mg/mL to 210±31.5 mg/ml of antibody; 100±15 mg/mL to 200±30 mg/ml of antibody; 110±16.5 mg/mL to 190±28.5 mg/ml of antibody; 120±18 mg/mL to 180±27 mg/ml of antibody; 130±19.5 mg/mL to 170±25.5 mg/ml of antibody; 140±21 mg/mL to 160±24 mg/mL of antibody; 150±22.5 mg/ml of antibody; or 175±26.25 mg/ml. For example, the formulations of the present disclosure may comprise about 5 mg/ml; about 10 mg/mL; about 15 mg/mL; about 20 mg/mL; about 25 mg/mL; about 30 mg/mL; about 35 mg/mL; about 40 mg/mL; about 45 mg/mL; about 50 mg/mL; about 55 mg/mL; about 60 mg/mL; about 65 mg/mL; about 70 mg/ml; about 75 mg/mL; about 80 mg/mL; about 85 mg/mL; about 90 mg/mL; about 95 mg/mL; about 100 mg/ml; about 105 mg/mL; about 110 mg/ml; about 115 mg/mL; about 120 mg/mL; about 125 mg/ml; about 130 mg/mL; about 135 mg/mL; about 140 mg/ml; about 145 mg/mL; about 150 mg/mL; about 155 mg/mL; about 160 mg/ml; about 165 mg/mL; about 170 mg/mL; about 175 mg/mL; about 180 mg/mL; about 185 mg/mL; about 190 mg/ml; about 195 mg/mL; about 200 mg/ml; about 205 mg/ml; about 210 mg/ml; about 215 mg/mL; about 220 mg/ml; about 225 mg/mL; about 230 mg/ml; about 235 mg/ml; about 240 mg/mL; about 245 mg/mL; or about 250 mg/mL of an antibody or an antigen-binding fragment thereof, that binds specifically to human PD-1.

### Excipients and pH

The pharmaceutical formulations of the present disclosure comprise one or more excipients. The term “excipient”, as used herein, means any non-therapeutic agent added to the formulation to provide a desired consistency, viscosity or stabilizing effect.

In certain embodiments, the pharmaceutical formulation of the disclosure comprises at least one organic cosolvent in a type and in an amount that stabilizes the human PD-1 antibody under conditions of rough handling or agitation, such as, e.g., vortexing. In some embodiments, what is meant by “stabilizes” is the prevention of the formation of more than 3% aggregated antibody of the total amount of antibody (on a molar basis) over the course of rough handling. In some embodiments, rough handling is vortexing a solution containing the antibody and the organic cosolvent for about 60 minutes or about 120 minutes.

In certain embodiments, the organic cosolvent is a non-ionic surfactant, such as an alkyl poly(ethylene oxide). Specific non-ionic surfactants that can be included in the formulations of the present disclosure include, e.g., polysorbates such as polysorbate 20, polysorbate 28, polysorbate 40, polysorbate 60, polysorbate 65, polysorbate 80, polysorbate 81, and polysorbate 85; poloxamers such as poloxamer 181, poloxamer 188, poloxamer 407; or polyethylene glycol (PEG). Polysorbate 20 is also known as TWEEN 20, sorbitan monolaurate and polyoxyethylenesorbitan monolaurate. Poloxamer 188 is also known as PLURONIC F68.

The amount of non-ionic surfactant contained within the pharmaceutical formulations of the present disclosure may vary depending on the specific properties desired of the formulations, as well as the particular circumstances and purposes for which the formulations are intended to be used. In certain embodiments, the formulations may contain 0.01%±0.005% to 0.5%±0.25% surfactant. For example, the formulations of the present disclosure may comprise about 0.005%; about 0.01%; about 0.02%; about 0.03%; about 0.04%; about 0.05%; about 0.06%; about 0.07%; about 0.08%; about 0.09%; about 0.1%; about 0.11%; about 0.12%; about 0.13%; about 0.14%; about 0.15%; about 0.16%; about 0.17%; about 0.18%; about 0.19%; about 0.20%; about 0.21%; about 0.22%; about 0.23%; about 0.24%; about 0.25%; about 0.26%; about 0.27%; about 0.28%; about 0.29%; about 0.30%; about 0.35%; about 0.40%; about 0.45%; about 0.46%; about 0.47%; about 0.48%; about 0.49%; about 0.50%; about 0.55%; or about 0.575% polysorbate 20 or polysorbate 80.

The pharmaceutical formulations of the present disclosure may also comprise one or more stabilizers in a type and in an amount that stabilizes the human PD-1 antibody under conditions of thermal stress. In some embodiments, what is meant by “stabilizes” is maintaining greater than about 91% of the antibody in a native conformation when the solution containing the antibody and the thermal stabilizer is kept at about 45° C. for up to about 28 days. In some embodiments, what is meant by “stabilizes” is wherein less than about 6% of the antibody is aggregated when the solution containing the antibody and the thermal stabilizer is kept at about 45° C. for up to about 28 days. As used herein, “native” means the major form of the antibody by size exclusion, which is generally an intact monomer of the antibody. The term “native” also refers to non-aggregated and non-degraded form of the antibody.

In certain embodiments, the thermal stabilizer is a sugar such as sucrose, the amount of which contained within the formulation can vary depending on the specific circumstances and intended purposes for which the formulation is used. In certain embodiments, the formulations may contain about 1% to about 15% sugar; about 2% to about 14% sugar; about 3% to about 13% sugar; about 4% to about 12% sugar; about 5% to about 12% sugar; about 6% to about 11% sugar; about 7% to about 10% sugar; about 8% to about 11% sugar; or about 9% to about 11% sugar. For example, the pharmaceutical formulations of the present disclosure may comprise 4%±0.8%; 5%±1%; 6%±1.2%; 7%±1.4%; 8%±1.6%; 9%±1.8%; 10%±2%; 11%±2.2%; 12%±2.4%; 13%±2.6%; or about 14%±2.8% sugar (e.g., sucrose).

The pharmaceutical formulations of the present disclosure may also comprise a buffer or buffer system, which serves to maintain a stable pH and to help stabilize the human PD-1 antibody. The term “buffer” as used herein denotes a pharmaceutically acceptable buffer which maintains a stable pH or resists changes in pH of the solution. In preferred embodiments, the buffer comprises histidine. In the context of this disclosure, “histidine buffer” or “buffer comprising histidine” is a buffer comprising the amino acid histidine. Examples of histidine buffers include histidine chloride, histidine acetate, histidine phosphate, and histidine sulfate. In a preferred embodiment, the histidine buffer is prepared by dissolving L-histidine and L-histidine hydrochloride (e.g. as monohydrate) in a defined amount and ratio. In one embodiment, the histidine buffer is prepared by titrating L-histidine (free base, solid) with diluted hydrochloric acid. The term “histidine” is used interchangeably with “histidine buffer” throughout this disclosure. In some embodiments, what is meant by “stabilizes” is wherein less than 4.5%±0.5% of the antibody is aggregated when the solution containing the antibody and the buffer is kept at about 45° C. for up to about 28 days. In some embodiments, what is meant by “stabilizes” is wherein less than 3%±0.5% or less than 2.5%±0.5% of the antibody is aggregated when the solution containing the antibody and the buffer is kept at about 37° C. for up to about 28 days. In some embodiments, what is meant by “stabilizes” is wherein at least 93%±0.5% or at least 94%±0.5% of the antibody is in its native conformation as determined by size exclusion chromatography when the solution containing the antibody and the buffer is kept at about 45° C. for up to about 28 days. In some embodiments, what is meant by “stabilizes” is wherein at least 94%±0.5% or at least 95%±0.5% of the antibody is in its native conformation as determined by size exclusion chromatography when the solution containing the antibody and the buffer is kept at about 37° C. for up to about 28 days. By “native” or “native conformation”, what is meant is the antibody fraction that is not aggregated or degraded. This is generally determined by an assay that measures the relative size of the antibody entity, such as a size exclusion chromatographic assay. The non-aggregated and non-degraded antibody elutes at a fraction that equates to the native antibody, and is generally the main elution fraction. Aggregated antibody elutes at a fraction that indicates a size greater than the native antibody. Degraded antibody elutes at a fraction that indicates a size less than the native antibody.

In some embodiments, what is meant by “stabilizes” is wherein at least 35%±0.5% of the antibody is in its main charge form as determined by cation exchange chromatography when the solution containing the antibody and the buffer is kept at about 45° C. for up to about 28 days. In some embodiments, what is meant by “stabilizes” is wherein at least 46%±0.5% or at least 39%±0.5% of the antibody is in its main charge form as determined by cation exchange chromatography when the solution containing the antibody and the buffer is kept at about 37° C. for up to about 28 days. By “main charge” or “main charge form”, what is meant is the fraction of antibody that elutes from an ion exchange resin in the main peak, which is generally flanked by more “basic” peaks on one side and more “acidic” peaks on the other side.

The pharmaceutical formulations of the present disclosure may have a pH of from about 5.2 to about 6.4. For example, the formulations of the present disclosure may have a pH of about 5.5; about 5.6; about 5.7; about 5.8; about 5.9; about 6.0; about 6.1; about 6.2; about 6.3; about 6.4; or about 6.5. In some embodiments, the pH is 6.0±0.4; 6.0±0.3; 6.0±0.2; 6.0±0.1; about 6.0; or 6.0.

In some embodiments, the buffer or buffer system comprises at least one buffer that has a buffering range that overlaps fully or in part the range of pH 5.5-7.4. In certain embodiments, the buffer comprises a histidine buffer. In certain embodiments, the histidine buffer is present at a concentration of 5 mM±1 mM to 15 mM±3 mM; 6 mM±1.2 mM to 14 mM±2.8 mM; 7 mM±1.4 mM to 13 mM±2.6 mM; 8 mM±1.6 mM to 12 mM±2.4 mM; 9 mM±1.8 mM to 11 mM±2.2 mM; 10 mM±2 mM; or about 10 mM. In certain embodiments, the buffer system comprises histidine at 10 mM±2 mM, at a pH of 6.0±0.3. In preferred embodiments, the histidine buffer comprises L-histidine and L-histidine monohydrochloride monohydrate. In one embodiment, the histidine buffer comprises L-histidine at a concentration of 4.8 mM±0.96 mM. In one embodiment, the histidine buffer comprises L-histidine monohydrochloride monohydrate at a concentration of 5.2 mM±1.04 mM. In one embodiment, the histidine buffer comprises L-histidine at a concentration of 4.8 mM±0.96 mM and L-histidine monohydrochloride monohydrate at a concentration of 5.2 mM±1.04 mM.

The pharmaceutical formulations of the present disclosure may also comprise one or more excipients that serve to maintain a reduced viscosity or to lower the viscosity of formulations containing a high concentration of anti-PD-1 antibody drug substance (e.g., generally z 150 mg/ml of antibody). In certain embodiments, the viscosity modifier is an amino acid. In one embodiment, the amino acid is proline. In one embodiment, the pharmaceutical formulation of the present disclosure contains proline, preferably as L-proline, at a concentration of 1%, 1.5%, 2%, 2.5%, 3%, 3.5%, 4%, 4.5% or 5%. The term “proline” is used interchangeably with “L-proline” throughout this disclosure. In some embodiments, the formulation comprises proline in an amount sufficient to maintain the viscosity of the liquid formulation at less than 20±3 cPoise, less than 15±2.25 cPoise, or less than 11±1.65 cPoise. In some embodiments, the formulation comprises proline in an amount sufficient to maintain the viscosity at or below 15±2.25 cPoise. In certain embodiments, formulations may contain about 1% to about 5% proline; about 2% to about 4% proline; or about 3% proline. For example, the pharmaceutical formulations of the present disclosure may comprise 1%±0.2%; 1.5%±0.3%; 2%±0.4%; 2.5%±0.5%; 3%±0.6%; 3.5%±0.7%; 4%±0.8%; 4.5%±0.9%; or about 5%±1% proline.

During the antibody purification process it may be desired or necessary to exchange one buffer for another to achieve appropriate excipient concentrations, antibody concentration, pH, etc. Buffer exchange can be accomplished, e.g., by ultrafiltration/diafiltration (UF/DF) using, e.g., a semi-permeable tangential flow filtration membrane. Use of such techniques, however, has the potential to cause the Gibbs-Donnan effect [Bolton et al., 2011, Biotechnol. Prog. 27 (1): 140-152]. The buildup of positive charge on the product side of the membrane during protein concentration is counterbalanced electrically by the preferential movement of positive ions to the opposite side of the membrane. The potential consequence of this phenomenon is that the final concentrations of certain components (e.g., histidine, L-proline, etc.) may be lower than the intended target concentrations of these components due to the electrostatic repulsion of positively charged diafiltration buffer excipients to the positively charged antibody protein during the UF/DF step. Thus, the present disclosure includes formulations in which the concentration of, e.g., histidine and/or L-proline vary from the recited amounts or ranges herein due to the Gibbs-Donnan effect.

Volume exclusion describes the behavior of highly concentrated samples in which a significant portion of the total volume of the solution is taken up by the solute, especially large molecules such as proteins, excluding the solvent from this space. This then decreases the total volume of solvent available for other solutes to be dissolved in, which may result in unequal partition across the ultrafiltration membrane. Thus, the present disclosure includes formulations in which the concentration of, e.g., histidine and/or L-proline may vary from the recited amounts or ranges herein due to the volume exclusion effect.

During the manufacture of the formulations of the present disclosure, variations in the composition of the formulation may occur. These variations may include the concentration of the active ingredient, the concentration of the excipients, and/or the pH of the formulation. Because changes in any of these parameters could potentially impact the stability or potency of the drug product, proven acceptable range (PAR) studies were conducted to assess whether variations in the composition, within the defined ranges, would impact the stability or potency of the antibody. Accordingly, the present disclosure includes formulations comprising anti-PD-1 antibodies which are stable and retain potency with up to 50% variation in the excipient concentration. For example, included herein are anti-PD-1 antibody formulations, wherein stability and potency of said formulations is unaffected by ±10%, ±20%, ±30%, ±40% or ±50% variation in the concentration of antibody, sucrose, histidine buffer and/or polysorbate.

### Stability and Viscosity of the Pharmaceutical Formulations

The pharmaceutical formulations of the present disclosure typically exhibit high levels of stability. The term “stable”, as used herein in reference to the pharmaceutical formulations, means that the antibodies within the pharmaceutical formulations retain an acceptable degree of chemical structure or biological function after storage under defined conditions. A formulation may be stable even though the antibody contained therein does not maintain 100% of its chemical structure or biological function after storage for a defined amount of time. Under certain circumstances, maintenance of about 90%, about 95%, about 96%, about 97%, about 98% or about 99% of an antibody's structure or function after storage for a defined amount of time may be regarded as “stable”.

Stability can be measured, inter alia, by determining the percentage of native antibody that remains in the formulation after storage for a defined amount of time at a defined temperature. The percentage of native antibody can be determined by, inter alia, size exclusion chromatography (e.g., size exclusion ultra performance liquid chromatography [SE-UPLC]), such that native means non-aggregated and non-degraded. An “acceptable degree of stability”, as that phrase is used herein, means that at least 90% of the native form of the antibody can be detected in the formulation after storage for a defined amount of time at a given temperature. In certain embodiments, at least about 90%, 91%, 92%, 93%, 94%, 95%, 96%, 97%, 98%, 99% or 100% of the native form of the antibody can be detected in the formulation after storage for a defined amount of time at a defined temperature. The defined amount of time after which stability is measured can be at least 14 days, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The defined temperature at which the pharmaceutical formulation may be stored when assessing stability can be any temperature from about −80° C. to about 45° C., e.g., storage at about −80° C., about −30° C., about −20° C., about 0° C., about 4°-8° C., about 5° C., about 25° C., about 35° C., about 37° C., or about 45° C. For example, a pharmaceutical formulation may be deemed stable if after 6 months of storage at 5° C., greater than about 95%, 96%, 97% or 98% of native antibody is detected by SE-UPLC. A pharmaceutical formulation may also be deemed stable if after 6 months of storage at 25° C., greater than about 95%, 96%, 97% or 98% of native antibody is detected by SE-UPLC. A pharmaceutical formulation may also be deemed stable if after 28 days of storage at 45° C., greater than about 89%, 90%, 91%, 92%, 93%, 94%, 95% or 96% of native antibody is detected by SE-UPLC. A pharmaceutical formulation may also be deemed stable if after 12 months of storage at −20° C., greater than about 96%, 97%, or 98% of native antibody is detected by SE-UPLC. A pharmaceutical formulation may also be deemed stable if after 12 months of storage at −30° C., greater than about 96%, 97% or 98% of native antibody is detected by SE-UPLC. A pharmaceutical formulation may also be deemed stable if after 12 months of storage at −80° C., greater than about 96%, 97% or 98% of native antibody is detected by SE-UPLC.

Stability can be measured, inter alia, by determining the percentage of antibody that forms in an aggregate within the formulation after storage for a defined amount of time at a defined temperature, wherein stability is inversely proportional to the percent aggregate that is formed. The percentage of aggregated antibody can be determined by, inter alia, size exclusion chromatography (e.g., size exclusion ultra performance liquid chromatography [SE-UPLC]). An “acceptable degree of stability”, as that phrase is used herein, means that at most 5% of the antibody is in an aggregated form (also denoted as the high molecular weight-HMW-form) detected in the formulation after storage for a defined amount of time at a given temperature. In certain embodiments an acceptable degree of stability means that at most about 5%, 4%, 3%, 2%, 1%, 0.5%, or 0.1% of the antibody can be detected in an aggregate in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 45° C., e.g., storage at about −80° C., about −30° C., about −20° C., about 0° C., about 4°-8° C., about 5° C., about 25° C., about 35° C., about 37° C. or about 45° C. For example, a pharmaceutical formulation may be deemed stable if after 12 months of storage at 5° C., less than about 2%, 1%, 0.5%, or 0.1% of the antibody is detected in an aggregated form. A pharmaceutical formulation may also be deemed stable if after three months of storage at 25° C., less than about 4%, 3%, 2%, 1%, 0.5%, or 0.1% of the antibody is detected in an aggregated form. A pharmaceutical formulation may also be deemed stable if after 28 days of storage at 45° C., less than about 10%, 9%, 8%, 7%, 6%, 5%, 4%, 3%, 2%, 1%, or 0.5%, of the antibody is detected in an aggregated form. A pharmaceutical formulation may also be deemed stable if after three months of storage at −20° C., −30° C., or −80° C. less than about 3%, 2%, 1%, 0.5%, or 0.1% of the antibody is detected in an aggregated form.

Stability can be measured, inter alia, by determining the percentage of antibody that migrates in a more acidic fraction during ion exchange (“acidic form”) than in the main fraction of antibody (“main charge form”), wherein stability is inversely proportional to the fraction of antibody in the acidic form. While not wishing to be bound by theory, deamidation of the antibody may cause the antibody to become more negatively charged and thus more acidic relative to the non-deamidated antibody (see, e.g., Robinson, N., Protein Deamidation, PNAS, Apr. 16, 2002, 99 (8): 5283-5288). The percentage of “acidified” antibody can be determined by, inter alia, ion exchange chromatography (e.g., cation exchange ultra performance liquid chromatography [CEX-UPLC]). An “acceptable degree of stability”, as that phrase is used herein, means that at most 45% of the antibody is in a more acidic form detected in the formulation after storage for a defined amount of time at a defined temperature. In certain embodiments an acceptable degree of stability means that at most about 40%, 35%, 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. In one embodiment, an acceptable degree of stability means that less than 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 45° C., e.g., storage at about −80° C., about −30° C., about −20° C., about 0° C., about 4°-8° C., about 5° C., about 25° C., or about 45° C. For example, a pharmaceutical formulation may be deemed stable if after three months of storage at −80° C., −30° C., or −20° C. less than about 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after six months of storage at 5° C., less than about 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after six months of storage at 25° C., less than about 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 28 days of storage at 45° C., less than about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody can be detected in a more acidic form.

Other methods may be used to assess the stability of the formulations of the present disclosure such as, e.g., differential scanning calorimetry (DSC) to determine thermal stability, controlled agitation to determine mechanical stability, and absorbance at about 350 nm or about 405 nm to determine solution turbidities. For example, a formulation of the present disclosure may be considered stable if, after 6 or more months of storage at about 5° C. to about 25° C., the change in OD405 of the formulation is less than about 0.05 (e.g., 0.04, 0.03, 0.02, 0.01, or less) from the OD405 of the formulation at time zero.

Measuring the biological activity or binding affinity of the antibody to its target may also be used to assess stability. For example, a formulation of the present disclosure may be regarded as stable if, after storage at e.g., 5° C., 25° C., 45° C., etc. for a defined amount of time (e.g., 1 to 12 months), the anti-PD-1 antibody contained within the formulation binds to PD-1 with an affinity that is at least 90%, 95%, or more of the binding affinity of the antibody prior to said storage. Binding affinity may be determined by e.g., ELISA or surface plasmon resonance. Biological activity may be determined by a PD-1 activity assay, such as e.g., contacting a cell that expresses PD-1 with the formulation comprising the anti PD-1 antibody. The binding of the antibody to such a cell may be measured directly, such as e.g., via FACS analysis. Alternatively, the downstream activity of the PD-1 system may be measured in the presence of the antibody, and compared to the activity of the PD-1 system in the absence of antibody. In some embodiments, the PD-1 may be endogenous to the cell. In other embodiments, the PD-1 may be ectopically expressed in the cell.

Additional methods for assessing the stability of an antibody in formulation are demonstrated in the Examples presented below.

The liquid pharmaceutical formulations of the present disclosure may, in certain embodiments, exhibit low to moderate levels of viscosity. “Viscosity” as used herein may be “kinematic viscosity” or “absolute viscosity”. “Kinematic viscosity” is a measure of the resistive flow of a fluid under the influence of gravity. When two fluids of equal volume are placed in identical capillary viscometers and allowed to flow by gravity, a viscous fluid takes longer than a less viscous fluid to flow through the capillary. For example, if one fluid takes 200 seconds to complete its flow and another fluid takes 400 seconds, the second fluid is twice as viscous as the first on a kinematic viscosity scale. “Absolute viscosity”, sometimes called dynamic or simple viscosity, is the product of kinematic viscosity and fluid density (Absolute Viscosity=Kinematic Viscosity×Density). The dimension of kinematic viscosity is L2/T where L is a length and T is a time. Commonly, kinematic viscosity is expressed in centistokes (cSt). The SI unit of kinematic viscosity is mm2/s, which is 1 cSt. Absolute viscosity is expressed in units of centipoise (cP). The SI unit of absolute viscosity is the milliPascal-second (mPa-s), where 1 cP=1 mPa-s.

As used herein, a low level of viscosity, in reference to a fluid formulation of the present disclosure, will exhibit an absolute viscosity of less than about 20 cPoise (cP). For example, a fluid formulation of the disclosure will be deemed to have “low viscosity”, if, when measured using standard viscosity measurement techniques, the formulation exhibits an absolute viscosity of about 20 cP, about 19 cP, about 18 cP, about 15 cP, about 12 cP, about 10 cP, about 9 cP, about 8 cP, or less. As used herein, a moderate level of viscosity, in reference to a fluid formulation of the present disclosure, will exhibit an absolute viscosity of between about 35 cP and about 20 cP. For example, a fluid formulation of the disclosure will be deemed to have “moderate viscosity”, if when measured using standard viscosity measurement techniques, the formulation exhibits an absolute viscosity of about 34 cP, about 33 cP, about 32 cP, about 31 cP, about 30 cP, about 29 cP, about 28 cP, about 27 cP, about 26 cP, about 25 cP, about 24 cP, about 23 cP, about 22 cP, about 21 cP, about 20 cP, about 19 cP, 18 cP, about 17 cP, about 16 cP, or about 15.1 cP.

As illustrated in the examples below, the present inventors have made the surprising discovery that low viscosity liquid formulations comprising high concentrations of an anti-human PD-1 antibody (e.g., from about 50 mg/ml up to 250 mg/mL) can be obtained by formulating the antibody with proline from about 1% to about 5% and sucrose at about 5%. Such formulations are stable to stress during handling and to storage at temperatures ranging from 45° C. to −80° C. (shown herein) and have low viscosity (have viscosity ranging from 7 to 15 cP).

### Exemplary Formulations

According to one aspect of the present disclosure, the pharmaceutical formulation is a stable, low viscosity, generally physiologically isotonic liquid formulation, which comprises: (i) a human antibody that specifically binds to human PD-1 (e.g., H4H7798N), at a concentration of up to 250 mg/mL+45 mg/ml; (ii) a histidine buffer system that provides sufficient buffering at about pH 6.0±0.3; (iii) an organic cosolvent, which protects the structural integrity of the antibody; (iv) a thermal stabilizer that is a sugar; and (iv) a viscosity modifier that is an amino acid, which serves to keep the viscosity manageable for injection in a convenient volume for subcutaneous administration.

According to one embodiment, the stable, low-viscosity pharmaceutical formulation comprises: (i) a human IgG4 antibody that specifically binds to human PD-1, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of up to 200 mg/ml±30 mg/mL; (ii) histidine buffer at 10 mM±2 mM, which buffers at pH 6.0±0.3; (iii) polysorbate 80 at 0.2% w/v±0.1% w/v; (iv) sucrose at 5%±1% w/v; and (v) L-proline at 1.5% (w/V)±0.3%.

According to one embodiment, the stable low-viscosity pharmaceutical formulation comprises: (i) a human IgG4 antibody that specifically binds to human PD-1, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 175 mg/ml±26.25 mg/ml; (ii) histidine buffer at 10 mM±2 mM, which buffers at pH 6.0±0.3; (iii) polysorbate 80 at 0.2% w/v±0.1% w/v; (iv) sucrose at 5%±1% w/v; and (v) L-proline at 1.5% (w/v)±0.3%.

According to one embodiment, the stable low-viscosity pharmaceutical formulation comprises: (i) a human IgG4 antibody that specifically binds to human PD-1, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 150 mg/ml±22.5 mg/mL; (ii) histidine buffer at 10 mM±2 mM, which buffers at pH 6.0±0.3; (iii) polysorbate 80 at 0.2% w/v±0.1% w/v; (iv) sucrose at 5%±1% w/v; and (v) L-proline at 1.5% (w/v)±0.3%.

According to one embodiment, the stable low-viscosity pharmaceutical formulation comprises: (i) a human IgG4 antibody that specifically binds to human PD-1, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 100 mg/mL±15 mg/mL; (ii) histidine buffer at 10 mM±2 mM, which buffers at pH 6.0±0.3; (iii) sucrose at 5% w/v±1% w/v; (iv) polysorbate 80 at 0.2% w/v±0.1%; and L-proline at 1.5% (w/v)±0.3%.

According to one embodiment, the stable low-viscosity pharmaceutical formulation comprises: (i) a human IgG4 antibody that specifically binds to human PD-1, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 50 mg/mL±7.5 mg/mL; (ii) histidine buffer at 10 mM±2 mM, which buffers at pH 6.0±0.3; (iii) sucrose at 5% w/v±1% w/v; (iv) polysorbate 80 at 0.2% w/v±0.1%; and L-proline at 1.5% (w/v)±0.3%.

According to one embodiment, the stable low-viscosity pharmaceutical formulation comprises: (i) a human IgG4 antibody that specifically binds to human PD-1, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 25 mg/mL±3.75 mg/ml; (ii) histidine buffer at 10 mM±2 mM, which buffers at pH 6.0±0.3; (iii) sucrose at 5% w/v±1% w/v; (iv) polysorbate 80 at 0.2% w/v±0.1%; and L-proline at 1.5% (w/v)±0.3%.

Additional non-limiting examples of pharmaceutical formulations encompassed by the present disclosure are set forth elsewhere herein, including the working Examples presented below.

### Containers and Methods of Administration

The pharmaceutical formulations of the present disclosure may be contained within any container suitable for storage of medicines and other therapeutic compositions. For example, the pharmaceutical formulations may be contained within a sealed and sterilized plastic or glass container having a defined volume such as a vial, ampule, syringe, cartridge, or bottle. Different types of vials can be used to contain the formulations of the present disclosure including, e.g., clear and opaque (e.g., amber) glass or plastic vials. Likewise, any type of syringe can be used to contain or administer the pharmaceutical formulations of the present disclosure.

The pharmaceutical formulations of the present disclosure may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than or equal to 500 parts per billion (ppb) of tungsten. The term “low tungsten” means that the syringe contains less than 500 ppb of tungsten. For example, a low tungsten syringe, according to the present disclosure, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial, or to preserve their stability. Thus, pharmaceutical formulations of the present disclosure, according to certain embodiments, may be contained within a syringe that comprises a coated plunger, or within a vial that is sealed with a coated rubber stopper. For example, the plunger or stopper may be coated with a fluorocarbon film. Examples of coated stoppers or plungers suitable for use with vials and syringes containing the pharmaceutical formulations of the present disclosure are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present disclosure are commercially available under the tradename “FluroTec®”, available from West Pharmaceutical Services, Inc. (Lionville, Pa.). FluroTec® is an example of a fluorocarbon coating used to minimize or prevent drug product from adhering to the rubber surfaces.

According to certain embodiments of the present disclosure, the pharmaceutical formulations may be contained within a low tungsten syringe that comprises a fluorocarbon-coated plunger.

In one embodiment, the container is a 20 mL type 1 clear borosilicate glass vial. In certain embodiments, the container is a 2 mL, 5 mL or 10 mL type 1 borosilicate glass vial with a chlorobutyl stopper, with a FluroTec® coating.

In one embodiment, the liquid pharmaceutical formulation of the present disclosure comprising about 25 mg/ml or 50 mg/ml of mAb1 is administered intravenously and may be contained in a glass vial.

In certain embodiments, the present disclosure provides an autoinjector comprising any of the liquid formulations described herein. In some embodiments, the present disclosure provides an autoinjector comprising a stable liquid formulation comprising about 50 mg/mL, about 100 mg/mL, about 150 mg/mL or about 175 mg/ml of mAb1, about 10 mM of histidine, at pH of about 6.0, about 5% sucrose, about 1.5% proline and about 0.2% polysorbate 80.

In certain embodiments, the present disclosure provides a prefilled syringe comprising any of the liquid formulations described herein. In some embodiments, the present disclosure provides a prefilled syringe comprising a stable liquid formulation comprising about 50 mg/mL, about 100 mg/mL, about 150 mg/mL or about 175 mg/ml of mAb1, about 10 mM of histidine, at pH of about 6.0, about 5% sucrose, about 1.5% proline and about 0.2% polysorbate 80. In certain embodiments, the syringe is a 1 mL or 2.25 mL long glass syringe filled with a 27-gauge thin wall needle, a fluorocarbon coated rubber plunger and a rubber needle shield.

In one embodiment, the liquid pharmaceutical formulation containing about 175 mg/mL+26.25 mg/ml mAb1 is administered in a volume of approximately up to 2 mL in a prefilled syringe. In certain embodiments, the syringe is a 1 mL or 2.25 mL long glass syringe filled with a 27-gauge thin wall needle, a fluorocarbon coated rubber plunger and a rubber needle shield. In one embodiment, the syringe is an OMPI 1 mL long glass syringe fitted with a 27-gauge needle, a FM27 rubber needle shield, and a FLUROTEC® coated 4023/50 rubber plunger.

In one embodiment, the liquid pharmaceutical formulation containing about 150 mg/mL+22.5 mg/ml anti-PD-1 antibody is administered in a volume of approximately up to 2 mL in a prefilled syringe. In one embodiment, the syringe is a 1 mL or 2.25 mL long glass syringe filled with a 27-gauge thin wall needle, a fluorocarbon coated rubber plunger and a rubber needle shield. In one embodiment, the syringe is an OMPI 1 mL long glass syringe fitted with a 27-gauge needle, a FM27 rubber needle shield, and a FLUROTEC® coated 4023/50 rubber plunger.

### Therapeutic Uses of the Pharmaceutical Formulations

The pharmaceutical formulations of the present disclosure are useful, inter alia, for the treatment, prevention or amelioration of any disease or disorder associated with PD-1 activity, including diseases or disorders mediated by PD-1. Exemplary, non-limiting diseases and disorders that can be treated or prevented by the administration of the pharmaceutical formulations of the present disclosure include viral infections, autoimmune diseases and various cancers such as, e.g., brain cancer, lung cancer, prostate cancer, colorectal cancer, head and neck cancer, skin cancer, various blood cancers, and endometrial cancers.

### EXAMPLES

The following examples are presented so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by mole, molecular weight is average molecular weight, temperature is in degrees Centigrade, and pressure is at or near atmospheric pressure.

### Example 1: Development of an Anti-PD-1 Antibody Formulation

The goals of the formulation activities were to develop a formulation with the following attributes:
A liquid formulation with a concentration of the anti-PD-1 antibody sufficient to deliver a dose of 250 mg or more by intravenous infusion;
A near iso-osmolar formulation that is stable upon dilution with commonly used diluents, e.g., 0.9% sodium chloride injection or 5% dextrose injection, for intravenous infusion;
A formulation that is compatible with and stable in Type 1 clear glass vial and standard serum stopper as packaging; and
A sterile drug product (DP) solution that supports long-term stability;
A formulation that minimizes antibody high molecular weight (HMW) species when subjected to handling and thermal stresses;
A formulation that minimizes changes in the relative distribution of antibody charged species when subjected to thermal stress; and
A formulation that maintains biological activity when subjected to handling and thermal stress.

Throughout formulation development, three primary protein stress conditions (representing extreme handling conditions beyond which the antibody drug product would not be subjected during handling, manufacturing, shipping, storing, and labeling) were employed to develop and optimize the antibody formulations and to evaluate the effects of potential real-world stresses on the stability of the drug product. These stress conditions included:
Agitation (vortexing) of the protein solution at room temperature. Vortexing in glass vials exceeds the agitation that occurs during the handling and manufacturing of the protein;
Incubating the protein solution at elevated temperature (37° C., 40° C. or 45° C.) relative to the proposed DP storage condition (2° C.-8° C.);
Subjecting the protein to multiple freeze thaw cycles. Since the protein will undergo at least one freeze thaw cycle during the manufacture of DP, multiple freeze thaw cycles simulate and exceed the actual stress the protein is expected to experience.

There were four main goals of the initial formulation development work:
Selection of buffer and pH: The choice of buffer and pH can have a large effect on the stability of proteins, hence deciding on the optimal buffer species and pH is an important process. Studies are presented in these sections that demonstrate the rationale for choice of the optimal buffer and pH for antibody.
Selection of surfactant or organic cosolvent: A surfactant or organic cosolvent, such as polysorbate, is typically required to prevent precipitation or aggregation of proteins when agitated. Soluble protein may be subjected to agitation when handled, filtered, mixed, manufactured, shipped, and administered. The antibody drug substance in a simple buffered solution can become visibly cloudy with excess agitation. Therefore, it was determined that stabilizing the protein to handling and agitation was important.
Identification/selection of stabilizing/tonicifying excipients: The addition of sugars, salts, and amino acids were examined for their ability to improve the stability of antibody to thermal stress and to increase the shelf life of the DP. The rationale for inclusion of these thermal stabilizers, as well as studies identifying the optimal concentrations in the final formulation are presented herein.
Selection of antibody concentration: The effect of antibody concentration on the stability of the drug product with the selected excipients was examined.

Initial formulation development activities were conducted using 5-50 mg/ml of the anti-PD-1 antibody and involved screening organic cosolvents, thermal stabilizers, and buffers in liquid formulations of anti-PD-1 antibodies to identify excipients that are compatible with the protein and enhance its stability, while maintaining near physiologic osmolality and low viscosity for intravenous and subcutaneous injection. Buffer conditions were also examined to determine the optimal pH for maximum protein stability (described in Examples 4, 6 and 7 herein).

Results from this initial formulation development work were used to develop an initial formulation that was suitable for Phase 1 clinical studies. The phase 1 formulation also provided a reference to optimize late phase clinical and commercial formulations.

With the knowledge gained from the initial formulation development, the late stage formulation development activities involved optimizing pH, surfactant concentration, and stabilizers to identify excipients that enhance protein stability at both low and high protein concentrations (up to 175 mg/ml mAb1) (described in Examples 5, 8, 9, and 10).

Throughout formulation development, the formulations were assessed for stress and storage stability. The methods used to assess stability in the formulation development studies are described in Example 3 herein. Examples 11 and 12 describe the storage and stress stability of the formulations.

Example 13 describes the stability of formulations when the excipients were varied within specific ranges.

Results generated from these studies were used to develop stable liquid formulations suitable for clinical use, for either intravenous (IV) or subcutaneous administration (SC). Example 14 describes containers used for the formulations herein. Examples 15, 16 and 17 describe the compatibility and stability of the formulations in glass vials, prefilled syringes and intravenous delivery devices. Such formulations met the objectives defined for formulation development:
The developed formulations are suitable for the developed doses;
A tonicity that is iso-osmolar to physiologic conditions;
The osmolality of the 50 mg/mL formulation is approximately 318 mOsm/kg;
Sterile DP solution that supports long-term stability in liquid state;
Minimal formation of antibody HMW species occurs upon long-term storage at 2-8° C.;
Little to no change in the relative distribution of antibody charged species occurs upon long-term storage at 2-8° C.; and
Minimal formation of subvisible particles was observed in antibody DP under accelerated storage and stress conditions, and upon storage at 5° C. for 12 months.

Other attributes of the formulations will be apparent from the description herein.

### Anti-PD-1 Antibodies:

Anti-PD-1 antibodies are described in US20150203579, incorporated herein in its entirety. The exemplary antibody used in the Examples below is a fully human anti-PD-1 antibody H4H7798N (as disclosed in US20150203579, known as “REGN2810” or “cemiplimab”) comprising a heavy chain comprising the amino acid sequence of SEQ ID NO: 9 and a light chain comprising the amino acid sequence of SEQ ID NO: 10; an HCVR/LCVR amino acid sequence pair comprising SEQ ID NOs: 1/2; and heavy and light chain CDR sequences comprising SEQ ID NOs: 3-8; and herein referred to as “mAb1”.

### Example 2: Exemplary Formulations

In certain embodiments, mAb1 is formulated as an aqueous buffered formulation containing from 5 mg/ml±0.75 mg/ml to 250 mg/ml±45.0 mg/ml mAb1, 10 mM±2 mM histidine buffer, 0.2%±0.1% w/v polysorbate, 1%±0.2% to 10%±2% w/v sucrose, and 1%±0.02% to 5%±1% w/v proline, at pH 6.0±0.3.

### Exemplary Formulations Include:

A stable low-viscosity pharmaceutical formulation comprising: 25 mg/ml±3.75 mg/ml mAb1, 10±2 mM histidine buffer, 0.2%±0.1% w/v polysorbate 80, 5%±1% w/v sucrose, and 1.5%±0.3% w/v L-proline, at pH 6.0±0.3.
A stable low-viscosity pharmaceutical formulation comprising: 50 mg/ml±7.5 mg/mL mAb1, 10±2 mM histidine buffer, 0.2%±0.1% w/v polysorbate 80, 5%±1% w/v sucrose, and 1.5%±0.3% w/v L-proline, at pH 6.0±0.3.
A stable low-viscosity pharmaceutical formulation comprising: 150±23 mg/ml mAb1, 10±2 mM histidine buffer, 0.2%±0.1% w/v polysorbate 80, 5%±1% w/v sucrose, and 1.5%±0.3% w/v L-proline, at pH 6.0±0.3.
A stable low-viscosity pharmaceutical formulation comprising: 175±27 mg/ml mAb1, 10±2 mM histidine buffer, 0.2%±0.1% w/v polysorbate 80, 5%±1% w/v sucrose, and 1.5%±0.3% w/v L-proline, at pH 6.0±0.3.

### Example 3: Methods Used to Assess Formulation Stability

The following assays were applied to assess formulation stability:
Color and appearance by visual inspection
pH.
Turbidity measured by increase in OD at 405 nm, or by nephelometry.
Particulate matter analysis performed by microflow imaging (MFI) (reported as particle counts obtained as is), and light obscuration (HIAC)
Protein concentration by reverse phase-ultra performance liquid chromatography (RP-UPLC)
Purity by size exclusion-ultra performance liquid chromatography (SE-UPLC), or by reduced and non-reduced microchip capillary electrophoresis sodium dodecyl sulfate (MCE-SDS) PAGE
Charge variant analysis by cation exchange chromatography-ultra performance liquid chromatography (CEX-UPLC), or by imaged capillary isoelectric focusing (iCIEF)
Potency by bioassay: The relative potency of each sample is determined using a bioassay and is defined as: (IC50 reference sample/IC50 sample)*100%. The measured potency of storage stability samples must be within 50% to 150% of the measured potency of the reference standard.

The physical stability of a formulation refers to properties such as color, appearance, pH, turbidity, and protein concentration. The presence of visible particulates in solution can be detected by visual inspection. A solution passes visual inspection if it is clear to slightly opalescent, essentially free from visible particulates, and colorless to pale yellow. In addition, turbidity, measured by OD at 405 nm, can also be used to detect particulates in solution. An increase in OD at 405 nm may indicate the presence of particulates, an increase in opalescence, or color change of the test articles. MFI is used to measure subvisible particulates that are ≥2 μm in size. The protein concentration of mAb1 is measured by a RP-UPLC assay and reported as percent protein recovery relative to the starting material. In the RP-UPLC assay, mAb1 is eluted from the RP column as a single peak. The protein concentration is determined from the mAb1 total peak area by comparing it with a calibration curve generated using mAb1 standards. Percent of recovery is calculated based on the measured protein concentration relative to the starting protein concentration.

Chemical stability refers to the formation of covalently modified forms (e.g. covalent aggregates, cleavage products, or charge variant forms) and non-covalently modified forms (e.g. non-covalent aggregates) of protein. Higher and lower molecular weight degradation products can be separated from native mAb1 by SE-UPLC and MCE-SDS methods. The percentage of degraded mAb1 in the SE-UPLC and MCE-SDS methods is calculated from the ratio of the area of all non-native peaks to the total area of all mAb1 peaks. Charge variant forms of mAb1 are resolved using CEX-UPLC and iCIEF. In the CEX-UPLC method, peaks with retention times earlier than that of the main peak are labeled as “acidic” peaks; the peaks with retention times later than that of the main peak are labeled as “basic” peaks. In the iCIEF method, peaks that are focused to a pI lower than that of the main peak are labeled “acidic” peaks, whereas those focused to a pI higher than that of the main peak are labeled “basic” peaks.

### Example 4: Effect of Different Buffers and pH

The effect of buffer and pH on the thermal stability of mAb1 was examined in liquid formulations by incubating 5 mg/mL mAb1 at 45° C. for 28 days in a series of buffer systems at varying pH ranges. The following pH and buffer systems were studied: acetate (pH 4.5, 5.0, 5.5), histidine (pH 5.5, 6.0, 6.5), and phosphate (pH 6.0, 6.5, 7.0). Based on results from SE-UPLC analysis, maximum protein stability was observed when mAb1 was formulated between pH 6.0 and 6.5 in histidine buffer (Table 1).

TABLE 1
Effect of Buffer and pH on the Stability of 5 mg/mL mAb1 Incubated at 45° C. for 28 Days

| Formulation | 5 mg/mL mAb1, 10 mM Buffer | | | | | | | | |
|---|---|---|---|---|---|---|---|---|---|
| Fill Volume | 0.4 mL | | | | | | | | |
| Container | 2 mL Type 1 borosilicate glass vial with a FluroTec [symbol] coated 4432/50 butyl rubber stopper | | | | | | | | |
| | | Turbidity | % | Change in Purity by | | | Change in Charge | | |
| | Color | (Increase | Protein | SE-UPLC [symbol] | | | Variants by CEX-UPLC [symbol] | | |
| | and | in OD at | Recovered by | % | % | % | % | % | % |
| pH/Buffer | Appearance | 405 nm) | RP-UPLC | HMW | Native | LMW | Acidic | Main | Basic |
| pH 4.5, Acetate | Pass | 0.00 | 87 | 5.2 | −7.2 | 2.0 | 11.1 | −15.4 | 4.3 |
| pH 5.0, Acetate | Pass | 0.00 | 82 | 5.0 | −5.9 | 0.9 | 7.5 | −10.9 | 3.4 |
| pH 5.5, Acetate | Pass | 0.00 | 90 | 4.7 | −5.4 | 0.7 | 12.8 | −13.7 | 0.9 |
| pH 5.5, Histidine | Pass | 0.00 | 97 | 5.6 | −6.4 | 0.8 | 10.8 | −12.4 | 1.6 |
| pH 6.0, Histidine | Pass | 0.00 | 86 | 1.9 | −2.4 | 0.6 | 13.4 | −12.6 | −0.7 |
| pH 6.5, Histidine | Pass | 0.00 | 84 | 1.2 | −1.8 | 0.7 | 25.4 | −21.3 | −4.1 |
| pH 6.0, Phosphate | Pass | 0.01 | 92 | 3.7 | −4.3 | 0.6 | 24.8 | −21.8 | −3.0 |
| pH 6.5, Phosphate | Pass | 0.03 | 91 | 4.9 | −5.8 | 0.9 | 49.6 | −40.3 | −9.3 |
| pH 7.0, Phosphate | Pass | 0.03 | 95 | 10.4 | −11.6 | 1.2 | 56.5 | −42.2 | −14.3 |

[symbol]

Reported as a relative change in purity relative to the starting material. The starting material (no incubation) contains ≥
97.2% native peak by SE-UPLC [symbol] ] ≥49.0% main peak by CEX-UPLC in all formulations.
CEX = Cation exchange;
DS = Drug substance;
HMW = High molecular weight;
LMW = Low moleessbar weight;
OD = Optical density;
RP = Reverse phase;
SE = Size exclusion;
UPLC = Ultra-performance liquid chromatography [symbol]
indicates data missing or illegible when filed

Based on results from CEX-UPLC analysis, maximum protein stability was observed when mAb1 was formulated between pH 5.5 and 6.0 in histidine buffer or between pH 5.0 and 5.5 in acetate buffer. These analyses also revealed that aggregation (i.e. formation of HMW species), fragmentation (i.e. formation of LMW species), and formation of charge variants were the main degradation pathways. Histidine buffer was selected as the formulation buffer because it provided the best overall level of protein stabilization with respect to formation of HMW and LMW species and formation of charge variants. A pH of 6.0 was chosen for the formulation because formation of HMW species and charge variants, which are the major degradation pathways, were minimized at this pH. Based on these results, 10 mM histidine buffer at pH 6.0 was chosen for the mAb1 formulation.

### Example 5: pH Screening in Histidine Buffers

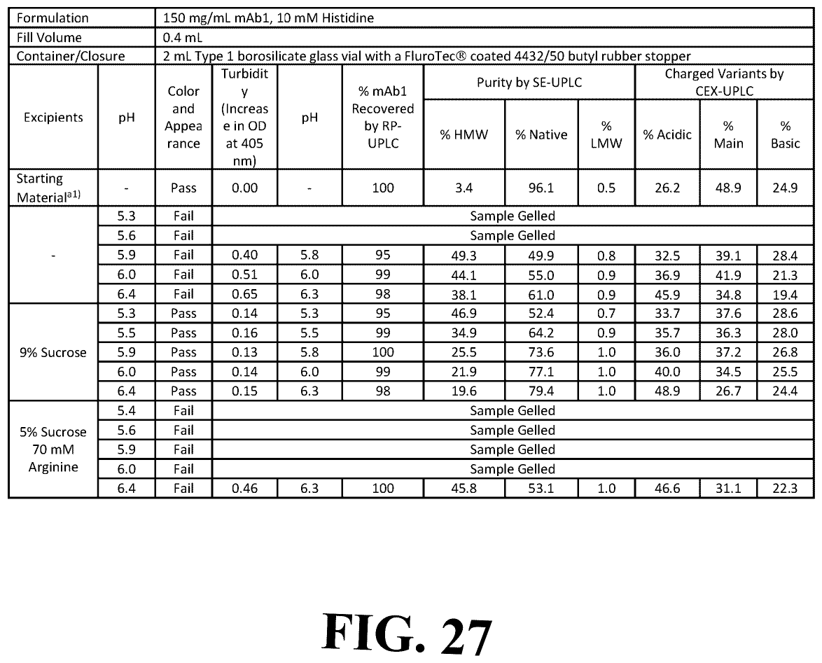

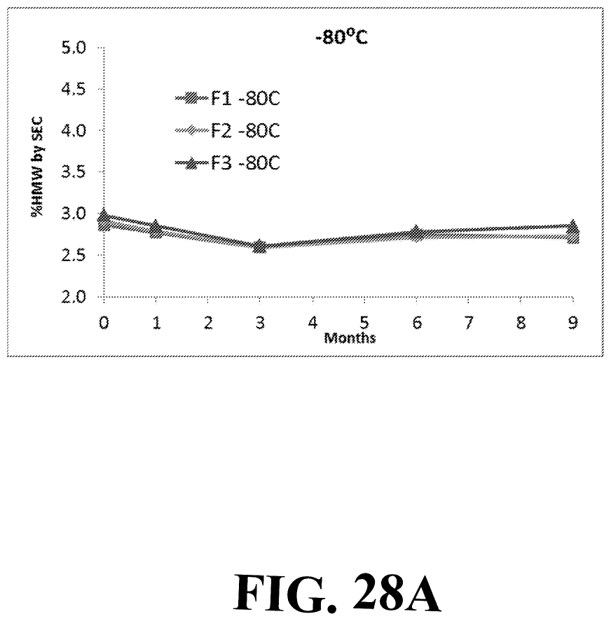

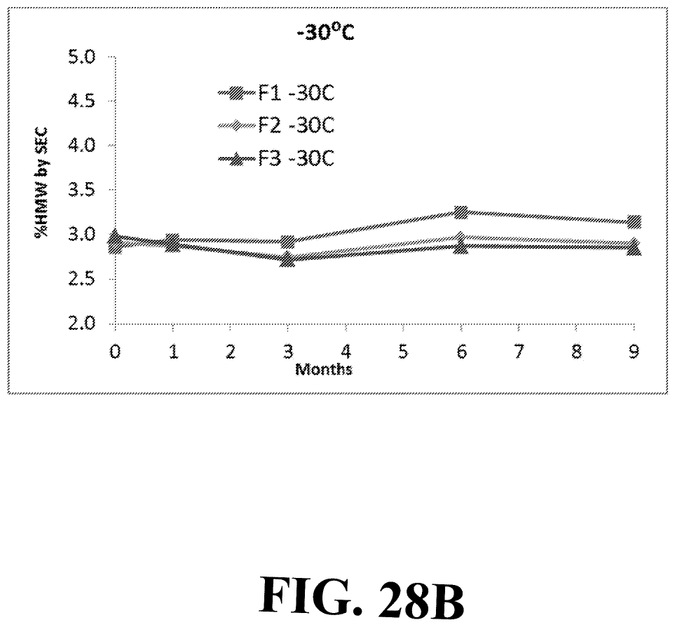

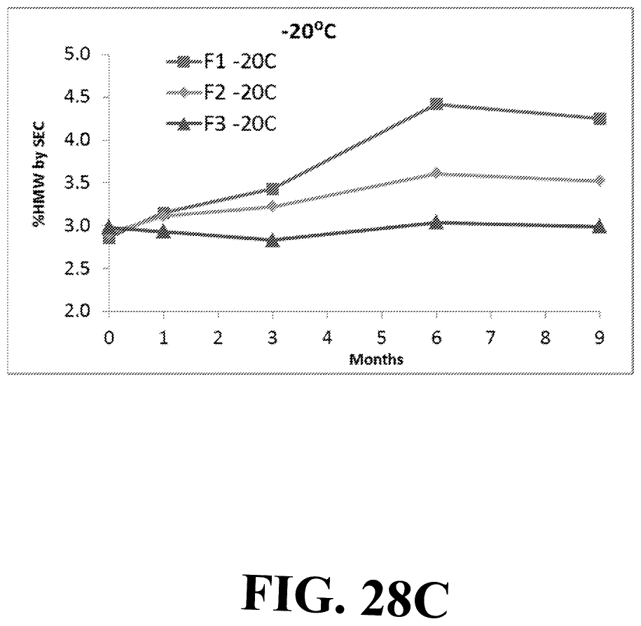

The effect of buffer and pH on the thermal stability of mAb1 was examined in high concentration liquid formulations. 150 mg/ml mAb1 was incubated at 45° C. for 28 days in a series of histidine buffers ranged at pH 5.3, 5.5, 5.8, 6.0 and 6.3 with and without thermal stabilizers. With 9% sucrose, based on results from SE-UPLC analysis, maximum protein stability was observed when mAb1 was formulated between pH 5.8 and 6.3 in histidine buffer (FIG. 27). Based on results from CEX-UPLC analysis, maximum protein stability was observed when mAb1 was formulated between pH 5.3 and 6.0 in histidine buffer (Table 2).

TABLE 2
Effect of pH on the Stability of 150 mg/mL mAb1 Incubated at 45° C. for 28 Days

| Formulation | 150 mg/mL mAb1, 10 mM histidine | | | | | | | | |
|---|---|---|---|---|---|---|---|---|---|
| Fill Volume | 0.4 mL | | | | | | | | |
| Container/Closure | 2 mL Type 1 borosilicate glass vial with a FluroTec ® coated 4432/50 butyl rubber stopper | | | | | | | | |
| | | Turbidity | % | Change in Purity by | | | Change in Charge | | |
| | Color | (Increase | Protein | SE-UPLC [symbol] | | | Variants by CEX-UPLC [symbol] | | |
| | and | in OD at | Recovered by | % | % | % | % | % | % |
| pH/Stabilizer | Appearance | 405 nm) | RP-UPLC | HMW | Native | LMW | Acidic | Main | Basic |
| pH 5.3/None | Fail [symbol] | NA | NA | NA | NA | NA | NA | NA | NA |
| pH 5.5/None | Fail [symbol] | NA | NA | NA | NA | NA | NA | NA | NA |
| pH 5.8/None | Fail | 0.40 | 95 | 46.2 | −46.4 | 0.3 | 6.4 | −10.2 | 3.9 |
| pH 6.0/None | Fail | 0.51 | 99 | 41.2 | −41.5 | 0.3 | 10.6 | −7.3 | −3.3 |
| pH 6.3/None | Fail | 0.65 | 98 | 35.0 | −35.4 | 0.4 | 19.6 | −14.3 | −5.3 |
| pH 5.3/ | Pass | 0.14 | 95 | 41.9 | −42.1 | 0.3 | 7.9 | −11.1 | 3.2 |
| 9% (w/v) | | | | | | | | | |
| Sucrose | | | | | | | | | |
| pH 5.5/ | Pass | 0.16 | 99 | 30.8 | −31.2 | 0.4 | 9.5 | −12.5 | 2.9 |
| 9% (w/v) | | | | | | | | | |
| Sucrose | | | | | | | | | |
| pH 5.8/ | Pass | 0.13 | 100 | 22.8 | −23.3 | 0.5 | 9.7 | −11.8 | 2.1 |
| 9% (w/v) | | | | | | | | | |
| Sucrose | | | | | | | | | |
| pH 6.0/ | Pass | 0.14 | 99 | 19.4 | −19.9 | 0.5 | 13.5 | −14.5 | 1.0 |
| 9% (w/v) | | | | | | | | | |
| Sucrose | | | | | | | | | |
| pH 6.3/ | Pass | 0.15 | 98 | 16.9 | −17.4 | 0.5 | 22.4 | −22.1 | 0.4 |
| 9% (w/v) | | | | | | | | | |
| Sucrose | | | | | | | | | |

^{a}Reported as a relative change in purity relative to the starting material. The starting material (no incubation) contains ≥94.0% native peak by SE-UPLC and ≥48.7% main peak by CEX-UPLC in all formulations.
^{b}Sample gelled. No futher analysis was performed.
NA = Not applicable
CEX, cation exchange;
HMW, high molecular weight;
LMW, low molecular weight;
OD, optical density;
RP, reverse phase;
SE, size exclusion;
UPLC, ultra performance liquid chromatography [symbol]
indicates data missing or illegible when filed

These analyses also revealed that aggregation (i.e. formation of HMW species), and formation of charge variants were the main degradation pathways. A pH of 6.0 was chosen for the DP formulation because formation of HMW species and charge variants, which are the major degradation pathways, were minimized at this pH. Based on these results, 10 mM histidine buffer at pH 6.0 was chosen for the mAb1 high concentration DP formulation.

### Example 6: Selection of Protectants Against Agitation Stress

Stabilizers such as surfactants and organic co-solvents are often added to the antibody formulations to protect the protein from agitation-induced aggregation. The effect of organic co-solvents and surfactants on the agitation stress stability and thermal stability of 5 mg/ml mAb1 was examined in liquid formulations. The following co-solvent and surfactants were evaluated: 0.1% polysorbate 20, 0.1% polysorbate 80, and 1.0% PEG3350. The results of agitation stress stability studies are summarized in Table 3.

TABLE 3
Effect of Organic Co-solvents and Surfactants on the Stability of 5 mg/ml mAb1
After Agitation (120 Min of Vortexing)

| Formulation | 5 mg/mL mAb1, 10 mM histidine, pH 6.0 | | | | | | | | | |
|---|---|---|---|---|---|---|---|---|---|---|
| Fill Volume | 0.4 mL | | | | | | | | | |
| Container | 2 mL Type 1 borosilicate glass vial with a FluorTec ® coated 4432/50 butyl rubber stopper | | | | | | | | | |
| | | Turbidity | | % | Change in Purity by | | | Change in Charge | | |
| | Color | (Increase | | Protein | SE-UPLC [symbol] | | | Variants by CEX-UPLC [symbol] | | |
| Co-solvent/ | and | in OD at | | Recovered by | % | % | % | % | % | % |
| Surfactant | Appearance | 405 nm) | pH | RP-UPLC | HMW | Native | LMW | Acidic | Main | Basic |
| No co-solvent/ | Fail | 1.69 | 6.0 | 76 | 13.5 | −23.7 | −8.4 | −2.3 | −1.7 | 3.9 |
| surfactant | | | | | | | | | | |
| 5% (w/v) | Fail | 1.75 | 6.0 | 64 | 9.4 | −12.8 | 3.4 | −1.7 | −0.1 | 1.8 |
| sucrose | | | | | | | | | | |
| 0.1% (w/v) | Pass | 0.00 | 6.0 | 102 | −0.1 | −0.8 | 0.8 | 0.2 | 0.2 | −0.3 |
| polysorbate 20 [symbol] | | | | | | | | | | |
| 0.1% (w/v) | Pass | 0.00 | 6.0 | 100 | −0.2 | −0.5 | 0.4 | 0.2 | −0.1 | −0.1 |
| polysorbate 80 [symbol] | | | | | | | | | | |
| 1% (w/v) | Fail | 0.20 | 6.0 | 93 | 0.3 | −0.5 | 0.2 | −0.2 | −0.3 | 0.4 |
| PEG3350 [symbol] | | | | | | | | | | |

^{a}Reported as a relative change in purity relative to the starting material. The starting material (no incubation) contains ≥98.2% native peak by SE-UPLC and ≥49.1% main peak by CEX-UPLC in all 5 formulations.
^{1:} The formulations also contains 5% sucrose.
CEX = Cation exchange;
HMW = High molecular weight;
LMW = Low molecular weight;
OD = Optical density;
RP = Reverse phase;
SE = Size exclusion;
UPLC = Ultra-performance liquid chromatography [symbol]
indicates data missing or illegible when filed

mAb1 was unstable when agitated by vortexing for 120 min in the absence of an organic co-solvent or surfactant. After agitation by vortexing in the absence of co-solvent or surfactant, the solution became cloudy, exhibited a substantial increase in turbidity, and had a 15.3% increase in aggregates as determined by SE-UPLC, as well as 24% loss in protein recovery by RP-UPLC (Table 3). 1% PEG3350 did not provide sufficient stabilization of mAb1 after 120 min of vortexing. In the presence of 1% PEG3350, the solution became cloudy, and exhibited an increase in turbidity (Table 3). In contrast, 0.1% polysorbate 20 and 0.1% polysorbate 80 both protected mAb1 from agitation-induced instability to the same extent (Table 3).

However, the formulation containing 0.1% polysorbate 80 exhibited a decreased amount of aggregates compared to the formulation containing 0.1% polysorbate 20 when incubated at 45° C. (Table 4). 0.1% polysorbate 80 was chosen as the surfactant for the mAb1 DP formulation because it stabilized the protein to agitation stress, had less negative effect on protein thermal stability than polysorbate 20 (as determined by both SE-UPLC and CEX-UPLC analyses), and has a safe history of use in monoclonal antibody formulations.

### Example 7: Selection of Protectants Against Thermal Stress

Stabilizers such as sucrose are often added to antibody formulations to increase the thermal stability of the protein in liquid formulations. Five (5) mg/ml mAb1 in a liquid formulation exhibited improved stability when formulated with 5% sucrose and incubated under accelerated conditions (Table 4).

TABLE 4
Effect of Organic Co-solvents and Surfactants on the Stability of 5 mg/ml mAb1
Incubated at 45° C. for 29 Days

| Formulation | 5 mg/mL mAb1, 10 mM histidine, pH 6.0 | | | | | | | | | |
|---|---|---|---|---|---|---|---|---|---|---|
| Fill Volume | 0.4 mL | | | | | | | | | |
| Container/Closure | 2 mL Type 1 borosilicate glass vial with a FluorTec ® coated 4432/50 butyl rubber stopper | | | | | | | | | |
| | | Turbidity | | % | Change in Purity by | | | Change in Charge | | |
| | Color | (Increase | | Protein | SE-UPLC [symbol] | | | Variants by CEX-UPLC [symbol] | | |
| Co-solvent/ | and | in OD at | | Recovered by | % | % | % | % | % | % |
| Surfactant | Appearance | 405 nm) | pH | RP-UPLC | HMW | Native | LMW | Acidic | Main | Basic |
| No co-solvent/ | Pass | 0.00 | 6.1 | 96 | 2.8 | −3.2 | 0.5 | 10.8 | −10.7 | −0.2 |
| surfactant | | | | | | | | | | |
| 5% (w/v) | Pass | 0.01 | 6.1 | 99 | 1.6 | −2.0 | 0.3 | 12.6 | −11.7 | −0.9 |
| sucrose | | | | | | | | | | |
| 0.1% (w/v) | Pass | 0.00 | 6.0 | 92 | 27.6 | −28.4 | 0.7 | 0.1 | −74.0 | 23.9 |
| polysorbate 20 [symbol] | | | | | | | | | | |
| 0.1% (w/v) | Pass | 0.00 | 6.1 | 96 | 12.8 | −14.1 | 0.9 | 4.4 | −20.4 | 15.9 |
| polysorbate 80 | | | | | | | | | | |
| 1% (w/v) | Pass | 0.00 | 6.1 | 90 | 8.4 | −8.0 | −0.4 | 0.4 | −25.0 | 24.5 |
| PEG3350 | | | | | | | | | | |

^{a}Reported as a relative change in purity relative to the starting material. The starting material (no incubation) contains ≥98.2% native peak by SE-UPLC and ≥49.1% main peak by CEX-UPLC in all 5 formulations.
^{b}The formulations also contains 5% sucrose.
CEX = Cation exchange;
HMW = High molecular weight;
LMW = Low molecular weight;
OD = Optical density;
RP = Reverse phase;
SE = Size exclusion;
UPLC = Ultra-performance liquid chromatography [symbol]
indicates data missing or illegible when filed

After incubation at 45° C. for 29 days, the relative amount of HMW species increased by 1.7% in the formulation containing 5% sucrose compared to a 2.8% increase in the control formulation without sucrose. For this reason, sucrose was chosen as the thermal stabilizer. To make the formulation isotonic and to maximize the thermal stability, the sucrose concentration was increased to 10% for the mAb1 formulation.

### Example 8: Optimization of Stabilizers

The goal of optimizing the thermal stabilizers was to identify the stabilizing components that could be used to develop a DP formulation supporting an antibody concentration of up to 200 mg/mL. 10% sucrose was selected in the initial formulation. It was found that with 10% sucrose, the viscosity of mAb1 was about 20 cP at 20° C. and was considered too high for a robust late stage and commercial product. Therefore, a modified mAb1 formulation was needed that exhibited both favorable stability and lower viscosity.

Sucrose was chosen as the thermal stabilizer for mAb1 during the low concentration formulation development. For the high concentration formulation development, different concentrations of sucrose and L-proline were evaluated on the stability and viscosity of the mAb1 at 150 and 175 mg/mL concentrations at 25° C. (Table 5) and at 40° C. for 1 month. The formation of HMW species decreased with increasing sucrose concentrations when the formulations were incubated at 40° C. for 28 days. 3% L-proline provided similar stabilization to 5% sucrose, and the maximum stabilization was observed with 9% of sucrose.

Although 9% sucrose provided slightly better stabilization comparing with 3% L-proline, it also increased the formulation viscosity. At 175 mg/mL, mAb1 formulation with 9% of sucrose has a viscosity of 27 centipoise, which pose manufacturing and administration challenges. The 175 mg/ml mAb1 formulation with 3% of proline has a viscosity of approximately 20 centipoise, which is manageable with the current manufacturing process.

TABLE 6
Effect of stabilizers on the stability of 50 mg/ml mAb1 after incubation at 45° C. for 28 days

| Formulation | 50 mg/mL mAb1, 10 mM L-histidine, 5% sucrose, pH 6.0, 0.2% polysorbate 80 | | | | | | | | | |
|---|---|---|---|---|---|---|---|---|---|---|
| Fill Volume | 0.6 mL | | | | | | | | | |
| Container/Closure | 2 mL Type 1 borosilicate glass vial with a FluorTec ® coated 4432/50 chlorobutyl stopper | | | | | | | | | |
| | | Turbidity | | % | Change in Purity by | | | Change in Charge | | |
| | Color | (Increase | | Protein | SE-UPLC [symbol] | | | Variants by CEX-UPLC [symbol] | | |
| Stabilizer | and | in OD at | | Recovered by | % | % | % | % | % | % |
| (% w/v) | Appearance | 405 nm) | pH | RP-UPLC | HMW | Native | LMW | Acidic | Main | Basic |
| — | Pass | 0.02 | 6.0 | 96 | 12.1 | −12.7 | 0.6 | 10.8 | −11.9 | 1.0 |
| 1.5% L-proline | Pass | 0.02 | 6.1 | 96 | 11.3 | −11.9 | 0.6 | 11.0 | −11.8 | 0.8 |
| 3.0% L-proline | Pass | 0.01 | 6.0 | 96 | 10.9 | −11.5 | 0.6 | 12.8 | −12.9 | 0.1 |

[symbol]

Reported as a change in purity relative to the starting material. The starting material (no incubation) contains ≥98.5% Monomer peak by SE-UPLC and ≥52.8% main peak by CEX-UPLC in all three formulations.
CEX, Cation exchange;
DS, drug substance;
HMW, High molecular weight;
LMW, Low molecular weight;
OD, Optical density;
RP, Reverse phase;
SE, Size exclusion;
UPLC, Ultra-performance liquid chromatography [symbol]
indicates data missing or illegible when filed

The effect of different stabilizers on the thermal stability of high concentrations (150 and 175 mg/mL) mAb1 was further examined in liquid formulations. The stabilizers evaluated were 9% (w/v) sucrose, 3% (w/V) L-proline, and 5% (w/v) sucrose with 1.5% (w/v) L-proline. The results of the accelerated stability study are summarized in Table 7.

TABLE 7
Effect of Stabilizers on the Stability of mAb1 after incubation at 25° C. and 40° C.

| Formulation | 150 or 175 mg/mL mAb1, 10 mM histidine, pH 6.0, 0.1% polysorbate 80 | | | | | | | | | | |
|---|---|---|---|---|---|---|---|---|---|---|---|
| Fill Volume | 0.5 mL | | | | | | | | | | |
| Container/Closure | 2 mL Type 1 borosilicate glass vial with a FluorTec ® coated 4432/50 butyl rubber stopper | | | | | | | | | | |
| | | | Turbidity | | % | Change Purity by | | | Change in Charge | | |
| | mAb1 | Color | (Increase | | Protein | SE-UPLC [symbol] | | | Variants by CEX-UPLC [symbol] | | |
| Stabilizer | Conc. | and | in OD at | | Recovered by | % | % | % | % | % | % |
| (% w/v) | (mg/mL) | Appearance | 405 nm) | pH | RP-UPLC | HMW | Native | LMW | Acidic | Main | Basic |
| 9% Sucrose | 150 | Pass | 0.02 | 6.2 | 110 | 2.6 | −2.8 | 0.2 | 6.0 | −4.2 | −1.9 |
| 3% L-proline | 175 | Pass | 0.00 | 6.2 | 112 | 2.4 | −2.5 | 0.2 | 6.5 | −4.1 | −2.4 |
| 5% Sucrose, | 175 | Pass | 0.00 | 6.2 | 110 | 2.3 | −2.6 | 0.2 | 6.0 | −3.7 | −2.4 |
| 1.5% L-proline | | | | | | | | | | | |
| Incubation | | 40° C. for 28 days | | | | | | | | | |
| | | | Turbidity | | % | Change Purity by | | | Change in Charge | | |
| | mAb1 | Color | (Increase | | Protein | SE-UPLC [symbol] | | | Variants by CEX-UPLC [symbol] | | |
| Stabilizer | Conc. | and | in OD at | | Recovered by | % | % | % | % | % | % |
| (% w/v) | (mg/mL) | Appearance | 405 nm) | pH | RP-UPLC | HMW | Native | LMW | Acidic | Main | Basic |
| 9% Sucrose | 150 | Pass | 0.04 | 6.1 | 102 | 6.9 | −7.5 | 0.5 | 9.4 | −9.7 | 0.3 |
| 3% L-proline | 175 | Pass | 0.03 | 6.2 | 103 | 11.7 | −12.3 | 0.6 | 12.1 | −10.7 | −1.4 |
| 5% Sucrose, | 175 | Pass | 0.03 | 6.1 | 103 | 8.8 | −9.4 | 0.7 | 11.4 | −10.2 | −1.2 |
| 1.5% L-proline | | | | | | | | | | | |

^{a}Reported as a change in purity relative to the starting material. The starting material (no incubation) contains ≥97.3% native peak by SE-UPLC and ≥49.6% main peak by CEX-UPLC in all three formulations.
CEX, Cation exchange;
HMW, High molecular weight;
LMW, Low molecular weight;
OD, Optical density;
RP, Reverse phase;
SE, Size exclusion;
UPLC, Ultra-performance liquid chromatography [symbol]
indicates data missing or illegible when filed

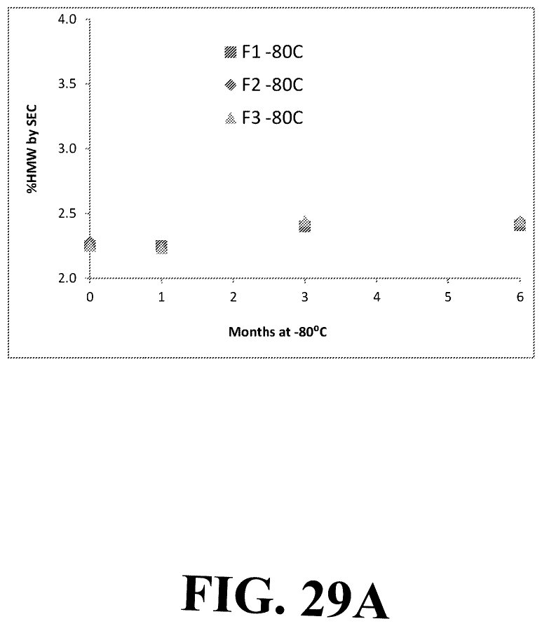

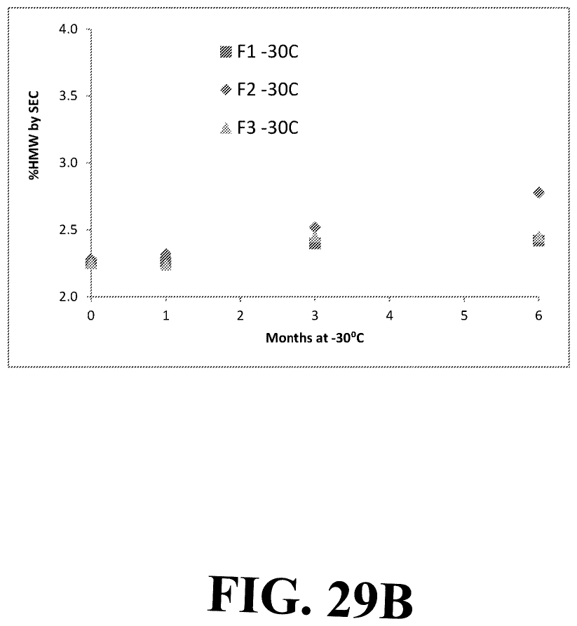

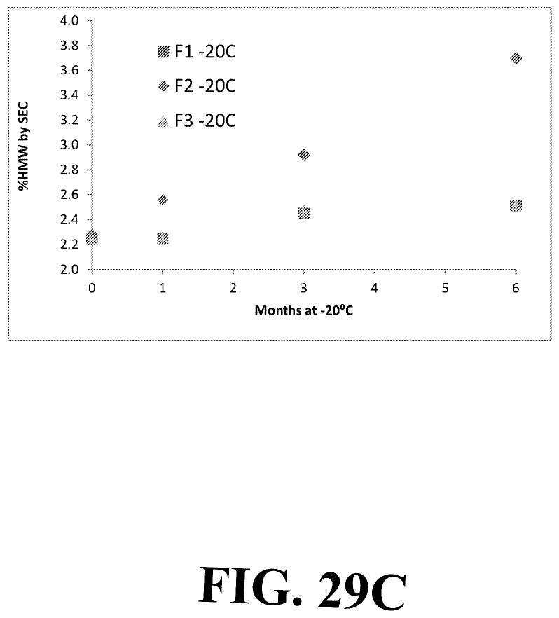

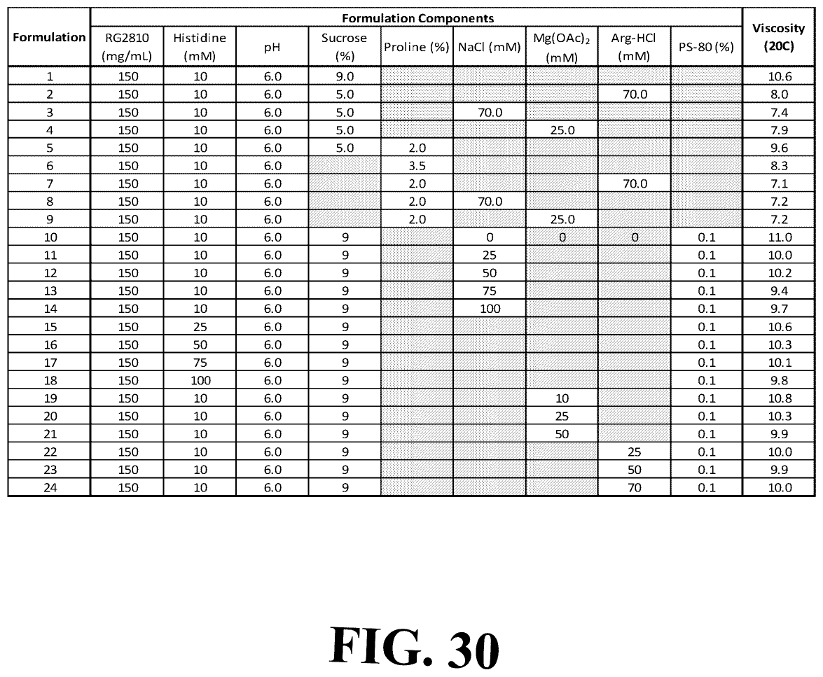

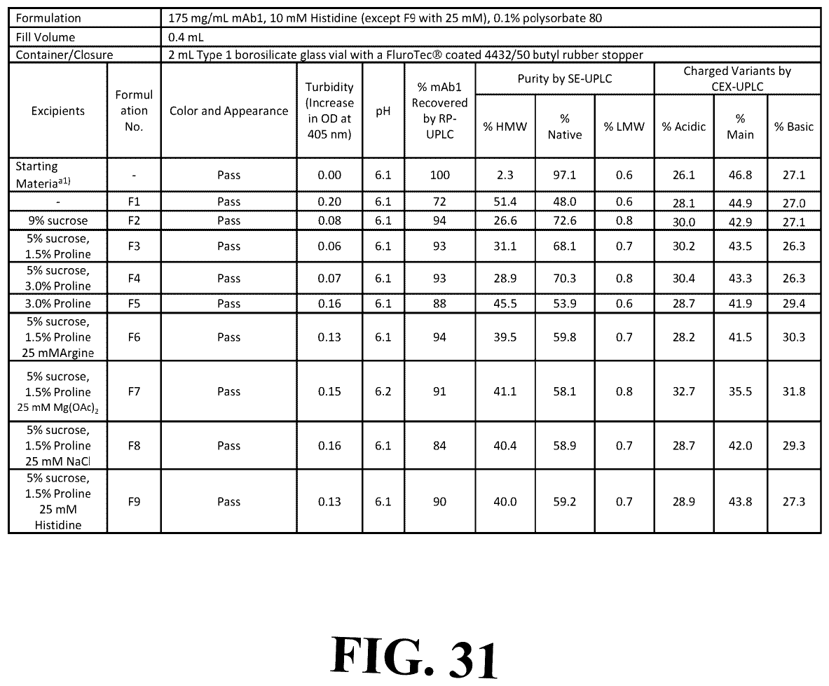

After incubation at 40° C. for 28 days, 9% sucrose provided the best stabilization and had the highest viscosity among the high concentration formulations. The 5% sucrose/1.5% L-proline formulation ranked second for stability after 28 days at 40° C. After incubation at 25° C. for three months, the stability of mAb1 was nearly the same in all of the formulations examined; however, the formulation with 5% sucrose/1.5% L-proline was slightly better than the other two formulations. However, upon incubation at −20° C., −30° C. and −80° C., sucrose at 5% and at 9% provided better stability than 3% proline (FIGS. 29A, 29B, and 29C). The viscosity of 175 mg/ml mAb1 with 5% sucrose/1.5% L-proline has a viscosity of 14 cP at 20° C. To provide a formulation that yields an isotonic solution and achieves the best balance between stability and viscosity, 5% sucrose/1.5% L-proline was selected for development of an antibody late phase DP formulation.

### Example 9: Selection of Viscosity Modifiers

The viscosity of protein formulations increases exponentially as the protein concentration increases. When the viscosity begins to exceed about 10 to 15 cP at 20° C., viscosity of the formulation must be taken into account when developing a formulation: this is simply because viscosity correlates with the ease of injection through a prefilled syringe (PFS) or other needle-based delivery device; more importantly maintaining a reasonably low viscosity is critical for the development of a delivery device, such as autoinjector. The effect of excipients on formulation viscosity was examined in liquid formulation with the following potential viscosity modifiers, proline, arginineHCI, histidineHCI, magnesium acetate and NaCl. FIG. 4 summarizes the viscosity of 150 mg/mL mAb1 with the viscosity modifiers. ArginineHCI, histidineHCI, magnesium acetate and NaCl at 25-100 mM lower the viscosity of 150 mg/ml mAb1 formulations.

Impact of the viscosity modifiers on stability of mAb1 formulation was also examined. 150 mg/ml mAb1 formulations with viscosity modifiers such as arginineHCI, histidineHCI, magnesium acetate and NaCl were prepared and incubated at 45° C. for 28 days. The results are shown in FIG. 5. It was found that maximum protein stability was observed when mAb1 was formulated without the viscosity modifiers; all these viscosity modifying salts negatively impact the mAb1 stability. Therefore salts were not included in the final formulation.

L-proline, as a stabilizer, minimized solution viscosity for antibody concentrations at or above 50 mg/mL. The results of the accelerated stability studies for high concentration antibody with varying amounts of L-proline, with and without sucrose, are summarized in Table 7. After incubation at 25° C. for three months, the formulation with 5% sucrose/1.5% L-proline provided slightly improved stability relative to the other two formulations with respect to formation of HMW species. After incubation at 40° C. for 28 days, the formulation containing 9% sucrose provided the best stabilization, and the formulation with 5% sucrose/1.5% L-proline formulation ranked second. The formulation with 9% sucrose has a viscosity of 20 cP at 175 mg/mL antibody, while the viscosity of 175 mg/mL formulation with 5% sucrose/1.5% L-proline has a viscosity of 14 cP at 20° C. Adding L-proline to the formulation was important for lowering the viscosity at elevated protein concentrations as well as stabilizing the antibody.

In summary, 5% sucrose/1.5% L-proline was selected for both 50 mg/mL and high concentration antibody formulations. This combination of excipients achieved an isotonic formulation with acceptable stability and viscosity at all antibody concentrations tested (up to 175 mg/mL).

### Example 10: Optimization of Polysorbate (PS) Concentration

During formulation development, higher order molecular weight species formation and an increase in turbidity was observed when the mAb1 formulation was agitated without surfactant. The protein was stabilized to agitation by addition of polysorbate 80 (PS 80). During high concentration mAb1 liquid formulation development, instability to agitation was observed as an increase in higher order molecular weight species. A study was carried out to determine the minimum amount of polysorbate 80 needed to protect up to 175 mg/ml mAb1 from agitation-induced instability. The formulations in this study contained 5% sucrose and 1.5% L-proline so that the effect of polysorbate 80 could be studied with a formulation composition that was more representative of the final formulation. The nominal polysorbate 80 concentrations included in the study were 0%, 0.02%, 0.04%, 0.06%, 0.08%, 0.1%, 0.15%, and 0.2% (w/v). In the absence of polysorbate 80, the solution became cloudy and exhibited a substantial increase in turbidity after agitation by vortexing. A polysorbate 80 concentration-dependent reduction in the amount of % HMW after 120 minutes of agitation was observed. A concentration of 0.15-0.2% polysorbate 80 was found to be sufficient to stabilize 150 mg/mL and 175 mg/mL mAb1 to agitation induced aggregation (Table 8). The addition of 0.2% (w/v) (nominal value) polysorbate 80 completely prevented formation of the HMW species after agitation for 120 minutes.

TABLE 8
Stability of 150 mg/mL and 175 mg/ml mAb1 with PS 80 after 120 min of agitation

| Fill Volume | 0.4 mL | | | | | |
|---|---|---|---|---|---|---|
| Container/Closure | 2 mL Type 1 borosilicate glass vial with a FluorTec ® coated 4432/50 butyl rubber stopper | | | | | |
| | | | Turbidity | | % mAb1 | |
| | | Color | (Increase | | Recovered | Increase in |
| | | and | in OD at | | by RP- | % HMW by |
| Formulation | [PS 80] | Appearance | 405 nm) | pH | UPLC | SE-UPLC |
| 150 mg/mL | 0.00 | Fail | 0.38 | 6.0 | Not Applicable | |
| mAb1 | 0.02 | Pass | 0.00 | 6.0 | 97 | 4.7 |
| 10 mM | 0.04 | Pass | 0.00 | 6.0 | 100 | 0.3 |
| histidine, pH | 0.06 | Pass | 0.00 | 6.0 | 102 | 0.3 |
| 6.0 | 0.08 | Pass | 0.04 | 6.0 | 99 | 0.0 |
| 0% sucrose | 0.10 | Pass | 0.01 | 6.0 | 101 | 0.1 |
| | 0.15 | Pass | 0.00 | 6.0 | 98 | 0.1 |
| | 0.20 | Pass | 0.00 | 6.0 | 106 | 0.1 |
| 175 mg/mL | 0.00 | Fail | 0.38 | 6.0 | Not Applicable | |
| mAb1 | 0.02 | Pass | 0.06 | 6.1 | 300 | 7.6 |
| 10 mM | 0.04 | Pass | 0.00 | 6.0 | 97 | 2.4 |
| histidine, pH | 0.06 | Pass | 0.01 | 6.1 | 700 | 2.3 |
| 6.0 | 0.08 | Pass | 0.03 | 6.1 | 98 | 0.9 |
| 3% sucrose | 0.10 | Pass | 0.00 | 6.1 | 102 | 0.4 |
| | 0.15 | Pass | 0.00 | 6.1 | 102 | 0.1 |
| | 0.20 | Pass | 0.00 | 6.1 | 101 | 0.1 |
| 175 mg/mL | 0.00 | Fail | 0.36 | 6.1 | Not Applicable | |
| mAb1 | 0.02 | Pass | 0.01 | 6.1 | 97 | 3.5 |
| 10 mM | 0.04 | Pass | 0.01 | 6.1 | 98 | 2.0 |
| histidine, pH | 0.06 | Pass | 0.01 | 6.1 | 100 | 1.3 |
| 6.0 | 0.08 | Pass | 0.01 | 6.1 | 99 | 1.0 |
| 5% sucrose | 0.10 | Pass | 0.01 | 6.0 | 102 | 0.3 |
| 1.5% proline | 0.15 | Pass | 0.00 | 6.1 | 101 | 0.2 |
| | 0.20 | Pass | 0.00 | 6.1 | 98 | 0.0 |

Table 9 details the effect of polysorbate 80 concentration on the stability of 175 mg/ml mAb1 after agitation (120 minutes of vortexing).

TABLE 9
Effect of Polysorbate 80 Concentration on the Stability of 175 mg/ml mAb1 after
Agitation (120 minutes of Vortexing)

| Formulation | 175 mg/mL mAb1, 10 mM histidine, pH 6.0, 5% (w/v) sucrose, 1.5% (w/v) L-proline | | | | | | | | | |
|---|---|---|---|---|---|---|---|---|---|---|
| Fill Volume | 0.4 mL | | | | | | | | | |
| Container/Closure | 2 mL Type 1 borosilicate glass vial with a FluorTec ® coated 4432/50 butyl rubber stopper | | | | | | | | | |
| | | Turbidity | | % | Change in Purity by | | | Change in Charge | | |
| Nominal PS | Color | (Increase | | Protein | SE-UPLC [symbol] | | | Variants by CEX-UPLC [symbol] | | |
| 80 Conc. | and | in OD at | | Recovered by | % | % | % | % | % | % |
| (% w/v) | Appearance | 405 nm) | pH | RP-UPLC | HMW | Native | LMW | Acidic | Main | Basic |
| 0.00% | Fail | 0.36 | 6.0 | NA | NA | NA | NA | NA | NA | NA |
| 0.02% | Pass | 0.01 | 6.0 | 97 | 1.5 | −3.5 | 0.0 | −0.6 | 0.8 | −0.1 |
| 0.04% | Pass | 0.01 | 6.0 | 98 | 2.0 | −2.0 | 0.0 | 0.1 | −0.2 | −0.2 |
| 0.06% | Pass | 0.01 | 6.0 | 100 | 1.3 | −1.3 | 0.0 | −0.2 | 0.3 | −0.1 |
| 0.08% | Pass | 0.01 | 6.0 | 99 | 1.0 | −1.0 | 0.0 | −0.2 | 0.0 | 0.0 |
| 0.10% | Pass | 0.02 | 6.0 | 102 | 0.3 | −0.3 | 0.0 | 0.2 | 0.0 | 0.0 |
| 0.15% | Pass | 0.00 | 6.1 | 101 | 0.2 | −0.2 | 0.0 | 0.2 | −0.3 | 0.1 |
| 0.20% | Pass | 0.00 | 6.1 | 98 | 0.0 | 0.0 | 0.0 | 0.0 | −0.1 | 0.2 |

^{a}Reported as a relative change in purity relative to the starting material. The starting material (no incubation) contains ≥97.4% native peak by SE-UPLC and ≥48.2% main peak by CEX-UPLC in all three formulations.
CEX, Cation exchange;
HMW, High molecular weight;
LMW, Low molecular weight;
NA, not available;
OD, Optical density;
RP, Reverse phase;
SE, Size exclusion;
UPLC, Ultra-performance liquid chromatography [symbol]
indicates data missing or illegible when filed

The ability of 0.2% (w/v) polysorbate 80 to protect mAb1 from agitation-induced instability was confirmed by another study with the final formulation at 50 mg/mL (Table 10).

TABLE 10
Effect of PS80 Concentration on the Stability of 50 mg/ml mAb1 after
Agitation (120 minutes of Vortexing)

| Formulation | 50 mg/ml mAb1, 10 mM L-histidine, pH 6.0, 5% (w/v) sucrose, 1.5% (w/v) L-proline | | | | | | | | | |
|---|---|---|---|---|---|---|---|---|---|---|
| Fill Volume | 0.6 mL | | | | | | | | | |
| Container/Closure | 2 mL Type 1 glass vial with a FluorTec ® coated 4432/50 chlorobutyl stopper | | | | | | | | | |
| | | Turbidity | | % | Change in Purity by | | | Change in Charge | | |
| Polysorbate | Color | (Increase | | Protein | SE-UPLC [symbol] | | | Variants by CEX-UPLC [symbol] | | |
| 80 Conc. | and | in OD at | | Recovered by | % | % | % | % | % | % |
| (% w/v) | Appearance | 405 nm) | pH | RP-UPLC | HMW | Monomer | LMW | Acidic | Main | Basic |
| 0.0% | Pass | 0.01 | 6.1 | 99 | 8.0 | −8.0 | 0.0 | 1.7 | −1.8 | −0.3 |
| 0.2% | Pass | 0.01 | 6.1 | 99 | 0.0 | 0.1 | −0.1 | −0.2 | 0.1 | −0.2 |

^{a}Reported as a relative change in purity relative to the starting material. The starting material (no incubation) contains ≥98.5%
Monomer peak by SE-UPLC and ≥52.8% main peak by CEX-UPLC in all five formulations.
CEX, Cation exchange;
DS, drug substance;
HMW, High molecular weight;
LMW, Low molecular weight;
NA, not available;
OD, Optical density;
RP, Reverse phase;
SE, Size exclusion;
UPLC, Ultra-performance liquid chromatography [symbol]
indicates data missing or illegible when filed

Based on these results, 0.2% (w/v) polysorbate 80 was selected as the surfactant because it provided sufficient stabilization to prevent formation of HMW species under agitation stress.

### Example 11: Storage and Stress Stability of Exemplary Formulations

The storage stability of 50 mg/mL and 175 mg/mL mAb1 formulations in glass vials are shown in Table 11 and Table 12, and the accelerated and stress stability data from the two formulations are shown in Table 13 and Table 14, respectively. Research stability studies demonstrated that the 50 mg/ml and 175 mg/ml mAb1 formulation in glass vials are stable for at least 24 months when stored at 2° C. to 8° C. In addition, the 50 mg/ml mAb1 formulation also exhibited excellent stability under accelerated and stress conditions. The formulation is stable when stored at 25° C. for at least 3 months and 40° C. for at least 7 days, demonstrating the compatibility of the 50 mg/mL formulation with the primary container closure components. No appreciable changes were observed in color or appearance, turbidity, particulate matter, pH, protein concentration, purity as measured by SE-UPLC or CEX-UPLC and iCIEF, and potency was maintained under these conditions.

TABLE 11
Research stability of 50 mg/mL formulation stored at 2-8° C.

| Formulation | | 50 mg/ml mAb1, 10 mM L-histidine, 5% (w/v) sucrose, 1.5% (w/v) L-proline, | | | | | | | | |
|---|---|---|---|---|---|---|---|---|---|---|
| | | and 0.2% (w/v) polysorbate 80, pH 6.0 | | | | | | | | |
| Fill Volume | | 1.2 mL | | | | | | | | |
| Container/Closure | | 3 mL Type 1 glass vial with a 13 min FluorTec ® coated 4432/50 chlorobutyl stopper | | | | | | | | |
| | | Length of Storage at 2-8° C. (months) | | | | | | | | |
| | | 0 | 1 | 3 | 6 | 9 | 12 | 18 | 24 | 36 |
| Color and Appearance | | Pass | Pass | Pass | Pass | Pass | Pass | Pass | Pass | [symbol] |
| Turbidity (increase in | | 0.00 | 0.00 | 0.00 | 0.00 | 0.00 | 0.00 | 0.00 | 0.00 | [symbol] |
| OD at 405 nm) | | | | | | | | | | |
| Turbidity (NTU by [symbol] | | 2.41 | 0.01 | 6.15 | 6.61 | 6.03 | 6.48 | 6.29 | 5.81 | [symbol] |
| pH | | 6.0 | 6.0 | 6.0 | 6.1 | 6.0 | 6.0 | 6.0 | 6.0 | [symbol] |
| Subvisible | ≥10 μm | 2 | NR | 21 | 8 | NR | 11 | 15 | 21 | [symbol] |
| Particulate | ≥25 μm | 0 | NR | 2 | 0 | NR | 1 | 1 | 1 | [symbol] |
| Analysis by HIAC | | | | | | | | | | |
| (N/mL) | | | | | | | | | | |
| Subvisible | 20 to 10 μm | 248 | NR | 887 | 1875 | NR | 607 | 1254 | 776 | [symbol] |
| Particulate | ≥10 μm | 15 | NR | 26 | 44 | NR | 15 | 19 | 8 | [symbol] |
| Analysis by MFI | ≥25 μm | 4 | NR | 3 | 17 | NR | 1 | 3 | 1 | [symbol] |
| (N/mL) | | | | | | | | | | |
| % Protection Recovered by RP-UPLC | | 100 | 100 | 98 | 103 | 101 | 105 | 98 | 98 | [symbol] |
| Purity by | Non-reduced, | 99.2 | NR | NR | 98.7 | NR | 98.9 | 99.2 | 99.2 | [symbol] |
| MCE-SDS | % main peak | | | | | | | | | |
| | Reduced, | 100 | NR | NR | 100 | NR | 99.6 | 99.8 | 99.9 | [symbol] |
| | % heavy + | | | | | | | | | |
| | light chain | | | | | | | | | |
| Purity by | % HMW | 6.5 | 6.3 | 0.3 | 0.4 | 0.4 | 0.5 | 0.5 | 0.5 | [symbol] |
| SE-PLC | % Monomer | 99.2 | 99.1 | 99.2 | 99.2 | 99.1 | 99.2 | 99.2 | 99.1 | [symbol] |
| | % LMW | 0.4 | 0.5 | 0.4 | 0.4 | 0.5 | 0.3 | 0.4 | 0.4 | [symbol] |
| Charge variant | % Acidic | 18.9 | 19.0 | 18.7 | 19.1 | 19.4 | 10.1 | 26.7 | 20.5 | [symbol] |
| Analysis by | % Main | 93.9 | 53.5 | 54.5 | 53.2 | 51.6 | 56.8 | 53.8 | 53.4 | [symbol] |
| CEX-UPLC | % Basic | 27.3 | 27.6 | 26.8 | 27.2 | 27.1 | 25.1 | 25.5 | 26.0 | [symbol] |
| Charge variant | % Acidic | 31.9 | NR | NR | 32.3 | NR | 33.9 | 33.1 | 34.0 | [symbol] |
| Analysis by | % Main | 54.7 | NR | NR | 54.2 | NR | 54.0 | 54.0 | 53.9 | [symbol] |
| iCIEF | % Basic | 13.5 | NR | NR | 13.5 | NR | 12.1 | 12.9 | 12.1 | [symbol] |

| | | | | | | | | | |
|---|---|---|---|---|---|---|---|---|---|
| % Relative Potency (bioassay) | 126 | NR | NR | 127 | NR | 125 | 119 | 104 | [symbol] |

CEX, Cation exchange;
DS, drug substance;
HMW, High molecular weight;
iCIEF, imaged capillary isoelectric-focusing;
LMW, Low molecular weight;
MFI, Microflow-imaging;
Monomer, intact antibody;
NR, Not required;
OD, Optical density;
RP, Reverse phase;
SE, Size exclusion;
UPLC, Ultra-performance liquid chromatography [symbol]
indicates data missing or illegible when filed

TABLE 12
Research stability of 175 mg/mL formulation stored at 2-8° C.

| Formulation | | 175 mg/ml mAb1, 10 mM L-histidine, 5% (w/v) sucrose, 1.5% (w/v) L-proline, | | | | | | | | |
|---|---|---|---|---|---|---|---|---|---|---|
| | | and 0.2% (w/v) polysorbate 80, pH 6.0 | | | | | | | | |
| Fill Volume | | 1.2 mL | | | | | | | | |
| Container/Closure | | 3 mL Type 1 glass vial with a 13 min FluorTec ® coated 4432/50 chlorobutyl stopper | | | | | | | | |
| | | Length of Storage at 2-8° C. (months) | | | | | | | | |
| | | 0 | 1 | 3 | 6 | 9 | 12 | 18 | 24 | 36 |
| Color and Appearance | | Pass | Pass | Pass | Pass | Pass | Pass | Pass | Pass | [symbol] |
| Turbidity (increase in | | 0.00 | 0.00 | 0.00 | 0.00 | 0.00 | 0.00 | 0.00 | 0.00 | [symbol] |
| OD at 405 nm) | | | | | | | | | | |
| Turbidity (NTU by [symbol] | | 6.72 | 0.93 | 0.57 | 6.54 | 6.62 | 7.01 | 6.67 | 6.87 | [symbol] |
| pH | | 6.0 | 6.0 | 6.0 | 6.0 | 6.0 | 6.0 | 6.0 | 6.1 | [symbol] |
| Subvisible | ≥10 μm | 10 | NR | 28 | 131 | NR | 36 | 35 | 55 | [symbol] |
| Particulate | ≥25 μm | 0 | NR | 11 | 56 | NR | 1 | 2 | 3 | [symbol] |
| Analysis by HIAC | | | | | | | | | | |
| (N/mL) | | | | | | | | | | |
| Subvisible | 20 to 10 μm | 144 | NR | 441 | 244 | NR | 263 | 319 | 117 | [symbol] |
| Particulate | ≥10 μm | 22 | NR | 36 | 22 | NR | 29 | 28 | 8 | [symbol] |
| Analysis by MFI | ≥25 μm | 1 | NR | 11 | 7 | NR | 5 | 10 | 1 | [symbol] |
| (N/mL) | | | | | | | | | | |
| % Protection Recovered by RP-UPLC | | 100 | 95 | 97 | 98 | 97 | 99 | 104 | 101 | [symbol] |
| Purity by | Non-reduced, | 97.9 | NR | 98.1 | 98.0 | NR | 97.9 | 98.0 | 97.9 | [symbol] |
| MCE-SDS | % main peak | | | | | | | | | |
| | Reduced, | 100 | NR | 99.6 | 100 | NR | 99.8 | 99.8 | 99.8 | [symbol] |
| | % heavy + | | | | | | | | | |
| | light chain | | | | | | | | | |
| Purity by | % HMW | 0.6 | 0.6 | 0.7 | 0.7 | 0.8 | 0.9 | 1.0 | 1.0 | [symbol] |
| SE-PLC | % Monomer | 99.1 | 98.9 | 99.1 | 98.7 | 98.7 | 98.7 | 98.6 | 98.6 | [symbol] |
| | % LMW | 0.4 | 0.5 | 0.3 | 0.5 | 0.5 | 0.5 | 0.4 | 0.4 | [symbol] |
| Charge variant | % Acidic | 18.7 | 18.3 | 18.1 | 18.7 | 19.1 | 19.6 | 19.0 | 20.3 | [symbol] |
| Analysis by | % Main | 55.6 | 55.0 | 54.6 | 53.3 | 53.6 | 55.5 | 54.3 | 53.8 | [symbol] |
| CEX-UPLC | % Basic | 27.8 | 26.7 | 27.4 | 28.1 | 27.3 | 25.5 | 26.8 | 25.9 | [symbol] |
| Charge variant | % Acidic | 11.8 | NR | 12.4 | 81.9 | NR | 33.9 | 32.3 | 32.0 | [symbol] |
| Analysis by | % Main | 54.6 | NR | 53.6 | 54.6 | NR | 54.8 | 54.6 | 54.3 | [symbol] |
| iCIEF | % Basic | 13.6 | NR | 13.1 | 13.2 | NR | 11.3 | 13.1 | 13.2 | [symbol] |

| | | | | | | | | | |
|---|---|---|---|---|---|---|---|---|---|
| % Relative Potency (bioassay) | 102 | NR | NR | 118 | NR | 110 | 91 | 107 | [symbol] |

CEX, Cation exchange;
DS, drug substance;
HMW, High molecular weight;
iCIEF, imaged capillary isoelectric-focusing;
LMW, Low molecular weight;
MFI, Microflow-imaging;
Monomer, intact antibody;
NR, Not required;
OD, Optical density;
RP, Reverse phase;
SE, Size exclusion;
UPLC, Ultra-performance liquid chromatography [symbol]
indicates data missing or illegible when filed

TABLE 13
Research stability of 50 mg/mL formulation stored at accelerated and stress
conditions

| Formulation | | 50 mg/mL mAb1, 10 mM L-histidine, 5% (w/v) sucrose, 1.5% (w/v) L-proline, | | | | | | |
|---|---|---|---|---|---|---|---|---|
| | | 0.2% (w/v) polysorbate 80, pH 6.0 | | | | | | |
| Fill Volume | | 1.2 mL | | | | | | |
| Container/Closure | | 3 mL Type 1 glass vials with a 13 mm FluroTex [symbol] coated 4432/50 chlorobutyl | | | | | | |
| | | stopper | | | | | | |
| | | | 25° C./60% RH Storage | | | | | |
| | | | (months) | | | 40° C. Storage (days) | | |
| Assay | | 0 | 1 | 3 | 6 | 7 | 14 | 28 |
| Color and appearance | | Pass | Pass | Pass | Pass | Pass | Pass | Pass |
| Turbidity (Increase in OD at 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.0 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced, | 99.2 | NR | 99.1 | 98.8 | NR | NR | 98.9 |
| MCE-SDS | % main peak | | | | | | | |
| | Reduced, | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| [symbol] IEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| [symbol] IEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 7.41 | 6.09 | 6.34 | 6.90 | 6.10 | 6.60 | 6.95 |
| pH | | 6.0 | 6.0 | 6.0 | 6.1 | 6.0 | 6.0 | 6.0 |
| Subvisible | ≥10 μm | 2 | NR | 17 | 21 | NR | NR | 18 |
| particulate | ≥25 μm | 0 | NR | 0 | 1 | NR | NR | 1 |
| analysis by | | | | | | | | |
| HLAC [symbol] | | | | | | | | |
| Subvisible | 2-10 μm | 248 | NR | 1618 | 1531 | NR | NR | 1543 |
| particulate | ≥10 μm | 15 | NR | 29 | 47 | NR | NR | 41 |
| analysis by MFT | ≥25 μm | 4 | NR | 2 | 15 | NR | NR | 15 |
| (g/mL) | | | | | | | | |
| % Protein recovered by RP-UPLC | | 100 | 100 | 100 | 100 | 103 | 102 | 98 |
| Purity by | Non-reduced; | 99.2 | NR | 99.1 | 98.8 | NR | NR | 98.9 |
| MCE-SDS | % main peak | | | | | | | |
| | Reduced; | 100 | NR | 99.5 | 100 | NR | NR | 99.5 |
| | % heavy + light | | | | | | | |
| Purity by | % HMW | 0.5 | 0.4 | 0.6 | 0.7 | 0.7 | 0.9 | 1.4 |
| SE-UPLC | % Monomer | 99.2 | 99.0 | 99.1 | 98.8 | 98.9 | 98.5 | 97.9 |
| | % LMW | 0.4 | 0.5 | 0.3 | 0.5 | 0.5 | 0.6 | 0.7 |
| Charge virtual | % Acidic | 18.9 | 19.3 | 21.8 | 27.4 | 19.9 | 22.3 | 27.2 |
| analysis by CEX- | % Main | 53.9 | 53.4 | 52.1 | 48.1 | 52.3 | 50.1 | 46.8 |
| UPLC | % Basic | 27.3 | 27.4 | 26.1 | 24.8 | 27.8 | 27.4 | 26.0 |
| Charge variant | % Acidic | 31.9 | NR | 38.1 | 43.7 | NR | NR | 45.4 |
| analysis by | % Main | 34.7 | NR | 48.6 | 44.3 | NR | NR | 42.1 |
| iCIEF | % Basic | 13.5 | NR | 13.3 | 12.0 | NR | NR | 12.5 |

| | | | | | | | |
|---|---|---|---|---|---|---|---|
| % Relative potency by bioassay | 126 | NR | NR | 120 | NR | NR | 99 |

CEX, Cation exchange;
DS, Drug substance;
HMW, high molecular weight;
iCiEF, imaged capilliary isoclectric-focusing;
LMW, Low molecular weight;
MFI, Microflow-imaging;
Monomer, intact antibody;
NR, Not required;
OD, Optical density;
RP, Reverse phase;
SE, Size excision;
UPLC, Ultra-performance liquid chromatography [symbol]
indicates data missing or illegible when filed

TABLE 14
Research stability of 175 mg/mL formulation stored at accelerated and stress
conditions

| Formulation | | 175 mg/mL mAb1, 10 mM L-histidine, 5% (w/v) sucrose, 1.5% (w/v) L-proline, | | | | | | |
|---|---|---|---|---|---|---|---|---|
| | | 0.2% (w/v) polysorbate 80, pH 6.0 | | | | | | |
| Fill Volume | | 1.2 mL | | | | | | |
| Container/Closure | | 3 mL Type 1 glass vials with a 13 mm FluroTec [symbol] coated 4432/50 chlorobutyl stopper | | | | | | |
| | | | 25° C./60% RH Storage | | | | | |
| | | | (months) | | | 40° C. Storage (days) | | |
| Assay | | 0 | 1 | 3 | 6 | 7 | 14 | 28 |
| Color and appearance | | Pass | Pass | Pass | Pass | Pass | Pass | Pass |
| Turbidity (Increase in OD as 405 nm) | | 0.00 | 0.00 | 0.00 | 0.00 | 0.00 | 0.00 | 0.00 |
| Turbidity (NTU by [symbol] ) | | 6.72 | 6.77 | 6.82 | 6.99 | 6.98 | 7.30 | 7.66 |
| pH | | 6.0 | 6.0 | 6.0 | 6.0 | 6.0 | 5.9 | 6.0 |
| Subvisible | ≥10 μm | 10 | NR | 28 | 58 | NR | NR | 97 |
| particulate | ≥25 μm | 0 | NR | 13 | 16 | NR | NR | 44 |
| analysis by | | | | | | | | |
| HLAC [symbol] | | | | | | | | |
| Subvisible | 2-10 μm | 144 | NR | 623 | 277 | NR | NR | 1131 |
| particulate | ≥10 μm | 22 | NR | 25 | 8 | NR | NR | 531 |
| analysis by MFT | ≥25 μm | 1 | NR | 3 | 2 | NR | NR | 3 |
| (g/mL) | | | | | | | | |
| % Protein recovered by RP-UPLC | | 100 | 95 | 97 | 99 | 98 | 99 | 95 |
| Purity by | Non-reduced, | 97.9 | NR | NR | 97.4 | NR | NR | 97.5 |
| MCE-SDS | % main peak | | | | | | | |
| | Reduced, | 100 | NR | NR | 99.7 | NR | NR | 99.4 |
| | % heavy + light | | | | | | | |
| Purity by | % HMW | 0.6 | 0.9 | 1.2 | 1.5 | 1.7 | 2.6 | 3.9 |
| SE-UPLC | % Monomer | 99.1 | 98.6 | 98.5 | 98.0 | 97.7 | 96.8 | 95.5 |
| | % LMW | 0.4 | 0.5 | 0.3 | 0.6 | 0.6 | 9.7 | 0.6 |
| Charge virtual | % Acidic | 18.7 | 18.9 | 21.3 | 26.2 | 39.7 | 22.0 | 23.9 |
| analysis by CEX- | % Main | 53.6 | 54.4 | 52.3 | 48.3 | 51.7 | 50.1 | 49.4 |
| UPLC | % Basic | 27.8 | 26.7 | 26.4 | 25.5 | 28.6 | 27.9 | 26.7 |
| Charge variant | % Acidic | 31.8 | NR | 37.0 | 45.0 | NR | NR | 47.3 |
| analysis by | % Main | 54.6 | NR | 50.3 | [symbol] | NR | NR | 39.2 |
| iCIEF | % Basic | 13.6 | NR | 12.7 | 11.7 | NR | NR | 13.5 |

| | | | | | | | |
|---|---|---|---|---|---|---|---|
| % Relative potency by bioassay | 102 | NR | NR | 123 | NR | NR | 86 |

CEX, Cation exchange;
DS, Drug substance;
HMW, high molecular weight;
iCiEF, imaged capilliary isoclectric-focusing;
LMW, Low molecular weight;
MFI, Microflow-imaging;
Monomer, intact antibody;
NR, Not required;
OD, Optical density;
RP, Reverse phase;
SE, Size excision;
UPLC, Ultra-performance liquid chromatography [symbol]
indicates data missing or illegible when filed

### Example 12: Stability of mAb1 Formulations Comprising Histidine

### Buffer, Sucrose and Polysorbate

Tables 15-24 summarize the storage stability of exemplary mAb1 formulations that comprise 10 mM histidine buffer, at pH 6.0, sucrose and polysorbate.

TABLE 15
Research stability of mAb1 formulation Stored at −80° C.

| Formulation | | 72.2 mg/mL mAb1, 10 mM histidine, pH 6.0, 5% (w/v) sucrose | | | | | |
|---|---|---|---|---|---|---|---|
| Fill Volume | | 1.0 mL | | | | | |
| Container/Closure | | 5 mL polycarbonate vial with silicone lined polypropylene screw cap | | | | | |
| | | Length of Storage at −80° C. (months) | | | | | |
| Assay | | 0 | 1 | 3 | 6 | 9 | 12 |
| Color and appearance | | Pass | Pass | Pass | Pass | Pass | Pass |
| Turbidity (Increase in OD as 405 nm) | | 0.00 | 0.00 | 0.00 | 0.00 | 0.00 | 0.00 |
| pH | | 6.2 | 6.3 | 6.2 | 6.1 | 6.2 | 6.1 |
| % Protein recovered by RP-UPLC | | 100 | 94 | 95 | 98 | 95 | 94 |
| Purity by | Non-reduced, | 99.5 | NR | NR | 99.5 | NR | 99.2 |
| MCE-SDS | % Main peak | | | | | | |
| | Reduced, | 100 | NR | NR | 100 | NR | 99.8 |
| | % heavy + light | | | | | | |
| | chain | | | | | | |
| Purity by | % HMW | 0.7 | 0.7 | 0.6 | 0.6 | 0.7 | 0.6 |
| SE-UPLC | % Native | 98.7 | 98.8 | 98.9 | 98.7 | 98.9 | 98.8 |
| | % LMW | 0.6 | 0.6 | 0.5 | 0.6 | 0.4 | 0.6 |
| Charge variant | % Acidic | 22.2 | 22.2 | 22.6 | 23.2 | 24.1 | 23.5 |
| analysis by CEX- | % Main | 49.7 | 49.8 | 48.9 | 46.8 | 45.2 | 44.1 |
| UPLC | % Basic | 28.1 | 28.0 | 28.5 | 30.0 | 30.7 | 32.4 |
| Charge variant | % Acidic | 38.9 | NR | NR | 39.1 | NR | 37.5 |
| analysis by | % Main | 56.5 | NR | NR | 56.2 | NR | 57.2 |
| iCIEF | % Basic | 4.6 | NR | NR | 4.7 | NR | 5.3 |

| | | | | | | |
|---|---|---|---|---|---|---|
| % Relative potency by bioassay | 95 | NR | NR | 81 | NR | 120 |

CEX = Cation exchange;
HMW = High molecular weight;
iCIEF = Imaged capillary isoelectric focusing;
LMW = Low molecular weight;
MCE-SDS = Microchip capillary electrophoresis-sodium dodecyl sulfate;
MFI = Microflow imaging;
NR = Not required;
OD = Optical density;
RH = Relative humidity;
RP = Reverse phase;
SE = Size exclusion;
UPLC = Ultra-performance liquid chromatography

TABLE 16
Research stability of mAb1 formulation Stored at −30° C.

| Formulation | | 72.2 mg/mL mAb1, 10 mM histidine, pH 6.0, 5% (w/v) sucrose | | | | | |
|---|---|---|---|---|---|---|---|
| Fill Volume | | 1.0 mL | | | | | |
| Container/Closure | | 5 mL polycarbonate vial with silicone lined polypropylene screw cap | | | | | |
| | | Length of Storage at −30° C. (months) | | | | | |
| Assay | | 0 | 1 | 3 | 6 | 9 | 12 |
| Color and appearance | | Pass | Pass | Pass | Pass | Pass | Pass |
| Turbidity (Increase in OD as 405 nm) | | 0.00 | 0.00 | 0.00 | 0.00 | 0.00 | 0.01 |
| pH | | 6.2 | 6.3 | 6.2 | 6.1 | 6.2 | 6.1 |
| % Protein recovered by RP-UPLC | | 100 | 93 | 95 | 100 | 96 | 96 |
| Purity by | Non-reduced, | 99.5 | NR | NR | 99.1 | NR | 99.2 |
| MCE-SDS | % Main peak | | | | | | |
| | Reduced, | 100 | NR | NR | 100 | NR | 99.8 |
| | % heavy + light | | | | | | |
| | chain | | | | | | |
| Purity by | % HMW | 0.7 | 0.7 | 0.0 | 0.7 | 0.7 | 0.6 |
| SE-UPLC | % Native | 98.7 | 98.8 | 99.0 | 98.7 | 98.9 | 98.8 |
| | % LMW | 0.6 | 0.5 | 0.4 | 0.6 | 0.4 | 0.6 |
| Charge variant | % Acidic | 22.2 | 22.5 | 22.5 | 23.4 | 24.2 | 23.4 |
| analysis by CEX- | % Main | 49.7 | 49.6 | 48.9 | 46.6 | 45.6 | 44.1 |
| UPLC | % Basic | 28.1 | 28.0 | 28.6 | 30.0 | 30.2 | 32.5 |
| Charge variant | % Acidic | 38.9 | NR | NR | 38.2 | NR | 37.8 |
| analysis by | % Main | 56.5 | NR | NR | 56.3 | NR | 56.6 |
| iCIEF | % Basic | 4.6 | NR | NR | 5.5 | NR | 5.7 |

TABLE 17
Research stability of mAb1 formulation Stored at −20° C.

| Formulation | | 72.2 mg/mL mAb1, 10 mM histidine, pH 6.0, 5% (w/v) sucrose | | | | | |
|---|---|---|---|---|---|---|---|
| Fill Volume | | 1.0 mL | | | | | |
| Container/Closure | | 5 mL polycarbonate vial with silicone lined polypropylene screw cap | | | | | |
| | | Length of Storage at −20° C. (months) | | | | | |
| Assay | | 0 | 1 | 3 | 6 | 9 | 12 |
| Color and appearance | | Pass | Pass | Pass | Pass | Pass | Pass |
| Turbidity (Increase in OD as 405 nm) | | 0.00 | 0.00 | 0.01 | 0.01 | 0.00 | 0.01 |
| pH | | 6.2 | 6.3 | 6.2 | 6.1 | 6.2 | 6.1 |
| % Protein recovered by RP-UPLC | | 100 | 95 | 97 | 101 | 98 | 98 |
| Purity by | Non-reduced, | 99.5 | NR | NR | 99.5 | NR | 99.3 |
| MCE-SDS | % Main peak | | | | | | |
| | Reduced, | 100 | NR | NR | 100 | NR | 99.9 |
| | % heavy + light | | | | | | |
| | chain | | | | | | |
| Purity by | % HMW | 0.7 | 0.7 | 0.7 | 0.7 | 0.8 | 0.7 |
| SE-UPLC | % Native | 98.7 | 98.8 | 98.9 | 98.6 | 98.8 | 98.8 |
| | % LMW | 0.6 | 0.5 | 0.5 | 0.7 | 0.4 | 0.6 |
| Charge variant | % Acidic | 22.2 | 22.2 | 22.3 | 22.4 | 24.6 | 23.4 |
| analysis by CEX- | % Main | 49.7 | 49.7 | 49.2 | 47.6 | 45.5 | 44.2 |
| UPLC | % Basic | 28.1 | 27.9 | 28.5 | 30.0 | 30.0 | 32.5 |
| Charge variant | % Acidic | 38.9 | NR | NR | 38.6 | NR | 38.6 |
| analysis by | % Main | 56.5 | NR | NR | 56.8 | NR | 56.2 |
| iCIEF | % Basic | 4.6 | NR | NR | 4.6 | NR | 5.3 |

| | | | | | | |
|---|---|---|---|---|---|---|
| % Relative potency by bioassay | 95 | NR | NR | 96 | NR | 111 |

TABLE 18
Research stability of mAb1 formulation-Effect of Accelerated Conditions

| Formulation | | 72.2 mg/mL mAb1, 10 mM histidine, pH 6.0, 5% (w/v) sucrose | | | | | | |
|---|---|---|---|---|---|---|---|---|
| Fill Volume | | 1.0 mL | | | | | | |
| Container/Closure | | 5 mL polycarbonate vial with silicone lined polypropylene screw cap | | | | | | |
| | | | 5° C. Storage | | 25° C./60% RH | | 40° C./75% RH | |
| | | | (days) | | Storage (days) | | Storage (days) | |
| Assay | | T = 0 | 28 | 56 | 14 | 28 | 14 | 28 |
| Color and appearance | | Pass | Pass | Pass | Pass | Pass | Pass | Pass |
| Turbidity (Increase in OD as 405 nm) | | 0.00 | 0.01 | 0.01 | 0.02 | 0.01 | 0.03 | 0.05 |
| pH | | 6.2 | 6.2 | 6.1 | 6.2 | 6.2 | 6.2 | 6.1 |
| % Protein recovered by RP-UPLC | | 100 | 96 | 100 | 97 | 100 | 105 | 113 |
| Purity by | Non-reduced, | 99.5 | NR | 99.1 | NR | 99.5 | NR | 99.1 |
| MCE-SDS | % Main peak | | | | | | | |
| | Reduced, | 100 | NR | 100 | NR | 99.2 | NR | 98.7 |
| | % heavy + light | | | | | | | |
| | chain | | | | | | | |
| Purity by | % HMW | 0.7 | 0.9 | 1.1 | 1.4 | 1.7 | 3.1 | 4.6 |
| SE-UPLC | % Native | 98.7 | 98.5 | 98.4 | 98.0 | 97.7 | 96.1 | 94.6 |
| | % LMW | 0.6 | 0.6 | 0.6 | 0.7 | 0.7 | 0.8 | 0.9 |
| Charge variant | % Acidic | 22.2 | 22.2 | 22.3 | 22.0 | 22.6 | 25.6 | 30.8 |
| analysis by CEX- | % Main | 49.7 | 49.8 | 48.9 | 49.6 | 49.1 | 46.0 | 41.7 |
| UPLC | % Basic | 28.1 | 28.1 | 28.8 | 28.4 | 28.3 | 28.3 | 27.3 |
| Charge variant | % Acidic | 38.9 | NR | 39.0 | NR | 41.1 | NR | 58.2 |
| analysis by | % Main | 56.5 | NR | 56.8 | NR | 53.8 | NR | 36.3 |
| iCIEF | % Basic | 4.6 | NR | 4.2 | NR | 5.1 | NR | 5.5 |

| | | | | | | | |
|---|---|---|---|---|---|---|---|
| % Relative potency by bioassay | 95 | NR | 95 | NR | 84 | NR | 87 |

TABLE 19
Research stability of mAb1 formulation Stored at −80° C.

| Formulation | | 25 mg/mL mAb1, 10 mM histidine, pH 6.0, 5% (w/v) sucrose | | | | | |
|---|---|---|---|---|---|---|---|
| Fill Volume | | 1.0 mL | | | | | |
| Container/Closure | | 5 mL polycarbonate vial with silicone lined polypropylene screw cap | | | | | |
| | | Length of Storage at −80° C. (months) | | | | | |
| Assay | | 0 | 1 | 3 | 6 | 9 | 12 |
| Color and appearance | | Pass | Pass | Pass | Pass | Pass | Pass |
| Turbidity (Increase in OD as 405 nm) | | 0.00 | 0.00 | 0.00 | 0.00 | 0.01 | 0.01 |
| pH | | 6.1 | 6.1 | 6.1 | 6.0 | 6.0 | 6.0 |
| % Protein recovered by RP-UPLC | | 100 | 100 | 97 | 95 | 100 | 99 |
| Purity by | Non-reduced, | 99.5 | NR | NR | 99.4 | NR | 99.5 |
| MCE-SDS | % Main peak | | | | | | |
| | Reduced, | 100 | NR | NR | 100 | NR | 100 |
| | % heavy + light | | | | | | |
| | chain | | | | | | |
| Purity by | % HMW | 0.8 | 0.8 | 0.8 | 0.8 | 0.8 | 0.8 |
| SE-UPLC | % Native | 98.6 | 98.7 | 98.6 | 98.6 | 98.8 | 98.7 |
| | % LMW | 0.6 | 0.5 | 0.6 | 0.6 | 0.5 | 0.5 |
| Charge variant | % Acidic | 22.8 | 23.0 | 23.3 | 23.5 | 23.9 | 23.4 |
| analysis by CEX- | % Main | 47.3 | 47.3 | 46.2 | 45.3 | 44.2 | 44.2 |
| UPLC | % Basic | 30.0 | 29.8 | 30.6 | 31.2 | 31.9 | 30.4 |
| Charge variant | % Acidic | 40.1 | NR | NR | 38.1 | NR | 41.3 |
| analysis by | % Main | 56.1 | NR | NR | 57.4 | NR | 54.1 |
| iCIEF | % Basic | 3.8 | NR | NR | 4.6 | NR | 4.6 |

| | | | | | | |
|---|---|---|---|---|---|---|
| % Relative potency by bioassay | 112 | NR | NR | 130 | NR | 107 |

TABLE 20
Research stability of mAb1 formulation Stored at −30° C.

| Formulation | | 25 mg/mL mAb1, 10 mM histidine, pH 6.0, 10% (w/v) sucrose, 0.1% polysorbate 80 | | | | | |
|---|---|---|---|---|---|---|---|
| Fill Volume | | 1.0 mL | | | | | |
| Container/Closure | | 5 mL polycarbonate vial with silicone lined polypropylene screw cap | | | | | |
| | | Length of Storage at −30° C. (months) | | | | | |
| Assay | | 0 | 1 | 3 | 6 | 9 | 12 |
| Color and appearance | | Pass | Pass | Pass | Pass | Pass | Pass |
| Turbidity (Increase in OD as 405 nm) | | 0.00 | 0.00 | 0.00 | 0.00 | 0.01 | 0.01 |
| pH | | 6.1 | 6.1 | 6.1 | 6.0 | 6.1 | 6.0 |
| % Protein recovered by RP-UPLC | | 100 | 100 | 102 | 97 | 100 | 102 |
| Purity by | Non-reduced, | 99.5 | NR | NR | 99.1 | NR | 99.5 |
| MCE-SDS | % Main peak | | | | | | |
| | Reduced, | 100 | NR | NR | 100 | NR | 100 |
| | % heavy + light | | | | | | |
| | chain | | | | | | |
| Purity by | % HMW | 0.8 | 0.8 | 0.8 | 0.8 | 0.8 | 0.8 |
| SE-UPLC | % Native | 98.6 | 98.7 | 98.6 | 98.7 | 98.8 | 98.7 |
| | % LMW | 0.6 | 0.5 | 0.6 | 0.6 | 0.4 | 0.6 |
| Charge variant | % Acidic | 22.8 | 21.1 | 22.9 | 23.5 | 23.7 | 25.1 |
| analysis by CEX- | % Main | 47.3 | 47.1 | 46.5 | 45.3 | 44.4 | 44.4 |
| UPLC | % Basic | 30.0 | 29.8 | 30.6 | 31.2 | 31.9 | 30.5 |
| Charge variant | % Acidic | 40.1 | NR | NR | 38.0 | NR | 41.3 |
| analysis by | % Main | 56.1 | NR | NR | 57.3 | NR | 53.3 |
| iCIEF | % Basic | 3.8 | NR | NR | 4.7 | NR | 5.4 |

TABLE 21
Research Stability of mAb1 Formulation Stored at −20° C.

| Formulation | | 25 mg/mL mAb1, 10 mM histidine, pH 6.0, 10% | | | | | |
|---|---|---|---|---|---|---|---|
| | | (w/v) sucrose, 0.1% polysorbate 80 | | | | | |
| Fill Volume | | 1.0 mL | | | | | |
| Container/Closure | | 5 mL polycarbonate vial with silicone lined | | | | | |
| | | polypropylene screw cap | | | | | |
| | | Length of Storage at −20° C. (months) | | | | | |
| Assay | | 0 | 1 | 3 | 6 | 9 | 12 |
| Color and appearance | | Pass | Pass | Pass | Pass | Pass | Pass |
| Turbidity (Increase in OD at 405 | | 0.00 | 0.00 | 0.00 | 0.00 | 0.01 | 0.00 |
| nm) | | | | | | | |
| pH | | 6.1 | 6.1 | 6.1 | 6.0 | 6.1 | 6.0 |
| % Protein recovered by RP-UPLC | | 100 | 99 | 102 | 97 | 106 | 102 |
| Purity by | Non-reduced; | 99.5 | NR | NR | 99.4 | NR | 99.4 |
| MCE-SDS | % Main peak | | | | | | |
| | Reduced; | 100 | NR | NR | 100 | NR | 99.0 |
| | % Heavy + light | | | | | | |
| | chain | | | | | | |
| Purity by | % HMW | 0.8 | 0.8 | 0.8 | 0.8 | 0.7 | 0.8 |
| SE-UPLC | % Native | 98.6 | 98.7 | 98.6 | 98.7 | 98.8 | 98.7 |
| | % LMW | 0.6 | 0.5 | 0.6 | 0.6 | 0.4 | 0.6 |
| Charge | % Acidic | 22.8 | 22.9 | 23.5 | 23.6 | 24.3 | 25.2 |
| variant | % Main | 47.3 | 47.3 | 46.0 | 45.2 | 43.8 | 43.9 |
| analysis by | % Basic | 30.0 | 29.8 | 30.6 | 31.2 | 31.9 | 30.8 |
| CEX-UPLC | | | | | | | |
| Charge | % Acidic | 40.1 | NR | NR | 38.4 | NR | 41.6 |
| variant | % Main | 56.1 | NR | NR | 57.9 | NR | 53.3 |
| analysis by | % Basic | 3.8 | NR | NR | 3.7 | NR | 5.1 |
| iCIEP | | | | | | | |

| | | | | | | |
|---|---|---|---|---|---|---|
| % Relative potency (Bioassay) | 112 | NR | NR | 120 | NR | 131 |

TABLE 22
Research Stability of mAb1 Formulation-Effect of Accelerated Conditions

| Formulation | | 25 mg/mL mAb1, 10 mM histidine, pH 6.0, 10% | | | | | | |
|---|---|---|---|---|---|---|---|---|
| | | (w/v) sucrose, 0.1% polysorbate 80 | | | | | | |
| Fill Volume | | 1.0 mL | | | | | | |
| Container/Closure | | 5 mL polycarbonate vial with silicone lined | | | | | | |
| | | polypropylene screw cap | | | | | | |
| | | | | | | | 40° C./75% RH | |
| | | No | 5° C. Storage | | 25° C./60% RH | | Storage | |
| | | Storage | (days) | | Storage (days) | | (days) | |
| Assay | | T = 0 | 28 | 56 | 14 | 28 | 14 | 28 |
| Color and appearance | | Pass | Pass | Pass | Pass | Pass | Pass | Pass |
| Turbidity (Increase in OD at 405 | | 0.00 | 0.00 | 0.00 | 0.00 | 0.00 | 0.01 | 0.03 |
| nm) | | | | | | | | |
| pH | | 6.1 | 6.1 | 6.0 | 6.1 | 6.1 | 6.0 | 6.0 |
| % Protein recovered by RP-UPLC | | 100 | 101 | 102 | 106 | 107 | 113 | 114 |
| Purity by | Non-reduced; | 99.5 | NR | 99.1 | NR | 99.5 | NR | 99.3 |
| MCE-SDS | % Main peak | | | | | | | |
| | Reduced; | 100 | NR | 100 | NR | 100 | NR | 99.1 |
| | % Heavy + light | | | | | | | |
| | chain | | | | | | | |
| Purity by | % HMW | 0.8 | 0.7 | 0.7 | 0.9 | 0.9 | 3.4 | 5.0 |
| SE-UPLC | % Native | 98.0 | 98.8 | 98.7 | 98.6 | 98.6 | 95.7 | 93.7 |
| | % LMW | 0.6 | 0.5 | 0.6 | 0.5 | 0.6 | 0.9 | 1.3 |
| Charge | % Acidic | 22.8 | 22.8 | 23.3 | 22.6 | 22.8 | 29.6 | 32.5 |
| variant | % Main | 47.3 | 47.3 | 46.1 | 47.2 | 47.0 | 39.5 | 36.4 |
| analysis by | % Basic | 30.0 | 29.9 | 30.6 | 30.2 | 30.2 | 30.9 | 31.1 |
| CEX-UPLC | | | | | | | | |
| Charge | % Acidic | 40.1 | NR | 39.8 | NR | 40.2 | NR | 53.7 |
| variant | % Main | 56.1 | NR | 57.0 | NR | 55.9 | NR | 42.2 |
| analysis by iCIEF | % Basic | 3.8 | NR | 3.2 | NR | 3.9 | NR | 4.0 |

| | | | | | | | |
|---|---|---|---|---|---|---|---|
| % Relative potency (Bioassay) | 112 | NR | 103 | NR | 91 | NR | 79 |

TABLE 23
Research Stability of mAb1 formulation Stored at 5° C.

| Formulation | | 25 mg/mL mAb1, 10 mM histidine, 10% (w/v) | | | | | |
|---|---|---|---|---|---|---|---|
| | | sucrose, 0.1% (w/v) polysorbate 80, pH 6.0 | | | | | |
| Fill Volume | | 1.0 mL | | | | | |
| Container/Closure | | 2 mL Type 1 borosilicate glass vials with a 13 mm | | | | | |
| | | FluroTec ® coated West S2-451 4432/50 GRY B2- | | | | | |
| | | 40 stoppers | | | | | |
| | | Length of Storage at 5° C. (months) | | | | | |
| Assay | | 0 | 1 | 3 | 6 | 9 | 12 |
| Color and appearance | | Pass | Pass | Pass | Pass | Pass | Pass |
| Turbidity (Increase in OD at 405 nm) | | 0.00 | 0.00 | 0.00 | 0.01 | 0.01 | 0.00 |
| pH | | 6.2 | 6.1 | 6.1 | 6.1 | 6.0 | 6.1 |
| Particulate | 2 to 10 μm | 823 | NR | NR | 2169 | NR | 29331 |
| analysis by MFI | ≥10 μm | 15 | NR | NR | 10 | NR | 56 |
| (particles/mL) | ≥25 μm | 0 | NR | NR | 1 | NR | 5 |
| % Protein recovered by RP-UPLC | | 100 | 100 | 98 | 101 | 102 | 90 |
| Purity by MCE- | Non-reduced; | 99.4 | NR | NR | 99.3 | NR | 99.2 |
| SDS | % main peak | | | | | | |
| | Reduced; | 100 | NR | NR | 100 | NR | 100 |
| | % heavy + light chain | | | | | | |
| Purity by | % HMW | 0.7 | 0.7 | 0.7 | 0.8 | 0.9 | 0.9 |
| SE-UPLC | % Native | 98.7 | 98.8 | 98.7 | 98.6 | 98.6 | 98.5 |
| | % LMW | 0.0 | 0.5 | 0.0 | 0.6 | 0.5 | 0.6 |
| Charge variant | % Acidic | 23.0 | 22.8 | 22.3 | 22.4 | 24.1 | 22.4 |
| analysis by | % Main | 48.5 | 48.6 | 46.2 | 47.2 | 44.4 | 45.5 |
| CEX-UPLC | % Basic | 28.6 | 28.6 | 30.4 | 30.4 | 31.5 | 32.1 |
| Charge variant | % Acidic | 39.8 | NR | NR | 39.2 | NR | 40.1 |
| analysis by | % Main | 56.7 | NR | NR | 56.5 | NR | 55.7 |
| iCIEF | % Basic | 3.4 | NR | NR | 4.3 | NR | 4.1 |

| | | | | | | |
|---|---|---|---|---|---|---|
| % Relative potency (bioassay) | 111 | NR | NR | 132 | NR | 124 |

TABLE 24
Research Stability of mAb1 formulation Stored at Accelerated Conditions

| Formulation | | 25 mg/mL mAb1, 10 mM histidine, 10% (w/v) | | | | | | |
|---|---|---|---|---|---|---|---|---|
| | | sucrose, 0.1% (w/v) polysorbate 80, pH 6.0 | | | | | | |
| Fill Volume | | 1.0 mL | | | | | | |
| Container/Closure | | 2 mL Type 1 borosilicate glass vials with a 13 mm | | | | | | |
| | | FluroTec ® coated West S2-451 4432/50 GRY B2-40 | | | | | | |
| | | stoppers | | | | | | |
| | | | 25° C./60% RH Storage | | | | | |
| | | | (months) | | | 45º C. Storage (days) | | |
| Assay | | 0 | 0.5 | 1 | 3 | 7 | 14 | 28 |
| Color and appearance | | Pass | Pass | Pass | Pass | Pass | Pass | Pass |
| Turbidity (Increase in OD at 405 nm) | | 0.00 | 0.01 | 0.01 | 0.00 | 0.01 | 0.00 | 0.02 |
| pH | | 6.2 | 6.1 | 6.1 | 6.1 | 6.2 | 6.1 | 6.1 |
| Particulate | 2 to 10 μm | 823 | NR | NR | 1056 | NR | NR | 523 |
| analysis by | ≥10 μm | 15 | NR | NR | 23 | NR | NR | 83 |
| MFI (particles/mL) | ≥25 μm | 0 | NR | NR | 3 | NR | NR | 4 |
| % Protein recovered by RP-UPLC | | 100 | 99 | 100 | 99 | 99 | 100 | 99 |
| Purity by | Non-reduced; | 99.4 | NR | NR | 99.4 | NR | NR | 99.2 |
| MCE-SDS | % main peak | | | | | | | |
| | Reduced; | 100 | NR | NR | 100 | NR | NR | 98.6 |
| | % heavy + high chain | | | | | | | |
| Purity by | % HMW | 0.7 | 0.8 | 0.9 | 1.1 | 1.6 | 3.2 | 8.5 |
| SE-UPLC | % Native | 98.7 | 98.6 | 98.5 | 98.1 | 97.6 | 95.9 | 89.7 |
| | % LMW | 0.6 | 0.6 | 0.6 | 0.8 | 0.8 | 1.0 | 1.8 |
| Charge variant | % Acidic | 23.0 | 22.6 | 23.1 | 25.8 | 24.7 | 27.3 | 34.0 |
| analysis by | % Main | 48.5 | 48.7 | 48.1 | 44.5 | 46.2 | 44.1 | 35.9 |
| CEX-UPLC | % Basic | 28.6 | 28.8 | 28.9 | 29.8 | 29.1 | 28.6 | 30.2 |
| Charge variant | % Acidic | 39.8 | NR | NR | 47.1 | NR | NR | 66.9 |
| analysis by | % Main | 56.7 | NR | NR | 49.5 | NR | NR | 30.2 |
| iCIEF | % Basic | 3.4 | NR | NR | 3.5 | NR | NR | 3.0 |

| | | | | | | | |
|---|---|---|---|---|---|---|---|
| % Relative potency by bioassay | 111 | NR | NR | 94 | NR | NR | 63 |

Tables 25-27 summarize the stress stability of exemplary formulations.

TABLE 25
Research Stability of mAb1 formulation-Effect of Stress Conditions

| Formulation | | 72.2 mg/mL mAb1, 10 mM histidine, pH 6.0, 5% (w/v) sucrose | | | | |
|---|---|---|---|---|---|---|
| Fill Volume | | 1.0 mL | | | | |
| Container/ | | 5 mL polycarbonate vial with silicone lined | | | | |
| Closure | | polypropylene screw cap | | | | |
| | | | | | Freeze/Thaw | |
| | | | Agitation (min) | | (cycles) | |
| Assay | | T = 0 | 10 | 15 | 4 | 8 |
| Color and appearance | | Pass | Pass | Pass | Pass | Pass |
| Turbidity (Increase in OD at | | 0.00 | 0.00 | 0.09 | 0.01 | 0.00 |
| 405 nm) | | | | | | |
| pH | | 6.2 | 6.2 | 6.2 | 6.3 | 6.3 |
| % Protein Recovered by | | 100 | 100 | 99 | 95 | 95 |
| RP-UPLC | | | | | | |
| Purity by | Non-reduced; | 99.5 | NR | 99.2 | NR | 99.4 |
| MCE-SDS | % Main peak | | | | | |
| | Reduced; | 100 | NR | 100 | NR | 100 |
| | % Heavy + | | | | | |
| | light chain | | | | | |
| Purity by | % HMW | 0.7 | 0.8 | 4.6 | 0.6 | 0.7 |
| SE-UPLC | % Native | 98.7 | 98.7 | 94.9 | 98.9 | 98.8 |
| | % LMW | 0.6 | 0.5 | 0.5 | 0.5 | 0.5 |
| Charge | % Acidic | 22.2 | 22.2 | 21.9 | 22.0 | 22.2 |
| variant | % Main | 49.7 | 49.6 | 49.9 | 49.7 | 49.6 |
| analysis by | % Basic | 28.1 | 28.2 | 28.2 | 28.3 | 28.2 |
| CEX-UPLC | | | | | | |
| Charge | % Acidic | 38.0 | NR | 40.8 | NR | 39.0 |
| variant | % Main | 56.5 | NR | 54.7 | NR | 56.5 |
| analysis by | % Basic | 4.6 | NR | 4.6 | NR | 4.5 |
| iCIEF | | | | | | |

| | | | | | |
|---|---|---|---|---|---|
| % Relative Potency (Bioassay) | 95 | NR | 87 | NR | 89 |

TABLE 26
Research Stability of mAb1 Formulation-Effect of Stress Conditions

| Formulation | | 25 mg/mL mab1, 10 mM histidine, pH 6.0, 10% (w/v) | | | | |
|---|---|---|---|---|---|---|
| | | sucrose, 0.1% (w/v) polysorbate 80 | | | | |
| Fill Volume | | 1.0 mL | | | | |
| Container/Closure | | 5 mL polycarbonate vial with silicone lined polypropylene screw cap | | | | |
| | | No Stress | Agitation (minutes) | | Freeze/Thaw (cycles) | |
| Assay | | T = 0 | 60 | 120 | 4 | 8 |
| Color and appearance | | Pass | Pass | Pass | Pass | Pass |
| Turbidity (Increase in | | 0.00 | 0.00 | 0.00 | 0.00 | 0.00 |
| OD at 405 nm) | | | | | | |
| pH | | 6.1 | 6.1 | 6.1 | 6.1 | 6.1 |
| % Protein recovered by | | 100 | 101 | 100 | 99 | 100 |
| RP-UPLC | | | | | | |
| Purity by | Non-reduced; | 99.3 | NR | 99.5 | NR | 99.6 |
| MCE-SDS | % Main peak | | | | | |
| | Reduced; | 100 | NR | 100 | NR | 100 |
| | % Heavy + light | | | | | |
| | chain | | | | | |
| Purity by | % HMW | 0.8 | 0.8 | 0.7 | 0.8 | 0.8 |
| SE-UPLC | % Native | 98.6 | 98.7 | 98.7 | 98.7 | 98.6 |
| | % LMW | 0.6 | 0.6 | 0.6 | 0.5 | 0.6 |
| Charge | % Acidic | 22.8 | 22.8 | 22.8 | 22.9 | 22.7 |
| variant | % Main | 47.3 | 47.2 | 47.3 | 47.1 | 47.2 |

TABLE 27
Research Stability of mAb1 formulation-Effect of Stress Conditions

| Formulation | | 25 mg/mL mAb1, 10 mM histidine, 10% (w/v) sucrose, 0.1% | | | | |
|---|---|---|---|---|---|---|
| | | (w/v) polysorbate 80, pH 6.0 | | | | |
| Fill Volume | | 1.0 mL | | | | |
| Container/Closure | | 2 mL Type 1 borosilicate glass vials with a 13 mm FluroTec ® | | | | |
| | | coated West S2-451 4432/50 GRY B2-40 stoppers | | | | |
| | | | Agitation | | Freeze/ | |
| | | | (min) | | Thaw (cycles) | |
| Assay | | 0 | 60 | 120 | 4 | 8 |
| Color and appearance | | Pass | Pass | Pass | Pass | Pass |
| Turbidity (Increase in OD at 405 nm) | | 0.00 | 0.00 | 0.00 | 0.00 | 0.00 |
| pH | | 6.2 | 6.2 | 6.2 | 6.2 | 6.2 |
| Particulate | 2 to 10 μm | 823 | NR | 1170 | NR | 1404 |
| analysis by MFI | ≥10 μm | 15 | NR | 38 | NR | 67 |
| (particles/mL) | ≥25 μm | 0 | NR | 0 | NR | 2 |
| % Protein recovered by RP-UPLC | | 100 | 100 | 101 | 101 | 101 |
| Purity by | Non-reduced; | 99.4 | NR | 99.4 | NR | 99.3 |
| MCE-SDS | % main peak | | | | | |
| | Reduced; | 100 | NR | 100 | NR | 100 |
| | % heavy + light chain | | | | | |
| Purity by | % HMW | 0.7 | 0.6 | 0.7 | 0.7 | 0.7 |
| SE-UPLC | % Native | 98.7 | 98.8 | 98.7 | 98.7 | 98.6 |
| | % LMW | 0.6 | 0.6 | 0.6 | 0.6 | 0.7 |
| Charge variant | % Acidic | 23.0 | 22.9 | 22.8 | 22.8 | 22.7 |
| analysis by | % Main | 48.5 | 48.3 | 48.4 | 48.4 | 48.5 |
| CEX-UPLC | % Basic | 28.6 | 28.8 | 28.8 | 28.8 | 28.8 |
| Charge variant | % Acidic | 39.8 | NR | 39.6 | NR | 39.6 |
| analysis by | % Main | 56.7 | NR | 56.9 | NR | 56.9 |
| iCIEF | % Basic | 3.4 | NR | 3.4 | NR | 3.6 |

| | | | | | |
|---|---|---|---|---|---|
| % Relative potency by bioassay | 111 | NR | 90 | NR | 129 |

TABLE 28
Accelerated and Stress Stability of 50 mg/mL and 25 mg/mL of mAb1 Formulations

| Formulation | | 50 mg/mL mAb1, 10 mM histidine, | | | 25 mg/mL mAb1, 10 mM histidine, | | |
|---|---|---|---|---|---|---|---|
| | | 10% (w/v) sucrose, 0.1% (w/v) | | | 10% (w/v) sucrose, 0.1% (w/v) | | |
| | | polysorbate 80, pH 6.0 | | | polysorbate 80, pH 6.0 | | |
| Fill Volume | | 1.0 mL | | | 1.0 mL | | |
| Container/Closure | | 2 mL Type 1 borosilicate glass vials | | | 2 mL Type 1 borosilicate glass vials | | |
| | | with a 13 mm FluroTec ® coated | | | with a 13 mm FluroTec ® coated | | |
| | | West S2-451 4432/50 GRY B2-40 | | | West S2-451 4432/50 GRY B2-40 | | |
| | | stoppers | | | stoppers | | |
| | | | 25° C./60% RH | 45° C. | | 25° C./60% RH | 45° C. |
| Assay | | T = 0 | 1 month | 28 days | T = 0 | 1 month | 28 days |
| Color and appearance | | Pass | Pass | Pass | Pass | Pass | Pass |
| Turbidity (Increase in OD at 405 nm) | | 0.00 | 0.01 | 0.02 | 0.00 | 0.01 | 0.02 |
| pH | | 6.0 | 6.0 | 6.1 | 6.2 | 6.1 | 6.1 |
| % Protein recovered by RP-UPLC | | 100 | 103 | 113 | 100 | 100 | 99 |
| Purity by | % HMW | 1.9 | 4.0 | 10.8 | 0.7 | 0.9 | 8.5 |
| SE-UPLC | % Monomer | 97.5 | 95.2 | 88.0 | 98.7 | 98.5 | 89.7 |
| | % LMW | 0.6 | 0.8 | 1.2 | 0.6 | 0.6 | 1.8 |
| Charge variant | % Acidic | 24.6 | 29.0 | 37.1 | 23.0 | 23.1 | 34.0 |
| analysis by | % Main | 49.9 | 42.5 | 33.2 | 48.5 | 48.1 | 35.9 |
| CEX-UPLC | % Basic | 25.6 | 28.5 | 29.7 | 28.6 | 28.9 | 30.2 |

### Example 13: Proven Acceptable Range (PAR) Study

During the manufacture of mAb1 drug product (DP), variations in the composition of the DP may occur. These variations may include the concentration of the active ingredient, the concentration of the excipients, and/or the pH of the formulation. Because changes in any of these parameters could potentially impact the stability or potency of the drug product, proven acceptable range (PAR) studies were conducted to assess whether variations in the DP composition, within the defined ranges, would impact the stability or potency of mAb1 DP.

Two Design-Of-Experiment (DOE) studies were used to evaluate the effect of each formulation parameter as well as the interactions on the formulation stability:
A fractional factorial design Pre-PAR study with accelerated and stress stability assessment to identify critical formulation parameters that may impact mAb1 DP stability;
A full factorial design PAR study including critical formulation parameters identified from the Pre-PAR study, with long-term shelf-life stability to demonstrate the acceptable ranges of the formulation parameters.

### Pre-PAR Study Design

To assess critical and/or interacting formulation parameters in the DP composition that might be important to product quality, a fractional factorial DOE was applied to examine the accelerated and stress stability of formulations by varying all formulation parameters, including protein concentration (±10%), buffer and stabilizer concentrations (±20%), surfactant concentration (±50%), and pH (±0.3 unit). The tested formulation parameter ranges were defined to be equal or wider than the specification acceptance criteria and manufacturing experience. The study was designed with a statistical software using a 2 ^(6-2) resolution IV fractional factorial experiment. Together with four target formulations as the center points, the study included 20 runs, as shown in Table 29.

TABLE 29
Formulations tested in the Pre-PAR study

| | | | % | % | % | |
|---|---|---|---|---|---|---|
| Formu- | [mAb1] | [histidine] | sucrose | L-proline | polysorbate | |
| lation | mg/mL | mM | (w/v) | (w/v) | 80 (w/v) | pH |
| 1 | 45 | 12 | 4 | 1.2 | 0.3 | 6.3 |
| 2 | 45 | 12 | 4 | 1.8 | 0.3 | 5.7 |
| 3 | 45 | 8 | 6 | 1.8 | 0.3 | 6.3 |
| 4 | 50 | 10 | 5 | 1.5 | 0.2 | 6.0 |
| 5 | 45 | 8 | 4 | 1.8 | 0.1 | 6.3 |
| 6 | 55 | 8 | 6 | 1.2 | 0.1 | 6.3 |
| 7 | 55 | 8 | 4 | 1.2 | 0.3 | 6.3 |
| 8 | 50 | 10 | 5 | 1.5 | 0.2 | 6.0 |
| 9 | 55 | 12 | 6 | 1.8 | 0.3 | 6.3 |
| 10 | 45 | 12 | 6 | 1.2 | 0.1 | 6.3 |
| 11 | 55 | 8 | 4 | 1.8 | 0.3 | 5.7 |
| 12 | 55 | 8 | 6 | 1.8 | 0.1 | 5.7 |
| 13 | 45 | 12 | 6 | 1.8 | 0.1 | 5.7 |
| 14 | 55 | 12 | 6 | 1.2 | 0.3 | 5.7 |
| 15 | 50 | 10 | 5 | 1.5 | 0.2 | 6.0 |
| 16 | 45 | 8 | 6 | 1.2 | 0.3 | 5.7 |
| 17 | 55 | 12 | 4 | 1.8 | 0.1 | 6.3 |
| 18 | 45 | 8 | 4 | 1.2 | 0.1 | 5.7 |
| 19 | 50 | 10 | 5 | 1.5 | 0.2 | 6.0 |
| 20 | 55 | 12 | 4 | 1.2 | 0.1 | 5.7 |

All 20 formulations at the accelerated conditions and the stress conditions (25° C., 37° C., freeze/thaw [F/T] and agitation) were characterized and assessed for physical/chemical properties and stability, including visual inspection, pH, turbidity, osmolality, conductivity, purity, protein concentration and recovery, charge variant analysis, and sub-visible particulate analysis.

### Pre-PAR Study Results

All 20 formulations showed no change after agitation or F/T stress. Results from 25° C. and 37° C. incubation were analyzed by a regression model (JMP fit model with standard least square personality and effect leverage emphasis). Statistical analysis of the main and interacting variables of all experimental formulations against the critical quality attributes revealed that pH, protein concentration and sucrose concentration were important to product quality. The two product quality attributes impacted were HMW species and acidic charge variants. Other formulation parameters, including histidine, proline or polysorbate 80 concentrations within the ranges tested, were found to have no statistically significant impact on the product quality. Under accelerated conditions, there was no secondary or higher interactions that impact formulation stability. The pre-PAR study results indicated that pH, mAb1 concentration, and sucrose concentration were critical to the mAb1 formulation stability, and were considered as the critical formulation parameters for the 50 mg/ml mAb1 formulation.

Although pH, mAb1 concentration and sucrose concentration were identified as the critical formulation parameters, the impact of these three factors on the quality attributes was minimal. Based on the statistical analysis, the change in pH range of 5.7-6.3, 45-55 mg/ml of mAb1, and/or 4-6% of sucrose likely had <15% impact on the formation of HMW species and acidic charge variants.

To confirm the impact on the long-term storage stability at the recommended DP storage condition, the following three critical formulation parameters: pH, mAb1 concentration, and sucrose concentration, were further evaluated in a PAR study with long-term storage stability.

### PAR Study Design

A full factorial DOE design was applied to examine the long-term shelf-life storage stability of formulations with varying pH (±0.3 unit), protein concentration (±10%), and sucrose concentration (±20%), resulting in eight experimental runs (Table 30); a reference formulation (formulation 3, the target formulation in Table 30) was included as the center point formulation.

TABLE 30
Formulations tested in PAR studies

| | | | | % L- | | |
|---|---|---|---|---|---|---|
| Formu- | [mAb1] | [histidine] | % sucrose | proline | % polysorbate | |
| lation | mg/mL | mM | (w/v) | (w/v) | 80 (w/v) | pH |
| 1 | 55 | 10 | 6 | 1.5 | 0.2 | 5.7 |
| 2 | 45 | 10 | 6 | 1.5 | 0.2 | 5.7 |
| 3 | 50 | 10 | 5 | 1.5 | 0.2 | 6.0 |
| 4 | 55 | 10 | 6 | 1.5 | 0.2 | 6.3 |
| 5 | 55 | 10 | 4 | 1.5 | 0.2 | 6.3 |
| 6 | 55 | 10 | 4 | 1.5 | 0.2 | 5.7 |
| 7 | 45 | 10 | 4 | 1.5 | 0.2 | 6.3 |
| 8 | 45 | 10 | 4 | 1.5 | 0.2 | 5.7 |
| 9 | 45 | 10 | 6 | 1.5 | 0.2 | 6.3 |

The full factorial study design allows the estimation of all main effect terms as well as the interaction terms. The tested formulation parameter ranges, defined to be equal or wider than the specification acceptance criteria and manufacturing experience, remained the same as in the Pre-PAR study.

The stability of the experimental formulations was compared to the stability of a reference formulation at pH 6.0 containing all formulation components at their nominal concentrations (F3). PAR studies utilized a mAb1 DS lot manufactured with the representative commercial manufacturing process. DP formulations were filled into 10 mL Schott type 1 borosilicate glass vials, with 20 mm FluroTec®-coated West S2-451 4432/50 GRY B2-40 stoppers (commercial DP representation) and assessed for long-term storage stability at 2-8° C. The formulations were studied according to the analysis plan in Table 31.

TABLE 31
Analysis plan for PAR study

| Text | Samples Analyzed | Quality Attributes |
|---|---|---|
| Appearance | All | Color, visible particulates |
| Turbidity | All | Color, particulates, clanty |
| (Increase in OD at | | |
| 405 nm) | | |
| Tubidity by | t = 0.6, 12, 18, | Clarity (Solution |
| Nephelometry | 24, and 36 | Precipitation, Particulates, |
| | months at 3° C. | Opulescence) |
| pH | All | pH |
| Total protein content | t = 0 | Protein concentration |
| by RP-UPLC | | |
| Osmolality | t = 0 | Solute concentration |
| Conductivity | t = 0 | Conductive property |
| Purity by SE-UPLC | All | Molecule weight |
| | | variants: % HMW, |
| | | % Monomer, % LWM |
| Charge Variant | 1 = 0, 6, 12, | Charges isoforms: |
| Analysis by | 18, 24, and 36 | % Acidic, % Main, |
| cIEF | months at 5° C. | % Basic |
| Particulate | t = 0, 6, 12, | Subvisible particulates. |
| Matter (Light | 18, 24, and 36 | Acceptance criteria set |
| Obscuration) | months at 5° C. | forth in USP <788> (<6000) |
| | | particles/container |
| | | for particles ≥10 μm |
| | | and <600 particles/container |
| | | for particles ≥25 μm) |
| Particulate | t = 0, 6, 32, 18, | Subvisible particulates |
| Matter (MicroFlow | 24, and 36 | (For information only) |
| Imaging, MFI) | months at 5° C. | |
| Bioassay | t = 0, 6, 12, 18, | Potency |
| | 24, and 36 | Acceptance criteria: |
| | months at 5° C. | 50-130% of reference |
| | | standard |

### PAR Study Results

### Effect on the HMW Species Formation

There was no meaningful increase in the % HMW as measured by SE-UPLC up to 12 months at 2-8° C. for all 9 formulations. All values were well below the upper specification, and no significant increase in % HMW over time was observed.

The HMW species formation for 50 mg/mL mAb1 DP at 2-8° C. was found to be extremely slow. For up to 12 months, the maximum change of relative amount in % HMW in the 9 formulation was ^{˜}0.2%. Since the change in % HMW was minimal, and monomer concentration could be considered as a constant, the aggregation from monomers to HMW species could be simplified as a zero order reaction. Therefore a simplified linear model was used to analyze the % HMW stability data. By linearly fitting the % HMW over time, the HMW species formation rate was derived for each formulation.

The rate was analyzed against the main factors as well as all interaction terms using a regression model (JMP fit model with standard least square personality and effect leverage emphasis). The resulted regression model was statistically significant with an R2 of 0.74. mAb1 concentration, pH, and time were statistically significant, but the effect on the % HMW species formation was statistically insignificant, only contributing up to 0.1%.

Therefore, these factors, pH at 5.7-6.3, mAb1 concentration at 45-55 mg/mL, and sucrose concentration at 4-6%, had no practical relevance to the % HMW stability at 2-8° C.

% HMW in all 9 formulations up to 12-month time-point were well below the defined acceptance criteria limit of 4% and thus within the release and end of shelf-life specifications. In addition, the linear models predicted that after 24 months of shelf-life storage at 2° C. . . . 8° C., the % HMW, ranging from 0.6% to 0.8%, would also be well below the specification limit.

Based on the long-term storage stability data, the variations of the critical formulation parameters within the studied ranges were found to have no significant impact on the mAb1 formulation stability. The 50 mg/ml mAb1 formulation was robust with regards to HMW species formation within the tested formulation composition range.

### Effect on the Acidic Charge Variants Formation

There was no meaningful increase in the % acidic charge variants measured up to 12 months at 2-8° C. for all 9 formulations. All values were below upper specification, and no significant increase in % acidic charge variants over time was observed.

The mAb1 formulation was considered to be robust with regard to acidic charge variants formation within the tested formulation composition range.

### Effect on General Quality Attributes

The effect of pH, mAb1 concentration, and sucrose, as well as storage time on other DP general quality attributes, including appearance, pH, turbidity, subvisible particulates, protein recovery, % monomer and % LMW by SEC, % main and % basic charge variants by iCIEF, and bioactivity were studied. All values were within specification, and no meaningful change over time or difference between the PAR formulations was observed:
No precipitate or visible particulate was detected by either visual inspection or turbidity measurements (OD at 405 nm and nephelometry);
No statistically significant changes in protein recovery were observed (RP-UPLC);
The pH of the formulations was stable;
No meaningful increases in subvisible particulates, and no meaningful differences were observed in the subvisible particulate counts between PAR study formulations.
For subvisible particulates measured by HIAC, all values were below the acceptable limits set by USP <788>, and no meaningful variation in subvisible particles between formulations was observed.
In additions, the subvisible particles were also measured by MFI. No meaningful variation in subvisible particles between formulations was observed.
The bioassay results were within the specification limit for all formulations during storage.

The results demonstrate that variations of the critical formulation parameters (pH, mAb1 concentration, and sucrose concentration) within the studied ranges have no significant impact on the mAb1 formulation stability. The 50 mg/ml mAb1 formulation is robust with regards to general quality attributes within the tested formulation composition range.

### Effect of Freezing and Thawing on the Stability of PAR Formulations

The physical and chemical stability of 50 mg/ml mAb1 formulation, examined following two freezing and thawing cycles, was unaffected by variation in critical formulation parameters, i.e. a ±0.3 pH unit change relative to the reference mAb1, a ±10% variation in mAb1 concentration, and/or a ±20% variation in sucrose.

The Following Effects were Observed:
No precipitate was detected by either visual inspection or turbidity measurements (OD at 405 nm);
No loss of protein was observed (RP-UPLC);
The pH of the formulations remained constant;
No meaningful differences were observed in the subvisible particulate counts, determined by light obscuration (HIAC) or micro flow imaging (MFI) between the study formulations. There was a slight increase in subvisible particles after 2 cycles of F/T, which could be removed by filtering through a 0.22 μm filter prior to filling the DP.
No appreciable changes in purity, as determined by SE-UPLC, were observed in all formulations following two freezing and thawing cycles;
No appreciable change in the distribution of charge variants, as determined by iCIEF, was observed in all formulations following two freezing and thawing cycles.
The bioassay results demonstrated that mAb1 activity was maintained in all formulations subjected to 2 cycles of freezing and thawing.

Design-Of-experiment (DOE)-based pre-PAR and PAR studies were used to evaluate the effect of formulation parameters as well as the interactions on the formulation stability. The pre-PAR study with accelerated and stress stability identified pH, mAb1 concentration, and sucrose concentration as the critical formulation parameters. A full factorial PAR study with long-term shelf-life stability demonstrated that variation in the critical formulation parameters, within the range studies, did not affect mAb1 DP quality.

Specifically, the stability and potency of 50 mg/ml mAb1 DP stored at 5° C. for 12 months were unaffected by a ±10% variation in protein concentration, a ±20% variation in sucrose, L-proline and/or histidine concentration, and/or ±50% variation in polysorbate 80 concentration, and/or a ±0.3 pH unit variation.

The robustness of mAb1 formulation was demonstrated by the PAR study. Overall, the results from the pre-PAR and PAR study supported that variability in the compositions of the mAb1 formulation within the ranges studied would not adversely impact the stability of the mAb1 DP under the recommended storage conditions (2 to 8° C.).

The 50 mg/ml mAb1 FDS samples were stable after two cycles of freezing and thawing (−30° C. freeze and room temperature thaw). The stability of mAb1 FDS to freeze/thaw stress was unaffected by a ±10% change in mAb1 concentration, a ±20% change in sucrose, and/or a ±0.3 pH unit change relative to the control mAb1 FDS (50 mg/mL). The results from these freeze/thaw studies provide support that 50 mg/ml mAb1 FDS can be frozen and thawed during the manufacture of mAb1 DP without adversely impacting the stability of the FDS.

### Example 14: Containers

The mAb1 formulations were developed in glass vials (for delivery by intravenous infusion). The container for mAb1 drug product intended for later clinical development and product commercialization is also a pre-filled syringe, which is presented as either a stand-alone syringe for self-injection or incorporated into an auto injector device for self-administration.

### Example 15: Stability of mAb1 Formulation in Glass Vials

Tables 32-35 summarize the stability of exemplary mAb1 formulations in 10 mL glass vials.

TABLE 32
Research stability of mAb1 formulation stored at 2-8° C.

| Formulation | | 50 mg/mL mAb1, 10 mM L-histidine, 5% (w/v) sucrose, 1.5% (w/v) | | | | | | | |
|---|---|---|---|---|---|---|---|---|---|
| | | L-proline, and 0.2% (w/v) polysorbate 80, pH 6.0 | | | | | | | |
| Fill Volume | | 5.5 mL | | | | | | | |
| Container/Closure | | 10 mL Type 1 glass vials with a 20 mm FluroTec ®-coated 4432/50 | | | | | | | |
| | | chlorobutyl stopper | | | | | | | |
| | | Length of Storage at 5° C. (months) | | | | | | | |
| Assay | | 0 | 1 | 3 | 6 | 9 | 12 | 18 | 24 |
| Color and Appearance | | Pass | Pass | Pass | Pass | Pass | Pass | Pass | |
| Turbidity (Increase in OD at 405 nm) | | 0.00 | 0.00 | 0.00 | 0.00 | 0.00 | 0.00 | 0.00 | |
| pH | | 6.0 | 6.0 | 6.0 | 6.0 | 6.0 | 6.0 | 6.0 | |
| Subvisible | 2 to 10 μm | 175 | NR | 61 | 772 | NR | 1730 | NR | |
| Particulate Analysis | ≥10 μm | 8 | NR | 3 | 144 | NR | 18 | NR | |
| by MPI (N/mL) | ≥25 μm | 0 | NR | 1 | 19 | NR | 4 | NR | |
| % Protein Recovered by SE-UPLC | | 100 | 102 | 103 | 101 | 105 | 103 | 104 | |
| Purity by | Non-reduced; | 99.1 | NR | NR | 99.1 | NR | 99.2 | NR | |
| MCE-SDS | % main peak | | | | | | | | |
| | Reduced; % | 100 | NR | NR | 100 | NR | 100 | NR | |
| | heavy + | | | | | | | | |
| | light chain | | | | | | | | |
| Purity by SE-UPLC | % HMW | 0.3 | 0.3 | 0.3 | 0.4 | 0.4 | 0.4 | 0.4 | |
| | % Monomer | 99.1 | 99.3 | 99.3 | 99.3 | 99.2 | 99.2 | 99.2 | |
| | % LMW | 0.0 | 0.4 | 0.4 | 0.4 | 0.4 | 0.4 | 0.4 | |
| Charge Variant | % Acidic | 18.9 | 18.9 | 18.9 | 19.8 | 20.5 | 19.8 | 18.9 | |
| Analysis by | % Main | 53.3 | 53.3 | 53.4 | 54.7 | 34.3 | 34.0 | 52.9 | |
| CEX-UPLC | % Basic | 27.5 | 27.8 | 27.7 | 25.6 | 25.2 | 26.2 | 28.2 | |
| Charge Variant | % Acidic | 33.8 | NR | 34.3 | 34.2 | NR | 34.9 | NR | |
| Analysis by iCIEX | % Main | 54.8 | NR | 54.2 | 54.3 | NR | 54.3 | NR | |
| | % Basic | 11.4 | NR | 11.4 | 11.4 | NR | 10.8 | NR | |

| | | | | | | | |
|---|---|---|---|---|---|---|---|
| % Relative Potency (bioassay) | 92 | NR | NR | 122 | NR | 112 | NR |

CEX, Cation exchange;
DS, Drug substance;
HMW, High molecular weight;
iCIEF, imaged capillary isoelectric-focusing;
LMW, Low molecular weight;
MFI, Microflow-imaging;
NR, Not required;
OD, Optical density;
RP, Reverse phase;
SE, Size exclusion;
UPLC, Ultra-performance liquid chromatography

TABLE 33
Research stability of mAb1 formulation at accelerated and stress conditions

| Formulation | | 50 mg/mL mAb1, 10 mM L-histidine, 5% (w/v) sucrose, | | | | | | |
|---|---|---|---|---|---|---|---|---|
| | | 1.5% (w/v) L-proline, and 0.2% (w/v) polysorbate 80, pH 6.0 | | | | | | |
| Fill Volume | | 5.5 mL | | | | | | |
| Container/Closure | | 10 mL Type 1 glass vials with a 20 mm FluroTec ®-coated 4432/50 | | | | | | |
| | | chlorobutyl stopper | | | | | | |
| | | | 25° C./60% RH Storage | | | | | |
| | | | (months) | | | 40° C. Storage (days) | | |
| Assay | | 0 | 1 | 3 | 6 | 7 | 14 | 28 |
| Color and appearance | | Pass | Pass | Pass | Pass | Pass | Pass | Pass |
| Turbidity (Increase in OD | | 0.00 | 0.00 | 0.00 | 0.00 | 0.00 | 0.00 | 0.00 |
| at 405 nm) | | | | | | | | |
| pH | | 6.0 | 6.0 | 6.0 | 6.0 | 6.0 | 6.0 | 6.0 |
| Subvisible | 2-10 μm | 176 | NR | 126 | 2083 | NR | NR | 288 |
| articulate | ≥10 μm | 8 | NR | 4 | 109 | NR | NR | 14 |
| analysis by | ≥25 pm | 0 | NR | 3 | 3 | NR | NR | 1 |
| MPI (N/mL) | | | | | | | | |
| % Protein recovered by SE-UPLC | | 100 | 102 | 104 | 101 | 100 | 101 | 102 |
| Purity by | Non-reduced; | 99.1 | NR | NR | 98.8 | NR | NR | 98.6 |
| MCE-SDS | % main peak | | | | | | | |
| | Reduced; | 100 | NR | NR | 99.7 | NR | NR | 99.7 |
| | % heavy + | | | | | | | |
| | light chain | | | | | | | |
| Purity by | % LMW | 0.3 | 0.4 | 0.5 | 0.6 | 0.5 | 0.8 | 1.2 |
| SE-UPLC | % Monomer | 99.1 | 99.1 | 99.1 | 99.9 | 98.7 | 98.5 | 98.1 |
| | % LMW | 0.6 | 0.5 | 0.4 | 0.4 | 0.7 | 0.7 | 0.7 |
| Charge | % Acidic | 18.9 | 19.4 | 22.1 | 28.3 | 20.0 | 22.0 | 22.1 |
| variant | % Main | 53.5 | 52.7 | 51.2 | 48.6 | 51.9 | 50.2 | 46.7 |
| analysis by | % Basic | 27.5 | 27.9 | 26.8 | 23.2 | 27.1 | 27.7 | 26.2 |
| CEX-UPLC | | | | | | | | |
| Charge | % Acidic | 33.8 | NR | NR | 43.9 | NR | NR | 46.4 |
| variant | % Main | 34.8 | NR | NR | 46.5 | NR | NR | 40.9 |
| analysis by | % Basic | 11.4 | NR | NR | 13.6 | NR | NR | 12.7 |
| iCIEX | | | | | | | | |

| | | | | | | | |
|---|---|---|---|---|---|---|---|
| % Relative potency by bioassay | 92 | NR | NR | 91 | NR | NR | 83 |

CEX, Cation exchange;
DS, Drug substance;
HMW, High molecular weight;
iCIEF, imaged capillary isoelectric-focusing;
LMW, Low molecular weight;
MFI, Microflow-imaging;
NR, Not required;
OD, Optical density;
RP, Reverse phase;
SE, Size exclusion;
UPLC, Ultra-performance liquid chromatography

TABLE 34
Research stability of mAb1 formulation stored at 2-8° C.

| Formulation | | 50 mg/mL mAb1, 10 mM L-histidine, 5% (w/v) sucrose, 1.5% (w/v) | | | | | | | |
|---|---|---|---|---|---|---|---|---|---|
| | | L-proline, and 0.2% (w/v) polysorbate 80, pH 6.0 | | | | | | | |
| Fill Volume | | 7.44 mL | | | | | | | |
| Container/Closure | | 10 mL Type 1 glass vials with a 20 mm FluroTec ®-coated 4432/50 | | | | | | | |
| | | chlorobutyl stopper | | | | | | | |
| | | Length of Storage at 2-8° C. (months) | | | | | | | |
| Assay | | 0 | 1 | 3 | 6 | 9 | 12 | 18 | 24 |
| Color and Appearance | | Pass | Pass | Pass | Pass | Pass | | | |
| Turbidity (Increase in OD at 405 nm) | | 0.00 | 0.00 | 0.00 | 0.00 | 0.00 | | | |
| pH | | 6.0 | 6.0 | 6.0 | 6.0 | 6.0 | | | |
| Subvisible | ≥10 μm | 3 | NR | 2 | 4 | NR | | | |
| Particulate | ≥25 μm | 0 | NR | 0 | 1 | NR | | | |
| Analysis by HLAC | | | | | | | | | |
| (N/mL) | | | | | | | | | |
| Subvisible | 2 to 10 μm | 183 | NR | 267 | 1044 | NR | | | |
| Particulate | ≥10 μm | 3 | NR | 7 | 19 | NR | | | |
| Analysis by MFI | ≥25 μm | 1 | NR | 3 | 5 | NR | | | |
| (N/mL) | | | | | | | | | |
| % Protein Recovered by KP-UPLC | | 100 | 99 | 102 | 102 | 98 | | | |
| Purity by | Non-reduced; | 99.2 | NR | 99.4 | 99.2 | NR | | | |
| MCE-SDS | % main peak | | | | | | | | |
| | Reduced; % | 100 | NR | 100 | 100 | NR | | | |
| | heavy + light | | | | | | | | |
| | chain | | | | | | | | |
| Purity by | % HMW | 0.7 | 0.6 | 0.5 | 0.5 | 0.6 | | | |
| SE-UPLC | % Monomer | 98.8 | 98.8 | 98.9 | 99.0 | 98.9 | | | |
| | % LMW | 0.6 | 0.6 | 0.5 | 0.4 | 0.5 | | | |
| Charge Variant | % Acidic | 21.2 | 21.2 | 22.3 | 19.6 | 21.5 | | | |
| Analysis by | % Main | 52.0 | 52.1 | 52.0 | 51.9 | 52.0 | | | |
| CEX-UPLC | % Basic | 26.8 | 26.8 | 25.7 | 28.6 | 26.6 | | | |
| Charge Variant | % Acidic | 34.3 | NR | 35.7 | 34.6 | NR | | | |
| Analysis by iCIEF | % Main | 32.1 | NR | 51.9 | 51.9 | NR | | | |
| | % Basic | 13.2 | NR | 13.4 | 13.4 | NR | | | |

| | | | | | |
|---|---|---|---|---|---|
| % Relative Potency (bioassay) | 113 | NR | 105 | 128 | NR |

CEX, Cation exchange;
DS, Drug substance;
HMW, High molecular weight;
iCIEF, imaged capillary isoelectric-focusing;
LMW, Low molecular weight;
MPI, Microflow-imaging;
NR, Not required;
OD, Optical density;
RP, Reverse phase;
SE, Size exclusion;
UPLC, Ultra-performance liquid chromatography

TABLE 35
Research stability of mAb1 formulation at accelerated and stress conditions

| Formulation | | 50 mg/mL mAb1, 10 mM L-histidine, 5% (w/v) sucrose, 1.5% (w/v) | | | | | | |
|---|---|---|---|---|---|---|---|---|
| | | L-proline, and 0.2% (w/v) polysorbate 80, pH 6.0 | | | | | | |
| Fill Volume | | 7.44 mL | | | | | | |
| Container/Closure | | 10 mL Type 1 glass vials with a 20 mm FluroTec ®-coated 4432/50 | | | | | | |
| | | chlorobutyl stopper | | | | | | |
| | | | 25 °C./60% RH Storage | | | | | |
| | | | (months) | | | 40° C. Storage (days) | | |
| Assay | | 0 | 1 | 3 | 6 | 7 | 14 | 28 |
| Color and appearance | | Pass | Pass | Pass | Pass | Pass | Pass | Pass |
| Turbidity (Increase in OD at 405 nm) | | 0.00 | 0.00 | 0.00 | 0.00 | 0.00 | 0.00 | 0.00 |
| pH | | 6.0 | 6.0 | 6.0 | 6.0 | 6.0 | 6.0 | 6.0 |
| Subvisible | ≥10 μm | 3 | NR | 4 | 11 | NR | NR | 5 |
| Particulate | ≥25 μm | 0 | NR | 0 | 2 | NR | NR | 0 |
| Analysis by | | | | | | | | |
| HLAC | | | | | | | | |
| Subvisible | 2-10 μm | 183 | NR | 540 | 1439 | NR | NR | 799 |
| particulate | ≥10 μm | 3 | NR | 18 | 16 | NR | NR | 92 |
| analysis by | ≥25 μm | 1 | NR | 1 | 1 | NR | NR | 1 |
| MFI (N/mL) | | | | | | | | |
| % Protein recovered by SE-UPLC | | 100 | 100 | 101 | 101 | 101 | 99 | 100 |
| Purity by | Non-reduced; | 99.2 | NR | 99.3 | 98.7 | NR | NR | 98.6 |
| MCE-SDS | % main peak | | | | | | | |
| | Reduced; | 100 | NR | 100 | 100 | NR | NR | 99.5 |
| | % heavy + | | | | | | | |
| | light chain | | | | | | | |
| Purity by | % HMW | 0.7 | 0.6 | 0.7 | 0.8 | 0.6 | 0.8 | 1.3 |
| SE-UPLC | % Monomer | 98.8 | 98.8 | 98.8 | 98.7 | 98.7 | 95.5 | 97.9 |
| | % LMW | 0.6 | 0.7 | 0.6 | 0.6 | 0.7 | 0.8 | 0.8 |
| Charge variant | % Acidic | 21.2 | 21.7 | 25.3 | 24.4 | 20.5 | 23.2 | 25.8 |
| anaylsis by | % Main | 52.0 | 52.1 | 51.4 | 50.3 | 52.4 | 51.0 | 49.7 |
| CEX-UPLC | % Basic | 26.8 | 26.2 | 23.3 | 25.1 | 27.0 | 25.9 | 24.6 |
| Charge | % Acidic | 34.3 | NR | 40.0 | 45.4 | NR | NR | 46.9 |
| variant | % Main | 52.1 | NR | 47.1 | 43.7 | NR | NR | 39.7 |
| analysis by | % Basic | 13.7 | NR | 12.9 | 11.0 | NR | NR | 13.3 |
| iCIEF | | | | | | | | |

| | | | | | | | |
|---|---|---|---|---|---|---|---|
| % Relative potency by bioassay | 113 | NR | 143 | 99 | NR | NR | 88 |

CEX, Cation exchange;
DS, Drug substance;
HMW, High molecular weight;
iCIEF, imaged capillary isoelectric-focusing;
LMW, Low molecular weight;
MFL, Microflow-imaging;
NR, Not required;
OD, Optical density;
RP, Reverse phase;
SE, Size exclusion;
UPLC, Ultra-performance liquid chromatography

The two formulations at different fill volumes were found to be stable to stress (40° C. 175% RH) (data not shown).

### Example 16: Stability of mAb1 Formulation in Pre-Filled Syringes

Tables 36-38 summarize the stability of high concentration mAb1 formulations in pre-filled syringes.

TABLE 36
Research Stability of mAb1 Drug Product in Pre-filled Syringe (PFS) Stored at 5*

| Formulation | | 175 mg/mL mAb1, 10 mM histidine, 5% (w/v) sucrose, 1.5% | | | | | |
|---|---|---|---|---|---|---|---|
| | | (w/v) L-proline, 0.2% (w/v) polysorbate 80, pH 6.0 | | | | | |
| Fill Volume | | 1.2 mL | | | | | |
| Container/Closure | | Nuova Ompi EZ Fill 2.25 mL glass syringe with a 27G 1/2 thin | | | | | |
| | | wall needle and FM30 needle shield closed with a FluroTec ® | | | | | |
| | | coated 4023/50 rubber plunger | | | | | |
| | | Length of Storage at 5° C. (months) | | | | | |
| Assay | | 0 | 1 | 3 | 6 | 9 | 12 |
| Color and appearance | | Pass | Pass | Pass | Pass | Pass | Pass |
| Turbidity (Increase in OD at 405 nm) | | 0.00 | 0.01 | 0.00 | 0.00 | 0.00 | 0.00 |
| pH | | 6.0 | 6.0 | 6.0 | 6.0 | 6.0 | 6.0 |
| Subvisible | ≥10 μm | 35 | NR | 17 | 59 | NR | 13 |
| particulate | ≥25 μm | 0 | NR | 4 | 1 | NR | 1 |
| analysis by | | | | | | | |
| HIAC (N/mL) | | | | | | | |
| Particulate | 2 to 10 μm | 21326 | NR | 8613 | NA | NR | NA |
| analysis by MFI | ≥10 μm | 82 | NR | 303 | NA | NR | NA |
| (particles/mL) | ≥25 μm | 3 | NR | 2 | NA | NR | NA |
| % Protein recovered by RP-UPLC | | 100 | 96 | 97 | 100 | 98 | 100 |
| Purity by MCE- | Non-reduced; | 97.6 | NR | NR | 97.7 | 97.1 | NA |
| SDS | % main peak | | | | | | |
| | Reduced; % heavy + | 99.6 | NR | NR | 99.8 | 99.8 | NA |
| | light chain | | | | | | |
| Purity by | % HMW | 0.6 | 0.6 | 0.7 | 0.7 | 0.8 | 0.9 |
| SE-UPLC | % Native | 99.1 | 98.9 | 99.1 | 98.7 | 98.7 | 98.6 |
| | % LMW | 0.1 | 0.5 | 0.2 | 0.5 | 0.5 | 0.5 |
| Charge variant | % Acidic | 18.6 | 18.5 | 18.2 | 18.6 | 19.3 | 18.8 |
| analysis by | % Main | 53.4 | 54.8 | 54.3 | 53.2 | 53.5 | 55.5 |
| CEX-UPLC | % Basic | 27.9 | 26.8 | 27.5 | 25.3 | 25.6 | 24.3 |
| Charge variant | % Acidic | 30.2 | NR | 30.2 | 32.9 | NR | NA |
| analysis by | % Main | 55.9 | NR | 55.9 | 53.4 | NR | NA |
| iCIEF | % Basic | 13.9 | NR | 13.9 | 13.7 | NR | NA |

| | | | | | | |
|---|---|---|---|---|---|---|
| % Relative potency (bioassay) | 87 | NR | NR | 141 | NR | NA |

TABLE 38
Research Stability of mAb1 Drug Product in Pre-filled Syringe
(PFS)-Effect of Stress Conditions

| Formulation | | 175 mg/ml mAb1, 10 mM | | |
|---|---|---|---|---|
| | | histidine, 5% (w/v) sucrose, | | |
| | | 1.5% (w/v) L-proline, 0.2% (w/v) | | |
| | | polysorbate 80, pH 6.0 | | |
| Fill Volume | | 1.2 mL | | |
| Container/Closure | | Nuova Ompi EZ Fill 2.25 mL | | |
| | | glass syringe with a 27G | | |
| | | ½ thin wall needle and FM30 | | |
| | | needle shield closed with | | |
| | | a Fluro Tec ® coated 4023/50 | | |
| | | rubber plunger | | |
| | | | Agitation (min) | |
| Assay | | 0 | 60 | 820 |
| Color and appearance | | Pass | Pass | Pass |
| Turbidity (Increase in OD at 405 mm) | | 0.00 | 0.00 | 0.00 |
| pH | | 6.0 | 6.0 | 6.0 |
| Subvisible | ≥10 μm | 3 | NR | 5 |
| particulate analysis | ≥25 μm | 1 | NR | 5 |
| by HLAC ( [symbol] ) | | | | |
| Particulate | 2 to 10 μm | 21326 | NR | 13896 |
| analysis by MFI | ≥10 μm | 82 | NR | 55 |
| (particles/mL) | ≥25 μm | 3 | NR | 3 |
| 5% Protein recovered by RP-UPLC | | 100 | 100 | 100 |
| Purity by | Non-reduced: | 97.6 | NR | 97.0 |
| MCE-SOS | % main peak | | | |
| | Reduced: % | 99.6 | NK | 99.3 |
| | heavy + | | | |
| | light chain | | | |
| Purity by | % HMW | 0.6 | 0.6 | 0.6 |
| SE-DPLC | % Native | 99.1 | 98.9 | 98.8 |
| | % LMW | 0.3 | 0.6 | 0.6 |
| Charge variant | % Acidic | 18.6 | 18.8 | 19.0 |
| analysis by | % Main | 53.4 | 53.2 | 53.5 |
| CEX-UPLC | % Basic | 27.9 | 28.1 | 27.5 |
| Charge variant | % Acidic | 30.2 | NR | 30.4 |
| analysis by | % Main | 55.9 | NR | 55.5 |
| iCIEF | % Basic | 13.9 | NR | 14.1 |

| | | | |
|---|---|---|---|
| % Relative potency by bioassay | 87 | NR | 100 |

[symbol]

indicates data missing or illegible when filed

### Example 17: Compatibility of mAb1 Formulations in Intravenous (IV) Delivery Devices

For the compatibility assessment, 50 mg/ml mAb1 formulation was added to a 100 mL IV bag, containing either 0.9% Sodium Chloride Injection or 5% Dextrose Injection, to assess whether mAb1 is stable when delivered intravenously. To support the variability in patient weights, two admixture concentrations, 1.0 mg/ml mAb1 and 25 mg/ml mAb1, were examined in this study to reflect the low and high dosing conditions. The following IV admixture components were used during the compatibility studies:
Drug Product
   a. 50 mg/ml mAb1 DP
Diluents
   a. 0.9% Sodium Chloride Injection
   b. 5% Dextrose Injection
IV Bags
   a. IV bags made of polyvinyl chloride (PVC) with di-(2-ethylhexyl) phthalate (DEHP) prefilled with 0.9% Sodium Chloride Injection
   b. IV bags made of polyvinyl chloride (PVC) with DEHP prefilled with 5% Dextrose Injection
   c. IV bags made of polyolefin (PO) prefilled with 0.9% Sodium Chloride Injection
   d. IV bags made of polyolefin (PO) prefilled with 5% Dextrose Injection
   e. IV bags made of polypropylene prefilled with 0.9% Sodium Chloride Injection
   f. IV bottles made of polypropylene prefilled with 0.9% Sodium Chloride Injection
IV Pumps
   a. Peristaltic pump
   b. Fluid displacement pump
IV Infusion Sets
   a. IV set made of PVC with DEHP
   b. IV set made of PVC with dioctyl terephthalate (DEHT)
   c. IV set made of PVC with trioctyl trimellitate (TOTM)
   d. IV set made of polyethylene lined PVC
   e. IV set made of polyurethane
Filters
   a. 0.2 μm polyethersulfone inline filter
   b. 1.2 μm polyethersulfone inline filter
   c. 5 μm polyethersulfone inline filter
   d. 15 μm polyethersulfone inline filter.

The DPs used in this study were GMP manufactured using a representative DP commercial manufacturing process. The IV bags containing the admixture were initially held for 24 hours at 5° C.; the bags were then incubated for at least 8 hours at 25° C. After these incubations, each of the infusion sets was connected to the IV bag, primed with the admixture and held for 1 hour at ambient room temperature. Each admixture was then pumped through the respective infusion sets at rates of 25 mL/h and 500 mL/h.

### Methods Used to Assess Admixture Compatibility:

The compatibility of the mAb1 admixture with materials used in the IV delivery device was assessed using the following assays:
Color and appearance by visual inspection
pH.
Turbidity measured by increase in Optical Density (OD) at 405 nm
Subvisible particulate analysis on admixture by light obscuration (HIAC)
Protein concentration of mAb1 by reversed-phase ultra performance liquid chromatography (RP-UPLC)
Purity by SE-UPLC
Charge variant analysis by CEX-UPLC
Potency, by bioassay: The relative potency of each sample is determined using the bioassay and is defined as: (IC50 Reference Sample/IC50 Sample)*100%. The measured potency of storage stability samples must be within 50-150% of the measured potency of the reference standard.

### Results and Conclusions

The 50 mg/ml mAb1 formulation, diluted in either 0.9% Sodium Chloride Injection or 5% Dextrose Injection to concentrations of either 1.0 mg/mL or 25 mg/mL was physically and chemically stable under all conditions tested within the proposed dose ranges and administration conditions. These data support the following conclusions pertaining to dose preparation and IV administration of mAb1 DP:
0.9% Sodium Chloride Injection and 5% Dextrose Injection IV bags made of PVC with DEHP, PO, and polypropylene are compatible with mAb1 IV administration.
mAb1 DP can be diluted to concentrations as low as 1.0 mg/mL in PVC, PO or polypropylene IV bags containing either 0.9% Sodium Chloride or 5% Dextrose for IV administration.
mAb1 DP can be diluted to concentrations as high as 25.0 mg/mL in PVC, PO or polypropylene IV bags containing either 0.9% Sodium Chloride or 5% Dextrose for IV administration.
mAb1 admixture in either 0.9% Sodium Chloride or 5% Dextrose was stable after incubation in a PVC, PO or polypropylene IV bag for periods of up to 24 hours at 5° C. and 8 hours at 25° C. The diluted mAb1 DP may be administered within 6 hours of preparation.
Diluted mAb1 can be administered using a standard infusion pump.
Diluted mAb1 can be administered with an infusion set composed of either PVC containing DEHP, PVC containing TO™, polyethylene or polyurethane.
mAb1 is compatible with the use of an inline 0.2 μm-5 μm polyethersulfone filter.
Diluted mAb1 can be administered at a rate ranging from 25 to 500 mL/hour.

Additional exemplary medicaments, drugs, and/or pharmaceutical formulations include: pharmaceuticals targeting Activin A and GDF8 (e.g., garetosmab and trevogrumab as described in U.S. Pat. No. 9,718,881, which is incorporated by reference herein); pharmaceuticals targeting C5 (e.g., pozelimab and cemdisiran, as described in US Publication 2021/0046182, which is incorporated by reference herein); pharmaceuticals targeting LEPR (e.g., mibavademab, as described in U.S. Pat. No. 10,023,644, which is incorporated by reference herein); pharmaceuticals targeting LAG3 (e.g., fianlimab, as described in U.S. Pat. No. 10,358,495, which is incorporated by reference herein); pharmaceuticals targeting BetV1 (e.g., antibodies disclosed in U.S. Pat. No. 10,793,624, which is incorporated by reference herein); pharmaceuticals targeting PCSK9 (e.g., alirocumab, as described in U.S. Pat. No. 8,795,669, which is incorporated by reference herein); pharmaceuticals targeting ANGPTL3 (e.g., evinacumab, as disclosed in US Publication 2020/0369760, which is incorporated by reference herein); pharmaceuticals targeting Ebola (e.g., atolivimab, maftivimab, odesivimab, as disclosed in US Publication 2021/0252146, which is incorporated by reference herein); pharmaceuticals targeting IL-6R (e.g., sarilumab, as disclosed in U.S. Pat. No. 9,173,880, which is incorporated by reference herein).

Additional exemplary medicaments, drugs, and/or pharmaceutical formulations include: RNAi therapeutic targeting APP for early-onset Alzheimer's disease (ALN-APP1); RNAi therapeutic targeting HSD17B13 for nonalcoholic steatohepatitis (“NASH”) (ALN-HSD); RNAi therapeutic targeting PNPLA3 for NASH (ALN-PNP1); PD-1 Antibody for First-line NSCLC, BNT116 combination (CEMIPLIMAB); Bispecific antibody targeting BCMA and CD3 for Multiple myeloma (LINVOSELTAMAB); TTR gene knockout using CRISPR/Cas9 for Transthyretin (“ATTR”) amyloidosis; Bispecific antibody targeting CD20 and CD3 for certain B-cell malignancies (ODRONEXTAMAB); Bispecific antibody targeting PSMA and CD3 for prostate cancer; Bispecific antibody targeting two distinct MET epitopes for MET-altered advanced NSCLC; Bispecific antibody-drug conjugate targeting two distinct MET epitopes for MET overexpressing advanced cancer; Agonist Antibody to NPR1/Reversal Agent to REGN5381 for reversal agent in healthy volunteers; Bispecific antibody targeting BCMA and CD3 for transplant desensitization in patients with chronic kidney disease; Bispecific antibody targeting MUC16 and CD28 for platinum-resistant ovarian cancer; Bispecific antibody targeting PSMA and CD28 for prostate cancer; Bispecific antibody targeting CD22 and CD28 for B-NHL; Antibody to GITR for solid tumors; Bispecific antibody targeting EGFR and CD28 for solid tumors; Antibody to IL2Rg for aplastic anemia; Antibody to Factor XI for thrombosis; Antibody to TMPRSS6 for transfusion dependent iron overload; Antibody to Factor XI for thrombosis; RNAi therapeutic targeting HSD17B13 for nonalcoholic steatohepatitis (“NASH”) (ALN-HSD); Antibody to PD-1 Neoadjuvant CSCC; for second-line cervical cancer, ISA101b combination (CEMIPLIMAB); Antibody to IL-4R alpha subunit for ulcerative colitis; Eosinophilic gastroenteritis (Phase 2/3) (DUPILUMAB); Antibody to LAG-3 for first-line advanced NSCLC (Phase 2/3) (pivotal study) (FIANLIMAB); Bispecific antibody targeting BCMA and CD3 for multiple myeloma (pivotal study) (LINVOSELTAMAB); Agonist antibody to leptin receptor (“LEPR”) for generalized lipodystrophy; Partial lipodystrophy (MIBAVADEMAB); Bispecific antibody targeting CD20 and CD3 for B-cell non-Hodgkin lymphoma (“B-NHL”) (pivotal study) (ODRONEXTAMAB); Antibody to C5; studied as monotherapy and in combination with cemdisiran for CD55-deficient protein-losing enteropathy (“CHAPLE”), monotherapy (potentially pivotal study) (POZELIMAB); Agonist Antibody to NPR1/Reversal Agent to REGN5381 for heart failure; Antibody to IL-6R for polyarticular-course juvenile idiopathic arthritis (“pcJIA”) (pivotal study); systemic juvenile idiopathic arthritis (“sJIA”) (pivotal study) (SARILUMAB); Bispecific antibody targeting MUC16 and CD3 for platinum-resistant ovarian cancer (UBAMATAMAB); Immune activator targeting TLR9 for solid tumors (VIDUTOLIMOD); VEGF-Trap for Wet AMD, DME (AFLIBERCEPT); Antibody to PCSK9 for HeFH in pediatrics (ALIROCUMAB); Antibody to PD-1 for adjuvant CSCC (CEMIPLIMAB); Antibody to IL-4R alpha subunit for EoE in pediatrics; chronic obstructive pulmonary disease (“COPD”); bullous pemphigoid; chronic spontaneous urticaria (“CSU”); chronic pruritis of unknown origin (DUPILUMAB); Antibody to LAG-3 for first-line metastatic melanoma; First-line adjuvant melanoma (FIANLIMAB); Antibody to Activin A for fibrodysplasia ossificans progressiva (“FOP”) (GARETOSMAB); Antibody to IL-33 for COPD (ITEPEKIMAB); Antibody to C5; studied as monotherapy and in combination with cemdisiran for myasthenia gravis, cemdisiran combination; paroxysmal nocturnal hemoglobinuria (“PNH”), cemdisiran combination (POZELIMAB); Multi-antibody therapy to Bet v 1 for birch allergy.

Notably, reference herein to “one embodiment,” or “an embodiment” means that a particular feature, structure, or characteristic described in connection with the embodiment may be included, employed and/or incorporated in one, some or all of the embodiments of the present disclosure. The usages or appearances of the phrase “in one embodiment” or “in another embodiment” in the specification are not referring to the same embodiment, nor are separate or alternative embodiments necessarily mutually exclusive of one or more other embodiments, nor limited to a single exclusive embodiment. The same applies to the terms “implementation,” and “example.” The present disclosure are neither limited to any single aspect nor embodiment thereof, nor to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein.

Further, as indicated above, an embodiment or implementation described herein as “exemplary” is not to be construed as preferred or advantageous, for example, over other embodiments or implementations; rather, it is intended convey or indicate the embodiment or embodiments are example embodiment(s).

The present disclosure is further described by the following non-limiting items:
Item 1. An injection device, comprising:
a housing;
a container disposed within the housing, the container enclosing a fluid and having a first end and a second end;
a conduit movable relative to the container, wherein the conduit is not in fluid communication with the fluid enclosed by the container while in a first position, and is in fluid communication with the fluid enclosed by the container and configured to deliver the fluid from the container to a patient while in a second position;
a lock that is removable from the housing, the lock having a first portion and a second portion, wherein:
   in a first configuration where the lock is coupled to the housing, the first portion of the lock is disposed exterior of the housing and the second portion of the lock is disposed within the housing between the container and the conduit;
   in the first configuration, the conduit is prevented from moving into fluid communication with the fluid enclosed by the container by the second portion of the lock; and
   in a second configuration where the lock is removed from the injection device, the conduit is able to move into fluid communication with the fluid enclosed by the container.
Item 2. The injection device of item 1, further including:
an adhesive disposed exterior of the housing; and
a cover disposed over the adhesive, wherein the cover is removable from the injection device to expose the adhesive,
wherein the lock is coupled to the cover such that the lock is removed from the injection device when the cover is removed from the injection device.
Item 3. The injection device of item 1, further including a controller and a skin sensor coupled to the controller, wherein:
the skin sensor is configured to detect when skin is in contact with an outer surface of the housing adjacent to the skin sensor;
the controller is configured to cause the conduit to move into fluid communication with the fluid enclosed by the container; and
when the lock is coupled to the housing in the first configuration, the first portion of the lock is configured to prevent skin from contacting the outer surface of the housing adjacent to the skin sensor.
Item 4. The injection device of item 1, wherein, while in the first configuration, the lock is configured to prevent inadvertent activation of the injection device when the injection device is dropped or subject to vibration.
Item 5. An injection device, comprising:
a housing;
a plunger coupled to the housing and movable relative to the housing;
one or more electronics components used during an injection performed by the injection device, the one or more electronics components being formed within an electrical circuit, wherein:
   in a first configuration, a first portion of the plunger is disposed within the housing, the electrical circuit is open, and the one or more electronics components are in a low-power sleep mode;
   in a second configuration, the plunger moves outward relative to the housing, and the first portion of the plunger extends exterior of the housing; and
   in the second configuration, the electrical circuit is closed, and the one or more electronics components are transitioned from the low-power sleep mode, to an active mode.
Item 6. The injection device of item 5, further comprising a biasing member movable from a resting position to a stressed position, wherein:
in the first configuration, the biasing member is in the stressed position; and
in the second configuration, the biasing member is in the resting position.
Item 7. The injection device of item 6, further including a stop configured to maintain the biasing member in the stressed position and the plunger in the first configuration when the stop is in a blocking position, wherein movement of the stop from the blocking position allows the biasing member to move to the resting position and the plunger to move to the second configuration.
Item 8. The injection device of item 7, wherein the stop includes a packaging of the injection device.
Item 9. The injection device of item 5, wherein the one or more electronics components include a switch, and transition of the plunger from the first configuration to the second configuration moves the switch to close the electrical circuit.
Item 10. The injection device of claim 5, wherein:
the plunger is movable from the second configuration toward the housing to a third configuration; and
the injection device is configured to initiate an injection by the injection device only after the plunger is moved to the third configuration and the one or more electronics components are in the active mode.
Item 11. The injection device of item 10, wherein:
the one or more electronics components include a skin sensor and the injection device further includes a controller configured to initiate the injection by the injection device;
the skin sensor is configured to detect a presence of skin in contact with an outer surface of the housing adjacent to the skin sensor only after the plunger has moved to the third configuration; and
the controller is configured to initiate the injection only after detection of skin by the skin sensor.
Item 12. The injection device of item 11, further including a motor configured to dispense medicament from the injection device during the injection, wherein:
during the injection, the plunger is movable from the third configuration, away from the housing, to a fourth configuration; and
the controller is configured to cease operation of the motor while the motor is operating and the controller senses the plunger has moved from the third configuration to the fourth configuration.
Item 13. The injection device of item 12, wherein the controller is configured to:
determine that skin has remained in contact with the outer surface of the housing adjacent to the skin sensor, while the plunger is in the fourth configuration; and
resume operation of the motor when the plunger moves from the fourth configuration to the third configuration, after the controller determines that skin has remained in contact with the outer surface of the housing adjacent to the skin sensor while the plunger was in the fourth configuration.
Item 14. An injection device, comprising:
a housing, wherein the housing includes a curved bottom surface that is concave when viewed from a point external to the housing that is closer to the bottom surface of the housing than a top surface of the housing;
a circuit board positioned adjacent to the bottom surface of the housing, wherein the circuit board includes a skin sensor configured to sense a presence of skin in contact with the bottom surface of the housing; and
a controller coupled to the circuit board, wherein the controller is configured to initiate an injection by the injection device only after the skin sensor senses the presence of skin in contact with the bottom surface of the housing.
Item 15. The injection device of item 14, wherein the skin sensor is configured to sense the presence of skin in contact with the bottom surface of the housing without skin directly contacting the skin sensor.
Item 16. The injection device of item 14, wherein the skin sensor is a capacitance sensor.
Item 17. The injection device of item 14, wherein:
the housing further includes an opening in the curved bottom surface;
the injection device further includes a needle extendable out of the housing through the opening; and
the skin sensor is positioned adjacent to the opening.
Item 18. The injection device of item 14, wherein the circuit board is the only circuit board disposed within the housing.
Item 19. A method of manufacturing an injection device, the method comprising:
depositing a first material onto a mold, the first material having a first opacity;
depositing a second material around the mold and the first material, the second material having a second opacity that is higher than the first opacity; and
positioning a container enclosing a medicament within the injection device and adjacent to a first portion of the injection device formed by the first material.
Item 20. The method of item 19, further including positioning one or more LEDs within the injection device and adjacent to one or more second portions of the injection device, separate from the first portion of the injection device, the one or more second portions of the injection device being formed by the first material.
Item 21. An injection device, comprising:
a container disposed within the housing, the container having a first end and a second end;
a piston configured to move from the first end of the container toward the second end of the container to dispense a medicament from the container;
a drive member configured to drive the piston through the container;
an emitter configured to emit a beam of light toward the container;
a detector positioned on an opposing side of the container from the emitter, wherein the detector is configured to receive the beam of light emitted from the emitter; and
a controller coupled to the drive member, the emitter, and the detector, the controller being configured to:
   receive a first signal from the detector while the emitter is off, the first signal corresponding to an ambient level of light surrounding the injection device;
   receive a second signal from the detector while the emitter is on;
   calculate a difference between light values represented by the first signal and the second signal; and
   cease operation of the drive member when the difference is less than a threshold value.
Item 22. An injection device, comprising:
a container disposed within the housing, the container having a first end and a second end;
a piston configured to move from the first end of the container toward the second end of the container to dispense a medicament from the container;
a drive member configured to drive the piston through the container;
an emitter configured to emit a beam of light toward the container;
a detector positioned on an opposing side of the container from the emitter, wherein the detector is configured to receive the beam of light emitted from the emitter; and
a controller coupled to the drive member, the emitter, and the detector, the controller being configured to:
   initiate the drive member and the emitter;
   receive a first signal from the detector while the emitter is on, the first signal being representative of an amount of light received by the detector;
   allow for continued operation of the drive member for a first period of time immediately after initiation of the drive member; and
   cease operation of the drive member upon determining (1) that the amount of light received by the detector is less than a first threshold light value and (2) before the amount of light received by the detector subsequently rises to or above the first threshold light value, that a current of the drive member is greater than a first threshold current value.
Item 23. The injection device of item 22, wherein the controller is further configured to:
continue operation of the drive member if the determined amount of light received by the detector is less than the first threshold value and, before the current of the drive member exceeds the first threshold current value, the amount of light received by the detector rises to or above the first threshold light value.
Item 24. An injection device, comprising:
a container disposed within the housing, the having a first end and a second end;
a piston configured to move from the first end of the container toward the second end of the container to dispense a medicament from the container;
a drive member configured to drive the piston through the container; and
a controller coupled to the drive member, the controller being configured to:
   maintain a speed of the drive member until a current of the drive member exceeds a first threshold; and
   after the current of the drive member exceeds the first threshold, reduce a voltage of the drive member to maintain the current of the drive member below a second threshold that is greater than or equal to the first threshold.
Item 25. The injection device of item 1, further including a controller and a skin sensor coupled to the controller, wherein:
the skin sensor is configured to detect when skin is in contact with an outer surface of the housing adjacent to the skin sensor;
the controller is configured to cause the conduit to move into fluid communication with the fluid enclosed by the container; and
the injection device further comprises a removable cover configured to prevent skin from contacting the outer surface of the housing adjacent to the skin sensor.
Item 26. The method of item 19, further comprising:
positioning a conduit movable relative to the container within the injection device such that the conduit is not in fluid communication with the fluid enclosed by the container;
wherein the first portion includes a first transparent window through which an interior of the injection device is visible; and
wherein the container is visible through the transparent window.
Item 27. The method of item 26, wherein the first portion further includes a plurality of second transparent windows arranged on a top surface of the injection device, wherein at least one LED is visible through each of the plurality of second transparent windows; and
wherein the first transparent window and the plurality of second transparent windows are collectively formed of a contiguous portion of the first material.

What is claimed is:

1. An injection device, comprising:
a needle movable from a retracted configuration to a deployed configuration;
a vial containing a medicament comprising one or more of dupilumab and cemiplimab;
a piston configured to move within the vial;
a motor configured to apply force against the piston; and
a controller coupled to the motor, wherein the controller is configured to:
receive an indication that the injection device is positioned in contact with a user; and
after receiving the indication, send a signal to the motor to apply force against the piston in a first direction, wherein applying force in the first direction causes (1) the needle to move from the retracted configuration to the deployed configuration, and (2) the medicament to be dispensed from the vial through the needle.

2. The injection device of claim 1, wherein the controller is further configured to:
without requiring any intervention by a user after receiving the indication, and after sending the signal to apply force against the motor in the first direction, automatically send a signal to the motor to apply force against the piston in a second direction to cause the needle to retract.

3. The injection device of claim 1, further including a housing enclosing the vial, the piston, the motor, the controller, and the needle when the needle is in the retracted configuration, wherein the needle extends out of the housing in the deployed configuration.

4. The injection device of claim 1, further including a cover or a shield that contains a distalmost portion of the needle in the retracted configuration.

5. The injection device of claim 1, further including an audio module, a visual module, and a haptic module, each of the modules being coupled to the controller and configured to provide feedback to a user of the injection device.

6. The injection device of claim 1, further including a top that seals an opening of the vial, the top including a portion including a rubber material that is permeable to a sterilant, wherein the needle includes a proximalmost portion configured to be coupled with the vial, and, before the needle and vial are in fluid communication with one another, the proximalmost portion of the needle is disposed within the portion formed of the rubber material.

7. The injection device of claim 1, further including a cantilever coupled to the controller, and movable by the needle, wherein, when the needle is in the retracted configuration, the cantilever forms part of an open circuit that signals to the controller that the needle is in the retracted configuration, and when the needle is in the deployed configuration, the cantilever forms part of a closed circuit that signals to the controller that the needle is in the deployed configuration.

8. The injection device of claim 1, further including one or more spring contacts configured to form an electrical connection between the motor to the controller.

9. The injection device of claim 1, further including an activating switch movable between an extended position and a depressed position;
wherein the indication is based at least in part on the activating switch being in the depressed position.

10. The injection device of claim 1, further including a touch sensor configured to detect contact with skin,
wherein the indication is based at least in part on the touch sensor detecting contact with skin.

11. The injection device of claim 10, wherein the touch sensor includes a capacitive sensing electrode and is configured to detect contact with skin based on a change in a capacitance of the capacitive sensing electrode.

12. The injection device of claim 2, further including:
an emitter configured to transmit light through the vial; and
a detector configured to receive the light and to detect interruption of the light by the piston.

13. The injection device of claim 12, wherein the controller is further configured to send the signal to the motor to apply force against the piston in the second direction in response to interruption of the light by the piston.

14. The injection device of claim 12, wherein the controller is further configured, after a predetermined delay, to send the signal to the motor to apply force against the piston in the second direction in response to interruption of the light by the piston.

15. The injection device of claim 1, further including a wireless communication module configured to transmit to a remote device one or more of: diagnostic information of the injection device, information indicative of an error state of the injection device, and information indicative of completion of an injection.

16. The injection device of claim 1, further including a wireless communication module configured to receive an activation command from a remote device, wherein the indication is based at least in part on the activation command.

17. The injection device of claim 1, wherein the vial is configured to be in fluid communication with the needle.

18. The injection device of claim 1, wherein in response to the motor driving the piston in the first direction, the needle is put in fluid communication with the vial.

19. An injection device, comprising:
a housing having a tissue-engaging surface and an opening in the tissue-engaging surface;
a needle movable through the opening from a retracted configuration to a deployed configuration;
an activating switch configured to be moved from a deactivated configuration to an activated configuration;
a vial configured to come into fluid communication with the needle, wherein the vial contains a medicament comprising one or more of dupilumab and cemiplimab;
a motor configured to cause the needle to move through the opening; and
a controller coupled to the motor, wherein the controller is configured to:
in response to a trigger, cause the motor to move the needle from the retracted configuration to the deployed configuration and to dispense the medicament from the vial through the needle;
wherein the trigger includes receiving a first indication that the activating switch has been moved from the deactivated configuration to the activated configuration.

20. The injection device of claim 19, further comprising:
a touch sensor disposed on the tissue-engaging surface and configured to detect contact with skin;
wherein the trigger further includes a second indication that the touch sensor is in contact with skin.

21. The injection device of claim 19, further comprising:
a lock that is removable from the housing, the lock having a first portion and a second portion, wherein:
in a first configuration where the lock is coupled to the housing, the first portion of the lock is disposed exterior of the housing and the second portion of the lock is disposed within the housing between the container and the conduit;
in the first configuration, the conduit is prevented from moving into fluid communication with the fluid enclosed by the container by the second portion of the lock; and
in a second configuration where the lock is removed from the injection device, the conduit is able to move into fluid communication with the fluid enclosed by the container.


SEQUENCE LISTING (374 sequences)

SEQ ID NO: 1 - protein, 124 aa, Homo sapiens
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 8 aa, Homo sapiens
GFTFRDYA

SEQ ID NO: 3 - protein, 8 aa, Homo sapiens
ISGSGGNT

SEQ ID NO: 4 - protein, 16 aa, Homo sapiens
AKDRLSITIRPRYYGL

SEQ ID NO: 5 - protein, 112 aa, Homo sapiens [sequence omitted]

SEQ ID NO: 6 - protein, 11 aa, Homo sapiens
QSLLYSIGYNY

SEQ ID NO: 7 - skipped

SEQ ID NO: 8 - protein, 9 aa, Homo sapiens
MQALQTPYT

SEQ ID NO: 9 - protein, 118 aa, Homo sapiens
EVQLVESGGGLVQPGRSLRLSCEASGFTFDDYAMHWVRQAPGKGLEWVSGLSRTSVSIGYADSVKGRFTISRDNAKNSLYLEMNSLRPEDTALYYCAKWGTRGYFDYWGQGTLVTVSS

SEQ ID NO: 10 - protein, 8 aa, Homo sapiens
GFTFDDYA

SEQ ID NO: 11 - protein, 8 aa, Homo sapiens
LSRTSVSI

SEQ ID NO: 12 - protein, 11 aa, Homo sapiens
AKWGTRGYFDY

SEQ ID NO: 13 - protein, 107 aa, Homo sapiens
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLEIK

SEQ ID NO: 14 - protein, 6 aa, Homo sapiens
QDISIW

SEQ ID NO: 15 - skipped

SEQ ID NO: 16 - protein, 9 aa, Homo sapiens
QQANSFPIT

SEQ ID NO: 17 - protein, 117 aa, Homo sapiens
QVQLVESGGGVVQPGRSLRLSCAASGFTFRSYGMHWVRQAPGKGLEWVAVISYDGSNKYYIDSVKGRFTISRDNSKNTLNLQMNSLRLEDTAVYYCAKEGRGGFDYWGQGTLVTVSS

SEQ ID NO: 18 - protein, 8 aa, Homo sapiens
GFTFRSYG

SEQ ID NO: 19 - protein, 8 aa, Homo sapiens
ISYDGSNK

SEQ ID NO: 20 - protein, 10 aa, Homo sapiens
AKEGRGGFDY

SEQ ID NO: 21 - protein, 107 aa, Homo sapiens
DIQMTQSPSSLSASVGDRVTITCRASQVINNYLAWFQQKPGKVPKSLIHAASSLQSGVPSKFSGSGSGTDFTLTISSLQPEDFATYYCQQYNSHPWTFGQGTKVEIK

SEQ ID NO: 22 - protein, 6 aa, Homo sapiens
QVINNY

SEQ ID NO: 23 - skipped

SEQ ID NO: 24 - protein, 9 aa, Homo sapiens
QQYNSHPWT

SEQ ID NO: 25 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGRAGIVARLTESLFLDLLGEENGGFCQQDMGESRLLPPSGSTSAHMPWDEFPSAGPKEAPPWGKEQPLHLEPSPPASPTQSPDNLTCTETPLVIAGNPAYRSFSNPLSQSPCPRELGPDPLLARHLEEVEPEMPCVPQLSEPTTVPQPEPETWEQILRRNVLQHGAAAAPVSAPTSGYREFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 26 - protein, 207 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 27 - DNA, 363 bp, synthetic construct
  misc_feature 1..363: note = Synthetic
  source 1..363: mol_type = other DNA
gaggtgcagctgttggagtctgggggaggcttggtacagcctggggggtccctgagactctcctgttcagcctctggattcacctttagcagctataccatgaactgggtccgccaggctccagggaaggggctggagtgggtctcaggtattagtgataccggtggtaacacatactacacagactccgtgaagggccggttcaccgtctccagagacaattccaagaacacactgtctctgcaaatgaacagcctgagagccgaggacacggccgtatattactgtgcgaaagatcagggtggaagttacccctattactttcactactggggccagggatccctggtcaccgtctcctca

SEQ ID NO: 28 - protein, 121 aa, synthetic construct
  REGION 1..121: note = Synthetic
EVQLLESGGGLVQPGGSLRLSCSASGFTFSSYTMNWVRQAPGKGLEWVSGISDTGGNTYYTDSVKGRFTVSRDNSKNTLSLQMNSLRAEDTAVYYCAKDQGGSYPYYFHYWGQGSLVTVSS

SEQ ID NO: 29 - DNA, 24 bp, synthetic construct
  misc_feature 1..24: note = Synthetic
  source 1..24: mol_type = other DNA
ggattcacctttagcagctatacc

SEQ ID NO: 30 - protein, 8 aa, synthetic construct
  REGION 1..8: note = Synthetic
GFTFSSYT

SEQ ID NO: 31 - DNA, 24 bp, synthetic construct
  misc_feature 1..24: note = Synthetic
  source 1..24: mol_type = other DNA
attagtgataccggtggtaacaca

SEQ ID NO: 32 - protein, 8 aa, synthetic construct
  REGION 1..8: note = Synthetic
ISDTGGNT

SEQ ID NO: 33 - DNA, 42 bp, synthetic construct
  misc_feature 1..42: note = Synthetic
  source 1..42: mol_type = other DNA
gcgaaagatcagggtggaagttacccctattactttcactac

SEQ ID NO: 34 - protein, 14 aa, synthetic construct
  REGION 1..14: note = Synthetic
AKDQGGSYPYYFHY

SEQ ID NO: 35 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaatttggtatcagcagaaaccagggacagcccctaagttcctgatctatgctgcatccagtttgcaaagtggggtcccatcaaggttcagtggctgtggatctgggacagatttcactctcaccatcagcagtctgcaacctgaagattttgcaacttactactgtcaacagagttacagtacccctccgatcaccttcggccaagggacacgactggagattaaa

SEQ ID NO: 36 - protein, 108 aa, synthetic construct
  REGION 1..108: note = Synthetic
DIQMTQSPSSLSASVGDRVTITCRASQSISSYLIWYQQKPGTAPKFLIYAASSLQSGVPSRFSGCGSGTDFTLTISSLQPEDFATYYCQQSYSTPPITFGQGTRLEIK

SEQ ID NO: 37 - DNA, 18 bp, synthetic construct
  misc_feature 1..18: note = Synthetic
  source 1..18: mol_type = other DNA
cagagcattagcagctat

SEQ ID NO: 38 - protein, 6 aa, synthetic construct
  REGION 1..6: note = Synthetic
QSISSY

SEQ ID NO: 39-40 - skipped (2 entries)

SEQ ID NO: 41 - DNA, 30 bp, synthetic construct
  misc_feature 1..30: note = Synthetic
  source 1..30: mol_type = other DNA
caacagagttacagtacccctccgatcacc

SEQ ID NO: 42 - protein, 10 aa, synthetic construct
  REGION 1..10: note = Synthetic
QQSYSTPPIT

SEQ ID NO: 43 - DNA, 366 bp, synthetic construct
  misc_feature 1..366: note = Synthetic
  source 1..366: mol_type = other DNA
gaggtgcagctggtggagtctggaggaggcttggtccagcctggggggtccctgagactctcctgtgcagcctctgggttcaccgtcagtaacaactacatgagctgggtccgccaggctccagggaaggggctggagtgggtctcagttatttatagcggtggtttcacatactacacagactccgtgaagggccgattcaccatctccagacacaattccaagaacacgctgtatcttcaaatgaacagcctgagagctgaggacacggccgtgtattactgtgcgaggtattactatgatactagtgattattggaccttctttgactactggggccagggaaccctggtcaccgtctcctca

SEQ ID NO: 44 - protein, 122 aa, synthetic construct
  REGION 1..122: note = Synthetic
EVQLVESGGGLVQPGGSLRLSCAASGFTVSNNYMSWVRQAPGKGLEWVSVIYSGGFTYYTDSVKGRFTISRHNSKNTLYLQMNSLRAEDTAVYYCARYYYDTSDYWTFFDYWGQGTLVTVSS

SEQ ID NO: 45 - DNA, 24 bp, synthetic construct
  misc_feature 1..24: note = Synthetic
  source 1..24: mol_type = other DNA
gggttcaccgtcagtaacaactac

SEQ ID NO: 46 - protein, 8 aa, synthetic construct
  REGION 1..8: note = Synthetic
GFTVSNNY

SEQ ID NO: 47 - DNA, 21 bp, synthetic construct
  misc_feature 1..21: note = Synthetic
  source 1..21: mol_type = other DNA
atttatagcggtggtttcaca

SEQ ID NO: 48 - protein, 7 aa, synthetic construct
  REGION 1..7: note = Synthetic
IYSGGFT

SEQ ID NO: 49 - DNA, 48 bp, synthetic construct
  misc_feature 1..48: note = Synthetic
  source 1..48: mol_type = other DNA
gcgaggtattactatgatactagtgattattggaccttctttgactac

SEQ ID NO: 50 - protein, 16 aa, synthetic construct
  REGION 1..16: note = Synthetic
ARYYYDTSDYWTFFDY

SEQ ID NO: 51 - DNA, 321 bp, synthetic construct
  misc_feature 1..321: note = Synthetic
  source 1..321: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagtagcctgcagtctggagattttgcagtttattactgtcagcagtataataactggccgctcactttcggcggagggaccaaggtggagatcaat

SEQ ID NO: 52 - protein, 107 aa, synthetic construct
  REGION 1..107: note = Synthetic
EIVMTQSPATLSVSPGERATLSCRASQSVSSNLAWYQQKPGQAPRLLIYGASTRATGIPARFSGSGSGTEFTLTISSLQSGDFAVYYCQQYNNWPLTFGGGTKVEIN

SEQ ID NO: 53 - DNA, 18 bp, synthetic construct
  misc_feature 1..18: note = Synthetic
  source 1..18: mol_type = other DNA
cagagtgttagcagcaac

SEQ ID NO: 54 - protein, 6 aa, synthetic construct
  REGION 1..6: note = Synthetic
QSVSSN

SEQ ID NO: 55-56 - skipped (2 entries)

SEQ ID NO: 57 - DNA, 27 bp, synthetic construct
  misc_feature 1..27: note = Synthetic
  source 1..27: mol_type = other DNA
cagcagtataataactggccgctcact

SEQ ID NO: 58 - protein, 9 aa, synthetic construct
  REGION 1..9: note = Synthetic
QQYNNWPLT

SEQ ID NO: 59 - DNA, 366 bp, synthetic construct; identical to SEQ ID NO: 43
  misc_feature 1..366: note = Synthetic
  source 1..366: mol_type = other DNA

SEQ ID NO: 60 - protein, 122 aa, synthetic construct; identical to SEQ ID NO: 44
  REGION 1..122: note = Synthetic

SEQ ID NO: 61 - DNA, 24 bp, synthetic construct
  misc_feature 1..24: note = Synthetic
  source 1..24: mol_type = other DNA
gggttcaccgtcagtaacaactac

SEQ ID NO: 62 - protein, 8 aa, synthetic construct
  REGION 1..8: note = Synthetic
GFTVSNNY

SEQ ID NO: 63 - DNA, 21 bp, synthetic construct
  misc_feature 1..21: note = Synthetic
  source 1..21: mol_type = other DNA
atttatagcggtggtttcaca

SEQ ID NO: 64 - protein, 7 aa, synthetic construct
  REGION 1..7: note = Synthetic
IYSGGFT

SEQ ID NO: 65 - DNA, 48 bp, synthetic construct; identical to SEQ ID NO: 49
  misc_feature 1..48: note = Synthetic
  source 1..48: mol_type = other DNA

SEQ ID NO: 66 - protein, 16 aa, synthetic construct
  REGION 1..16: note = Synthetic
ARYYYDTSDYWTFFDY

SEQ ID NO: 67 - DNA, 321 bp, synthetic construct; identical to SEQ ID NO: 51
  misc_feature 1..321: note = Synthetic
  source 1..321: mol_type = other DNA

SEQ ID NO: 68 - protein, 107 aa, synthetic construct; identical to SEQ ID NO: 52
  REGION 1..107: note = Synthetic

SEQ ID NO: 69 - DNA, 18 bp, synthetic construct
  misc_feature 1..18: note = Synthetic
  source 1..18: mol_type = other DNA
cagagtgttagcagcaac

SEQ ID NO: 70 - protein, 6 aa, synthetic construct
  REGION 1..6: note = Synthetic
QSVSSN

SEQ ID NO: 71-72 - skipped (2 entries)

SEQ ID NO: 73 - DNA, 27 bp, synthetic construct
  misc_feature 1..27: note = Synthetic
  source 1..27: mol_type = other DNA
cagcagtataataactggccgctcact

SEQ ID NO: 74 - protein, 9 aa, synthetic construct
  REGION 1..9: note = Synthetic
QQYNNWPLT

SEQ ID NO: 75 - DNA, 357 bp, synthetic construct
  misc_feature 1..357: note = Synthetic
  source 1..357: mol_type = other DNA
caggtgcagctggtggagtctgggggaggcgtggtccagtctgggaggtccctgagactctcctgtgcagcgtctggattcaccttcagtagctatggcatgcactgggtccgccaggctccaggcaaggggctggagtgggtggcagttatatggtatgatggaagtaatatatactattcagactccgtgaagggccgattcaccatctccagagccaattccaagaacacgctgtatctgcaaatgaacagcctgagagccgaggacacggctgtttattactgtgcgagaccgggacactggaactacttctttgaatactggggccagggaaccctggtcaccgtctcctca

SEQ ID NO: 76 - protein, 119 aa, synthetic construct
  REGION 1..119: note = Synthetic
QVQLVESGGGVVQSGRSLRLSCAASGFTFSSYGMHWVRQAPGKGLEWVAVIWYDGSNIYYSDSVKGRFTISRANSKNTLYLQMNSLRAEDTAVYYCARPGHWNYFFEYWGQGTLVTVSS

SEQ ID NO: 77 - DNA, 24 bp, synthetic construct
  misc_feature 1..24: note = Synthetic
  source 1..24: mol_type = other DNA
ggattcaccttcagtagctatggc

SEQ ID NO: 78 - protein, 8 aa, synthetic construct
  REGION 1..8: note = Synthetic
GFTFSSYG

SEQ ID NO: 79 - DNA, 24 bp, synthetic construct
  misc_feature 1..24: note = Synthetic
  source 1..24: mol_type = other DNA
atatggtatgatggaagtaatata

SEQ ID NO: 80 - protein, 8 aa, synthetic construct
  REGION 1..8: note = Synthetic
IWYDGSNI

SEQ ID NO: 81 - DNA, 36 bp, synthetic construct
  misc_feature 1..36: note = Synthetic
  source 1..36: mol_type = other DNA
gcgagaccgggacactggaactacttctttgaatac

SEQ ID NO: 82 - protein, 12 aa, synthetic construct
  REGION 1..12: note = Synthetic
ARPGHWNYFFEY

SEQ ID NO: 83 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattaacaactatttaaattggtatcagcagaaaccagggaaagcccctaagctcctgatctatactgcatccagtttgcaaagtggggtcccatcaaggttcagtggcagtggatctgggacagatttcactctcaccatcagcagtctgcaacctgaagattttgcaacttactactgtcaacagagttacagtacccctccgctcaccttcggccaagggacacaactggagattaaa

SEQ ID NO: 84 - protein, 108 aa, synthetic construct
  REGION 1..108: note = Synthetic
DIQMTQSPSSLSASVGDRVTITCRASQSINNYLNWYQQKPGKAPKLLIYTASSLQSGVPSRFSGSGSGTDFTLTISSLQPEDFATYYCQQSYSTPPLTFGQGTQLEIK

SEQ ID NO: 85 - DNA, 18 bp, synthetic construct
  misc_feature 1..18: note = Synthetic
  source 1..18: mol_type = other DNA
cagagcattaacaactat

SEQ ID NO: 86 - protein, 6 aa, synthetic construct
  REGION 1..6: note = Synthetic
QSINNY

SEQ ID NO: 87-88 - skipped (2 entries)

SEQ ID NO: 89 - DNA, 30 bp, synthetic construct
  misc_feature 1..30: note = Synthetic
  source 1..30: mol_type = other DNA
caacagagttacagtacccctccgctcacc

SEQ ID NO: 90 - protein, 10 aa, synthetic construct
  REGION 1..10: note = Synthetic
QQSYSTPPLT

SEQ ID NO: 91 - DNA, 366 bp, synthetic construct
  misc_feature 1..366: note = Synthetic
  source 1..366: mol_type = other DNA
gaggtgcagctggtggagtctgggggaggcttggtacagcctggggggtccctgagactctcctgtggagcctctggattcaccttcaggaactacgacatgcactgggtccgccaaattacaggaaaaggtctggagtgggtctcagctattggtagtgctggtgacacatactatccagactccgtgaagggccgattcaccatctccagagaaaatgccaagaactccttgtatcttcaaatgaacagcctgagagtcggggacacggctgtgtattactgtacaagagatatccattgtagtagtaccaggtgctacggtatggacgtctggggccaagggaccacggtcaccgtctcctca

SEQ ID NO: 92 - protein, 122 aa, synthetic construct
  REGION 1..122: note = Synthetic
EVQLVESGGGLVQPGGSLRLSCGASGFTFRNYDMHWVRQITGKGLEWVSAIGSAGDTYYPDSVKGRFTISRENAKNSLYLQMNSLRVGDTAVYYCTRDIHCSSTRCYGMDVWGQGTTVTVSS

SEQ ID NO: 93 - DNA, 24 bp, synthetic construct
  misc_feature 1..24: note = Synthetic
  source 1..24: mol_type = other DNA
ggattcaccttcaggaactacgac

SEQ ID NO: 94 - protein, 8 aa, synthetic construct
  REGION 1..8: note = Synthetic
GFTFRNYD

SEQ ID NO: 95 - DNA, 21 bp, synthetic construct
  misc_feature 1..21: note = Synthetic
  source 1..21: mol_type = other DNA
attggtagtgctggtgacaca

SEQ ID NO: 96 - protein, 7 aa, synthetic construct
  REGION 1..7: note = Synthetic
IGSAGDT

SEQ ID NO: 97 - DNA, 48 bp, synthetic construct
  misc_feature 1..48: note = Synthetic
  source 1..48: mol_type = other DNA
acaagagatatccattgtagtagtaccaggtgctacggtatggacgtc

SEQ ID NO: 98 - protein, 16 aa, synthetic construct
  REGION 1..16: note = Synthetic
TRDIHCSSTRCYGMDV

SEQ ID NO: 99 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcaactatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 100 - protein, 108 aa, synthetic construct
  REGION 1..108: note = Synthetic
DIQMTQSPSSLSASVGDRVTITCRASQSISNYLNWYQQKPGKAPKLLIYAASSLQSGVPSRFSGSGSGTDFTLTISSLQPEDFATYYCQQSYSTPPITFGQGTRLEIK

SEQ ID NO: 101 - DNA, 18 bp, synthetic construct
  misc_feature 1..18: note = Synthetic
  source 1..18: mol_type = other DNA
cagagcattagcaactat

SEQ ID NO: 102 - protein, 6 aa, synthetic construct
  REGION 1..6: note = Synthetic
QSISNY

SEQ ID NO: 103-104 - skipped (2 entries)

SEQ ID NO: 105 - DNA, 30 bp, synthetic construct; identical to SEQ ID NO: 41
  misc_feature 1..30: note = Synthetic
  source 1..30: mol_type = other DNA

SEQ ID NO: 106 - protein, 10 aa, synthetic construct
  REGION 1..10: note = Synthetic
QQSYSTPPIT

SEQ ID NO: 107 - DNA, 390 bp, synthetic construct
  misc_feature 1..390: note = Synthetic
  source 1..390: mol_type = other DNA
gaggtgcagctggtggagtctgggggaggcttggtaaagcctggggggtcccttcgactctcctgtgcagcctctggattcaaattcagtaatgaatggatgagctgggtccgccaggctccagggaaggggctggagtgggttggccgtattaaaagcaaaactgatggtgggacaacagactacgctgcacccgtgaaaggcagattcaccatctcaagagatgattcaaaaaatacgctgtatctgcaaatgaacagcctgaaaaccgaggacacagccgtgtattactgtaccacagatcaagatttttggagtggttattataccggggctgactactacggtatggacgtctggggccaagggaccatggtcaccgtctcctca

SEQ ID NO: 108 - protein, 130 aa, synthetic construct
  REGION 1..130: note = Synthetic
EVQLVESGGGLVKPGGSLRLSCAASGFKFSNEWMSWVRQAPGKGLEWVGRIKSKTDGGTTDYAAPVKGRFTISRDDSKNTLYLQMNSLKTEDTAVYYCTTDQDFWSGYYTGADYYGMDVWGQGTMVTVSS

SEQ ID NO: 109 - DNA, 24 bp, synthetic construct
  misc_feature 1..24: note = Synthetic
  source 1..24: mol_type = other DNA
ggattcaaattcagtaatgaatgg

SEQ ID NO: 110 - protein, 8 aa, synthetic construct
  REGION 1..8: note = Synthetic
GFKFSNEW

SEQ ID NO: 111 - DNA, 30 bp, synthetic construct
  misc_feature 1..30: note = Synthetic
  source 1..30: mol_type = other DNA
attaaaagcaaaactgatggtgggacaaca

SEQ ID NO: 112 - protein, 10 aa, synthetic construct
  REGION 1..10: note = Synthetic
IKSKTDGGTT

SEQ ID NO: 113 - DNA, 63 bp, synthetic construct
  misc_feature 1..63: note = Synthetic
  source 1..63: mol_type = other DNA
accacagatcaagatttttggagtggttattataccggggctgactactacggtatggacgtc

SEQ ID NO: 114 - protein, 21 aa, synthetic construct
  REGION 1..21: note = Synthetic
TTDQDFWSGYYTGADYYGMDV

SEQ ID NO: 115 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 116 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 117 - DNA, 18 bp, synthetic construct
  misc_feature 1..18: note = Synthetic
  source 1..18: mol_type = other DNA
cagagcattagcagctat

SEQ ID NO: 118 - protein, 6 aa, synthetic construct
  REGION 1..6: note = Synthetic
QSISSY

SEQ ID NO: 119-120 - skipped (2 entries)

SEQ ID NO: 121 - DNA, 30 bp, synthetic construct; identical to SEQ ID NO: 41
  misc_feature 1..30: note = Synthetic
  source 1..30: mol_type = other DNA

SEQ ID NO: 122 - protein, 10 aa, synthetic construct
  REGION 1..10: note = Synthetic
QQSYSTPPIT

SEQ ID NO: 123 - DNA, 348 bp, synthetic construct
  misc_feature 1..348: note = Synthetic
  source 1..348: mol_type = other DNA
cagatgcagctccaacagtggggcgcaggactattgaagccttcggagaccctgtccctcacctgcgttgtctatggtgggtccctcaatggatactattggagctggatccgccagtcccccgggaaggggctggagtggattggggaaatcgatcatagtggaagcaccaactacaacccgtccctcaagaatcgagtcaccatgtcagtagacacgtctaagattcagttctccctgaaactgacctctgtgaccgtcgcggacacggctgtgtattactgtgcgagagaaggattattaccctttgactattggggccagggaaccctggtcaccgtctcctca

SEQ ID NO: 124 - protein, 116 aa, synthetic construct
  REGION 1..116: note = Synthetic
QMQLQQWGAGLLKPSETLSLTCVVYGGSLNGYYWSWIRQSPGKGLEWIGEIDHSGSTNYNPSLKNRVTMSVDTSKIQFSLKLTSVTVADTAVYYCAREGLLPFDYWGQGTLVTVSS

SEQ ID NO: 125 - DNA, 24 bp, synthetic construct
  misc_feature 1..24: note = Synthetic
  source 1..24: mol_type = other DNA
ggtgggtccctcaatggatactat

SEQ ID NO: 126 - protein, 8 aa, synthetic construct
  REGION 1..8: note = Synthetic
GGSLNGYY

SEQ ID NO: 127 - DNA, 21 bp, synthetic construct
  misc_feature 1..21: note = Synthetic
  source 1..21: mol_type = other DNA
atcgatcatagtggaagcacc

SEQ ID NO: 128 - protein, 7 aa, synthetic construct
  REGION 1..7: note = Synthetic
IDHSGST

SEQ ID NO: 129 - DNA, 30 bp, synthetic construct
  misc_feature 1..30: note = Synthetic
  source 1..30: mol_type = other DNA
gcgagagaaggattattaccctttgactat

SEQ ID NO: 130 - protein, 10 aa, synthetic construct
  REGION 1..10: note = Synthetic
AREGLLPFDY

SEQ ID NO: 131 - DNA, 324 bp, synthetic construct
  misc_feature 1..324: note = Synthetic
  source 1..324: mol_type = other DNA
gaaattgtgttgacgcagtctccaggcaccctgtctttgtctccaggggaaagagtcaccctctcctgcagggccagtcagagtgtttacagcaactacttagcctggtaccagcagaatcctggccaggctcccaggctcctcatctatgctgcatccaacagggccactggcatcccagacaggttcagtggcagtgggtctgggacagacttcactctcaccatcagcagactggagcctgaagattttgcggtgtattactgtcatcagtatgctacctcaccttggacgttcggccaagggaccaaggtggaaatcaaa

SEQ ID NO: 132 - protein, 108 aa, synthetic construct
  REGION 1..108: note = Synthetic
EIVLTQSPGTLSLSPGERVTLSCRASQSVYSNYLAWYQQNPGQAPRLLIYAASNRATGIPDRFSGSGSGTDFTLTISRLEPEDFAVYYCHQYATSPWTFGQGTKVEIK

SEQ ID NO: 133 - DNA, 21 bp, synthetic construct
  misc_feature 1..21: note = Synthetic
  source 1..21: mol_type = other DNA
cagagtgtttacagcaactac

SEQ ID NO: 134 - protein, 7 aa, synthetic construct
  REGION 1..7: note = Synthetic
QSVYSNY

SEQ ID NO: 135-136 - skipped (2 entries)

SEQ ID NO: 137 - DNA, 27 bp, synthetic construct
  misc_feature 1..27: note = Synthetic
  source 1..27: mol_type = other DNA
catcagtatgctacctcaccttggacg

SEQ ID NO: 138 - protein, 9 aa, synthetic construct
  REGION 1..9: note = Synthetic
HQYATSPWT

SEQ ID NO: 139 - DNA, 369 bp, synthetic construct
  misc_feature 1..369: note = Synthetic
  source 1..369: mol_type = other DNA
cagctgcagctgcaggagtcgggcccagatctggtgaagccttcggataccctgtccctcacctgcactgtctctgatgactccatcagcagtactacttactactgggcctggatccgccagcccccagggaaggggctggaatggattggcagtatgtcttataatgggaacaactactacaacccgtccctcaagagtcgagtcgccatatccgcaggcacgtcccagaaacagttctccctgaaactgacctctgtgactgccgcagacacggctgtttatcactgtgcgagacatcttggatataacggcaactggtacccctttgacttctggggccagggaattctggtcaccgtctcctct

SEQ ID NO: 140 - protein, 123 aa, synthetic construct
  REGION 1..123: note = Synthetic
QLQLQESGPDLVKPSDTLSLTCTVSDDSISSTTYYWAWIRQPPGKGLEWIGSMSYNGNNYYNPSLKSRVAISAGTSQKQFSLKLTSVTAADTAVYHCARHLGYNGNWYPFDFWGQGILVTVSS

SEQ ID NO: 141 - DNA, 30 bp, synthetic construct
  misc_feature 1..30: note = Synthetic
  source 1..30: mol_type = other DNA
gatgactccatcagcagtactacttactac

SEQ ID NO: 142 - protein, 10 aa, synthetic construct
  REGION 1..10: note = Synthetic
DDSISSTTYY

SEQ ID NO: 143 - DNA, 21 bp, synthetic construct
  misc_feature 1..21: note = Synthetic
  source 1..21: mol_type = other DNA
atgtcttataatgggaacaac

SEQ ID NO: 144 - protein, 7 aa, synthetic construct
  REGION 1..7: note = Synthetic
MSYNGNN

SEQ ID NO: 145 - DNA, 45 bp, synthetic construct
  misc_feature 1..45: note = Synthetic
  source 1..45: mol_type = other DNA
gcgagacatcttggatataacggcaactggtacccctttgacttc

SEQ ID NO: 146 - protein, 15 aa, synthetic construct
  REGION 1..15: note = Synthetic
ARHLGYNGNWYPFDF

SEQ ID NO: 147 - DNA, 324 bp, synthetic construct
  misc_feature 1..324: note = Synthetic
  source 1..324: mol_type = other DNA
gaaattgtgttgacgcagtctccaggcaccctgtctttgtctccaggagaaagagccaccctctcctgcagggccagtcagagtgttagtagtagttatttagcctggtaccagcagaaacctggccaggctcccaggctcctcatctatggtgcatccagcaggaccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 148 - protein, 108 aa, synthetic construct
  REGION 1..108: note = Synthetic
EIVLTQSPGTLSLSPGERATLSCRASQSVSSSYLAWYQQKPGQAPRLLIYGASSRTTGIPDRFSGSGSGTDFTLTISRLEPEDFAVYYCQQYGSSPWTFGQGTKVEIK

SEQ ID NO: 149 - DNA, 21 bp, synthetic construct
  misc_feature 1..21: note = Synthetic
  source 1..21: mol_type = other DNA
cagagtgttagtagtagttat

SEQ ID NO: 150 - protein, 7 aa, synthetic construct
  REGION 1..7: note = Synthetic
QSVSSSY

SEQ ID NO: 151-152 - skipped (2 entries)

SEQ ID NO: 153 - DNA, 27 bp, synthetic construct
  misc_feature 1..27: note = Synthetic
  source 1..27: mol_type = other DNA
cagcagtatggtagctcaccttggacg

SEQ ID NO: 154 - protein, 9 aa, synthetic construct
  REGION 1..9: note = Synthetic
QQYGSSPWT

SEQ ID NO: 155 - DNA, 366 bp, synthetic construct
  misc_feature 1..366: note = Synthetic
  source 1..366: mol_type = other DNA
gaagtgcaggtggtagagtctgggggcggcttggtcgagcctggcaggtccctgagactctcctgtaaagcctctggattcacctttgatgattatgccatgcactgggtccgacaaactccagggaaggccctggagtgggtctcgggtattaattggagtggtaataacataggctatgcggactctgtgaagggccgattcaccatctccaaggacgacgccaagaactccctgtatctgcaaatgaacagtctgagacctgaggacacggccttatattactgtacaaaagatataagtataactggaaccctcgatgcttttgatgtctggggccaagggacaatggtcaccgtctcttca

SEQ ID NO: 156 - protein, 122 aa, synthetic construct
  REGION 1..122: note = Synthetic
EVQVVESGGGLVEPGRSLRLSCKASGFTFDDYAMHWVRQTPGKALEWVSGINWSGNNIGYADSVKGRFTISKDDAKNSLYLQMNSLRPEDTALYYCTKDISITGTLDAFDVWGQGTMVTVSS

SEQ ID NO: 157 - DNA, 24 bp, synthetic construct
  misc_feature 1..24: note = Synthetic
  source 1..24: mol_type = other DNA
ggattcacctttgatgattatgcc

SEQ ID NO: 158 - protein, 8 aa, synthetic construct
  REGION 1..8: note = Synthetic
GFTFDDYA

SEQ ID NO: 159 - DNA, 24 bp, synthetic construct
  misc_feature 1..24: note = Synthetic
  source 1..24: mol_type = other DNA
attaattggagtggtaataacata

SEQ ID NO: 160 - protein, 8 aa, synthetic construct
  REGION 1..8: note = Synthetic
INWSGNNI

SEQ ID NO: 161 - DNA, 45 bp, synthetic construct
  misc_feature 1..45: note = Synthetic
  source 1..45: mol_type = other DNA
acaaaagatataagtataactggaaccctcgatgcttttgatgtc

SEQ ID NO: 162 - protein, 15 aa, synthetic construct
  REGION 1..15: note = Synthetic
TKDISITGTLDAFDV

SEQ ID NO: 163 - DNA, 321 bp, synthetic construct
  misc_feature 1..321: note = Synthetic
  source 1..321: mol_type = other DNA
gacatccagatgacccagtctccaatttccgtgtctgcatctgtaggagacagagtcaccatcacttgtcgggcgagtcagggtattagcaactggttagcctggtatcagcagaaaccagggatagcccctaaactcctgatctattctgcatccagtttacaaagtggggtcccatcaaggttcagaggcagtggatctgggacagacttcactctcaccatcggcagcctgcagcctgaagattttgcaacttactattgtcaacaggctcacagtttcccgctcactttcggcggagggaccaaggtggagatcaaa

SEQ ID NO: 164 - protein, 107 aa, synthetic construct
  REGION 1..107: note = Synthetic
DIQMTQSPISVSASVGDRVTITCRASQGISNWLAWYQQKPGIAPKLLIYSASSLQSGVPSRFRGSGSGTDFTLTIGSLQPEDFATYYCQQAHSFPLTFGGGTKVEIK

SEQ ID NO: 165 - DNA, 18 bp, synthetic construct
  misc_feature 1..18: note = Synthetic
  source 1..18: mol_type = other DNA
cagggtattagcaactgg

SEQ ID NO: 166 - protein, 6 aa, synthetic construct
  REGION 1..6: note = Synthetic
QGISNW

SEQ ID NO: 167-168 - skipped (2 entries)

SEQ ID NO: 169 - DNA, 27 bp, synthetic construct
  misc_feature 1..27: note = Synthetic
  source 1..27: mol_type = other DNA
caacaggctcacagtttcccgctcact

SEQ ID NO: 170 - protein, 9 aa, synthetic construct
  REGION 1..9: note = Synthetic
QQAHSFPLT

SEQ ID NO: 171 - DNA, 348 bp, synthetic construct
  misc_feature 1..348: note = Synthetic
  source 1..348: mol_type = other DNA
caggtgcaattagtggagtctgggggaggcgtggtccagcctgggaggtccctgagactctcctgtgcagcgtctggattcaccttcagtagctatggcatgcactgggtccgccaggctccaggcaaggggctggagtgggtggcaattatatggtctgatggagatagtgaatataatctagactccgtaaagggccgattcaccatctccagagacaattccaagaacacgctgtatctgcaaatgaacagtctgagagtcgaagactcggctgtatattactgtgcgagagatcgagaccttgaggatatctggggccaagggacaatggtcaccgtctcttca

SEQ ID NO: 172 - protein, 116 aa, synthetic construct
  REGION 1..116: note = Synthetic
QVQLVESGGGVVQPGRSLRLSCAASGFTFSSYGMHWVRQAPGKGLEWVAIIWSDGDSEYNLDSVKGRFTISRDNSKNTLYLQMNSLRVEDSAVYYCARDRDLEDIWGQGTMVTVSS

SEQ ID NO: 173 - DNA, 24 bp, synthetic construct
  misc_feature 1..24: note = Synthetic
  source 1..24: mol_type = other DNA
ggattcaccttcagtagctatggc

SEQ ID NO: 174 - protein, 8 aa, synthetic construct
  REGION 1..8: note = Synthetic
GFTFSSYG

SEQ ID NO: 175 - DNA, 24 bp, synthetic construct
  misc_feature 1..24: note = Synthetic
  source 1..24: mol_type = other DNA
atatggtctgatggagatagtgaa

SEQ ID NO: 176 - protein, 8 aa, synthetic construct
  REGION 1..8: note = Synthetic
IWSDGDSE

SEQ ID NO: 177 - DNA, 27 bp, synthetic construct
  misc_feature 1..27: note = Synthetic
  source 1..27: mol_type = other DNA
gcgagagatcgagaccttgaggatatc

SEQ ID NO: 178 - protein, 9 aa, synthetic construct
  REGION 1..9: note = Synthetic
ARDRDLEDI

SEQ ID NO: 179 - DNA, 321 bp, synthetic construct
  misc_feature 1..321: note = Synthetic
  source 1..321: mol_type = other DNA
gacatccagatgacccagtctccatcctccctgtctgcatctgtcggagacagagtcaccatcacttgccgggcaagtcagggcattagaaatgatttaggctggtatcagcagaaaccagggaaagcccctaagcgcctgatctatgctgcatccaatttgcaaagtggggtcccatcaaggttcagcggcagtggatctgggacagagttcactctcacaatcagcagcctgcagcctgaagattttgcaacttattactgtctacagcataatagttatccgctcactttcggcggagggaccaaggtggagatcaaa

SEQ ID NO: 180 - protein, 107 aa, synthetic construct
  REGION 1..107: note = Synthetic
DIQMTQSPSSLSASVGDRVTITCRASQGIRNDLGWYQQKPGKAPKRLIYAASNLQSGVPSRFSGSGSGTEFTLTISSLQPEDFATYYCLQHNSYPLTFGGGTKVEIK

SEQ ID NO: 181 - DNA, 18 bp, synthetic construct
  misc_feature 1..18: note = Synthetic
  source 1..18: mol_type = other DNA
cagggcattagaaatgat

SEQ ID NO: 182 - protein, 6 aa, synthetic construct
  REGION 1..6: note = Synthetic
QGIRND

SEQ ID NO: 183-184 - skipped (2 entries)

SEQ ID NO: 185 - DNA, 27 bp, synthetic construct
  misc_feature 1..27: note = Synthetic
  source 1..27: mol_type = other DNA
ctacagcataatagttatccgctcact

SEQ ID NO: 186 - protein, 9 aa, synthetic construct
  REGION 1..9: note = Synthetic
LQHNSYPLT

SEQ ID NO: 187 - DNA, 351 bp, synthetic construct
  misc_feature 1..351: note = Synthetic
  source 1..351: mol_type = other DNA
gaggtgcagttgttggagtctgggggagttttggtacagcctggggggtccctgagactctcctgtgcagcctctggattcacctttagtaattttggcatgacgtgggtccgccaggctccagggaagggactggagtgggtctcaggtattagtggtggcggtcgtgacacatacttcgcagactccgtgaagggccggttcaccatctccagagacaattccaagaatacgttgtatctacagatgaacagcctgaaaggcgaggacacggccgtatattactgtgtgaagtggggaaatatttactttgactactggggccagggaaccctggtcaccgtctcatca

SEQ ID NO: 188 - protein, 117 aa, synthetic construct [sequence omitted]

SEQ ID NO: 189 - DNA, 24 bp, synthetic construct
  misc_feature 1..24: note = Synthetic
  source 1..24: mol_type = other DNA
ggattcacctttagtaattttggc

SEQ ID NO: 190 - protein, 8 aa, synthetic construct
  REGION 1..8: note = Synthetic
GFTFSNFG

SEQ ID NO: 191 - DNA, 24 bp, synthetic construct
  misc_feature 1..24: note = Synthetic
  source 1..24: mol_type = other DNA
attagtggtggcggtcgtgacaca

SEQ ID NO: 192 - protein, 8 aa, synthetic construct
  REGION 1..8: note = Synthetic
ISGGGRDT

SEQ ID NO: 193 - DNA, 30 bp, synthetic construct
  misc_feature 1..30: note = Synthetic
  source 1..30: mol_type = other DNA
gtgaagtggggaaatatttactttgactac

SEQ ID NO: 194 - protein, 10 aa, synthetic construct
  REGION 1..10: note = Synthetic
VKWGNIYFDY

SEQ ID NO: 195 - DNA, 321 bp, synthetic construct
  misc_feature 1..321: note = Synthetic
  source 1..321: mol_type = other DNA
gacatccagatgacccagtctccatcctccctgtctgcatctgtcggagacagcatcaccatcacttgccgggcgagtctgtccattaacacctttttaaattggtatcagcagaaaccagggaaagcccctaacctcctgatctatgctgcgtccagtttacatggtggggtcccatcaaggttcagtggcagcggctctgggacagatttcactctcaccatcagaactcttcaacctgaagattttgcaacttactactgtcaacagagttccaataccccattcactttcggccctgggaccgtagtggatttcaga

SEQ ID NO: 196 - protein, 107 aa, synthetic construct [sequence omitted]

SEQ ID NO: 197 - DNA, 18 bp, synthetic construct
  misc_feature 1..18: note = Synthetic
  source 1..18: mol_type = other DNA
ctgtccattaacaccttt

SEQ ID NO: 198 - protein, 6 aa, synthetic construct
  REGION 1..6: note = Synthetic
LSINTF

SEQ ID NO: 199-200 - skipped (2 entries)

SEQ ID NO: 201 - DNA, 27 bp, synthetic construct
  misc_feature 1..27: note = Synthetic
  source 1..27: mol_type = other DNA
caacagagttccaataccccattcact

SEQ ID NO: 202 - protein, 9 aa, synthetic construct
  REGION 1..9: note = Synthetic
QQSSNTPFT

SEQ ID NO: 203 - DNA, 363 bp, synthetic construct
  misc_feature 1..363: note = Synthetic
  source 1..363: mol_type = other DNA
gaggtgcagctggtggagtctgggggaggagtggtacggccgggggggtccctgagactctcctgtgcagcctctggattcacttttgatgactatggcatgagttgggtccgccaagttccagggaaggggctggagtgggtctcaggtattagttggaatgatggtaagacagtttatgcagagtctgtgaagggccgattcatcatctccagagacaacgccaagaactccctgtatctggaaatgaatagtctgagagccgaggacacggccttatattactgtgcgagagattggcagtacttgatagagcggtactttgactactggggccagggaaccctggtcaccgtctcctca

SEQ ID NO: 204 - protein, 121 aa, synthetic construct
  REGION 1..121: note = Synthetic
EVQLVESGGGVVRPGGSLRLSCAASGFTFDDYGMSWVRQVPGKGLEWVSGISWNDGKTVYAESVKGRFIISRDNAKNSLYLEMNSLRAEDTALYYCARDWQYLIERYFDYWGQGTLVTVSS

SEQ ID NO: 205 - DNA, 24 bp, synthetic construct
  misc_feature 1..24: note = Synthetic
  source 1..24: mol_type = other DNA
ggattcacttttgatgactatggc

SEQ ID NO: 206 - protein, 8 aa, synthetic construct
  REGION 1..8: note = Synthetic
GFTFDDYG

SEQ ID NO: 207 - DNA, 24 bp, synthetic construct
  misc_feature 1..24: note = Synthetic
  source 1..24: mol_type = other DNA
attagttggaatgatggtaagaca

SEQ ID NO: 208 - protein, 8 aa, synthetic construct
  REGION 1..8: note = Synthetic
ISWNDGKT

SEQ ID NO: 209 - DNA, 42 bp, synthetic construct
  misc_feature 1..42: note = Synthetic
  source 1..42: mol_type = other DNA
gcgagagattggcagtacttgatagagcggtactttgactac

SEQ ID NO: 210 - protein, 14 aa, synthetic construct
  REGION 1..14: note = Synthetic
ARDWQYLIERYFDY

SEQ ID NO: 211 - DNA, 324 bp, synthetic construct
  misc_feature 1..324: note = Synthetic
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 212 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 213 - DNA, 21 bp, synthetic construct
  misc_feature 1..21: note = Synthetic
  source 1..21: mol_type = other DNA
cagtcagtctctagctcttat

SEQ ID NO: 214 - protein, 7 aa, synthetic construct
  REGION 1..7: note = Synthetic
QSVSSSY

SEQ ID NO: 215-216 - skipped (2 entries)

SEQ ID NO: 217 - DNA, 27 bp, synthetic construct
  misc_feature 1..27: note = Synthetic
  source 1..27: mol_type = other DNA
caacagtacggaagcagcccgtggacg

SEQ ID NO: 218 - protein, 9 aa, synthetic construct
  REGION 1..9: note = Synthetic
QQYGSSPWT

SEQ ID NO: 219 - DNA, 363 bp, synthetic construct
  misc_feature 1..363: note = Synthetic
  source 1..363: mol_type = other DNA
gaggtgcagctggtggagtctgggggaggtgtggtacggcctggggggtccctgagactctcctgtacagcctctggattcacctttgatgattatggcatgagctgggtccgccaagctccagggaaggggctggagtggatctctggtattggttggactggtggtcggtcaagttatgcagactctgtgaggggccgattcaccatctccagagacaacgccaagaattccctgtatctgcaaatgaacagtctgggagccgaggacacggccttgtattattgtgcaagagatcggcagtggctggtgcagtggtactttgactactggggccagggaaccctggtcaccgtctcctca

SEQ ID NO: 220 - protein, 121 aa, synthetic construct
  REGION 1..121: note = Synthetic
EVQLVESGGGVVRPGGSLRLSCTASGFTFDDYGMSWVRQAPGKGLEWISGIGWTGGRSSYADSVRGRFTISRDNAKNSLYLQMNSLGAEDTALYYCARDRQWLVQWYFDYWGQGTLVTVSS

SEQ ID NO: 221 - DNA, 24 bp, synthetic construct
  misc_feature 1..24: note = Synthetic
  source 1..24: mol_type = other DNA
ggattcacctttgatgattatggc

SEQ ID NO: 222 - protein, 8 aa, synthetic construct
  REGION 1..8: note = Synthetic
GFTFDDYG

SEQ ID NO: 223 - DNA, 24 bp, synthetic construct
  misc_feature 1..24: note = Synthetic
  source 1..24: mol_type = other DNA
attggttggactggtggtcggtca

SEQ ID NO: 224 - protein, 8 aa, synthetic construct
  REGION 1..8: note = Synthetic
IGWTGGRS

SEQ ID NO: 225 - DNA, 42 bp, synthetic construct
  misc_feature 1..42: note = Synthetic
  source 1..42: mol_type = other DNA
gcaagagatcggcagtggctggtgcagtggtactttgactac

SEQ ID NO: 226 - protein, 14 aa, synthetic construct
  REGION 1..14: note = Synthetic
ARDRQWLVQWYFDY

SEQ ID NO: 227 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 228 - protein, 108 aa, synthetic construct; identical to SEQ ID NO: 116
  REGION 1..108: note = Synthetic

SEQ ID NO: 229 - DNA, 18 bp, synthetic construct
  misc_feature 1..18: note = Synthetic
  source 1..18: mol_type = other DNA
cagagcattagcagctat

SEQ ID NO: 230 - protein, 6 aa, synthetic construct
  REGION 1..6: note = Synthetic
QSISSY

SEQ ID NO: 231-232 - skipped (2 entries)

SEQ ID NO: 233 - DNA, 30 bp, synthetic construct; identical to SEQ ID NO: 41
  misc_feature 1..30: note = Synthetic
  source 1..30: mol_type = other DNA

SEQ ID NO: 234 - protein, 10 aa, synthetic construct
  REGION 1..10: note = Synthetic
QQSYSTPPIT

SEQ ID NO: 235 - DNA, 363 bp, synthetic construct
  misc_feature 1..363: note = Synthetic
  source 1..363: mol_type = other DNA
gaggtgcagctggtggagtctgggggaagagtggtacggccgggggggtccctgagactctcctgtgcagcctctggattcacttttgatgactatggcatgagttgggtccgccaacttccagggaagggcctggagtgggtcgcaggtattagttggaatgatggtaagacagtttatgcagagtctgtgaagggccgattcatcatctccagagacaacgccaagaactccctgcatctggagatgaacagtctgagagccgaggacacggccttatattactgtgcgcgagattggcaatacttaatagatcgttactttgacttctggggtcagggaaccctggtcaccgtctcctca

SEQ ID NO: 236 - protein, 121 aa, synthetic construct
  REGION 1..121: note = Synthetic
EVQLVESGGRVVRPGGSLRLSCAASGFTFDDYGMSWVRQLPGKGLEWVAGISWNDGKTVYAESVKGRFIISRDNAKNSLHLEMNSLRAEDTALYYCARDWQYLIDRYFDFWGQGTLVTVSS

SEQ ID NO: 237 - DNA, 24 bp, synthetic construct
  misc_feature 1..24: note = Synthetic
  source 1..24: mol_type = other DNA
ggattcacttttgatgactatggc

SEQ ID NO: 238 - protein, 8 aa, synthetic construct
  REGION 1..8: note = Synthetic
GFTFDDYG

SEQ ID NO: 239 - DNA, 24 bp, synthetic construct
  misc_feature 1..24: note = Synthetic
  source 1..24: mol_type = other DNA
attagttggaatgatggtaagaca

SEQ ID NO: 240 - protein, 8 aa, synthetic construct
  REGION 1..8: note = Synthetic
ISWNDGKT

SEQ ID NO: 241 - DNA, 42 bp, synthetic construct
  misc_feature 1..42: note = Synthetic
  source 1..42: mol_type = other DNA
gcgcgagattggcaatacttaatagatcgttactttgacttc

SEQ ID NO: 242 - protein, 14 aa, synthetic construct
  REGION 1..14: note = Synthetic
ARDWQYLIDRYFDF

SEQ ID NO: 243 - DNA, 363 bp, synthetic construct
  misc_feature 1..363: note = Synthetic
  source 1..363: mol_type = other DNA
gaagtgcagctggtggagtctgggggaggcttggtgcagcctggcgggtccctgagactctcctgtgcagcctctggattcacctttgatgattatgccatgcactgggtccggcaagctccagggaagggcctggagtgggtctcaggtattggttggagtagtggtagcataggctatgcggactctgtgaagggccgattcaccatctccagagacaacgccaagaactccttgtatctgcaaatggacagtctgagacctgaggactcagccttatattactgtgcaaaagcctatacatttatgattaccctctactttgactactggggccagggaaccctggtcaccgtctcctca

SEQ ID NO: 244 - protein, 121 aa, synthetic construct
  REGION 1..121: note = Synthetic
EVQLVESGGGLVQPGGSLRLSCAASGFTFDDYAMHWVRQAPGKGLEWVSGIGWSSGSIGYADSVKGRFTISRDNAKNSLYLQMDSLRPEDSALYYCAKAYTFMITLYFDYWGQGTLVTVSS

SEQ ID NO: 245 - DNA, 24 bp, synthetic construct
  misc_feature 1..24: note = Synthetic
  source 1..24: mol_type = other DNA
ggattcacctttgatgattatgcc

SEQ ID NO: 246 - protein, 8 aa, synthetic construct
  REGION 1..8: note = Synthetic
GFTFDDYA

SEQ ID NO: 247 - DNA, 24 bp, synthetic construct
  misc_feature 1..24: note = Synthetic
  source 1..24: mol_type = other DNA
attggttggagtagtggtagcata

SEQ ID NO: 248 - protein, 8 aa, synthetic construct
  REGION 1..8: note = Synthetic
IGWSSGSI

SEQ ID NO: 249 - DNA, 42 bp, synthetic construct
  misc_feature 1..42: note = Synthetic
  source 1..42: mol_type = other DNA
gcaaaagcctatacatttatgattaccctctactttgactac

SEQ ID NO: 250 - protein, 14 aa, synthetic construct
  REGION 1..14: note = Synthetic
AKAYTFMITLYFDY

SEQ ID NO: 251 - DNA, 363 bp, synthetic construct
  misc_feature 1..363: note = Synthetic
  source 1..363: mol_type = other DNA
gaagtgcagctggtggagtctgggggaggcttggtacagcctggcaggtccctgagactctcctgtgcagcctctggattcacctttgatgattatgacatgcactgggtccggcaagctccagggaagggcctggagtgggtgtcagggagtggttggaataggggtagtttaggctatgcggattctgtgaagggccgattcaccatctccagagacaacgccaagaagtccctgtatctgcaaatgaacagtgtgagagttgaggacacggccttgtattactgtgcaaaaggctttgtagtggtatcagctgcttactttgactactggggccagggaaccctggtcaccgtctcctca

SEQ ID NO: 252 - protein, 121 aa, synthetic construct
  REGION 1..121: note = Synthetic
EVQLVESGGGLVQPGRSLRLSCAASGFTFDDYDMHWVRQAPGKGLEWVSGSGWNRGSLGYADSVKGRFTISRDNAKKSLYLQMNSVRVEDTALYYCAKGFVVVSAAYFDYWGQGTLVTVSS

SEQ ID NO: 253 - DNA, 24 bp, synthetic construct
  misc_feature 1..24: note = Synthetic
  source 1..24: mol_type = other DNA
ggattcacctttgatgattatgac

SEQ ID NO: 254 - protein, 8 aa, synthetic construct
  REGION 1..8: note = Synthetic
GFTFDDYD

SEQ ID NO: 255 - DNA, 24 bp, synthetic construct
  misc_feature 1..24: note = Synthetic
  source 1..24: mol_type = other DNA
agtggttggaataggggtagttta

SEQ ID NO: 256 - protein, 8 aa, synthetic construct
  REGION 1..8: note = Synthetic
SGWNRGSL

SEQ ID NO: 257 - DNA, 42 bp, synthetic construct
  misc_feature 1..42: note = Synthetic
  source 1..42: mol_type = other DNA
gcaaaaggctttgtagtggtatcagctgcttactttgactac

SEQ ID NO: 258 - protein, 14 aa, synthetic construct
  REGION 1..14: note = Synthetic
AKGFVVVSAAYFDY

SEQ ID NO: 259 - DNA, 363 bp, synthetic construct
  misc_feature 1..363: note = Synthetic
  source 1..363: mol_type = other DNA
caggtgcagctggtgcagtctggggctgaggtgaagaggcctgggtcctcggtgaaggtctcctgcaaggtatctggagtcaccttcaggaattttgctatcatctgggtgcgacaggcccctggacaagggcttgagtggatgggaggaatcatccctttctttagtgcagcaaattacgcacagagcttccagggcagagtcacgattaccccggacgaatccacgagcacagccttcatggagctggccagtctgagatctgaggacacggccgtttattattgtgcgagagagggggaacgtggacacacctatgggtttgactactggggccagggaaccctggtcaccgtctcctca

SEQ ID NO: 260 - protein, 121 aa, synthetic construct
  REGION 1..121: note = Synthetic
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSS

SEQ ID NO: 261 - DNA, 24 bp, synthetic construct
  misc_feature 1..24: note = Synthetic
  source 1..24: mol_type = other DNA
ggagtcaccttcaggaattttgct

SEQ ID NO: 262 - protein, 8 aa, synthetic construct
  REGION 1..8: note = Synthetic
GVTFRNFA

SEQ ID NO: 263 - DNA, 24 bp, synthetic construct
  misc_feature 1..24: note = Synthetic
  source 1..24: mol_type = other DNA
atcatccctttctttagtgcagca

SEQ ID NO: 264 - protein, 8 aa, synthetic construct
  REGION 1..8: note = Synthetic
IIPFFSAA

SEQ ID NO: 265 - DNA, 42 bp, synthetic construct
  misc_feature 1..42: note = Synthetic
  source 1..42: mol_type = other DNA
gcgagagagggggaacgtggacacacctatgggtttgactac

SEQ ID NO: 266 - protein, 14 aa, synthetic construct
  REGION 1..14: note = Synthetic
AREGERGHTYGFDY

SEQ ID NO: 267 - DNA, 363 bp, synthetic construct
  misc_feature 1..363: note = Synthetic
  source 1..363: mol_type = other DNA
gaagtgcagctggtggagtctgggggaggcttggtacagtctggcaggtccctgagactctcctgtgcagcctctggattcacctttgatgattatgccatgcactgggtccgacaacctccagggaagggcctggaatgggtctcaggtattaactggaatagaggtaggacaggctatgcggactctgtgaagggccgattcaccatctccagagacaacgccaagaactccctgtatctgcaaatgaacgatctgagagttgaggatacggccttgtattactgtgcaaaagccgaacagtggctggacgagggatactttgactactggggccagggaaccctggtcaccgtctcctca

SEQ ID NO: 268 - protein, 121 aa, synthetic construct
  REGION 1..121: note = Synthetic
EVQLVESGGGLVQSGRSLRLSCAASGFTFDDYAMHWVRQPPGKGLEWVSGINWNRGRTGYADSVKGRFTISRDNAKNSLYLQMNDLRVEDTALYYCAKAEQWLDEGYFDYWGQGTLVTVSS

SEQ ID NO: 269 - DNA, 24 bp, synthetic construct
  misc_feature 1..24: note = Synthetic
  source 1..24: mol_type = other DNA
ggattcacctttgatgattatgcc

SEQ ID NO: 270 - protein, 8 aa, synthetic construct
  REGION 1..8: note = Synthetic
GFTFDDYA

SEQ ID NO: 271 - DNA, 24 bp, synthetic construct
  misc_feature 1..24: note = Synthetic
  source 1..24: mol_type = other DNA
attaactggaatagaggtaggaca

SEQ ID NO: 272 - protein, 8 aa, synthetic construct
  REGION 1..8: note = Synthetic
INWNRGRT

SEQ ID NO: 273 - DNA, 42 bp, synthetic construct
  misc_feature 1..42: note = Synthetic
  source 1..42: mol_type = other DNA
gcaaaagccgaacagtggctggacgagggatactttgactac

SEQ ID NO: 274 - protein, 14 aa, synthetic construct
  REGION 1..14: note = Synthetic
AKAEQWLDEGYFDY

SEQ ID NO: 275 - DNA, 363 bp, synthetic construct
  misc_feature 1..363: note = Synthetic
  source 1..363: mol_type = other DNA
gaggtgcagctggtggagtctgggggaggcttggtgcagcggggggggtccctgagactctcctgtgcagcctctggattcagctttagcagctatgccatgaactgggtccgccaggctccagggaaggggctggagtgggtctcaactattagtgatagtggtggtagtacatactacgcagactccgtgaagggccggttcaccatttccagagacaattccaagaacacgctgtctctgcaaatgaacagcctgagagccgaggacacggccgtatattactgtgcgaaagatcagggtgggagttacccctactactttcactactggggccagggaaccctggtcaccgtctcctca

SEQ ID NO: 276 - protein, 121 aa, synthetic construct
  REGION 1..121: note = Synthetic
EVQLVESGGGLVQRGGSLRLSCAASGFSFSSYAMNWVRQAPGKGLEWVSTISDSGGSTYYADSVKGRFTISRDNSKNTLSLQMNSLRAEDTAVYYCAKDQGGSYPYYFHYWGQGTLVTVSS

SEQ ID NO: 277 - DNA, 24 bp, synthetic construct
  misc_feature 1..24: note = Synthetic
  source 1..24: mol_type = other DNA
ggattcagctttagcagctatgcc

SEQ ID NO: 278 - protein, 8 aa, synthetic construct
  REGION 1..8: note = Synthetic
GFSFSSYA

SEQ ID NO: 279 - DNA, 24 bp, synthetic construct
  misc_feature 1..24: note = Synthetic
  source 1..24: mol_type = other DNA
attagtgatagtggtggtagtaca

SEQ ID NO: 280 - protein, 8 aa, synthetic construct
  REGION 1..8: note = Synthetic
ISDSGGST

SEQ ID NO: 281 - DNA, 42 bp, synthetic construct
  misc_feature 1..42: note = Synthetic
  source 1..42: mol_type = other DNA
gcgaaagatcagggtgggagttacccctactactttcactac

SEQ ID NO: 282 - protein, 14 aa, synthetic construct
  REGION 1..14: note = Synthetic
AKDQGGSYPYYFHY

SEQ ID NO: 283 - DNA, 363 bp, synthetic construct
  misc_feature 1..363: note = Synthetic
  source 1..363: mol_type = other DNA
gaagtgcagctggtggagtctgggggaggcttggtacagcctggcaggtccctgagactctcctgtgcagcctctggattcacctttgaggattatgccatgcactgggtccggcaagctccagggaagggcctggagtgggtctcaggtattggttggagtaatgtaaagataggctatgcggactctgtgaagggccgattcaccatctccagagacaatgtcaggaactccctatatctgcaaatgaacagtctgagaactgaggacacggccttctattactgtgtaaaagcctatacatctatgcttaccctctactttgactattggggccagggaaccctggtcaccgtctcctca

SEQ ID NO: 284 - protein, 121 aa, synthetic construct
  REGION 1..121: note = Synthetic
EVQLVESGGGLVQPGRSLRLSCAASGFTFEDYAMHWVRQAPGKGLEWVSGIGWSNVKIGYADSVKGRFTISRDNVRNSLYLQMNSLRTEDTAFYYCVKAYTSMLTLYFDYWGQGTLVTVSS

SEQ ID NO: 285 - DNA, 24 bp, synthetic construct
  misc_feature 1..24: note = Synthetic
  source 1..24: mol_type = other DNA
ggattcacctttgaggattatgcc

SEQ ID NO: 286 - protein, 8 aa, synthetic construct
  REGION 1..8: note = Synthetic
GFTFEDYA

SEQ ID NO: 287 - DNA, 24 bp, synthetic construct
  misc_feature 1..24: note = Synthetic
  source 1..24: mol_type = other DNA
attggttggagtaatgtaaagata

SEQ ID NO: 288 - protein, 8 aa, synthetic construct
  REGION 1..8: note = Synthetic
IGWSNVKI

SEQ ID NO: 289 - DNA, 42 bp, synthetic construct
  misc_feature 1..42: note = Synthetic
  source 1..42: mol_type = other DNA
gtaaaagcctatacatctatgcttaccctctactttgactat

SEQ ID NO: 290 - protein, 14 aa, synthetic construct
  REGION 1..14: note = Synthetic
VKAYTSMLTLYFDY

SEQ ID NO: 291 - DNA, 354 bp, synthetic construct
  misc_feature 1..354: note = Synthetic
  source 1..354: mol_type = other DNA
caggtgcagctggtgcagtctggggctgaggtgaagaggcctggggcctcagtgaaggtttcctgcaaggcatctggatacaccttcaccagcttctatatgtactgggtgcgacaggcccctggacaagggcttgagtggatgggaataatcaaccctagtgatggtagcacaagcaacgcacagaagttccagggcagagtcaccatgaccagggacacgtccacgagtacagtctacatggagctgagcagcctgagatctgaggacacggccgtgtattactgtgcgagacgggtggctggggatatttttgatatctggggccaagggacaatggtcaccgtctcttca

SEQ ID NO: 292 - protein, 118 aa, synthetic construct
  REGION 1..118: note = Synthetic
QVQLVQSGAEVKRPGASVKVSCKASGYTFTSFYMYWVRQAPGQGLEWMGIINPSDGSTSNAQKFQGRVTMTRDTSTSTVYMELSSLRSEDTAVYYCARRVAGDIFDIWGQGTMVTVSS

SEQ ID NO: 293 - DNA, 24 bp, synthetic construct
  misc_feature 1..24: note = Synthetic
  source 1..24: mol_type = other DNA
ggatacaccttcaccagcttctat

SEQ ID NO: 294 - protein, 8 aa, synthetic construct
  REGION 1..8: note = Synthetic
GYTFTSFY

SEQ ID NO: 295 - DNA, 24 bp, synthetic construct
  misc_feature 1..24: note = Synthetic
  source 1..24: mol_type = other DNA
atcaaccctagtgatggtagcaca

SEQ ID NO: 296 - protein, 8 aa, synthetic construct
  REGION 1..8: note = Synthetic
INPSDGST

SEQ ID NO: 297 - DNA, 33 bp, synthetic construct
  misc_feature 1..33: note = Synthetic
  source 1..33: mol_type = other DNA
gcgagacgggtggctggggatatttttgatatc

SEQ ID NO: 298 - protein, 11 aa, synthetic construct
  REGION 1..11: note = Synthetic
ARRVAGDIFDI

SEQ ID NO: 299 - DNA, 357 bp, synthetic construct
  misc_feature 1..357: note = Synthetic
  source 1..357: mol_type = other DNA
caggtgcagctgcaggagtcgggcccaggactggtgaagccttcggagaccctgtccctcacctgcactgtctctggtggctccatcagtagttaccactggaactggatccggcagagtccagggaagggactggaatggattggatatatctattatattgggagcaccgactataatccctccctcgagagtcgagtcaccatatcagtagacacgtccaagaaccagttctccctgaagctgagttctgtgaccgctgcggacacggccgtgtattactgtgcgagagtccccgtgggagctacaggggcttctgatgtctggggccaagggacaatggtcaccgtctcttca

SEQ ID NO: 300 - protein, 119 aa, synthetic construct
  REGION 1..119: note = Synthetic
QVQLQESGPGLVKPSETLSLTCTVSGGSISSYHWNWIRQSPGKGLEWIGYIYYIGSTDYNPSLESRVTISVDTSKNQFSLKLSSVTAADTAVYYCARVPVGATGASDVWGQGTMVTVSS

SEQ ID NO: 301 - DNA, 24 bp, synthetic construct
  misc_feature 1..24: note = Synthetic
  source 1..24: mol_type = other DNA
ggtggctccatcagtagttaccac

SEQ ID NO: 302 - protein, 8 aa, synthetic construct
  REGION 1..8: note = Synthetic
GGSISSYH

SEQ ID NO: 303 - DNA, 21 bp, synthetic construct
  misc_feature 1..21: note = Synthetic
  source 1..21: mol_type = other DNA
atctattatattgggagcacc

SEQ ID NO: 304 - protein, 7 aa, synthetic construct
  REGION 1..7: note = Synthetic
IYYIGST

SEQ ID NO: 305 - DNA, 39 bp, synthetic construct
  misc_feature 1..39: note = Synthetic
  source 1..39: mol_type = other DNA
gcgagagtccccgtgggagctacaggggcttctgatgtc

SEQ ID NO: 306 - protein, 13 aa, synthetic construct
  REGION 1..13: note = Synthetic
ARVPVGATGASDV

SEQ ID NO: 307 - DNA, 363 bp, synthetic construct
  misc_feature 1..363: note = Synthetic
  source 1..363: mol_type = other DNA
gaggtgcagctggtggagtctgggggaagtgtggttcgacctggggggtccctgagactctcctgtgtagtctctggattcacctttgaggattatggtttgagctgggtccgccaaattccagggaaaggactggagtgggtctctggtattagttggactggtggtaacacaggttatgcagactctgtgaagggccgcttcaccatctccagagacaacgccaagaactccctgtatctgcaaatgaacagtctgagagccgaagacacggccctgtatcactgtacgagagatcgacagtggctgatgcagtggtattttgactattggggccagggaaccctggtcaccgtctcctca

SEQ ID NO: 308 - protein, 121 aa, synthetic construct
  REGION 1..121: note = Synthetic
EVQLVESGGSVVRPGGSLRLSCVVSGFTFEDYGLSWVRQIPGKGLEWVSGISWTGGNTGYADSVKGRFTISRDNAKNSLYLQMNSLRAEDTALYHCTRDRQWLMQWYFDYWGQGTLVTVSS

SEQ ID NO: 309 - DNA, 24 bp, synthetic construct
  misc_feature 1..24: note = Synthetic
  source 1..24: mol_type = other DNA
ggattcacctttgaggattatggt

SEQ ID NO: 310 - protein, 8 aa, synthetic construct
  REGION 1..8: note = Synthetic
GFTFEDYG

SEQ ID NO: 311 - DNA, 24 bp, synthetic construct
  misc_feature 1..24: note = Synthetic
  source 1..24: mol_type = other DNA
attagttggactggtggtaacaca

SEQ ID NO: 312 - protein, 8 aa, synthetic construct
  REGION 1..8: note = Synthetic
ISWTGGNT

SEQ ID NO: 313 - DNA, 42 bp, synthetic construct
  misc_feature 1..42: note = Synthetic
  source 1..42: mol_type = other DNA
acgagagatcgacagtggctgatgcagtggtattttgactat

SEQ ID NO: 314 - protein, 14 aa, synthetic construct
  REGION 1..14: note = Synthetic
TRDRQWLMQWYFDY

SEQ ID NO: 315 - DNA, 357 bp, synthetic construct
  misc_feature 1..357: note = Synthetic
  source 1..357: mol_type = other DNA
caggtgcagctggtggagtctgggggaggcgtggtccagcctgggaggtccctgagactctcctgttcagcctctggattcaccttcagtgcctatgccatgcactgggtccgccaggctccaggcaaggggctggaatgggtggcagctatctcatatggtggaagtgataaatactatgcagactccgtgaagggccgattcaccatctccagagacaattccaagaacacgctatatctgcaaatgaacagcctgagaactgacgacacggctgtgtattactgtgcgaaatccgctcactggaacttcttctttgactactggggccagggaaccctggtcactgtctcctca

SEQ ID NO: 316 - protein, 119 aa, synthetic construct
  REGION 1..119: note = Synthetic
QVQLVESGGGVVQPGRSLRLSCSASGFTFSAYAMHWVRQAPGKGLEWVAAISYGGSDKYYADSVKGRFTISRDNSKNTLYLQMNSLRTDDTAVYYCAKSAHWNFFFDYWGQGTLVTVSS

SEQ ID NO: 317 - DNA, 24 bp, synthetic construct
  misc_feature 1..24: note = Synthetic
  source 1..24: mol_type = other DNA
ggattcaccttcagtgcctatgcc

SEQ ID NO: 318 - protein, 8 aa, synthetic construct
  REGION 1..8: note = Synthetic
GFTFSAYA

SEQ ID NO: 319 - DNA, 24 bp, synthetic construct
  misc_feature 1..24: note = Synthetic
  source 1..24: mol_type = other DNA
atctcatatggtggaagtgataaa

SEQ ID NO: 320 - protein, 8 aa, synthetic construct
  REGION 1..8: note = Synthetic
ISYGGSDK

SEQ ID NO: 321 - DNA, 36 bp, synthetic construct
  misc_feature 1..36: note = Synthetic
  source 1..36: mol_type = other DNA
gcgaaatccgctcactggaacttcttctttgactac

SEQ ID NO: 322 - protein, 12 aa, synthetic construct
  REGION 1..12: note = Synthetic
AKSAHWNFFFDY

SEQ ID NO: 323 - DNA, 363 bp, synthetic construct
  misc_feature 1..363: note = Synthetic
  source 1..363: mol_type = other DNA
gaagtgcagctggtggagtctgggggaggcttggtacagcctggcaggtccctgagactctcctgtgtagcctctggattcgcccttcatgattatgccatgcactgggtccggcaagttccagggaagggcctggagtgggtctcaagtattagttggaatagtggtgtcataggctatgcggactctctgaagggccgcttcaccatctccagagacaacgccaagaactccctgtatctgcaaatgaacagtctgagagcagaggacacggccttatactactgtgcaaaaggtagtgggagctactacgtcagttggttcgacccctggggccagggaaccctggtcaccgtctcctca

SEQ ID NO: 324 - protein, 121 aa, synthetic construct
  REGION 1..121: note = Synthetic
EVQLVESGGGLVQPGRSLRLSCVASGFALHDYAMHWVRQVPGKGLEWVSSISWNSGVIGYADSLKGRFTISRDNAKNSLYLQMNSLRAEDTALYYCAKGSGSYYVSWFDPWGQGTLVTVSS

SEQ ID NO: 325 - DNA, 24 bp, synthetic construct
  misc_feature 1..24: note = Synthetic
  source 1..24: mol_type = other DNA
ggattcgcccttcatgattatgcc

SEQ ID NO: 326 - protein, 8 aa, synthetic construct
  REGION 1..8: note = Synthetic
GFALHDYA

SEQ ID NO: 327 - DNA, 24 bp, synthetic construct
  misc_feature 1..24: note = Synthetic
  source 1..24: mol_type = other DNA
attagttggaatagtggtgtcata

SEQ ID NO: 328 - protein, 8 aa, synthetic construct
  REGION 1..8: note = Synthetic
ISWNSGVI

SEQ ID NO: 329 - DNA, 42 bp, synthetic construct
  misc_feature 1..42: note = Synthetic
  source 1..42: mol_type = other DNA
gcaaaaggtagtgggagctactacgtcagttggttcgacccc

SEQ ID NO: 330 - protein, 14 aa, synthetic construct
  REGION 1..14: note = Synthetic
AKGSGSYYVSWFDP

SEQ ID NO: 331 - DNA, 369 bp, synthetic construct
  misc_feature 1..369: note = Synthetic
  source 1..369: mol_type = other DNA
cagctgcagctgcaggagtcgggcccaggactggttcagccttcggagaccctgtccctcacctgcactgtctctggtgactccatcagtagtactgcttaccactgggactggatccgccagccccccgggaagggactggagtggattgggaccatcacttataatgggaacacctacttcaacccgtccctcaagagtcgagtcaccatatccgttgacacgtccaagaaccagttctccctgaagctactctctatgaccgccgcagaaacggctgttttttactgtgcgcgacatctaggatataacagtgacttctttccctttgacttctggggccagggaaccctggtcactgtctcctca

SEQ ID NO: 332 - protein, 123 aa, synthetic construct
  REGION 1..123: note = Synthetic
QLQLQESGPGLVQPSETLSLTCTVSGDSISSTAYHWDWIRQPPGKGLEWIGTITYNGNTYFNPSLKSRVTISVDTSKNQFSLKLLSMTAAETAVFYCARHLGYNSDFFPFDFWGQGTLVTVSS

SEQ ID NO: 333 - DNA, 30 bp, synthetic construct
  misc_feature 1..30: note = Synthetic
  source 1..30: mol_type = other DNA
ggtgactccatcagtagtactgcttaccac

SEQ ID NO: 334 - protein, 10 aa, synthetic construct
  REGION 1..10: note = Synthetic
GDSISSTAYH

SEQ ID NO: 335 - DNA, 21 bp, synthetic construct
  misc_feature 1..21: note = Synthetic
  source 1..21: mol_type = other DNA
atcacttataatgggaacacc

SEQ ID NO: 336 - protein, 7 aa, synthetic construct
  REGION 1..7: note = Synthetic
ITYNGNT

SEQ ID NO: 337 - DNA, 45 bp, synthetic construct
  misc_feature 1..45: note = Synthetic
  source 1..45: mol_type = other DNA
gcgcgacatctaggatataacagtgacttctttccctttgacttc

SEQ ID NO: 338 - protein, 15 aa, synthetic construct
  REGION 1..15: note = Synthetic
ARHLGYNSDFFPFDF

SEQ ID NO: 339 - DNA, 357 bp, synthetic construct
  misc_feature 1..357: note = Synthetic
  source 1..357: mol_type = other DNA
gaggtgcagctggtggagtctgggggaggcctggtacggccgggggggtccctgagactctcctgtgcagcctctggattcacctttagcacctatgccatggcctgggtccgccagactccagggaaggggctggagggggtctcagctattgggggtagtggtgatagtacctattatgtcgactccgtgaagggccggttcaccatctccagggacaactccaagagcacgctttttctgcaaatgaatagcctgagagccgaggacacggccgtttattactgtgtgaaagtccggaattacgacggttcttttgatatctggggccaagggacaatggtcaccgtctcttca

SEQ ID NO: 340 - protein, 119 aa, synthetic construct
  REGION 1..119: note = Synthetic
EVQLVESGGGLVRPGGSLRLSCAASGFTFSTYAMAWVRQTPGKGLEGVSAIGGSGDSTYYVDSVKGRFTISRDNSKSTLFLQMNSLRAEDTAVYYCVKVRNYDGSFDIWGQGTMVTVSS

SEQ ID NO: 341 - DNA, 24 bp, synthetic construct
  misc_feature 1..24: note = Synthetic
  source 1..24: mol_type = other DNA
ggattcacctttagcacctatgcc

SEQ ID NO: 342 - protein, 8 aa, synthetic construct
  REGION 1..8: note = Synthetic
GFTFSTYA

SEQ ID NO: 343 - DNA, 24 bp, synthetic construct
  misc_feature 1..24: note = Synthetic
  source 1..24: mol_type = other DNA
attgggggtagtggtgatagtacc

SEQ ID NO: 344 - protein, 8 aa, synthetic construct
  REGION 1..8: note = Synthetic
IGGSGDST

SEQ ID NO: 345 - DNA, 36 bp, synthetic construct
  misc_feature 1..36: note = Synthetic
  source 1..36: mol_type = other DNA
gtgaaagtccggaattacgacggttcttttgatatc

SEQ ID NO: 346 - protein, 12 aa, synthetic construct
  REGION 1..12: note = Synthetic
VKVRNYDGSFDI

SEQ ID NO: 347 - protein, 174 aa, synthetic construct
  REGION 1..174: note = hPD-1-MMH
  REGION 1..174: note = 1-146: aa 25-170 of NP_005009.2 with C93S
  REGION 1..174: note = 147-174: myc-myc-hexahistidine
LDSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQFQTLVEQKLISEEDLGGEQKLISEEDLHHHHHH

SEQ ID NO: 348 - protein, 174 aa, synthetic construct
  REGION 1..174: note = MfPD-1-MMH
  REGION 1..174: note = 1-146: M.fascicularis PD-1 with C93S
  REGION 1..174: note = 147-174: myc-myc-hexahistidine
LESPDRPWNPPTFSPALLLVTEGDNATFTCSFSNASESFVLNWYRMSPSNQTDKLAAFPEDRSQPGRDSRFRVTQLPNGRDFHMSVVRARRNDSGTYLCGAISLAPKAQIKESLRAELRVTERRAEVPTAHPSPSPRPAGQFQALVEQKLISEEDLGGEQKLISEEDLHHHHHH

SEQ ID NO: 349 - protein, 379 aa, synthetic construct
  REGION 1..379: note = hPD-1-mFc
  REGION 1..379: note = 1-146: aa 25-170 of NP-005009.2 with C93S
  REGION 1..379: note = 147-379: mFc: aa 98-330 of P01863
LDSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQFQTLVEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 350 - protein, 373 aa, synthetic construct
  REGION 1..373: note = hPD-1-hFc
  REGION 1..373: note = 1-146: aa 25-170 of NP_005009.2 with C93S
  REGION 1..373: note = 147-373: hFc: aa 104-330 of P01857
LDSPDRPWNPPTFSPALLVVTEGDNATFTCSFSNTSESFVLNWYRMSPSNQTDKLAAFPEDRSQPGQDSRFRVTQLPNGRDFHMSVVRARRNDSGTYLCGAISLAPKAQIKESLRAELRVTERRAEVPTAHPSPSPRPAGQFQTLVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 351 - protein, 448 aa, synthetic construct
  REGION 1..448: note = hPD-L1-hFc
  REGION 1..448: note = 1-221: aa 19-239 of NP_054862.1
  REGION 1..448: note = 222-448: hFc: aa 104-330 of P01857
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 352 - protein, 454 aa, synthetic construct
  REGION 1..454: note = hPD-L1-mFc
  REGION 1..454: note = 1-221: aa 19-239 of NP_054862.1
  REGION 1..454: note = 222-454: mFc: aa 98-330 of P01863
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 353 - protein, 288 aa, synthetic construct
  REGION 1..288: note = hPD-1 NP_005009.2
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 354 - protein, 290 aa, synthetic construct
  REGION 1..290: note = hPD-L1 NP_054862.1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 355 - protein, 379 aa, synthetic construct
  REGION 1..379: note = aa1-146: M.fascicularis PD-1 (with C93S change) aa147-379: mFc tag (aa 98-330 of P01863)
LESPDRPWNPPTFSPALLLVTEGDNATFTCSFSNASESFVLNWYRMSPSNQTDKLAAFPEDRSQPGRDSRFRVTQLPNGRDFHMSVVRARRNDSGTYLCGAISLAPKAQIKESLRAELRVTERRAEVPTAHPSPSPRPAGQFQALVEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 356 - protein, 444 aa, synthetic construct
  REGION 1..444: note = H4H7798N aa1-117: HCVR aa118-444: HC constant
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 357 - protein, 214 aa, synthetic construct
  REGION 1..214: note = H4H7798N aa1-107: LCVR aa108-214: LC constant
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 358 - protein, 449 aa, synthetic construct
  REGION 1..449: note = H4H7795N2 aa1-122: HCVR aa123-449: HC constant
EVQVVESGGGLVEPGRSLRLSCKASGFTFDDYAMHWVRQTPGKALEWVSGISWSGNNIGYADSVKGRFTISKDDAKNSLYLQMNSLRPEDTALYYCTKDISITGTLDAFDVWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 359 - protein, 214 aa, synthetic construct
  REGION 1..214: note = H4H7795N2 aa1-107: LCVR aa108-214: LC constant
DIQMTQSPISVSASVGDRVTITCRASQGISNWLAWYQQKPGIAPKLLIYSASSLQSGVPSRFRGSGSGTDFTLTIGSLQPEDFATYYCQQAHSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 360 - protein, 446 aa, synthetic construct
  REGION 1..446: note = H4H9008P aa1-119: HCVR aa120-446: HC constant
EVQLVESGGGLVRPGGSLRLSCAASGFTFSTYAMAWVRQTPGKGLEGVSAIGGSGDSTYYVDSVKGRFTISRDNSKSTLFLQMNSLRAEDTAVYYCVKVRNYDGSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 361 - protein, 215 aa, synthetic construct
  REGION 1..215: note = H4H9008P aa1-108: LCVR aa109-215: LC constant
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 362 - protein, 448 aa, synthetic construct
  REGION 1..448: note = H4H9048P2 aa1-121: HCVR aa122-448: HC constant
QVQLVQSGAEVKRPGSSVKVSCKVSGVTFRNFAIIWVRQAPGQGLEWMGGIIPFFSAANYAQSFQGRVTITPDESTSTAFMELASLRSEDTAVYYCAREGERGHTYGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 363 - protein, 215 aa, synthetic construct
  REGION 1..215: note = H4H9048P2 aa1-108: LCVR aa109-215: LC constant
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 364 - protein, 117 aa, synthetic construct [sequence omitted]

SEQ ID NO: 365 - protein, 107 aa, synthetic construct [sequence omitted]

SEQ ID NO: 366 - protein, 8 aa, synthetic construct
  REGION 1..8: note = synthetic
GFTFSNFG

SEQ ID NO: 367 - protein, 8 aa, synthetic construct
  REGION 1..8: note = synthetic
ISGGGRDT

SEQ ID NO: 368 - protein, 10 aa, synthetic construct
  REGION 1..10: note = synthetic
VKWGNIYFDY

SEQ ID NO: 369 - protein, 6 aa, synthetic construct
  REGION 1..6: note = synthetic
LSINTF

SEQ ID NO: 370 - skipped

SEQ ID NO: 371 - protein, 9 aa, synthetic construct
  REGION 1..9: note = synthetic
QQSSNTPFT

SEQ ID NO: 372 - protein, 444 aa, synthetic construct; identical to SEQ ID NO: 356
  REGION 1..444: note = aa 1-117: HCVR aa 118-444: HC constant

SEQ ID NO: 373 - protein, 214 aa, synthetic construct; identical to SEQ ID NO: 357
  REGION 1..214: note = aa 1-107: LCVR aa 108-214: LC constant

SEQ ID NO: 374 - protein, 443 aa, synthetic construct
  REGION 1..443: note = HC (minus C-terminal Lysine)
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG